(19) United States
(12) Patent Application Publication
(10) Pub. No.: US 2006/0183909 A1
(43) Pub. Date: Aug. 17, 2006

(54) Compositions derived from quinoline and quinoxaline, preparation and use thereof

(76) Inventors: Martine Schmitt, Strasbourg (FR); Evelyne Klotz, Mutzig (FR); Jean-Paul Macher, Bergholtz-Zell (FR); Jean-Jacques Bourguignon, Hipsheim (FR); Mustapha Abarghaz, Strasbourg (FR); Patrick Wagner, Brumath (FR); Gael Ronsin, Strassbourg (FR)

Correspondence Address: NIXON & VANDERHYE, PC, 901 NORTH GLEBE ROAD, 11TH FLOOR, ARLINGTON, VA, 22203, US

(21) Appl. No.: 10/499,403

(22) PCT Filed: Jul. 19, 2002
(86) PCT No.: PCT/FR02/02594

§ 371 Date: Mar. 1, 2005

(30) Foreign Application Priority Data
Jul. 20, 2001 (FR) 0109730

(51) Int. Cl. C07D 215/36

(52) U.S. Cl. 546/156; 544/354; 514/249; 514/312

(57) ABSTRACT

The present invention concerns compounds derived from quinoline and quinoxaline, their preparation and their uses, particularly in the field of therapeutics and vaccines or for developing active compounds. The inventive compounds are of general formula (I), 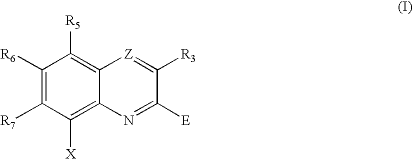 and their pharmaceutically acceptable salts.

The present invention concerns compounds derived from quinoline and quinoxaline, their preparation and their uses, particularly in the field of therapeutics and vaccines or for developing active compounds.

The present invention concerns in particular the use of compounds represented by general formula (I): 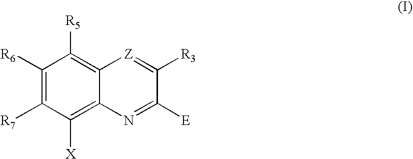
and their pharmaceutically acceptable salts for treating nervous system pathologies, the novel derivatives of formula (I), their methods of preparation and the pharmaceutical compositions containing them.

The compound of formula (I) wherein E is COOH, Z is C—OH, R₅, R₄, R₆ and R₇ are hydrogen atoms and X is OH (xanthurenic acid) is a known metabolite of tryptophan (Bioorg. Med. Chem. Letters, 1999, 17, 2607). This same compound interacts with bovine serum albumin (Chem. Pharm. Bull., 1980, 28, 10, 2960-2966) and is useful in the treatment of dermatoses (Farmaco Ed., 1981, 36, 7, 557-564).

Other compounds represented by formula (I) wherein Z is C—OH, R₃ is hydrogen and E is COOH, COOC₂H₅ or COOCH₃ are mentioned in DE 2130408 for the treatment of asthma, urticaria and autoimmune diseases.

Compounds represented by formula (I) wherein E is COOH, Z is CR₄, R₄ is hydrogen, methoxy or p-chlorophenyl, R₃, R₅ and R₇ are hydrogen atoms, R₆ and X are chlorine atoms or wherein E is CHO, Z is CR₄, R₄ is hydrogen, R₈ is chloro, R₃, R₅, R₆ and R₇ are hydrogen are described in J. Med. Chem., 1972, 15, 490-493 for the treatment of malaria.

Compounds represented by formula (I) wherein E is COOH or COOC₂H₅, Z is CR₄, R₄ is OH, R₇ is chloro, R₈ is methyl, R₃, R₅ and R₆ are hydrogen are described in WO94/17042 as anticonvulsants and inhibitors of the glycine site of the NMDA receptor.

Compounds represented by formula (I) wherein E is CHO, CH₂OH or COOH, Z is CR₄, R₄ is hydrogen, R₃ and R₆ are hydrogen, R₅ and R₇ are chloro and R₈ is benzyloxy are described in U.S. Pat. No. 3,682,927 as antiseptics and antifungals.

The present invention follows from the demonstration that compounds represented by formula (I) have particularly advantageous biological and therapeutic properties. The invention results in particular from the demonstration that compounds modulating the activity of xanthurenic acid may be used for treating disorders of the nervous system, particularly central. The invention more specifically results from the synthesis, development and characterization of compounds modulating the activity of xanthurenic acid, which may be used to modulate neurotransmission, particularly dopaminergic. Such compounds may be used in particular for treating central nervous system pathologies such as mental, neurological or traumatic disorders. The compounds are more particularly aimed at treating anxiety, depression, the depressive component of bipolar disorder, ADH syndrome, fibromyalgia, impairment of memory or social interactions, as sedative or hypnotic, disorders of sleep or concentration, for treating neurodegenerative diseases, such as Parkinson's disease, Alzheimer's disease or ALS, schizophrenia, epilepsy, some drug dependencies, particularly opioid, or pain. The compounds may also be used for treating obesity.

Within the scope of the invention, the term “treatment” denotes preventive, curative, palliative treatment as well as management of patients (alleviating suffering, prolonging survival, improving quality of life, slowing disease progression, etc.). Furthermore, the treatment may be carried out in combination with other agents or treatments, particularly addressing the late events of the disease, or with other active substances.

The invention is therefore based on using compounds represented by general formula (I) such as described hereinabove for preparing a pharmaceutical composition for treating nervous system pathologies.

In general formula (I)

E is a COOH, COOR₁, CH₂OH, CHO, CH₂COOH, CH₂COOR₁ group or a group chosen from among the following:

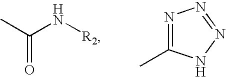

R₁ represents (i) a (C₁-C₁₂) alkyl group or (ii) a (C₆-C₁₈)aryl(C₁-C₁₂)alkyl group;

R₂ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a hydroxyl group;

R₃ is (i) a hydrogen atom, (ii) a halogen atom, (iii) a hydroxyl group, (iv) a (C₁-C₁₂) alkyl group, (v) a (C₆-C₁₈) aryl group, (vi) a (C₆-C₁₈)aryl(C₁-C₁₂)alkyl group or (vii) a (C₃-C₁₇) heteroaryl group;

Z is (i) a nitrogen atom or (ii) a CR₄ group;

R₄ represents (a) a hydrogen atom, (b) a (C₁-C₁₂) alkyl group, (c) a (C₂-C₁₂) alcyn-1-yl group, (d) a (C₆-C₁₈) aryl group, (e) a (C₆-C₁₈)aryl(C₁-C₁₂) alkyl group, (f) a OR₈ group, (g) a NR₉R_{9′} group, (h) a (C₁-C₁₇) heteroaryl group or (i) a (C₂-C₁₂) alcen-1-yl group;

R₅, R₆ and R₇ represent, independently of each other, (i) a hydrogen atom, (ii) a halogen atom, (iii) a (C₁-C₁₂) alkyl group, (iv) a (C₆-C₁₈) aryl group, (v) a (C₆-C₁₈)aryl(C₁-C₁₂)alkyl group, (vi) a NR₉R_{9′} group (vii) a COR₁₀ group, (viii) a (C₂-C₁₂) alcen-1-yl group, (ix) a (C₂-C₁₂) alcyn-1-yl group, (x) a (C₁-C₁₇) heteroaryl group, (xi) a (C₃-C₁₇)heteroaryl(C₁-C₁₂)alkyl group, (xii) a cyano group or (xiii) a nitro group;

R₈ represents (i) a hydrogen atom, (ii) a (C₁-C₁₂) alkyl group, (iii) a (C₆-C₁₈)aryl(C₁-C₁₂)alkyl group;

R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) an acyl group, (vi) a tert-butyloxycarbonyl group, (vii) a (C₁-C₁₇) heteroaryl group or (viii) a (C₆-C₁₈) arylsulfonyl or (C₁-C₁₂) alkylsulfonyl group;

R₉ which can be the same as or different from R₉, represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) an acyl group, (vi) a tert-butyloxycarbonyl group, (vii) a (C₁-C₁₇) heteroaryl group or (viii) a (C₆-C₁₈) arylsulfonyl or (C₁-C₁₂) alkylsulfonyl group;

NR₉R_{9′} can also represent a cycloheteroalkyl group of the type: 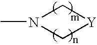
with n=2 or 3,
m=2 or 3 and
Y represents a CH₂, SO₂, or NR₁₁ group or else an oxygen or sulfur atom,

R₁₀ represents (i) a hydrogen atom, (ii) a (C₁-C₁₂) alkyl group or (iii) a (C₆-C₁₈) aryl group or (iv) a NHR₂ group;

R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;

X is (i) a halogen atom, (ii) a OR₈ group, (iii) a NR₉R_{9′} group, (iv) a (C₆-C₁₈) aryl group, (v) a (C₆-C₁₈)aryl(C₁-C₁₂)alkyl group, (vi) a (C₃-C₁₂) alkyl group, (vii) a (C₂-C₁₂)alcen-1-yl group, (viii) a (C₂-C₁₂)alcyn-1-yl group, (ix) a (C₁-C₁₇) heteroaryl group, (x) a COR₁₀ group, (xi) a cyano group or (xii) a nitro group.

The compounds represented by formula (I) which contain one or more asymmetrical carbon atoms may be in the form of racemates, enantiomers and diastereomers. Such forms are also encompassed by the invention.

The compounds represented by formula (I) wherein Z is CR₄, R₄ is OR₈ and R₈ is hydrogen may also be present as tautomers. Such tautomers are encompassed by the invention.

The invention is also based on using compounds represented by general formula (I) such as described hereinabove for preparing a pharmaceutical composition for modulating the activity of xanthurenic acid.

The invention is further based on using compounds represented by general formula (I) such as described hereinabove for preparing a pharmaceutical composition for modulating dopaminergic neurotransmission.

As will be demonstrated in the examples, the inventive compounds are able to antagonize the binding of XA to its receptor, or, in contrast, to mimic this binding (agonist). Furthermore, some compounds of the invention are allosteric modulators of XA, that is to say, they are capable of enhancing the binding of XA to its receptors.

The invention also concerns pharmaceutical compositions, compounds represented by formula (I) and treatment methods using them.

Xanthurenic acid (XA) is preferably excluded from the present invention. Derivatives of xanthurenic acid are preferred and in particular compounds represented by formula (I) wherein at least one of the groups R₃, R₅, R₆ and R₇ is different from the hydrogen atom and advantageously wherein X represents a hydroxyl group and Z represents COH.

Within the scope of the invention, the described groups are preferably defined as follows:

“Alcen-1-yl” groups are preferably linear or branched hydrocarbons having 2 to 12 carbon atoms and containing a double bond in position −1. They preferably contain from 2 to 6 carbon atoms. Such groups are not substituted or may be substituted by one or more substituents, which are the same or different, preferably chosen from among OR₈, aryl, NR₉R_{9′} groups, R₈, R₉ and R_{9′} being defined hereinabove.

“Alcyn-1-yl” groups are preferably linear or branched hydrocarbons having 2 to 12 carbon atoms and containing a triple bond in position 1. They preferably contain from 2 to 6 carbon atoms. Such groups are not substituted or may be substituted by one or more substituents, which are the same or different, preferably chosen from among OR₈, aryl, NR₉R_{9′} groups, R₈, R₉ and R_{9′} being defined hereinabove.

“Alkyl” denotes linear or branched hydrocarbons having from 1 to 12 carbon atoms such as methyl, ethyl, propyl, isopropyl, butyl, isobutyl, tert-butyl, pentyl, neopentyl, n-hexyl, n-decyl, n-dodecyl, etc. C1-C4 groups are preferred. The alkyl groups are not substituted or may be substituted by one or more substituents, which are the same or different, preferably chosen from among aryl, OR₈, —NR₉R_{9′}, CONHR₂, R₈, R₉ and R_{9′} being defined as hereinabove. In the case where R₄ represents a substituted alkyl group, the substituents are preferably chosen from among the groups aryl, OR₈, —NR₉R_{9′}, CONHR₂, R₉ and R_{9′} being defined as hereinabove and R₈ represents (ii) a (C₁-C₁₂) alkyl group or (iii) a (C₆-C₁₈)aryl(C₁-C₁₂)alkyl group.

“Alkoxy” corresponds to the alkyl groups defined hereinabove, linked to the rest of the molecule by means of an —O— (ether) bond. Methoxy or benzyloxy groups are especially preferred.

“Aryl” groups are mono-, bi- or tri-cyclic aromatic hydrocarbon systems, preferably mono- or bi-cyclic aromatic hydrocarbon systems having from 6 to 18 carbon atoms, even more preferably 6 carbon atoms. Examples include phenyl, naphthyl and biphenyl groups. The aryl groups may possibly be substituted by one or more substituents, which are the same or different, preferably chosen from among the halogen atoms and (C₁-C₁₂) alkyl and (C₁-C₁₂) alkoxy groups, a cyano group, a CONHR₂ or NR₉R_{9′} group.

“Acyl” groups correspond to the alkyl or aryl groups defined hereinabove, linked to the rest of the molecule by means of a —CO— (carbonyl) bond. In other words the acyl groups are —CO—(C₁-C₁₂) alkyl or —CO—(C₆-C₁₈)aryl groups. The acetyl and benzoyl groups are examples.

“Arylsulfonyl” and “alkylsulfonyl” groups are aryl or alkyl groups linked to the rest of the molecule by means of a SO₂ bond. Such groups are exemplified by the p-tolylsulfonyl group.

“Heteroaryl” groups denote aryl groups in which 1 to 4 carbon atoms are replaced by heteroatoms chosen from among N, O, S and containing from 1 to 17 atoms and particularly from 1 to 10 carbon atoms. Examples include pyridyl, thienyl, benzothienyl, benzofuryl, pyrimidinyl, pyridazinyl, isoquinolinyl, thiazolyl, furyl, pyranyl, pyrrolyl, 2H-pyrrolyl, imidazolyl, benzimidazolyl, pyrazolyl, isothiazolyl, isoxazolyl, indolyl and tetrazolyl groups. These may possibly be substituted by halogen atoms or (C₁-C₁₂) alkyl or (C₁-C₁₂) alkoxy groups.

The term “arylalkyl” refers to an alkyl group substituted by an aryl group. Examples of arylalkyl groups include the benzyl, phenethyl and phenethylpropyl groups.

The term “heteroarylalkyl” denotes an alkyl group substituted by a heteroaryl group. Examples of heteroarylalkyl groups include 2-pyridinylethyl, 3-pyridinylethyl groups.

“Cycloheteroalkyl” denotes nitrogenated cycles with 4 to 7 carbon atoms containing a nitrogen atom and in which one carbon atom may be replaced by a heteroatom chosen from among O, S, SO₂ or NR₁₁. In the case where the heteroatom is NR₁₁, it may be substituted or not. Examples of cycloheteroalkyl groups include morpholino, thiomorpholino, piperidin-1-yl, piperazin-1-yl, 4-methyl-piperazin-1-yl, 4-(2-pyrimidine)-piperazin-1-yl and 4-phenyl-piperazin-1-yl groups.

“Halogen” signifies a fluorine, chlorine, bromine or iodine atom. In the case where X represents a halogen atom, the bromine atom is preferred.

In general formula (I) hereinabove, preferred compounds are those wherein:

E is a COOH, COOR₁, CHO, CH₂COOH, CH₂COOR₁ group or a group chosen from among the following: 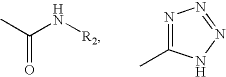
and/or

R₁ represents a (C₁-C₁₂) alkyl or (C₆-C₁₈)aryl(C₁-C₁₂)alkyl group; and/or

R₂ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group or (v) a hydroxyl group; and/or

R₃ is (i) a hydrogen atom, (ii) a halogen atom, (iii) a hydroxyl group, (iv) a (C₁-C₁₂) alkyl group, (vi) a (C₆-C₁₈) aryl(C₁-C₁₂) alkyl group; and/or

Z is (i) a nitrogen atom or (ii) a CR₄ group; and/or

R₄ represents (a) a hydrogen atom, (b) a (C₁-C₁₂) alkyl group, (c) a (C₂-C₁₂) alcyn-1-yl group, (d) a (C₆-C₁₈) aryl group, (e) a OR₈ group in which R₈ represents hydrogen, (f) a (C₁-C₁₇) heteroaryl group or (g) a NR₉R_{9′} group in which R₉ represents hydrogen, acyl or (C₆-C₁₈) arylsulfonyl and R_{9′} represents hydrogen, acyl or NR₉R_{9′} represents a cycloheteroalkyl group of the type: 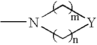
with n=2 or 3,
m=2 or 3 and
Y represents a CH₂, SO₂, or NR₁₁ group or else an oxygen or sulfur atom,
and/or

R₅, R₆, R₇ represent, independently of each other, (i) a hydrogen atom, (ii) a halogen atom, (iii) a (C₁-C₁₂) alkyl group, (iv) a (C₆-C₁₈) aryl group, (v) a NR₉R_{9′} group in which R₉ represents hydrogen, and R_{9′} represents hydrogen, acyl or (C₆-C₁₈) arylsulfonyl or NR₉R_{9′} represents a cycloheteroalkyl group of the type: 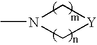
with n=2 or 3,
m=2 or 3 and
Y represents a CH₂, SO₂, or NR₁₁ group or else an oxygen or sulfur atom,
(vi) a COR₁₀ group in which R₁₀ represents hydrogen, (vii) a (C₂-C₁₂) alcen-1-yl group, (viii) a (C₂-C₁₂) alcyn-1-yl group, (ix) a (C₁-C₁₇) heteroaryl group, (x) a (C₆-C₁₈)aryl(C₁-C₁₂)alkyl group, (xi) a cyano group; and/or

X is (i) a halogen atom, (ii) a OR₈ group in which R₈ is a hydrogen atom, a (C₁-C₁₂) alkyl or (C₆-C₁₈)aryl(C₁-C₁₂)alkyl group, (iii) a (C₆-C₁₈)aryl(C₁-C₁₂)alkyl group, (iv) a (C₆-C₁₈) aryl group, (v) a (C₁-C₁₇) heteroaryl group, (vi) a NR₉R_{9′} group in which R₉ is hydrogen, (C₆-C₁₈)aryl(C₁-C₁₂)alkyl or acyl or (C₁-C₁₂) alkylsulfonyl and (C₆-C₁₈) arylsulfonyl and R_{9′} represents hydrogen, acyl or (C₆-C₁₈) arylsulfonyl or NR₉R_{9′} which represents a cycloheteroalkyl group of the type: 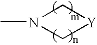
with n=2 or 3,
m=2 or 3 and
Y represents a CH₂, SO₂, or NR₁₁ group or else an oxygen or sulfur atom,

R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;

and their pharmaceutically acceptable salts.

A preferred family of compounds is that wherein R₁ is an unsubstituted alkyl or benzyl group, preferably when E represents a COOR₁ group.

Another preferred family is that wherein E represents a CONHR₂ group in which R₂ is a hydroxyl group.

Another preferred family is that wherein R₃ is (i) a hydrogen atom, (ii) a (C₁-C₁₂) alkyl group not substituted or substituted by amino or alkylamino, or (iii) a (C₆-C₁₈) aryl group.

A further preferred family is that wherein Z is (i) a nitrogen atom or (ii) a CR₄ group in which R₄ represents (a) a hydrogen atom, (b) a (C₁-C₁₂) alkyl group not substituted or substituted by phenyl or NR₉R_{9′} in which R₉ is hydrogen or tert-butyloxycarbonyl and R_{9′} is hydrogen, or else by NR₉R_{9′} which represents a cycloheteroalkyl group of the type: 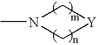
with n=2 or 3,
m=2 or 3 and
Y represents a CH₂, SO₂, or NR₁₁ group or else an oxygen or sulfur atom,
(c) a (C₂-C₁₂) alcyn-1-yl group not substituted or substituted by phenyl, hydroxyl, benzyloxy or NR₉R_{9′} with R₉ representing tert-butyloxycarbonyl and R_{9′} hydrogen, or NR₉R_{9′} represents a cycloheteroalkyl group of the type: 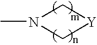
with n=2 or 3,
m=2 or 3 and
Y represents a CH₂, SO₂, or NR₁₁ group or else an oxygen or sulfur atom,
(d) a (C₆-C₁₈) aryl group not substituted or substituted by halogen, (e) an OR₈ group in which R₈ represents hydrogen, (f) a NR₉R_{9′} group in which R₉ represents hydrogen or tosyl and R_{9′} represents hydrogen or else a NR₉R_{9′} group which represents a cycloheteroalkyl group of the type: 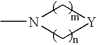
with n=2 or 3,
m=2 or 3 and
Y represents a CH₂, SO₂, or NR₁₁ group or else an oxygen or sulfur atom.

Even more preferred is the use, for treating nervous system pathologies, of compounds represented by formula (I) wherein;

E is a COOH, COOR₁, CHO, CH₂COOH, CH₂COOR₁ group or a group chosen from among the following: 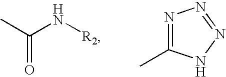
R₁ represents an unsubtituted (C₁-C₁₂) alkyl group or benzyl group; or

R₂ represents a hydroxyl group; and/or

Z is (i) a nitrogen atom or (ii) a CR₄ group in which R₄ represents (a) a hydrogen atom, (b) a (C₁-C₁₂) alkyl group not substituted or substituted by phenyl, hydroxyl or by NR₉R_{9′} with R₉ being hydrogen or tert-butyloxycarbonyl and R_{9′} hydrogen or else by NR₉R_{9′} which represents a cycloheteroalkyl group of the type: 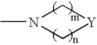
with n=2 or 3,
m=2 or 3 and
Y represents a CH₂, SO₂, or NR₁₁ group or else an oxygen or sulfur atom,
(c) a (C₂-C₁₂) alcyn-1-yl group not substituted or substituted by phenyl, hydroxyl, benzyloxy or by NR₉R_{9′} with R₉ representing tert-butyloxycarbonyl and R_{9′} hydrogen, or else by NR₉R_{9′} which represents a cycloheteroalkyl group of the type: 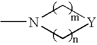
with n=2 or 3,
m=2 or 3 and
Y represents a CH₂, SO₂, or NR₁₁ group or else an oxygen or sulfur atom,
(d) a (C₆-C₁₈) aryl group not substituted or substituted by halogen, (e) a OR₈ group in which R₈ represents hydrogen, (f) a NR₉R_{9′} in which R₉ represents hydrogen or tosyl and R_{9′} is hydrogen; or a NR₉R_{9′} group which represents a cycloheteroalkyl group of the type: 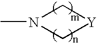
with n=2 oru 3,
m=2 or 3 and
Y represents a CH₂, SO₂, or NR₁₁ group or else an oxygen or sulfur atom,

R₃ is (i) a hydrogen atom, (ii) a halogen atom, (iii) a hydroxyl group, (iv) a (C₁-C₁₂) alkyl group not substituted or substituted by amino, alkylamino, (v) a (C₆-C₁₈) aryl group;

R₅, R₆, R₇ represent, independently of each other, (i) a hydrogen atom, (ii) a halogen atom, (iii) a (C₁-C₁₂) alkyl group not substituted or substituted by hydroxyl, phenyl or NR₉R_{9′} in which R₉ is hydrogen or tert-butyloxycarbonyl and R_{9′} is hydrogen, (iv) a phenyl group not substituted or substituted by halogen, alkoxy, alkyl, (v) a (C₆-C₁₈)aryl(C₁-C₁₂)alkyl group, (vi) a NR₉R_{9′} group in which R₉ represents hydrogen and R_{9′} is hydrogen, (vii) a COR₁₀ group in which R₁₀ represents hydrogen, (viii) an unsubstituted (C₂-C₁₂) alcen-1-yl group, (ix) a (C₂-C₁₂) alcyn-1-yl group not substituted or substituted by phenyl, NR₉R_{9′} in which R₉ is hydrogen or tert-butyloxycarbonyl and R_{9′} is hydrogen, OR₈ in which R₈ is hydrogen or tert-butoxycarbonyl, (x) a pyridyl group;

X is (i) a halogen atom, (ii) a OR₈ group in which R₈ is hydrogen, (C₁-C₆) alkyl or benzyl, (iii) a NR₉R_{9′} group in which R₉ is hydrogen, acetyl or benzoyl and R_{9′} is hydrogen, acetyl or benzoyl, or (iv) phenyl.

and their pharmaceutically acceptable salts.

Even more preferably, in the compounds represented by formula (I) and the subfamilies defined hereinabove, Z is a CR₄ group.

According to another variant of the invention, in the compounds represented by formula (I) and the subfamilies defined hereinabove, Z represents CR₄ where R₄ is a hydroxyl group, X represents a halogen atom, preferably bromine, and in particular at least one of the groups R₃, R₅, R₆ and R₇ is different from the hydrogen atom, avantageously with R₅ and R₇ not representing a halogen atom.

According to yet another variant of the invention, in the compounds represented by formula (I) and the subfamilies defined hereinabove, Z represents CR₄ in which R₄ is a hydroxyl group, X represents a hydroxyl group and advantageously at least one of the groups R₃, R₅, R₆ and R₇ is different from the hydrogen atom.

Among the compounds represented by formula (I), the following compounds are preferably used:
Methyl-4-hydroxy-8-benzyloxy-quinoline-2-carboxylate
Methyl-4-hydroxy-5-bromo-8-benzyloxy-quinoline-2-carboxylate
Methyl-4,8-dihydroxy-5-bromo-quinoline-2-carboxylate
Methyl-4-hydroxy-5-bromo-8-methoxy-quinoline-2-carboxylate
Methyl-4-hydroxy-5-methyl-8-methoxy-quinoline-2-carboxylate
Methyl-4-hydroxy-5-(1-hydroxy-ethyl)-8-methoxy-quinoline-2-carboxylate
Methyl-4-hydroxy-5-hydroxymethyl-8-benzyloxy-quinoline-2-carboxylate
Methyl-4,8-dihydroxy-5-hydroxymethyl-quinoline-2-carboxylate
Methyl-4-hydroxy-5,7-dichloro-8-methoxy-quinoline-2-carboxylate
Methyl-4-hydroxy-6-iodo-8-methoxy-quinoline-2-carboxylate
Methyl-4-hydroxy-6-bromo-8-methoxy-quinoline-2-carboxylate
Methyl-4-hydroxy-6-bromo-8-benzyloxy-quinoline-2-carboxylate
Methyl-4,8-dihydroxy-6-bromo-quinoline-2-carboxylate
Methyl-4-hydroxy-6-methyl-8-methoxy-quinoline-2-carboxylate
Methyl-4-hydroxy-6-formyl-8-methoxy-quinoline-2-carboxylate
Methyl-4-hydroxy-6-amino-8-methoxy-quinoline-2-carboxylate
Methyl-4-hydroxy-8-amino-quinoline-2-carboxylate
Methyl-4-hydroxy-5-methyl-8-amino-quinoline-2-carboxylate
Methyl-4-hydroxy-6-methyl-8-amino-quinoline-2-carboxylate
Methyl-4-hydroxy-8-bromo-quinoline-2-carboxylate
4,8-dihydroxy-quinoline-2-carboxylic acid
4,8-dihydroxy-5-bromo-quinoline-2-carboxylic acid
4,8-dihydroxy-5-methyl-quinoline-2-carboxylic acid
4,8-dihydroxy-5-hydroxymethyl-quinoline-2-carboxylic acid
4,8-dihydroxy-5,7-dichloro-quinoline-2-carboxylic acid
4,8-dihydroxy-6-iodo-quinoline-2-carboxylic acid
4,8-dihydroxy-6-bromo-quinoline-2-carboxylic acid
4,8-dihydroxy-6-methyl-quinoline-2-carboxylic acid
4,8-dihydroxy-6-formyl-quinoline-2-carboxylic acid
4-hydroxy-8-amino-quinoline-2-carboxylic acid
4-hydroxy-5-methyl-8-amino-quinoline-2-carboxylic acid
4-hydroxy-6-methyl-8-amino-quinoline-2-carboxylic acid
4-hydroxy-8-bromo-quinoline-2-carboxylic acid
4-hydroxy-8-benzyloxy-quinoline-2-carboxylic acid
8-hydroxy-5-bromo-quinoline-2-carboxylic acid
8-benzyloxy-7-bromo-quinoline-2-carboxylic acid
8-hydroxy-7-bromo-quinoline-2-carboxylic acid
3-methyl-8-methoxy-quinoline-2-carboxylic acid
8-amino-quinoline-2-carboxylic acid
8-methoxy-5-bromo-quinoline-2-carboxylic acid
Benzyl-8-benzyloxy-quinoline-2-carboxylate
Benzyl-8-benzyloxy-5-bromo-quinoline-2-carboxylate
Methyl-8-methoxy-5-bromo-quinoline-2-carboxylate
Benzyl-8-benzyloxy-7-bromo-quinoline-2-carboxylate
Methyl-8-methoxy-3-methyl-quinoline-2-carboxylate
Methyl-8-amino-quinoline-2-carboxylate
Methyl-4-chloro-8-benzyloxy-quinoline-2-carboxylate
Methyl-8-methoxy-4-methyl-quinoline-2-carboxylate
Methyl-8-methoxy-4-phenyl-quinoline-2-carboxylate
Methyl-8-methoxy-4-(4-chloro-phenyl)-quinoline-2-carboxylate
8-hydroxy-4-methyl-quinoline-2-carboxylic acid
8-hydroxy-4-phenyl-quinoline-2-carboxylic acid
8-hydroxy-4-(4-chloro-phenyl)-quinoline-2-carboxylic acid
Methyl-8-benzyloxy-4-phenylethynyl-quinoline-2-carboxylate
Methyl-8-benzyloxy-4-(3-tert-butoxycarbonylaminoprop-1-ynyl)-quinoline-2-carboxylate
Methyl-8-benzyloxy-4-(3-benzyloxyprop-1-ynyl)-quinoline-2-carboxylate
Methyl-8-hydroxy-4-phenethyl-quinoline-2-carboxylate
Methyl-8-hydroxy-4-(3-tert-butoxycarbonylamino-propyl)-quinoline-2-carboxylate
Methyl-8-hydroxy-4-(3-hydroxy-propyl)-quinoline-2-carboxylate
8-hydroxy-4-phenethyl-quinoline-2-carboxylic acid
8-hydroxy-4-(3-amino-propyl)-quinoline-2-carboxylic acid
8-hydroxy-4-(3-hydroxy-propyl)-quinoline-2-carboxylic acid
Methyl-8-benzyloxy-4-(toluene-4-sulfonylamino)-quinoline-2-carboxylate
Methyl-8-hydroxy-4-(toluene-4-sulfonylamino)-quinoline-2-carboxylate
Methyl-8-amino-4-(toluene-4-sulfonylamino)-quinoline-2-carboxylate
8-hydroxy-4-(toluene-4-sulfonylamino)-quinoline-2-carboxylic acid
Methyl-4-amino-8-hydroxy-quinoline-2-carboxylate
Methyl-4,8-diamino-quinoline-2-carboxylate
4-amino-8-hydroxy-quinoline-2-carboxylic acid
4,8-diamino-quinoline-2-carboxylic acid
Methyl-4-hydroxy-8-benzyloxyl-5-phenyl-quinoline-2-carboxylate
Methyl-4,8-dihydroxy-5-phenyl-quinoline-2-carboxylate
Methyl-4-hydroxy-8-methoxy-5-(4-chloro-phenyl)-quinoline-2-carboxylate
Methyl-4-hydroxy-6-phenyl-8-methoxy-quinoline-2-carboxylate
Methyl-4-hydroxy-6-(4-methoxy-phenyl)-8-methoxy-quinoline-2-carboxylate
Methyl-4,8-dihydroxy-6-(4-methoxy-phenyl)-quinoline-2-carboxylate
Methyl-4-hydroxy-6-(3-methyl-phenyl)-8-benzyloxy-quinoline-2-carboxylate
Methyl-4,8-dihydroxy-6-(3-methyl-phenyl)-quinoline-2-carboxylate
Methyl-4-hydroxy-6-(4-chloro-phenyl)-8-benzyloxy-quinoline-2-carboxylate
Methyl-4,8-dihydroxy-6-(4-chloro-phenyl)-quinoline-2-carboxylate
Methyl-8-benzyloxy-6-(3,4-dichloro-phenyl)-4-hydroxy-quinoline-2-carboxylate
Methyl-4,8-dihydroxy-6-(3,4-dichloro-phenyl)-quinoline-2-carboxylate
Methyl-8-benzyloxy-4-hydroxy-6-(pyridin-3-yl)-quinoline-2-carboxylate
Methyl-4,8-dihydroxy-6-(pyridin-3-yl)-quinoline-2-carboxylate
Methyl-4-hydroxy-8-methoxy-6-propenyl-quinoline-2-carboxylate
Methyl-4-hydroxy-8-methoxy-6-propyl-quinoline-2-carboxylate
Methyl-4-hydroxy-8-phenyl-quinoline-2-carboxylate
Methyl-8-Methoxy-5-phenyl-quinoline-2-carboxylate
4,8-dihydroxy-5-phenyl-quinoline-2-carboxylic acid
4,8-dihydroxy-5-(4-chloro-phenyl)-quinoline-2-carboxylic acid
4,8-dihydroxy-6-phenyl-quinoline-2-carboxylic acid
4,8-dihydroxy-6-(4-methoxy-phenyl)-quinoline-2-carboxylic acid
4,8-dihydroxy-6-(3-methyl-phenyl)-quinoline-2-carboxylic acid
4,8-dihydroxy-6-(4-chlorophenyl)-quinoline-2-carboxylic acid
4,8-dihydroxy-6-(3,4-dichlorophenyl)-quinoline-2-carboxylic acid
4,8-dihydroxy-6-(pyridin-3-yl)-quinoline-2-carboxylic acid
4,8-dihydroxy-6-propyl-quinoline-2-carboxylic acid
4-hydroxy-8-phenyl-quinoline-2-carboxylic acid
8-hydroxy-5-phenyl-quinoline-2-carboxylic acid
Methyl-4-hydroxy-8-methoxy-6-phenylethynyl-quinoline-2-carboxylate
Methyl-4-hydroxy-8-methoxy-6-(hept-1-ynyl)-quinoline-2-carboxylate
Methyl-8-benzyloxy-6-(3-tert-butoxycarbonylamino-prop-1-ynyl)-4-hydroxy-quinoline-2-carboxylate
Methyl-8-benzyloxy-6-(3-benzyloxy-prop-1-ynyl)-6-hydroxy-quinoline-2-carboxylate
Methyl-4-hydroxy-8-methoxy-6-phenethyl-quinoline-2-carboxylate
Methyl-4-hydroxy-8-methoxy-6-heptyl-quinoline-2-carboxylate
Methyl-4,8-dihydroxy-6-(3-tert-butoxycarbonylamino-propyl)-quinoline-2-carboxylate
Methyl-4,8-dihydroxy-6-(3-hydroxy-propyl)-quinoline-2-carboxylate
4,8-dihydroxy-6-phenethyl-quinoline carboxylic acid
4,8-dihydroxy-6-heptyl-quinoline-2-carboxylic acid
4,8-dihydroxy-6-(3-amino-propyl)quinoline-2-carboxylic acid
4,8-dihydroxy-6-(3-hydroxy-propyl)-quinoline-2-carboxylic acid
Methyl-8-benzyloxy-6-benzyl-4-hydroxy-quinoline-2-carboxylate
Methyl-4,8-dihydroxy-6-benzyl-quinoline-2-carboxylate
4,8-dihydroxy-6-benzyl-quinoline-2-carboxylic acid
Methyl-6-(benzylamino-methyl)-4-hydroxy-8-methoxy-quinoline-2-carboxylate 4,8-dihydroxy-6-(benzylamino-methyl)-quinoline-2-carboxylic acid
8-acetylamino-4-hydroxy-quinoline-2-carboxylic acid
8-pivaloylamino-4-hydroxy-quinoline-2-carboxylic acid
8-benzoylamino-4-hydroxy-quinoline-2-carboxylic acid
Methyl-8-benzylamino-4-hydroxy-quinoline-2-carboxylate
8-benzylamino-4-hydroxy-quinoline-2-carboxylic acid
8-benzyloxy-4-hydroxy-quinoline-2-carboxylic acid hydroxyamide
4,8 dihydroxy-quinoline-2-carboxylic acid hydroxyamide
8-methoxy-2-(2H-tetrazol-1-yl)-quinoline
8-hydroxy-2-(2H-tetrazol-1-yl)-quinoline
8-benzyloxy-quinoxazoline-2-carbaldehyde
8-hydroxy-quinoxazoline-2-carbaldehyde
(8-benzyloxy-quinoxazolin-2-yl)-methanol
(8-hydroxy-quinoxazolin-2-yl)-methanol
Methyl-8-methoxy-3-methylaminomethyl-quinoline-2-carboxylate
8-hydroxy-3-methylaminomethyl-quinoline-2-carboxylic acid
Methyl-3-benzyl-8-methoxy-quinoline-2-carboxylate
3-benzyl-8-methoxy-quinoline-2-carboxylic acid
Methyl-4-hydroxy-8-bromo-quinoline-2-carboxylate
Methyl-8-benzyloxy-5,7-dichloro-4-hydroxyquinoline-2-carboxylate
Methyl-8-benzyloxy-7-bromo-4-hydroxyquinoline-2-carboxylate
Methyl-6-bromo-8-cyano-4-hydroxy-quinoline-2-carboxylate
Ethyl-8-hydroxy-3-oxo-3,4-dihydroquinoxaline-2-carboxylate
Ethyl[8-(hydroxy)-3-oxo-3,4-dihydroquinoxaline-2(1H)-ylilene]acetate
Methyl-5,7-dichloro-4,8-dihydroxyquinoline-2-carboxylate
Methyl-3-bromo-4,8-dihydroxy-quinoline-2-carboxylate
Methyl-3,7-dibromo-4,8-dihydroxy-quinoline-2-carboxylate
8-bromo-4-hydroxy-6-isopropyl-quinoline-2-carboxylic acid
8-benzyloxy-6-bromo-4-hydroxy-quinoline-2-carboxylic acid
8-benzyloxy-7-bromo-4-hydroxyquinoline-2-carboxylic acid
8-benzyloxy-3-bromo-4-hydroxy-quinoline-2-carboxylic acid
[8-(hydroxy)-3-oxo-3,4-dihydroquinoxaline-2(1H)-yl]acetic acid
6-bromo-8-cyano-4-hydroxy-quinoline-2-carboxylic acid
8-cyano-4-hydroxy-6-phenyl-quinoline-2-carboxylic acid
8-cyano-4-hydroxy-6-phenenyl-quinoline-2-carboxylic acid
3-bromo-8-cyano-4-hydroxy-quinoline-2-carboxylic acid
8-cyano-4-hydroxy-3-phenylethynyl-quinoline-2-carboxylic acid
8-cyano-6-ethyl-4-hydroxy-quinoline-2-carboxylic acid
Benzyl-4-benzyloxy-8-bromo-quinoline-2-carboxylate
Benzyl-4,8-dibenzyloxy-6-bromo-quinoline-2-carboxylate
Benzyl-4,8-dibenzyloxy-7-bromo-quinoline-2-carboxylate
Benzyl-3-bromo-4,8-dibenzyloxy-quinoline-2-carboxylate
Methyl-6-bromo-8-cyano-4-benzyloxy-quinoline-2-carboxylate
Benzyl-3-bromo-4-benzyloxy-8-cyano-quinoline-2-carboxylate
Methyl-8-benzyloxy-6-(furo-2-yl)-4-hydroxyquinoline-2-carboxylate
Benzyl-4,8-dibenzyloxy-6-(2-chlorophenyl)-quinoline-2-carboxylate
Benzyl-4,8-dibenzyloxy-6-(3-chlorophenyl)-quinoline-2-carboxylate
Benzyl-4,8-dibenzyloxy-7-phenyl-quinoline-2-carboxylate
Benzyl-4,8-dibenzyloxy-6-(2-methoxyphenyl)-quinoline-2-carboxylate
Benzyl-4,8-dibenzyloxy-3-phenyl-quinoline-2-carboxylate
Methyl-8-cyano-4-hydroxy-6-phenyl-quinoline-2-carboxylate
Benzyl-4-benzyloxy-8-(hex-1-ynyl)-quinoline-2-carboxylate
Benzyl-4,8-dibenzyloxy-6-(3-benzyloxy-prop-1-ynyl)-quinoline-2-carboxylate Benzyl-4,8-dibenzyloxy-7-(3-benzyloxy-prop-1-ynyl)-quinoline-2-carboxylate Benzyl-4,8-dibenzyloxy-7-phenylethynyl-quinoline-2-carboxylate
Methyl-4-benzyloxy-8-cyano-6-phenylethynyl-quinoline-2-carboxylate
Benzyl-4-benzyloxy-8-cyano-3-phenylethynyl-quinoline-2-carboxylate
Methyl-8-cyano-4-hydroxy-6-[(trimethylsilyl)ethynyl]-quinoline-2-carboxylate
8-hexyl-4-hydroxy-quinoline-2-carboxylic acid
4,8-dihydroxy-6-(3-hydroxy-propyl)-quinoline-2-carboxylic acid
4,8-dihydroxy-7-(3-hydroxy-propyl)-quinoline-2-carboxylic acid
Methyl-8-cyano-4-hydroxy-6-phenethyl-quinoline-2-carboxylate
Methyl-8-cyano-6-ethyl-4-hydroxy-quinoline-2-carboxylate
4,8-dihydroxy-6-(furo-2-yl)-quinoline-2-carboxylic acid
4-hydroxy-8-phenyl-quinoline-2-carboxylic acid
4,8-dihydroxy-6-(2-chlorophenyl)-quinoline-2-carboxylic acid
4,8-dihydroxy-6-(3-chlorophenyl)-quinoline-2-carboxylic acid
4,8-dihydroxy-7-phenyl-quinoline-2-carboxylic acid
4,8-dihydroxy-6-(2-methoxyphenyl)-quinoline-2-carboxylic acid
4,8-dihydroxy-3-phenyl-quinoline-2-carboxylic acid
4,8-dihydroxy-4-piperidin-2-yl-quinoline-2-carboxylic acid hydrochloride
Sodium 8-hexyl-4-hydroxy-quinoline-2-carboxylate
Sodium 4,8-dihydroxy-7-phenyl-quinoline-2-carboxylate
Sodium 4,8-dihydroxy-3-phenyl-quinoline-2-carboxylate
Methyl-8-benzyloxy-3-bromo-4-hydroxy-quinoline-2-carboxylate
Methyl-8-benzyloxy-3,7-dibromo-4-hydroxyquinoline-2-carboxylate
Methyl-3-bromo-8-cyano-4-hydroxyquinoline-2-carboxylate
Methyl-4-hydroxy-8-(2H-tetrazol-5-yl)-quinoline-2-carboxylate
Ethyl-8-(benzyloxy)-3-oxo-3,4-dihydroquinoxaline-2-carboxylate
Ethyl[8-(benzyloxy)-3-oxo-3,4-dihydroquinoxaline-2(1H)-ylilene]acetate
Benzyl-4,8-dibenzyloxy-6-piperidin-2yl-quinoline-2-carboxylate
Methyl-8-cyano-6-ethynyl-4-hydroxy-quinoline-2-carboxylate
Methyl-5-bromo-8-nitro-4-hydroxy-quinoline-2-carboxylate
Methyl-6-benzyloxy-8-nitro-4-hydroxy-quinoline-2-carboxylate
Methyl-6-chloro-8-nitro-4-hydroxy-quinoline-2-carboxylate
Methyl-6-bromo-8-nitro-4-hydroxy-quinoline-2-carboxylate
Methyl-8-cyano-4-hydroxy-quinoline-2-carboxylate
Methyl-8-fluoro-4-hydroxy-quinoline-2-carboxylate
Methyl-8-carboxamide-4-hydroxy-quinoline-2-carboxylate de methyle
Methyl-3-phenylethyl-8-amino-4-hydroxy-quinoline-2-carboxylate
3-(3′-N-tert-butoxycarbonyl-propyl)-8-amino-4-hydroxy-quinoline-2-carboxylic acid
3-(3-hydroxypropyl)-8-amino-4-hydroxy-quinoline-2-carboxylic acid
3-ethyl-8-amino-4-hydroxy-quinoline-2-carboxylic acid
Methyl-5-phenyl-8-amino-4-hydroxy-quinoline-2-carboxylate
Methyl-5-phenylethyl-8-amino-4-hydroxy-quinoline-2-carboxylate
Methyl-5-(3′-N-(terbutoxycarbonyl)aminopropyl)-8-amino-4-hydroxy-quinoline-2-carboxylate
Methyl-5-hydroxypropyl-8-amino-4-hydroxy quinoline-2-carboxylate
Methyl-5-(N-piperidinyl)-8-amino-4-hydroxy-quinoline-2-carboxylate
Methyl-5-piperazinyl-8-amino-4-hydroxy-quinoline-2-carboxylate
Methyl-6-hydroxy-8-amino-4-hydroxy-quinoline-2-carboxylate
Methyl-6-phenyl ethyl-8-amino-4-hydroxy-quinoline-2-carboxylate
Methyl-6-(3′-hydroxypropyl)-8-amino-4-hydroxy-quinoline-2-carboxylate
Methyl-6-(3′-N-(tert-butoxycarbonyl)aminopropyl)-8-amino-4-hydroxy-quinoline-2-carboxylate
Methyl-6-(3′-pyridinyl)ethyl-8-amino-4-hydroxy-quinoline-2-carboxylate
Methyl-6-(5′-cyanopentyl)-8-amino-4-hydroxy-quinoline-2-carboxylate
Methyl-6-cyano-8-amino-4-hydroxy-quinoline-2-carboxylate
6-N-(N-methylpipeerazinyl)-8-amino-4-hydroxy-quinoline-2-carboxylic acid hydrochloride
Methyl-6-N-piperidinyl-8-amino-4-hydroxy-quinoline-2-carboxylate
Methyl-6-piperazinyl-8-amino-4-hydroxy-quinoline-2-carboxylate
7-phenyl-8-amino-4-hydroxy-quinoline-2-carboxylic acid
4-(N-methylamino)-8-amino-quinoline-2-carboxylic acid hydrochloride
Methyl-8-dimethylamino-4-hydroxy-quinoline-2-carboxylate
3-(N-morpholinomethyl)-4,8-dihydroxy-quinoline-2-carboxylic acid
3-(N-pyrolidinomethyl)-4,8-dihydroxy-quinoline-2-carboxylic acid
Methyl 4-(N-methylamino)-8-amino-quinoline-2-carboxylate
6-hydroxy-8-amino-4-hydroxy-quinoline-2-carboxylic acid
6-chloro-8-amino-4-hydroxy-quinoline-2-carboxylic acid
6-phenylethyl-8-amino-4-hydroxy-quinoline-2-carboxylic acid
6-(3′-hydroxypropyl)-8-amino-4-hydroxy-quinoline-2-carboxylic acid
6-(3′-aminopropyl)-8-amino-4-hydroxy-quinoline-2-carboxylic acid
8-fluoro-4-hydroxy-quinoline-2-carboxylic acid
8-carboxamide-4-hydroxy-quinoline-2-carboxylic acid
3-bromo-8-amino-4-hydroxy-quinoline-2-carboxylic acid
8-cyano-4-hydroxy-quinoline-2-carboxylate acid
5-phenyl-8-amino-4-hydroxy-quinoline-2-carboxylic acid
6-(5′-cyanopentyl)-8-amino-4-hydroxy-quinoline-2-carboxylic acid
6-cyano-8-amino-4-hydroxy-quinoline-2-carboxylic acid
6-N-(N-methylpiperazinyl)-8-nitro-4-benzyloxy-quinoline-2-carboxylic acid hydrochloride
8-dimethylamino-4-hydroxy-quinoline-2-carboxylic acid
Methyl-5-bromo-8-nitro-4-benzyloxy-quinoline-2-carboxylate
Methyl-6-bromo-8-nitro-4-benzyloxy-quinoline-2-carboxylate
Methyl-7-bromo-4,8-dibenzyloxy-quinoline-2-carboxylate
Methyl-4-(N-methyl-toluene-4-sulfonamino)-8-nitro-quinoline-2-carboxylate
Methyl-8-dimethylamino-4-benzyloxy-quinoline-2-carboxyalte
Benzyl-7-phenyl-8-amino-4-benzyloxy-quinoline-2-carboxylate
Methyl-5-phenyl-8-nitro-4-benzyloxy-quinoline-2-carboxylate
Methyl-3-trimethylsilylethynyl-8-nitro-4-hydroxy-quinoline-2-carboxylate
Methyl-3-phenylethynyl-8-nitro-4-hydroxy-quinoline-2-carboxylate
Benzyl-3-(3′-benzyloxypropyn-1′-yl)-8-nitro-4-hydroxy-quinoline-2-carboxylate
Benzyl-3-(3′-N-(terbutoxycarbonyl)aminoprop-1′-ynyl)-8-nitro-4-hydroxy-quinoline-2-carboxylate
Methyl-5-phenylethynyl-8-nitro-4-benzyloxy-quinoline-2-carboxylate
Methyl-5-(3′-benzyloxypropyn-1′-yl)-8-nitro-4-benzyloxy-quinoline-2-carboxylate
Methyl-5-(3′-N-(terbutoxycarbonyl)aminoprop-1′-ynyl)-8-nitro-4-benzyloxy-quinoline-2-carboxylate
Methyl-6-phenylethynyl-8-nitro-4-benzyloxy-quinoline-2-carboxylate
Methyl-6-(3′-pyridyl)ethynyl-8-nitro-4-benzyloxy-quinoline-2-carboxylate
Methyl-6-(5′-cyanopent-1′-ynyl)-8-nitro-4-benzyloxy-quinoline-2-carboxylate
Methyl-6-(3′-benzyloxypropyn-1′-yl)-8-nitro-4-benzyloxy-quinoline-2-carboxylate
Methyl-6-(3′-N-(terbutoxycarbonyl)aminoprop-1′-ynyl)-8-nitro-4-benzyloxy-quinoline-2-carboxylate
Methyl-4-methylamino-8-nitro-quinoline-2-carboxylate
Sodium 7-bromo-4,8-dihydroxy-quinoline-2-carboxylate
Methyl-6-(N-(N-methyl-piperazinyl))-8-nitro-4-benzyloxy-quinoline-2-carboxylate
Methyl-6-(N-(N-benzyl-piperazinyl))-8-nitro-4-benzyloxy-quinoline-2-carboxylate
Methyl-6-(N-piperidinyl)-8-nitro-4-benzyloxy-quinoline-2-carboxylate
Methyl-6-(N-diphenylimine)-8-nitro-4-benzyloxy-quinoline-2-carboxylate
Methyl-6-(N-anilino)-8-nitro-4-benzyloxy-quinoline-2-carboxylate
Methyl-5-(N-piperidinyl)-8-nitro-4-benzyloxy-quinoline-2-carboxylate
Methyl-5-(N-(N-benzyl-piperazinyl))-8-nitro-4-benzyloxy-quinoline-2-carboxylate
Methyl-3-bromo-8-amino-4-hydroxy-quinoline-2-carboxylate
Methyl-6-bromo-8-amino-4-hydroxy-quinoline-2-carboxylate
Methyl-6-chloro-8-amino-4-hydroxy-quinoline-2-carboxylate
Benzyl-8-amino-4-benzyloxy-quinoline-2-carboxylate
Benzyl-7-iodo-8-amino-4-benzyloxy-quinoline-2-carboxylate
Methyl-6-cyano-8-nitro-4-hydroxy-quinoline-2-carboxylate
Methyl-3-bromo-4-hydroxy-8-nitro-quinoline-2-carboxylate
Methyl-6-amino-8-nitro-4-benzyloxy-quinoline-2-carboxylate
3-ethynyl-8-nitro-4-hydroxy-quinoline-2-carboxylic acid
Benzyl-8-nitro-4-benzyloxy-quinoline-2-carboxylate
3-(N-morpholinomethyl)-8-benzyloxy-4-hydroxy-quinoline-2-carboxylic acid
3-(N-pyrolidinomethyl)-8-benzyloxy-4-hydroxy-quinoline-2-carboxylic acid
Methyl-5-piperazinyl-8-amino-4-hydroxy-quinoline-2-carboxylate
3-phenylethyl-8-amino-4-hydroxy-quinoline-2-carboxylic acid
3-(3′-aminopropyl)-8-amino-4-hydroxy-quinoline-2-carboxylic acid hydrochloride
5-phenylethyl-8-amino-4-hydroxy-quinoline-2-carboxylic acid
Methyl-5-(3′-aminopropyl)-8-amino-4-hydroxy-quinoine-2-carboxylate acid hydrochloride
5-hydroxypropyl-8-amino-4-hydroxy quinoline-2-carboxylic acid
5-(N-piperidinyl)-8-amino-4-hydroxy-quinoline-2-carboxylic acid hydrochloride
5-piperazinyl-8-amino-4-hydroxy-quinoline-2-carboxylic acid hydrochloride
6-(3′-pyridinyl)ethyl-8-amino-4-hydroxy-quinoline-2-carboxylic acid hydrochloride
6-N-piperidinyl-8-amino-4-hydroxy-quinoline-2-carboxylic acid hydrochloride
6-piperazinyl-8-amino-4-hydroxy-quinoline-2-carboxylic acid hydrochloride
6-anilino-8-amino-4-hydroxy-quinoline-2-carboxylic acid hydrochloride
6,8-diamino-4-hydroxy-quinoline-2-carboxylic acid hydrochloride
Methyl-6-anilino-8-amino-4-hydroxy-quinoline-2-carboxylate
Methyl-6,8-diamino-4-hydroxy-quinoline-2-carboxylate
Methyl-4-hydroxy-8-nitro-6-phenyl-quinoline-2-carboxylate
Methyl-8-amino-4-hydroxy-6-phenyl-quinoline-2-carboxylate
Methyl-8-hydroxy-4-(piperazin-1-yl)-quinoline-2-carboxylate
Methyl-8-amino-4-phenyl-quinoline-2-carboxylate
Methyl-8-amino-4-(hex-1-yl)-quinoline-2-carboxylate
Methyl-8-amino-4-(2-phenyleth-1-yl)-quinoline-2-carboxylate
Methyl-8-amino-4-(3-tert-butoxycarbonylamino-prop-1-yl)-quinoline-2-carboxylate
Methyl-8-amino-4-(3-hydroxy-prop-1-yl)-quinoline-2-carboxylate
Methyl-4-(3-acetyl-aminoprop-1-ynyl)-8-amino-quinoline-2-carboxylate
Methyl-8-hydroxy-4-(morpholin-1-yl)-quinoline-2-carboxylate
Methyl-8-benzyloxy-4-(piperidin-1-yl)-quinoline-2-carboxylate
Methyl-8-amino-4-(piperidin-1-yl)-quinoline-2-carboxylate
Methyl-4-hydroxy-8-(piperazin-1-yl)-quinoline-2-carboxylate
Methyl-8-hydroxy-4-(4-methyl-piperazin-1-yl)-quinoline-2-carboxylate
Methyl-7-(acetylamino)-4-hydroxy-quinoline-2-carboxylate
Methyl-4-(3-benzoyl-aminoprop-1-yl)-8-hydroxyquinoline-2-carboxylate
5-(4-chlorophenyl)-8-hydroxy-quinoline-2-carboxylic acid
8-amino-4-(hex-1-yl)-quinoline-2-carboxylic acid hydrochloride
8-amino-4-(2-phenyleth-1-yl)-quinoline-2-carboxylic acid hydrochloride
8-amino-4-(3-tert-butoxycarbonylaminoprop-1-yl)-quinoline-2-carboxylic acid hydrochloride
8-amino-4-(3-hydroxy-prop-1-yl)-quinoline-2-carboxylic acid hydrochloride
4-(3-acetylaminoprop-1-ynyl)-8-amino-quinoline-2-carboxylic acid hydrochloride
8-hydroxy-4-(morpholin-1-yl)-quinoline-2-carboxylic acid hydrochloride
8-hydroxy-4-(piperidin-1-yl)-quinoline-2-carboxylic acid hydrochloride
8-amino-4-(piperidin-1-yl)-quinoline-2-carboxylic acid hydrochloride
4-hydroxy-8-(piperazin-1-yl)-quinoline-2-carboxylic acid hydrochloride
8-hydroxy-4-(4-methyl-piperazin-1-yl)-quinoline-2-carboxylic acid hydrochloride
4-hydroxy-8-phenylethyl-quinoline-2-carboxylic acid
4-(3-(benzoylamino)prop-1-yl)-8-hydroxyquinoline-2-carboxylic acid
8-amino-4-hydroxy-6-phenyl-quinoline-2-carboxylic acid
Methyl-8-nitro-4-oxytrimethanelsulfonyl-quinoline-2-carboxylate
Methyl-5-(4-chlorophenyl)-8-methoxy-quinoline-2-carboxylate
Benzyl-4,8-dibenzyloxy-6-(3,5-dichlorophenyl)-quinoline-2-carboxylate
Benzyl-4,8-dibenzyloxy-6-(4-fluorophenyl)-quinoline-2-carboxylate
Methyl-8-nitro-4-phenyl-quinoline-2-carboxylate
Benzyl-8-benzyloxy-5-phenylethynyl-quinoline-2-carboxylate
Methyl-8-benzyloxy-4-(hex-1-ynyl)-quinoline-2-carboxylate
Methyl-8-benzyloxy-4-(5-benzyloxy-pent-1-ynyl)-quinoline-2-carboxylate
Methyl-8-benzyloxy-7-(3-tert-butoxycarbonylamino-prop-1-ynyl)-4-hydroxy-quinoline-2-carboxylate
Benzyl-4,8-dibenzyloxy-7-(hex-1-ynyl)-quinoline-2-carboxylate
Methyl-8-amino-4-(hex-1-ynyl)-quinoline-2-carboxylate
Methyl-8-amino-4-phenylethynyl-quinoline-2-carboxylate
Methyl-8-nitro-4-(3-tert-butoxycarbonylamino-prop-1-ynyl)-quinoline-2-carboxylate
Methyl-4-(3-benzyloxy-prop-1-ynyl)-8-nitro-quinoline-2-carboxylate
Methyl-4-(3-acetyl-aminoprop-1-ynyl)-8-nitro-quinoline-2-carboxylate
Methyl-4-benzyloxy-8-phenylethynyl-quinoline-2-carboxylate
8-hydroxy-5-phenylethyl-quinoline-2-carboxylic acid
Methyl-8-benzyloxy-4-(hex-1-yl)-quinoline-2-carboxylate
Methyl-8-benzyloxy-4-(5-hydroxy-pent-1-yl)-quinoline-2-carboxylate
Methyl-8-benzyloxy-4-(3-tert-butoxycarbonylaminoprop-1-yl)-quinoline-2-carboxylate
Methyl-4,8-dihydroxy-7-(3-tert-butoxycarbonylaminoprop-1-yl)-quinoline-2-carboxylate
4,8-dihydroxy-7-(hex-1-yl)-quinoline-2-carboxylic acid
Methyl-4-hydroxy-8-phenylethyl-quinoline-2-carboxylate
8-hydroxy-5-phenylethyl-quinoline-2-carboxylic acid
Methyl-8-benzyloxy-4-(hex-1-yl)-quinoline-2-carboxylate
Methyl-8-benzyloxy-4-(5-hydroxy-pent-1-yl)-quinoline-2-carboxylate
Methyl-8-benzyloxy-4-(3-tert-butoxycarbonylaminoprop-1-yl)-quinoline-2-carboxylate
Methyl-4,8-dihydroxy-7-(3-tert-butoxycarbonylaminoprop-1-yl)-quinoline-2-carboxylate
4,8-dihydroxy-7-(hex-1-yl)-quinoline-2-carboxylic acid
Methyl-4-hydroxy-8-phenylethyl-quinoline-2-carboxylate
Sodium 4-(hex-1-yl)-8-hydroxy-quinoline-2-carboxylate
8-amino-4-hydroxy-6-phenyl-quinoline-2-carboxylic acid
Methyl-8-benzyloxy-4-(4-benzyl-piperazin-1-yl)-quinoline-2-carboxylate 87a
Methyl-8-benzyloxy-4-(morpholin-1-yl)-quinoline-2-carboxylate
Methyl-8-benzyloxy-4-(piperidin-1-yl)-quinoline-2-carboxylate
Methyl-8-nitro-4-(piperidin-1-yl)-quinoline-2-carboxylate
Benzyl-4-benzyloxy-8-(piperidin-1-yl)-quinoline-2-carboxylate
Benzyl-4-benzyloxy-8-[benzyl(methyl)amino]-quinoline-2-carboxylate
Methyl-4-benzyloxy-8-(morpholin-1-yl)-quinoline-2-carboxylate
Methyl-4-benzyloxy-8-(4-benzyl-piperazin-1-yl)-quinoline-2-carboxylate
Methyl-4-benzyloxy-8-[phenyl(methyl)amino]-quinoline-2-carboxylate
Methyl-4-benzyloxy-8-(4-methyl-piperazin-1-yl)-quinoline-2-carboxylate
Methyl-4-benzyloxy-8-(pyridin-2-ylamino)-quinoline-2-carboxylate
Methyl-8-benzyloxy-4-(4-methyl-piperazin-1-yl)-quinoline-2-carboxylate
Methyl-8-benzyloxy-4-(3-aminoprop-1-yl)-quinoline-2-carboxylate
Methyl-8-benzyloxy-4-(3-benzoyl-aminoprop-1-yl)-quinoline-2-carboxylate
4-(hex-1-yl)-8-hydroxy-quinoline-2-carboxylic acid
8-hydroxy-4-(5-hydroxy-pent-1-yl)-quinoline-2-carboxylic acid
6-(3,5-dichlorophenyl)-4,8-dihydroxy-quinoline-2-carboxylic acid
6-(4-fluorophenyl)-4,8-dihydroxy-quinoline-2-carboxylic acid
4-hydroxy-8-(piperidin-1-yl)-quinoline-2-carboxylic acid hydrochloride
8-(4-benzyl-piperazin-1-yl)-4-hydroxy-quinoline-2-carboxylic acid hydrochloride
8-[phenyl(methyl)amino]-4-hydroxy-quinoline-2-carboxylic acid hydrochloride
8-(4-methyl-piperazin-1-yl)-4-hydroxy-quinoline-2-carboxylic acid hydrochloride
   and their pharmaceutically acceptable salts.

The following compounds are more particularly preferred:
4,8-dihydroxy-5-hydroxymethyl-quinoline-2-carboxylic acid
4,8-dihydroxy-6-bromo-quinoline-2-carboxylic acid
4-hydroxy-8-amino-quinoline-2-carboxylic acid
4-hydroxy-8-bromo-quinoline-2-carboxylic acid
4-hydroxy-8-benzyloxy-quinoline-2-carboxylic acid
8-benzyloxy-7-bromo-quinoline-2-carboxylic acid
8-hydroxy-4-phenyl-quinoline-2-carboxylic acid
8-hydroxy-4-(4-chloro-phenyl)-quinoline-2-carboxylic acid
4,8-dihydroxy-6-phenyl-quinoline-2-carboxylic acid
4,8-dihydroxy-6-(4-methoxy-phenyl)-quinoline-2-carboxylic acid
4,8-dihydroxy-6-(3-methyl-phenyl)-quinoline-2-carboxylic acid
4,8-dihydroxy-6-(4-chlorophenyl)-quinoline-2-carboxylic acid
4,8-dihydroxy-6-(3,4-dichlorophenyl)-quinoline-2-carboxylic acid
4,8-dihydroxy-6-(pyridin-3-yl)-quinoline-2-carboxylic acid
4-hydroxy-8-phenyl-quinoline-2-carboxylic acid
4,8-dihydroxy-6-(3-amino-propyl)quinoline-2-carboxylic acid
3-bromo-4,8-dihydroxy-quinoline-2-carboxylic acid
5,7-dichloro-4,8-dihydroxy-quinoline-2-carboxylic acid
3,7-dibromo-4,8-dihydroxy-quinoline-2-carboxylic acid
8-hydroxy-3-oxo-3,4-dihydroquinoxaline-2-carboxylic acid
4,8-dihydroxy-7-phenethyl-quinoline-2-carboxylic acid
4-hydroxy-8-(2H-tetrazol-5-yl)-quinoline-2-carboxylic acid hydrochloride
8-hydroxy-4-(morpholin-1-yl)-quinoline-2-carboxylic acid hydrochloride
8-hydroxy-4-(piperidin-1-yl)-quinoline-2-carboxylic acid hydrochloride
8-Amino-4-(piperidin-1-yl)-quinoline-2-carboxylic acid hydrochloride
4-hydroxy-8-(piperazin-1-yl)-quinoline-2-carboxylic acid hydrochloride
8-hydroxy-4-(4-methyl-piperazin-1-yl)-quinoline-2-carboxylic acid hydrochloride
4-hydroxy-8-phenylethyl-quinoline-2-carboxylic acid
8-hydroxy-4-(aminoprop-1-ynyl)-quinoline-2-carboxylic acid hydrochloride
Sodium 8-hydroxy-4-(piperazin-1-yl)-quinoline-2-carboxylate acid
4,8-dihydroxy-7-(3-aminoprop-1-yl)-quinoline-2-carboxylic acid hydrochloride
4,8-dihydroxy-5-trifluoromethyl-quinoline-2-carboxylic acid
4-hydroxy-8-[benzyl(methyl)amino]-quinoline-2-carboxylic acid hydrochloride
4-hydroxy-8-(morpholin-1-yl)-quinoline-2-carboxylic acid hydrochloride
4-hydroxy-8-(pyridin-2-yl-amino)-quinoline-2-carboxylic acid hydrochloride
8-hydroxy-4-[2-(1-piperazinyl)pyrimidinyl]-quinoline-2-carboxylic acid hydrochloride.

The compounds of the invention may be used in the form of pharmaceutically acceptable salts, particularly acid or base salts, preferably compatible with pharmaceutical use. Non-limiting examples of pharmaceutically acceptable acids include hydrochloric, hydrobromic, sulfuric, phosphoric, acetic, trifluoroacetic, lactic, pyruvic, malonic, succinic, glutaric, fumaric, tartaric, maleic, citric, ascorbic, methane or ethanesulfonic, camphoric acids, etc. Non-limiting examples of pharmaceutically acceptable bases include sodium hydroxide, potassium hydroxide, triethylamine, tert-butylamine, etc.

The invention also concerns the use of compounds represented by formula (I) for preparing a pharmaceutical composition for treating nervous system pathologies, especially mental or neurological disorders, particularly anxiety, depression, impairment of memory or social interactions, as sedative or hypnotic, for treating neurodegenerative diseases, such as Parkinson's disease, Alzheimer's disease or ALS, schizophrenia, some drug dependencies, particularly opioid, pain or obesity.

In particular, the invention concerns the use of compounds represented by formula (I) for preparing a pharmaceutical composition for treating mental or neurological disorders, particularly anxiety, depression, impairment of memory or social interactions, as sedative or hypnotic, or for treating neurodegenerative diseases, such as Parkinson's disease, Alzheimer's disease or ALS, some drug dependencies, particularly opioid.

The invention further concerns pharmaceutical compositions, particularly a medicament for treating central nervous system pathologies, containing as active principle at least one compound represented by formula (I) such as defined hereinabove or their pharmaceutically acceptable salts and a pharmaceutically acceptable vehicle or excipient.

The invention equally concerns novel compounds represented by formula (I) as such. More particularly these are compounds wherein R₁ is an unsubstituted alkyl or benzyl group or R₂ is a hydroxyl group, and preferably R₃ is (i) a hydrogen atom, (ii) a halogen atom, (iii) a hydroxyl group, (iv) a (C₁-C₁₂) alkyl group not substituted or substituted by amino or alkylamino, or (v) a (C₆-C₁₈) aryl group and/or Z is (i) a nitrogen atom or (ii) a CR₄ group in which R₄ represents (a) a hydrogen atom, (b) a (C₁-C₁₂) alkyl group not substituted or substituted by phenyl, hydroxyl or NR₉R_{9′} where R₉ is hydrogen or tert-butyloxycarbonyl and R_{9′} is hydrogen, (c) a (C₂-C₁₂) alcyn-1-yl group not substituted or substituted by phenyl, hydroxyl, benzyloxy or NR₉R_{9′} where R₉ represents tert-butyloxycarbonyl and R_{9′} is hydrogen, (d) a (C₆-C₁₈) aryl group not substituted or substituted by a halogen, (e) a OR₈ group in which R₈ represents hydrogen, (f) a NR₉R_{9′} group in which R₉ represents hydrogen or tosyl and R_{9′} is hydrogen, preferably a CR₄ group such as defined hereinabove.

The compounds or compositions of the invention may be administered in different ways and in different forms. For instance, they may be administered systemically, by the oral route, by inhalation or by injection, such as for example by the intravenous, intramuscular, subcutaneous, transdermal, intra-arterial route, etc., the intravenous, intramuscular, subcutaneous, oral and inhalation routes being preferred. For injections, the compounds are generally prepared in the form of liquid suspensions, which may be injected through syringes or by infusion, for instance. In this respect, the compounds are generally dissolved in pharmaceutically compatible saline, physiologic, isotonic, buffered solutions and the like, known to those skilled in the art. For instance, the compositions may contain one or more agents or vehicles chosen from among dispersives, solubilizers, stabilizers, preservatives, and the like. Agents or vehicles that may be used in the liquid and/or injectable formulations comprise in particular methylcellulose, hydroxymethylcellulose, carboxymethylcellulose, polysorbate 80, mannitol, gelatin, lactose, vegetable oils, acacia, and the like.

The compounds may also be administered in the form of gels, oils, tablets, suppositories, powders, capsules, gelules, aerosols, and the like, possibly by means of pharmaceutical forms or devices allowing sustained and/or delayed release. For this type of formulation, an agent such as cellulose, carbonates or starches is advantageously used.

It is understood that the injection rate and/or injected dose may be adapted by those skilled in the art according to the patient, the pathology, the mode of administration, etc. Typically, the compounds are administered at doses ranging from 0.1 to 500 mg/kg of body weight, more generally from 0.3 to 100 mg/kg, typically between 3 and 50 mg/kg. Furthermore, repeated injections may be given, as the case may be. In addition, in the case of chronic treatments, delayed or sustained release systems may be advantageous.

The inventive compounds are especially useful for modulating the activity of XA on the nervous system. As a matter of fact, the invention results from the demonstrated role of XA as a neurotransmitter. The compounds of the invention may also be used to selectively modulate neurotransmission, particularly dopaminergic neurotransmission. As illustrated in the examples, the compounds of the invention may be used either to inhibit XA activity, or to mimic or increase such activity. Thus, the results presented show that the inventive compounds are XA antagonists, XA agonists, or allosteric modulators of XA. For instance, compound 19f displays an activity which is ten-fold more potent than that of XA.

Furthermore, compounds 3x and 22c for example are capable of increasing XA binding to its receptor by more than 100-fold. The compounds particularly capable of increasing XA binding to its receptor preferably correspond to formula (I) wherein Z represents CR₄ in which R₄ is a hydroxyl group, X represents a halogen atom, preferably bromine, or a cyano group, and in particular at least one of the groups R₃, R₅, R₆ and R₇ is different from the hydrogen atom, advantageously with R₅ and R₇ not representing a halogen atom. Such compounds are also able to selectively increase dopamine levels in the prefrontal cortex (experiments carried out in rats in particular with measurements obtained by microdialysis). In particular they may be used to enhance memory and therefore to treat disorders of memory. They may thus be used to treat pathologies linked to senescence or neurodegeneration or even psychoses, such as schizophrenia. Lastly, other compounds such as compound 3u are potent inhibitors of XA binding to its site.

Compounds represented by formula (I) may be obtained from intermediates available commercially or possibly modified by carrying out a combination of chemical reactions known to those skilled in the art such as those described hereinbelow for functionalization of the products.

Compounds represented by formula (I) wherein Z is a nitrogen atom and E is a COOR₁ group or a CH₂COOR₁ group may be prepared according to the following reaction route: 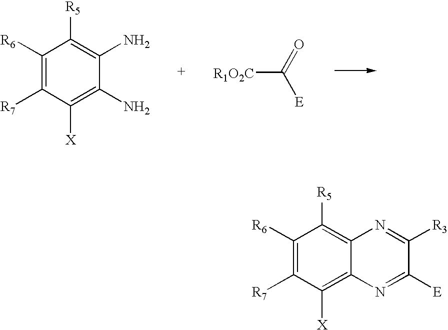
in such formulas, R₁, R₃, R₅, R₆, R₇ and X are defined as in formula (I) and E is COOR₁ or CH₂COOR₁.

This reaction is generally carried out according to the conditions described in J. Org. Chem., 1990, 55, 2820-2822. Preferably, the reaction is carried out in an inert solvent such as benzene or toluene in the presence of para-toluene sulfonic acid under reflex of the solvent.

Compounds represented by formula (I) wherein Z is a C—R₄ group, R₄ represents hydrogen and E is COOH, may be prepared according to the following reaction route: 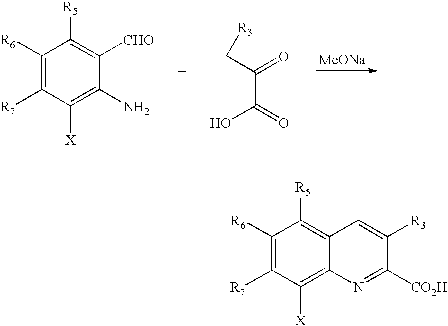
in these formulas, R₃, R₅, R₆, R₇ and X are defined as in formula (I).

This reaction is generally carried out in basic medium according to the conditions described in Tetrahedron Lett., 1984, 25, 923-926 or J. Am. Chem. Soc., 1998, 6, 1218-1222. Preferably it is carried out in the presence of sodium methylate, in an inert solvent such as an aliphatic alcohol like methanol, at a temperature of 60° C.

Compounds represented by formula (I) wherein Z is a C—R₄ group, R₄ is OR₈, R₈ is hydrogen, E is COOR₁ and R₁ is methyl, may be prepared according to the following reaction route: 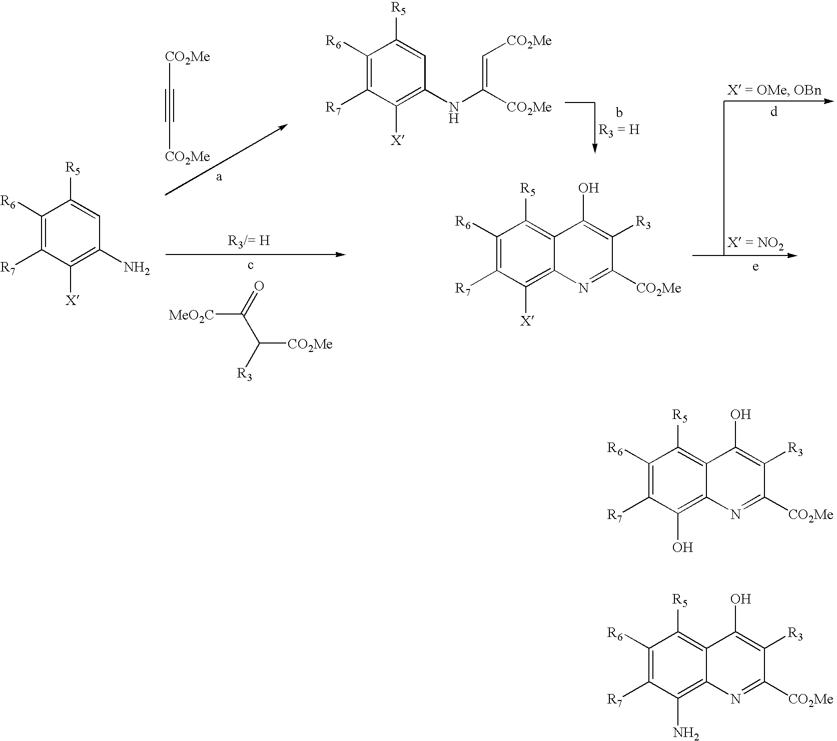
in these formulas, R₅, R₆ and R₇ are defined as in formula (I), X′ has the same definition as X and may also represent a nitro group, Me represents a methyl group and Bn represents a benzyl group.

The reaction is carried out according to the conditions described in J. Med. Chem., 1985, 28, 298-302 or J. Org. Chem. 1966, 31, 3369-3374. Preferably, an aliphatic alcohol such as methanol or ethanol is used, at the boiling temperature of the reaction medium.

Reactions b and c are generally carried out in diphenyl ether, at a temperature of 250° C. or in polyphosphoric acid at 100° C.

Deprotection of the hydroxyl group (reaction d) is carried out either when X′ is benzyloxy by catalytic hydrogenation in the presence of Pd/charcoal, in an inert solvent such as ethyl acetate, an aliphatic alcohol (methanol, ethanol for example) or acetic acid, at room temperature or with AlCl₃ in an inert solvent such as a chlorinated solvent (dichloroethane for example), in the presence of dimethylaniline, at room temperature or under reflux in a mixture of acetic acid/37% hydrochloric acid and, in this case, the ester is hydrolyzed simultaneously, or, when X′ is methoxy, in H₃PO₄ medium in the presence of KI and, in this case, hydrolysis of the ester occurs simultaneously.

Reduction of the nitro group (reaction e) is a catalytic reduction with hydrogen, in the presence of Pd/charcoal, in an inert solvent such as an aliphatic alcohol (methanol for example), at room temperature. However in the case where there is also a halogen present on the quinoline nucleus, the reduction of the nitro group to an amino group is carried out as described in J.O.C., 1985, 50, 26, 5782-5789 and, preferably, in THF with Na₂S₂O₄ at room temperature (approximately 18° C. to 25° C.) or at 60° C.

Compounds represented by formula (I) wherein Z is a C—R₄ group, R₄ is a halogen atom and E is a COOR₁ group may be prepared by reacting a corresponding compound represented by formula (I) in which Z is a C—R₄ group and R₄ is a hydroxyl group, with a halogenating agent.

It is preferable to use a phosphorus oxyhalogenide as halogenating agent (phosphorus oxychloride, phosphorus oxybromide, phosphorus oxyfluoride) according to the conditions described in Chem. Pharm. Bull., 1990, 2919-2925. Preferably the reaction is carried out at the boiling temperature of the reaction medium.

Compounds represented by formula (I) wherein Z is a CR₄ group, R₄ is a hydrogen atom, R₃ is a hydrogen atom and E is a COOR₁ group and the other substitutents are not halogen may be prepared by catalytic hydrogenation of compounds having formula (I) in which Z is a C—R₄ group, R₄ is a halogen atom.

Such hydrogenation is carried out with hydrogen, preferably, in the presence of Pd/charcoal, in an inert solvent such as an aliphatic alcohol (methanol for example) at room temperature and at variable pressure.

Compounds represented by formula (I) wherein Z is a C—R₄ group, R₄ is an aryl, heteroaryl or arylalkyl group and E is COOR₁ and/or one of the substituents R₅, R₆, R₇ or X is an aryl, heteroaryl or arylalkyl group may be prepared by reacting a corresponding compound represented by formula (I) in which Z is a C—R₄ group, R₄ is a halogen atom and/or one of the substituents R₅, R₆, R₇ or X is a halogen atom, with a derivative of formula R_{4′}B(OH)₂ for which R_{4′} is an aryl, heteroaryl or arylalkyl group. The halogen atoms are preferably chlorine or bromine atoms.

This reaction is preferably carried out in the presence of Pd(PPh₃)₄ and K₃PO₄, in an inert solvent such as dimethylformamide or toluene, at a temperature of 90° C. to 115° C.

Compounds represented by formula (I) wherein Z is a CR₄ group, R₄ is an alcyn-1-yl or (C₂-C₁₂)alkyl group and E is COOR₁ may be prepared by the following reaction route: 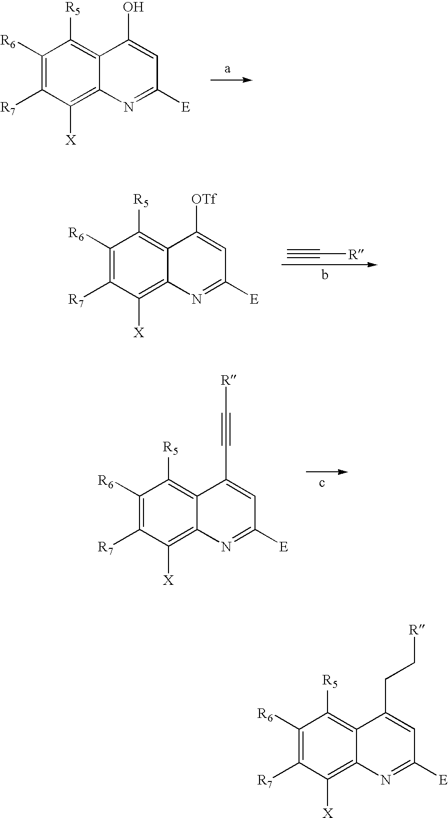
in these formulas R₅, R₆, R₇, X are defined as in formula (I), E is COOR₁, OTf represents a triflate group, R″ represents a hydrogen atom, an alkyl group, an aryl group or a heteroaryl group.

Reaction a is carried out with triflic anhydride in a chlorinated solvent such as dichloromethane, in the presence of pyridine, at a temperature ranging from 0° C. to 25° C.

Reaction b is generally carried out in the presence of a palladium halogenide such as PdCl₂, copper iodide, triphenylphosphine and triethylamine, in an inert solvent such as acetonitrile, at a temperature comprised between 30° C. and 60° C.

The reduction in reaction c is generally carried out by means of hydrogen, in the presence of Pd/charcoal, in an inert solvent such as a chlorinated solvent (dichloromethane for example), an aliphatic alcohol (methanol for example), or acetic acid, at room temperature.

Compounds represented by formula (I) wherein Z is a CR₄ group, R₄ is a NR₉R_{9′} group in which R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group or (v) a (C₁-C₁₇) heteroaryl group and R_{9′} which may be the same as or different from R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group or (v) a (C₁-C₁₇) heteroaryl group;
or NR₉R_{9′} represents a cycloheteroalkyl group of the type: 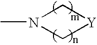
with n=2 or 3,
m=2 or 3
and Y represents CH₂, O, S, SO₂, or NR₁₁

R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;
and E is COOR₁ may be prepared according to the following reaction route:

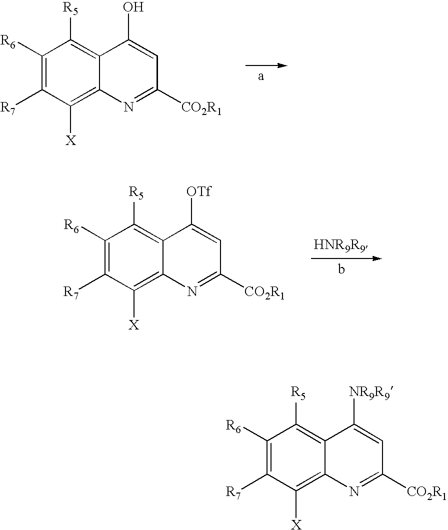

Reaction a is carried out by means of triflic anhydride, in a chlorinated solvent such as dichloromethane, in the presence of pyridine, at a temperature ranging from 0° C. to 25° C.

Reaction b is generally carried out in the presence of a palladium (0) (Pd₂(dba)₃) or palladium (II) complex such as Pd(OAc)₂ in the presence of the amine HNR₉R_{9′}, cesium carbonate and racemic BINAP in an inert solvent such as toluene, at a temperature comprised between 30° C. and 60° C.

Compounds represented by formula (I) wherein Z is a CR₄ group, R₄ is a OR₈ group and R₅, R₆, R₇ and/or X represents a NR₉R_{9′} group in which R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group or (v) a (C₁-C₁₇) heteroaryl group and R_{9′} which may be the same as or different from R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group or (v) a (C₁-C₁₇) heteroaryl group;
or NR₉R_{9′} represents a cycloheteroalkyl group of the type: 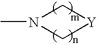
with n=2 or 3,
m=2 or 3,
and Y represents CH₂, O, S, SO₂, or NR₁₁

R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;
and E is COOR₁ may be prepared according to the following reaction route:

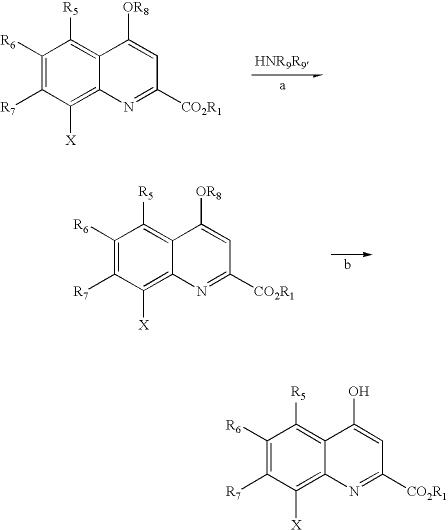

Reaction a is carried out with triflic anhydride, in a chlorinated solvent such as dichloromethane, in the presence of pyridine, at a temperature ranging from 0° C. to 25° C.

Reaction b is generally carried out in the presence of a palladium (0) (Pd₂(dba)₃) or palladium (II) complex such as Pd(OAc)₂ in the presence of the amine HNR₉R_{9′}, cesium carbonate and racemic BINAP in an inert solvent such as toluene, at a temperature comprised between 30° C. and 60° C.

The reduction in reaction c is generally carried out with hydrogen, in the presence of Pd/charcoal, in an inert solvent such as a chlorinated solvent (dichloromethane for example), an aliphatic alcohol (methanol for example), or acetic acid, at room temperature.

Compounds represented by formula (I) wherein Z is a CR₄ group, R₄ is a OR₈ group and R₅, R₆, R₇ and/or X represent a cyano group;
and E is COOR₁ may be prepared according to the following reaction route:

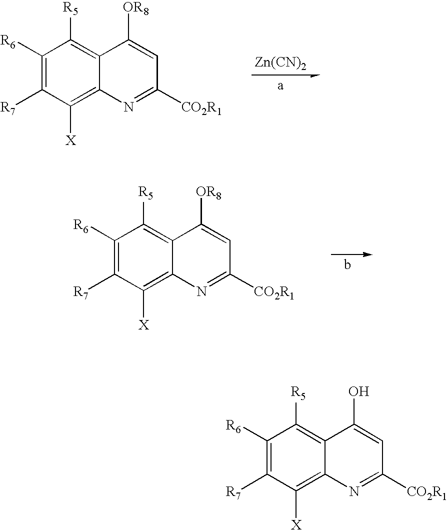

Reaction a is generally carried out in the presence of a palladium (II) complex (Pd(OAc)₂) in the presence of zinc cyanide amine, zinc powder, cesium carbonate and DPPF in an inert solvent such as DMA, at a temperature of 120° C.

The reduction in reaction a is generally carried out with hydrogen, in the presence of Pd/deactivated charcoal, in an inert solvent such as a chlorinated solvent (dichloromethane for example), an aliphatic alcohol (meethanol for example), or acetic acid, at room temperature.

Compounds represented by formula (I) wherein Z is a CR₄ group, R₄ is a NR₉R_{9′} group, R₉ is hydrogen or arylsulfonyl and R_{9′} is hydrogen and E is COOR₁ may be prepared according to the following reaction route: 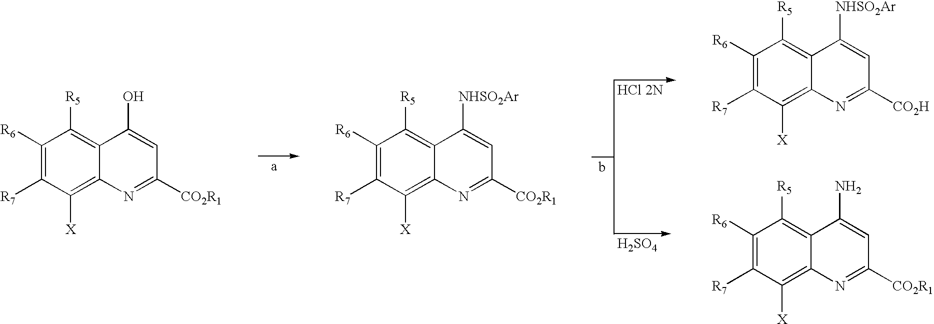
in these formulas, R₅, R₆, R₇, X are defined as in formula (I), E is COOR₁ and SO₂Ar is arylsulfonyl. Reaction a is carried out by reacting an arylsulfonylisocyanate according to the method described in J. Med. Chem., 1990, 33, 3130-3133. An inert solvent is preferably used, such as a chlorinated solvent (dichloromethane for example), at the boiling temperature of the reaction medium.

Deprotection reaction b is carried out in sulfuric acid at 0° C. When deprotection is carried out using hydrochloric acid, hydrolysis of the ester occurs and the compound in which Z is C—NR₉R_{9′} and R₉ is arylsulfonyl and R_{9′} is hydrogen and E is COOH is obtained.

Compounds represented by formula (I) wherein E is a CHO group may be prepared by oxidation of a compound represented by the formula: 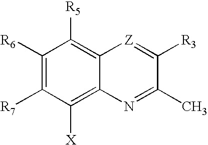
in which Z, R₃, R₅, R₆, R₇ and X are defined as in formula (I)

This oxidation is carried out by any method of oxidizing a methyl group to an aldehyde known to those skilled in the art and allowing the rest of the molecule to remain intact, such as the method described in Tetrahedron, 1996, 52, 4659-4672. Preferably, SeO₂ is used in an inert solvent such as dioxane, at a temperature of 80° C.

The methylated intermediates may be prepared according to one of the following reaction routes:
A 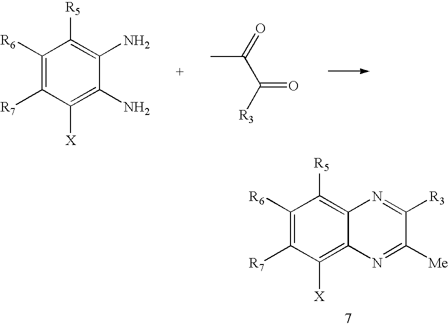
in these formulas, R₃, R₅, R₆, R₇ and X are defined as in formula (I) and Me is methyl.

This reaction is carried out according to the conditions described in J. Med. Chem., 1998, 41, 4062-4077 or J. Chem. Soc. 1946, 56-57. This condensation preferably takes place in acetic acid in the presence of sodium acetate at 60° C.

B— 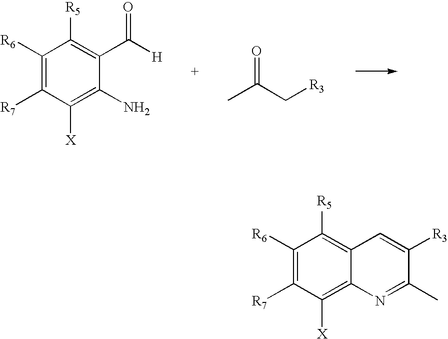
in these formulas, R₃, R₅, R₆, R₇ and X are defined as in formula (I). This reaction is carried out as described in Org. Prep. Proced. Int, 1991, 386-387. The reaction is preferably carried out in aqueous ethanol in the presence of a base such as KOH or piperidine at the boiling temperature of the reaction medium. 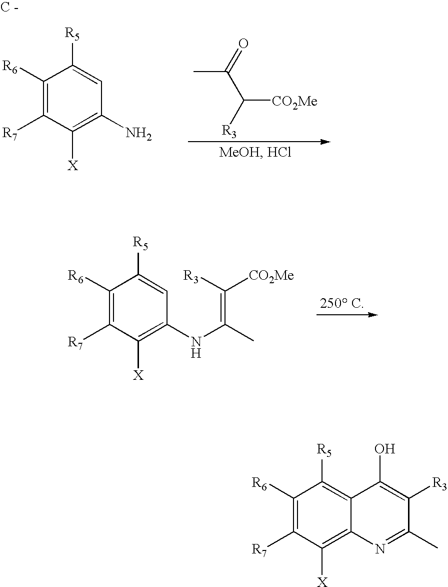
in these formulas, R₃, R₅, R₆, R₇ and X are defined as in formula (I).

This reaction is carried out in the conditions described in J. Chem. Soc, 1951, 1521-1527. Preferably, formation of the enamine is carried out in methanol in the presence of HCl. The second step takes place in diphenyl ether at 250° C.

The intermediates for which Z is CR₄ other than C—H and C—OH are then obtained by the methods described herein for preparing compounds represented by formula (I).

Compounds represented by formula (I) wherein E is a —CH₂OH group may be prepared by reducing a corresponding compound represented by formula (I) in which E is CHO.

This reduction is carried out by any method of oxidizing a methyl group to an aldehyde known to those skilled in the art and allowing the rest of the molecule to remain intact and, preferably, by means of sodium borohydride, in an inert solvent such as methanol or ethanol, at a temperature ranging from 0° C. to 25° C.

Compounds represented by formula (I) in which E is a COOH group may also be prepared by oxidizing a corresponding compound having formula (I) in which E is CHO.

This oxidation is carrried out under the conditions described in Tetrahedron, 1996, 52, 4659-4672. It is preferably carried out in H₂NSO₃H in the presence of aqueous sodium hypochlorite solution, in an inert organic solvent such as THF, at room temperature.

Compounds represented by formula (I) wherein E is a COOH group may be prepared by hydrolyzing corresponding compounds having formula (I) in which E is a COOR₁ group.

This reaction is carried out by any method known to those skilled in the art by which to convert an ester to the corresponding acid. Preferably the reaction occurs in acidic medium for example with hydrochloric acid in acetic acid or in alcoholic medium, at a temperature comprised between 20° C. and the boiling temperature of the reaction medium. The reaction may also be run in basic medium for example with LiOH, in an inert solvent such as water or tetrahydrofuran, at room temperature.

Compounds represented by formula (I) wherein E is a COOR₁ group and R₁ is alkyl may also be prepared by esterification of a corresponding compound having formula (I) in which E is a COOH group.

This esterification is carried out by any method of esterification of an acid known to those skilled in the art. In particular, an aliphatic acid (C₁-C₁₂ linear or branched) is reacted in the presence of an acid such as sulfuric acid or hydrochloric acid, at the boiling temperature of the reaction medium.

Compounds represented by formula (I) wherein E is a COOR₁ group and R₁ is arylalkyl may be prepared by reacting a corresponding compound having formula (I) in which E is a COOH group, with an arylalkyl halogenide.

This reaction is generally carried out in an inert solvent such as dimethylformamide, in the presence of NaH, at room temperature.

Compounds represented by formula (I) wherein E represents a CO—NHR₂ group may be prepared by reacting a corresponding compound having formula (I) in which E is a COOH or COOR₁ group and an amine H₂NR₂ in which R₂ is defined as in formula (I).

This reaction is generally carried out in an inert solvent such as THF in the presence of isobutyl chloroformiate at temperatures ranging from 0° C. to 25° C.

Compounds represented by formula (I) wherein E represents a tetrazolyl group may be prepared according to the following reaction route: 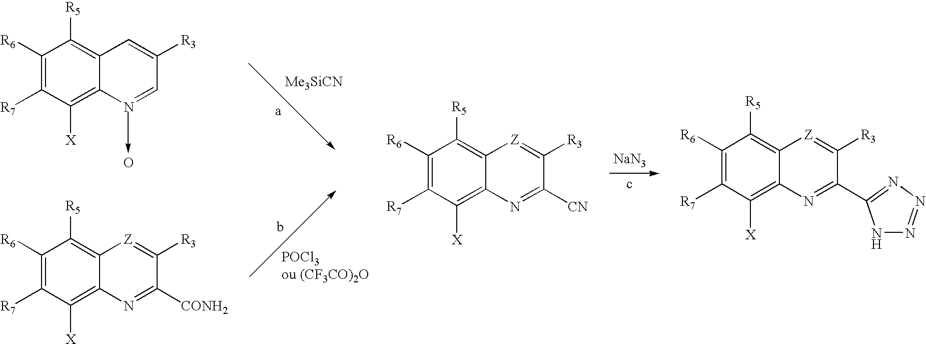
in these formulaes, R₃, R₅, R₆, R₇ and X are defined as in formula (I). Me is methyl.

Reaction a is carried out according to the conditions described in Synthesis, 1983, 4, 316-319. Preferably one uses acetonitrile under reflux of the solvent.

Dehydration of the amide (reaction b) is carried out by means of phosphorus oxychloride according to the method described in J. Med. Chem., 1988, 31, 84-91. The reaction is preferably carried out in DMF at 20° C. This dehydration may also be carried out using the conditions described in Org. Prep. Proc. Int, 1994, 26, 4, 429-438 and, preferably, in dichloromethane in the presence of trifluoroacetic anhydride and triethylamine at room temperature. Reaction c is carried out in DMF in the presence of ammonium chloride at a temperature of up to 120° C. (J. Med. Chem, 1979, 22, 7, 816-823).

Compounds represented by formula (I) wherein X represents a tetrazolyl group and R₁, R₃, R₅, R₆, R₇ and X are defined as in formula (I), may be prepared by reacting with sodium nitride in acetic acid medium at a temperature of up to 115° C.

Compounds represented by formula (I) wherein Z is a CR₄ group, R₄ is a hydrogen atom and R₃ is an arylmethyl group or methyl substituted by NR₉R_{9′} may be prepared by the following reaction route: 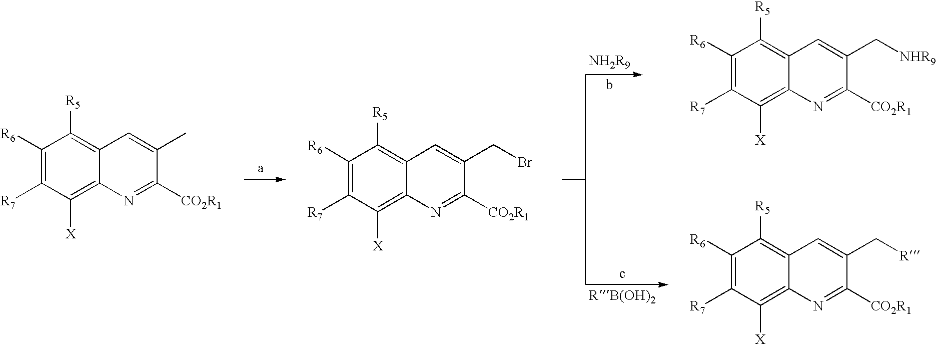
in these formulas, R₁, R₅, R₆, R₇, R₉ and X are defined as in formula (I), R′″ is an aryl group.

The bromination reaction a is carried out as described in J. Am. Chem. Soc., 1998, 120, 1218-1222. Preferably, 1,3-dibromo-5,5-dimethylhydantoin (DBH) is used in an inert solvent such as CCl₄ under reflux of the solvent.

Reaction b, in the case of aliphatic amines, is carried out according to the conditions described in Indian J. Chem. Sect B, EN 1984, 23, 1 33-39 and, preferably, in a solvent such as ethanol in the presence of NEt₃ under reflux. In the case of aromatic amines, the reaction may be carried out as described in J. Chem. Soc. Chem. Comm, EN, 1992, 18, 1300-1302 or J. Med. Chem. 1991, 2209-2218 and, preferably, in acetonitrile or benzene in the presence of K₂CO₃ or DMF, at temperatures ranging from 50° C. to 80° C.

Reaction c is carried out under the conditions described in Tetrahedron Lett. 1999, 40, 43, 7599-7603. One preferably uses Pd(PPh₃)₄ in an inert solvent such as DME in the presence of Na₂CO₃ under reflux of the solvent.

Compounds represented by formula (I) wherein one of the substituents R₃, R₅, R₆, R₇ or X is an alcyn-1-yl, (C₆-C₁₈)aryl(C₂-C₁₂)alcyn-1-yl, (C₄-C₁₂)heteroaryl(C₂-C₁₂)alcyn-1-yl, (C₂-C₁₂) alkyl, (C₆-C₁₈)aryl(C₂-C₁₂)alkyl, (C₄-C₁₂)heteroaryl(C₂-C₁₂) alkyl group, may be prepared by reacting a corresponding compound having formula (I) in which one of the substituents R₃, R₅, R₆, R₇ or X is a halogen atom, and preferably a bromine, with a derivative of formula:
≡—R″
in which R″ represents a hydrogen atom or an alkyl group, an aryl group or a heteroaryl group possibly followed by a reduction.

This reaction is generally carried out in the presence of a palladium halogenide such as PdCl₂, copper iodide, triphenylphosphine and triethylamine, in an inert solvent such as acetonitrile, at a temperature comprised between 30° C. and 60° C. The reduction is preferably carried out by means of hydrogen, in the presence of Pd/charcoal, in an inert solvent such as a chlorinated solvent (dichloromethane for example), an aliphatic alcohol (methanol for example) or acetic acid, at room temperature.

Compounds represented by formula (I) wherein one of the substituents R₃, R₅, R₆, R₇ or X is an alkyl or arylalkyl group may be prepared by reacting a corresponding compound having formula (I) in which one of the substituents R₃, R₅, R₆, R₇ or X is a halogen atom and, preferably, a bromine atom, with a derivative R′″ZnX″ in which R′″ is an alkyl or arylalkyl group and X″ is bromine or iodine.

This reaction is generally carried out in the presence of Pd(PPh₃)₄, in an inert solvent such as THF or DMF at a temperature ranging from 50° C. to 100° C.

Compounds represented by formula (I) wherein Z is C—R₄, R₄, R₃, R₆, R₇ are hydrogen, X is OR₈, R₈ is benzyl, E is COOR₁, R₁ is benzyl and R₅ is a bromine atom may be prepared by direct bromination of the corresponding compound having formula (I) in which Z is C—R₄, R₄, R₃, R₅, R₆, R₇ are hydrogen, X is OR₈, R₈ is benzyl, E is COOR₁, R₁ is benzyl.

This reaction is carried out as described in J. Chem. Soc, 1971, 3682-3653. It is preferably carried out with 2,4,4,6-tetrabromocyclohexa-2,5-dienone, in a chlorinated solvent such as dichloromethane at a temperature ranging from −10° C. to 25° C.

Compounds represented by formula (I) wherein Z is C—R₄, R₄ is hydroxyl and R₅, R₆, R₇ are hydrogen, X is OR₈, R₈ is benzyl, E is COOR₁, R₁ is methyl and R₃ is a bromine atom may be prepared by direct bromination of the corresponding compound having formula (I) in which Z is C—R₄, R₄, R₃, R₅, R₆, R₇ are hydrogen, X est OR₈, R₈ is benzyl, E is COOR₁, R₁ is methyl.

This reaction is preferably carried out by means of N-bromosuccinimide in the presence of diisopropylamine, in a chlorinated solvent such as dichloromethane at a temperature ranging from −10° C. to 25° C. or in carbon tetrachloride under reflux.

Compounds represented by formula (I) wherein one of the substitueants R₅, R₆, R₇ or X is a (C1)alkyl group substituted by NR₉R_{9′}, R₉ is alkyl, aryl or arylalkyl and R_{9′} is hydrogen, alkyl, aryl or arylalkyl may be prepared by reacting a corresponding compound in which one of the substituents R₃, R₅, R₆, R₇ or X is a CHO group with an amine corresponding to the formula HNR₉R_{9′}, R₉ is alkyl, aryl or arylalkyl and R_{9′} is hydrogen, alkyl, aryl or arylalkyl.

The intermediates for which X represents CHO may be obtained by the hereinabove methods.

This reaction is generally carried out in the presence of NaBH₄, in a protic solvent such as methanol, at a temperature of 0° C.

Compounds represented by formula (I) wherein one of the substituents R₅, R₆, R₇ or X is a NR₉R_{9′} group in which R₉ is COR₁₀ and R_{9′} is hydrogen or COR₁₀ may be prepared by reacting a corresponding compound having formula (I) in which one of the substituents R₅, R₆, R₇ or X is a NR₉R_{9′} group and R₉ and R_{9′} are hydrogen, with a derivative R₁₀COCl in which R₁₀ is defined as in formula (I).

This reaction is generally carried out in the presence of NEt₃ in acetonitrile at room temperature.

Compounds represented by formula (I) wherein one of the substituents R₅, R₆, R₇ or X is a NR₉R_{9′} group and R₉ is an alkyl group possibly substituted by aryl or arylalkyl and R_{9′} is hydrogen, alkyl, aryl or arylalkyl may be prepared by reacting a corresponding compound having formula (I) in which one of the substituents R₅, R₆, R₇ or X is a NR₉R_{9′} group and R₉ is hydrogen and R_{9′} is hydrogen with an aldehyde R_{9″}CHO in which R_{9″} is a hydrogen atom or an alkyl group possibly substituted by aryl or arylalkyl.

This reaction is generally carried out in the presence of NaBH₄, in an inert solvent such as an aliphatic alcohol and preferably methanol, at a temperature of 0° C.

The compounds containing an OR₈ where R₈ is alkyl or aryl may be prepared by reacting a corresponding compound having formula (I) containing an OR₈ group where R₈ is hydrogen with a derivative R₁₂Br in which R₁₂ is an alkyl or arylalkyl group.

This reaction is carried out in a solvent such as DMF using NaH as base at room temperature.

Compounds represented by formula (I) containing a NR₉R_{9′} group in which R₉ is hydrogen and R_{9′} is hydrogen may be prepared by reducing a corresponding compound containing a nitro group.

This reaction is generally carried out by any known method of reducing a nitro group. Preferably hydrogen is used, in the presence of Pd/charcoal, in an inert solvent such as an aliphatic alcohol (methanol for example), at room temperature. However, in the case where a halogen is also present, the reduction of the nitro group to amino is carried out as described in J.O.C., 1985, 50, 26, 5782-5789. THF is generally used with Na₂S₂O₄ at 60° C.

The intermediates containing a nitro group may be obtained by any of the hereinabove methods for preparing compounds represented by formula (I) from nitrated intermediates.

Compounds represented by formula (I) wherein one of the substituents R₅, R₆, R₇ or X is iodine may be prepared by reacting a corresponding compound having formula (I) in which one of the substituents R₅, 6, R₇ or X is a NR₉R_{9′} group in which R₉ is hydrogen and R_{9′} is hydrogen, with KI.

The reaction is carried out in aqueous medium in the presence of sulfuric acid, sodium nitrite and KI at a temperature ranging from 0° C. and 70° C.

In the hereinabove methods and in the examples, room temperature means a temperature comprised between 15° C. and 25° C.

The synthesis of some compounds represented by formula (I) may require the use of protector groups for certain functions which could interfere with the reaction. Such protector groups are chosen from among groups commonly used in organic synthesis.

For protection of amines, the benzyl, diphenylmethyl or butoxycarbonyl groups may be used for example. For protection of OH, one may use benzyl, tert-butyl or trialkylsilyl groups in particular.

The compounds obtained by the methods described hereinabove are then separated and purified by conventional methods (evaporation, chromatography, distillation, and the like).

The following examples are non-limiting illustrations of the invention. In the examples, the nitrated derivatives are intermediates. FIG. 1 illustrates the activity of the inventive compounds.

### EXAMPLE 1

### Methyl-4-hydroxy-6-bromo-8-methoxyquinoline-2-carboxylate 2k

### 1.1: Methyl 2-[(4-bromo-2-methoxyphenyl)amino]but-2-enedioate 1k

To a solution of 2-methoxy-4-bromoaniline (3.2 g, 15.84 mmol) in methanol (5 ml), under an inert atmosphere, add dropwise methyl acetylene dicarboxylate (2.81 g, 19.8 mmol). Heat under reflux for 1 hour, allow to cool and filter the resulting compound. Yield: 93%. ¹H-NMR (200 MHz, CDCl₃): δ 9.58 (broad s, 1H, NH), 7.01-6.96 (m, 2H arom.), 6.63 (d, 1H, J=9 Hz, 1H arom.), 5.44 (s, 1H vinylic), 3.85 (s, 3H, OCH₃), 3.75 (s, 3H, OCH₃) 3.74 (s, 3H, OCH₃).

### 1.2: Methyl-4-hydroxy-6-bromo-8-methoxyquinoline-2-carboxylate 2k

To diphenyl ether (10 ml) heated at 250° C., add the intermediate compound methyl-2-[(4-bromo-2-methoxyphenyl)amino]but-2-enedioate 1k (1 g, 2.91 mmol), and allow to react for 5 to 15 minutes. Then add 40 ml of petroleum ether and stir for 2 hours. Filter to obtain the abovenamed product 2k. Yield: 64%. ¹H-NMR (200 MHz, CDCl₃): δ 9.36 (broad s, 1H, OH), 8.06 (m, 1H arom.), 7.18 (m, 1H arom.), 6.99 (m, 1H arom.), 4.05 (s, 6H, 2 OCH₃).

### Methyl-4-hydroxy-8-benzyloxy-quinoline-2-carboxylate 2a

By replacing 2-methoxy-4-bromoaniline in example 1.1 by 2-benzyloxyaniline, the intermediate product methyl-2-[(2-benzyloxyphenyl)amino]-but-2-enedioate 1a is obtained. Yield: 86%. ¹H-NMR (200 MHz, CDCl₃): δ 9.77 (s, 1H, NH), 7.49-7.29 (m, 5H arom.), 7.05-6.78 (m, 4H arom.), 5.41 (s, 1H vinylic), 5.15 (s, 2H, OCH₂), 3.75 (s, 3H, OCH₃), 3.68 (s, 3H, OCH₃). Then, by replacing intermediate 1k in example 1.2 by the compound methyl-2-[(2-benzyloxyphenyl)amino]-but-2-enedioate 1a (obtained previously), the abovenamed product is obtained. Yield: 58%. ¹H-NMR (300 MHz, CDCl₃): δ 9.49 (broad s, 1H, OH), 7.91 (d, 1H, J=7 Hz, 1H arom.), 7.43 (m, 5H arom.), 7.26 (m, 1H arom.), 7.16 (d, 1H, J=7 Hz, 1H arom.), 6.99 (s, 1H arom.), 5.30 (s, 2H, OCH₂), 4.03 (s, 3H, OCH₃).

### Methyl-4-hydroxy-5-bromo-8-benzyloxy-quinoline-2-carboxylate 2b

By replacing 2-methoxy-4-bromoaniline in example 1.1 by 2-benzyloxy-5-bromo-aniline, the intermediate product methyl-2-[(2-benzyloxy-5-bromo-phenyl)amino]-but-2-enedioate 1b is obtained. Yield: 81%. ¹H-NMR (300 MHz, CDCl₃): δ 9.72 (broad s, 1H, NH), 7.44-7.32 (m, 5H arom.), 7.08 (d, 1H, J=8 Hz, 1H arom.), 6.90 (s, 1H arom.), 6.77 (d, 1H, J=8 Hz, 1H arom.), 5.48 (s, 1H vinylic), 5.11 (s, 2H, CH₂), 3.75 (s, 3H, OCH₃), 3.73 (s, 3H, OCH₃). Then, by replacing intermediate 1k in example 1.2 by the compound methyl-2-[(2-benzyloxy-5-bromo-phenyl)amino]-2-enedioate 1b (obtained previously), the abovenamed product methyl-4-hydroxy-5-bromo-8-benzyloxy-quinoline-2-carboxylate 2b is obtained. Yield: 71%. ¹H-NMR (300 MHz, CDCl₃): δ 9.37 (broad s, 1H, OH), 7.45 (m, 6H arom.), 6.94 (m, 2H arom.), 5.28 (s, 2H, OCH₂), 4.02 (s, 3H, OCH₃).

### Methyl-4-hydroxy-5-bromo-8-methoxy-quinoline-2-carboxylate 2d

By replacing 2-methoxy-4-bromoaniline in example 1.1 by 2-methoxy-5-bromoaniline, the intermediate product methyl-2-[(2-methoxy-5-bromo-phenyl)amino]-but-2-enedioate 1d is obtained. Yield: 92%. ¹H-NMR (200 MHz, CDCl₃): δ 9.75 (broad s, 1H, NH), 7.97 (m, 1H arom.), 7.60 (d, 1H, J=2 Hz, 1H arom.), 6.93 (d, 1H, J=8 Hz, 1H arom.), 5.60 (s, 1H, H vinylic), 4.00 (s, 3H, OCH₃), 3.78 (s, 3H, OCH₃).

Then, by replacing intermediate 1k in example 1.2 by the compound methyl-2-[(2-methoxy-5-bromo-phenyl)amino]-but-2-enedioate 1d (obtained previously), the abovenamed product is obtained. Yield: 92%. ¹H-NMR (200 MHz, CDCl₃): δ 9.44 (broad s, 1H, OH), 7.34 (d, 1H, J=8 Hz, 1H arom.), 7.02 (m, 2H arom.), 4.12 (s, 3H, OCH₃), 4.06 (s, 3H, OCH₃).

### Methyl-4-hydroxy-5-methyl-8-methoxy-quinoline-2-carboxylate 2e

By replacing 2-methoxy-4-bromoaniline in example 1.1 by 2-methoxy-5-methyl-aniline, the intermediate product methyl-2-[(2-methoxy-5-methyl-phenyl)amino]-but-2-enedioate 1e is obtained. Yield: 82%. ¹H-NMR (200 MHz, CDCl₃): δ 9.63 (broad s, 1H, NH), 6.85-6.61 (m, 3H arom.), 5.37 (s, 1H vinylic), 3.81 (s, 3H, OCH₃), 3.74 (s, 3H, OCH₃), 3.72 (s, 3H, OCH₃), 2.24 (s, 3H, CH₃).

Then, by replacing intermediate 1k in example 1.2 by the compound methyl-2-[(2-methoxy-5-methyl-phenyl)amino]-but-2-enedioate 1e (obtained previously), the abovenamed product is obtained. Yield: 83%. ¹H-NMR (200 MHz, CDCl₃): δ 9.30 (broad s, 1H, OH), 8.39 (m, 1H arom.), 6.95-6.93 (m, 1H arom.), 6.86 (m, 1H arom.), 4.02 (s, 3H, OCH₃), 3.99 (s, 3H, OCH₃), 2.82 (s, 3H, CH₃).

### Methyl-4-hydroxy-5-(1-hydroxy-ethyl)-8-methoxy-quinoline-2-carboxylate 2f

By replacing 2-methoxy-4-bromoaniline in example 1.1 by 2-methoxy-5-ethanol-aniline, the intermediate product methyl-2-[(2-methoxy-5-ethanol-phenyl)amino]-but-2-enedioate 1f is obtained in the same manner. Yield: 98%. ¹H-NMR (200 MHz, CDCl₃): δ 9.68 (broad s, 1H, NH), 7.02-6.81 (m, 3H arom), 5.40 (s, 1H vinylic), 4.79 (q, 1H, J=6 Hz, CH), 3.84 (s, 3H, OCH₃), 3.75 (s, 6H, 2×OCH₃), 1.44 (d, 3H, J=6 Hz, CH₃).

Then, by replacing intermediate 1k in example 1.2 by the compound methyl-2-[(2-methoxy-5-ethanol-phenyl)amino]-but-2-enedioate 1f (obtained previously), the abovenamed product is obtained in the same manner. Yield: 20%. ¹H-NMR (300 MHz, CDCl₃): δ 9.71 (broad s, 1H, OH), 7.29-7.02 (m, 3H arom), 5.21 (q, 1H, J=6 Hz, CH), 4.07 (s, 3H, OCH₃), 4.05 (s, 3H, OCH₃), 1.60 (d, 3H, J=6 Hz, CH₃).

### Methyl-4-hydroxy-5-hydroxymethyl-8-benzyloxy-quinoline-2-carboxylate 2g

By replacing 2-methoxy-4-bromoaniline in example 1.1 by 2-benzyloxy-5-hydroxymethyl-aniline and proceeding in the same manner, the intermediate product methyl-2-[(2-benzyloxy-5-hydroxymethyl-phenyl)amino]-but-2-enedioate 1g is obtained. Yield: 96%. ¹H-NMR (200 MHz, CDCl₃): δ 9.76 (broad s, 1H, NH), 7.43-7.34 (m, 5H arom.), 6.95-6.83 (m, 3H arom.), 5.42 (s, 1H vinylic), 5.13 (s, 2H, OCH₂), 4.56 (d, 2H, J=6 Hz, CH₂), 3.74 (s, 3H, OCH₃), 3.69 (s, 3H, OCH₃), 1.61 (t, 1H, J=6 Hz, OH).

Then, by replacing intermediate 1k in example 1.2 by the compound methyl-2-[(2-benzyloxy-5-hydroxymethyl-phenyl)amino]-but-2-enedioate 1g (obtained previosly), the abovenamed product is obtained in the same manner. Yield: 16%. ¹H-NMR (200 MHz, CDCl₃): δ 9.71 (broad s, 1H, OH), 7.74 (m, 5H arom.), 7.16-7.06 (m, 3H arom.), 5.31 (s, 2H, OCH₂), 4.84 (s, 2H, CH₂), 4.05 (s, 3H, OCH₃).

### Methyl-4-hydroxy-5,7-dichloro-8-methoxy-quinoline-2-carboxylate 2i

By replacing 2-methoxy-4-bromoaniline in example 1.1 by 2-methoxy-3,5-dichloroaniline, the intermediate product methyl-2-[(2-methoxy-3,5-dichloro-phenyl)amino]-but-2-enedioate 1i is obtained. Yield: 64%. ¹H-NMR (200 MHz, CDCl₃): δ 9.63 (s, 1H, NH), 7.06 (d, 1H, J=2 Hz, 1H arom.), 6.71 (d, 1H, J=2 Hz, 1H arom.), 5.60 (s, 1H vinylic), 3.78 (s, 3H, OCH₃), 3.77 (s, 3H, OCH₃), 3.76 (s, 3H, OCH₃).

Then, by replacing intermediate 1k in example 1.2 by the compound methyl-2-[(2-methoxy-3,5-dichloro-phenyl)amino]-but-2-enedioate 1i (obtained previously), the abovenamed product is obtained. Yield: 80%. ¹H-NMR (300 MHz, DMSO-d₆): δ 7.60 (s, 1H arom.), 6.85 (broad s, 1H arom.), 4.04 (s, 3H, OCH₃), 3.95 (s, 3H, OCH₃).

### Methyl-4-hydroxy-6-bromo-8-benzyloxy-quinoline-2-carboxylate 2l

By reaplacing 2-methoxy-4-bromoaniline in example 1.1 by 2-benzyloxy-4-bromoaniline, the intermediate product methyl-2-[(2-benzyloxy-4-bromophenyl)amino]-but-2-enedioate 1l is obtained. Yield: 98%. ¹H-NMR (200 MHz, CDCl₃): δ 9.66 (broad s, 1H, NH), 7.43-7.34 (m, 5H arom.), 7.07 (d, 1H, J=2 Hz, 1H arom.), 7.03-6.98 (dd, 1H, J=2 Hz, J=8 Hz, 1H arom.), 6.65 (d, 1H, J=8 Hz, 1H arom.), 5.45 (s, 1H, H vinylic), 5.11 (s, 2H, OCH₂), 3.74 (s, 3H, OCH₃), 3.68 (s, 3H, OCH₃).

Then, by replacing intermediate 1k in example 1.2 by the compound methyl-2-[(4-bromo-2-benzyloxyphenyl)amino]-but-2-enedioate 1l, the abovenamed product is obtained. Yield: 74%. ¹H-NMR (300 MHz, CDCl₃): δ 9.37 (broad s, 1H, OH), 8.07 (m, 1H arom.), 7.45 (m, 6H arom.), 6.98 (m, 1H arom.), 5.27 (s, 2H, OCH₂), 4.03 (s, 3H, OCH₃).

### Methyl-4-hydroxy-6-methyl-8-methoxy-quinoline-2-carboxylate 2n

By replacing 2-methoxy-4-bromoaniline in example 1.1 by 2-methoxy-4-methyl-aniline, the intermediate product methyl-2-[(2-methoxy-4-methylphenyl)amino]-but-2-enedioate 1n is obtained. Yield: 53%. ¹H-NMR (300 MHz, CDCl₃): δ 9.60 (broad s, 1H, NH), 6.68 (m, 3H arom.), 5.34 (s, 1H vinylic), 3.83 (s, 3H, OCH₃), 3.74 (s, 3H, OCH₃), 3.72 (s, 3H, OCH₃), 2.32 (s, 3H, CH₃).

Then, by replacing intermediate 1k in example 1.2 by the compound methyl-2-[(2-methoxy-4-methyl phenyl)amino]-but-2-enedioate 1n, the abovenamed product is obtained. Yield: 89%. ¹H-NMR (300 MHz, CDCl₃): δ 9.38 (broad s, 1H, OH), 7.70 (s, 1H arom.), 6.96-6.92 (m, 2H arom.), 4.03 (s, 3H, OCH₃), 4.02 (s, 3H, OCH₃), 2.47 (s, 3H, CH₃).

### Methyl-4-hydroxy-6-formyl-8-methoxy-quinoline-2-carboxylate 2o

By replacing 2-methoxy-4-bromoaniline in example 1.1 by 2-methoxy-4-formyl-aniline, the intermediate product methyl-2-[(2-methoxy-4-formylphenyl)amino]-but-2-enedioate 1o is obtained Yield: 81%. ¹H-NMR (200 MHz, CDCl₃): δ 9.85 (s, 2H, CHO and NH), 7.41-7.35 (m, 2H arom.), 6.76 (d, 1H, J=8 Hz, 1H arom.), 5.62 (s, 1H vinylic), 3.97 (s, 3H, OCH₃), 3.80 (s, 3H, OCH₃), 3.78 (s, 3H, OCH₃).

Then, by replacing intermediate 1k in example 1.2 by the compound methyl-2-[(2-methoxy-4-formylphenyl)amino]-but-2-enedioate 1o, the abovenamed product is obtained. Yield: 96%. ¹H-NMR (200 MHz, CDCl₃): δ 10.03 (s, 1H, CHO), 9.52 (broad s, 1H, OH), 8.39 (m, 1H arom.), 7.59 (m, 1H arom.), 7.05 (m, 1H arom.), 4.12 (s, 3H, OCH₃), 4.07 (s, 3H, OCH₃).

### Methyl-4-hydroxy-6-nitro-8-methoxy-quinoline-2-carboxylate 2p

By replacing 2-methoxy-4-bromoaniline in example 1.1 by 2-methoxy-4-nitroaniline, the intermediate product methyl-2-[(2-methoxy-4-nitro-phenyl)amino]-but-2-enedioate 1p is obtained. Yield: 78%. ¹H-NMR (200 MHz, DMSO-d₆): δ 9.91 (s, 1H, NH), 7.92 (m, 2H arom.), 7.02 (d, 1H, J=9 Hz, 1H arom.), 5.70 (s, 1H vinylic), 4.05 (s, 3H, OCH₃), 3.86 (s, 3H, OCH₃), 3.79 (s, 3H, OCH₃).

Then, by replacing intermediate 1k in example 1.2 by the compound methyl-2-[(2-methoxy-4-nitro-phenyl)amino]-but-2-enedioate 1p (obtained previously), the abovenamed product is obtained. Yield: 63%. ¹H-NMR (200 MHz, DMSO-d₆): δ 10.50 (broad s, 1H, OH), 8.46 (d, 1H, J=2 Hz, 1H arom.), 7.96 (d, 1H, J=2 Hz, 1H arom.), 6.89 (broad s, 1H, 1H arom.), 4.16 (s, 3H, OCH₃), 3.98 (s, 3H, OCH₃).

### Methyl 4-hydroxy-8-nitro-quinoline-2-carboxylate 2r

By replacing 2-methoxy-4-bromoaniline in example 1.1 by 2-nitroaniline, the intermediate product methyl-2-[(2-nitro-phenyl)amino]-but-2-enedioate 1r is obtained. Yield: 34%. ¹H-NMR (300 MHz, CDCl₃): δ 11.12 (broad s, 1H, NH), 8.13 (d, 1H, J=8 Hz, 1H arom.), 7.46 (m, 1H, 1H arom.), 7.08 (m, 1H, 1H arom.), 6.75 (d, 1H, J=8 Hz, 1H arom.), 5.84 (s, 1H vinylic), 3.81 (s, 3H, OCH₃), 3.75 (s, 3H, OCH₃).

Then, by replacing intermediate 1k in example 1.2 by the compound methyl-2-[(2-nitro-phenyl)amino]-but-2-enedioate 1r (obtained previously), the abovenamed product is obtained. Yield: 58%. ¹H-NMR (300 MHz, CDCl₃): δ 11.79 (s, 1H, OH), 8.74 (m, 2H arom.), 7.50 (m, 1H arom.), 7.08 (s, 1H arom.), 4.09 (s, 3H, OCH₃).

### Methyl-4-hydroxy-5-methyl-8-nitro-quinoline-2-carboxylate 2s

By replacing 2-methoxy-4-bromoaniline in example 1.1 by 2-nitro-5-methylaniline, the intermediate product methyl-2-[(2-nitro-5-methyl-phenyl)amino]-but-2-enedioate 1s is obtained. Yield: 69%. ¹H-NMR (200 MHz, CDCl₃): δ 11.13 (broad s, 1H, NH), 8.07 (d, 1H, J=8 Hz, 1H arom.), 6.90 (d, 1H, J=8 Hz, 1H arom.), 6.54 (s, 1H arom.), 5.80 (s, 1H vinylic), 3.88 (s, 3H, OCH₃), 3.82 (s, 3H, OCH₃), 2.30 (s, 3H, CH₃).

Then, by replacing intermediate 1k in example 1.2 by the compound methyl-2-[(2-nitro-5-methyl-phenyl)amino]-but-2-enedioate 1s (obtained previously), the abovenamed product is obtained. Yield: 46%. ¹H-NMR (200 MHz, CDCl₃): δ 12.06 (broad s, 1H, OH), 8.56 (d, 1H, J=9 Hz, 1H arom.), 7.17 (d, 1H, J=9 Hz, 1H arom.), 6.99 (s, 1H arom.), 4.07 (s, 3H, OCH₃), 3.00 (s, 3H, CH₃).

### Methyl-4-hydroxy-6-methyl-8-nitro-quinoline-2-carboxylate 2t

By replacing 2-methoxy-4-bromoaniline in example 1.1 by 2-nitro-4-methylaniline, the intermediate product methyl-2-[(2-nitro-4-methyl-phenyl)amino]-but-2-enedioate 1t is obtained. Yield: 70%. ¹H-NMR (200 MHz, CDCl₃): δ 11.02 (broad s, 1H, NH), 7.95 (s, 1H arom.), 7.20 (d, 1H, J=7 Hz, 1H arom.), 6.70 (d, 1H, J=7 Hz, 1H arom.), 5.77 (s, 1H vinylic), 3.81 (s, 3H, OCH₃), 3.82 (s, 3H, OCH₃), 2.37 (s, 3H, CH₃).

Then, by replacing intermediate 1k in example 1.2 by the compound methyl-2-[(2-nitro-4-methyl-phenyl)amino]-but-2-enedioate 1s (obtained previously), the abovenamed product is obtained. Yield: 23%. ¹H-NMR (200 MHz, CDCl₃): δ 11.65 (s, 1H, OH), 8.51 (broad s, 2H arom.), 7.01 (s, 1H arom.), 4.06 (s, 3H, OCH₃), 2.53 (s, 3H, CH₃).

### Methyl-4-hydroxy-8-bromo-quinoline-2-carboxylate 2x

By replacing 2-methoxy-4-bromoaniline in example 1.1 by 2-bromoaniline, the intermediate product methyl-2-[(2-bromo-phenyl)amino]-but-2-enedioate 1x is obtained. Yield: 43%. ¹H-NMR (200 MHz, CDCl₃): δ 9.75 (s, 1H, NH), 7.56 (d, 1H, J=8 Hz, 1H arom.), 7.19 (m, 1H arom.), 6.94 (m, 1H arom.), 6.76 (d, 1H, J=8 Hz, 1H arom.), 5.55 (s, 1H vinylic), 3.77 (s, 3H, OCH₃), 3.71 (s, 3H, OCH₃).

Then, by replacing intermediate 1k in example 1.2 by the compound methyl-2-[(2-bromo-phenyl)amino]-but-2-enedioate 1x (obtained previously), the abovenamed product is obtained. Yield: 94%. ¹H-NMR (300 MHz, CDCl₃): δ 9.37 (broad s, 1H, OH), 8.31 (d, 1H, J=7 Hz, 1H arom.), 7.90 (d, 1H, J=7 Hz, 1H arom.), 7.27 (m, 1H arom.), 7.00 (s, 1H arom.), 4.08 (s, 3H, OCH₃).

### Methyl 4-hHydroxy-6-isopropyl-8-bromo-quinoline-2-carboxylate 42b

By replacing 2-methoxy-4-bromoaniline in example 1.1 by 2-bromo-4-isopropylaniline, the intermediate product methyl-2-[(2-bromo-4-isopropylphenyl)amino]-but-2-enedioate 42a is obtained. Yield: 90%. ¹H-NMR (200 MHz, CDCl₃): δ 9.70 (s, 1H, NH), 7.43-7.42 (d, 1H, J=1.96 Hz, 1H arom.), 7.07-7.02 (dd, J=8 Hz & J=1.96 Hz, 1H arom.), 6.73-6.69 (d, J=8 Hz, 1H arom.), 5.49 (s, 1H vinylic), 3.76 (s, 3H, OCH₃), 3.72 (s, 3H, OCH₃), 2.95-2.75 (sept, 1H, CH(CH3)₂), 1.24 and 1.20 (2s, 6H, CH(CH₃)₂).

Then, by replacing intermediate 1k in example 1.2 by the compound methyl-2-[(2-bromo-4-isopropylphenyl)amino]-but-2-enedioate 42a (obtained previously), the abovenamed product is obtained. Yield: 71%. ¹H-NMR (300 MHz, CDCl₃): δ 9.35 (broad s, 1H, OH), 8.16 (d, 1H, J=1.89 Hz, 1H arom.), 7.80 (d, 1H, J=1.86 Hz, 1H arom.), 7.27 (m, 1H arom.), 6.98 (d, J=1.89 Hz, 1H arom.), 4.06 (s, 3H, OCH₃), 3.07-2.98 (sept, 1H, CH(CH₃)₂), 1.32 and 1.3 (2s, 6H, CH(CH₃)₂).

### Methyl-8-benzyloxy-5,7-dichloro-4-hydroxyquinoline-2-carboxylate 44b

By replacing 2-methoxy-4-bromoaniline in example 1.1 by 2-benzyloxy-3,5-dichloroaniline, the intermediate product methyl-2-[(2-benzyloxy-3,5-dichlorophenyl)amino]-but-2-enedioate 44a is obtained Yield: 62%. ¹H-NMR (300 MHz, CDCl₃): δ 9.60 (broad s, 1H, NH), 7.44-7.28 (m, 5H arom.), 7.10 (d, 1H arom.), 6.73 (d, 1H arom.), 5.54 (s, 1H vinylic), 4.93 (s, 2H, OCH₂), 3.75 (s, 3H, OCH₃), 3.70 (s, 3H, OCH₃).

Then, by replacing intermediate 1k in example 1.2 by the compound methyl-2-[(2-benzyloxy-3,5-dichlorophenyl)amino]-but-2-enedioate 44a (obtained previously), the abovenamed product is obtained. Yield: 48%. ¹H-NMR (300 MHz, CDCl₃): δ 8.98 (broad s, 1H, OH), 7.50-7.36 (m, 5H arom.), 7.34 (s, 1H arom.), 6.80 (d, 1H arom.), 5.23 (s, 2H, OCH₂), 3.97 (s, 3H, OCH₃).

### Methyl-7-bromo-8-benzyloxy-4-hydroxy-quinoline-2-carboxylate 50b

By replacing 2-methoxybromoaniline in example 1.1 by 3-bromo-2-benzyloxyaniline, methyl-2-[(3-bromo-2-benzyloxyphenyl)amino]but-2-enedioate 50a is obtained as a yellow solid. Yield: 58%. ¹H NMR (200 MHz, CDCl₃): δ 9.66 (s, 1 HN), 7.53 (m, 2H, 2H arom.), 7.36 (m, 4H, 4H arom.), 6.93 (t, 1H, J=8 Hz, H⁵), 6.81 (dd, 1H, J=2 and 8 Hz, H arom.), 5.50 (s, 1H vinylic), 4.96 (s, 2H, CH₂benz), 3.76 (s, 3H, OCH₃), 3.68 (s, 3H, OCH₃). By replacing intermediate 1k in example 1.2 by the compound methyl-2-[(3-bromo-2-benzyloxyphenyl)amino]but-2-enedioate 50a, the abovenamed product is obtained as a yellow solid. Yield: 42%. ¹H NMR (300 MHz, CDCl₃): δ 9.00 (s, 1 HO), 7.96 (d, 1H, J=8 Hz, H⁶), 7.43 (d, 1H, J=8 Hz, H⁵), 7.42 (m, 5H, 5H arom.), 6.87 (s, 1H³), 5.24 (s, 2H, CH₂benz), 4.10 (s, 3H, OCH₃).

### Methyl-6-bromo-8-cyano-4-hydroxy-quinoline-2-carboxylate 63b

By rerplacing 2-methoxy-4-bromoaniline in example 1.1 by 4-bromo-2-cyanoaniline, the intermediate product methyl-2-[(4-bromo-2-cyano-phenyl)amino]-but-2-enedioate 63a is obtained Yield: 80%. ¹H-NMR (200 MHz, CDCl₃): δ 9.90 (broad s, 1H, NH), 7.70-7.69 (d, 1H, J=2 Hz, 1H arom.), 7.58-7.53 (dd, 1H, J=2 Hz and 9 Hz, 1H arom.), 6.72-6.68 (d, 1H, J=9 Hz, 1H arom.), 5.80 (s, 1H vinylic), 3.79 (s, 3H, OCH₃), 3.78 (s, 3H, OCH₃).

Then, by replacing intermediate 1k in example 1.2 by the compound methyl-2-[(4-bromo-2-cyano-phenyl)amino]-but-2-enedioate 63a (obtained previously), the abovenamed product is obtained. Yield: 94%. ¹H-NMR (200 MHz, CDCl₃): δ 12.72 (broad s, 1H, OH), 8.67-8.58 (m, 2H arom.), 7.57 (s, 1H arom.), 3.97 (s, 3H, OCH₃).

### Methyl-5-bromo-8-nitro-4-hydroxy-quinoline-2-carboxylate 72b

By replacing 2-methoxybromoaniline in example 1.1 by 5-bromo-2-nitroaniline, methyl-2-[(5-bromo-2-nitrophenyl)amino]but-2-enedioate 72a is obtained as an orange solid. Yield: 33%. ¹H NMR (200 MHz, CDCl₃): δ 11.13 (s, 1 HN), 8.03 (d, 1H, J=10 Hz, H³), 7.19 (dd, 1H, J=2 and 10 Hz, H⁴), 6.90 (d, 1H, J=2 Hz, H⁶), 5.94 (s, 1H vinylic), 3.82 (s, 6H, 2 OCH₃).

Then, by replacing intermediate 1k in example 1.2 by the methyl-2-[(4-bromo-2-nitrophenyl)amino]but-2-enedioate 72a, the abovenamed product is obtained as a yellow solid. Yield: 28%. ¹H NMR (300 MHz, CDCl₃): □ 12.05 (s, 1 HO), 8.45 (d, 1H, J=9 Hz, H⁷), 7.70 (d, 1H, J=9 Hz, H⁵), 7.05 (s, 1H³), 4.09 (s, 3H, OCH₃).

### Methyl-6-benzyloxy-8-nitro-4-hydroxy-quinoline-2-carboxylate 79b

By replacing 2-methoxybromoaniline in example 1.1 by 4-benzyloxy-2-nitroaniline, methyl-2-[(4-benzyloxy-2-nitrophenyl)amino]but-2-eedioate 79a is obtained as a red solid. Yield: 82%. ¹H NMR (300 MHz, CDCl₃): δ 10.92 (s, 1 HN), 7.72 (s, 1H³), 7.42 (m, 5H arom.), 7.15 (d, 1H, J=8 Hz, H⁵), 6.75 (d, 1H, J=8 Hz, H⁶), 5.75 (s, 1H vinylic), 5.10 (s, 2H benz.), 3.81 (s, 3H, OCH₃), 3.74 (s, 3H, OCH₃).

Then, by replacing intermediate 1k in example 1.2 by the methyl-2-[(4-benzyloxy-2-nitrophenyl)amino]but-2-enedioate 79a, one obtains, after purification by silica chromatography (eluent: EtOAc/CH₂Cl₂, 92:8), the abovenamed product is obtained as an orange solid. ¹H NMR (300 MHz, CDCl₃): δ 11.64 (s, 1 HO), 8.40 (d, 1H, J=3 Hz, H⁵), 8.29 (d, 1H, J=3 Hz, H⁷), 7.40 (m, 5H arom.), 7.02 (s, 1H³), 5.24 (s, 2H benz.), 4.09 (s, 3H, OCH₃).

### Methyl-6-chloro-8-nitro-4-hydroxy-quinoline-2-carboxylate 117b

By replacing 2-methoxybromoaniline in example 1.1 by 4-chloro-2-nitroaniline, methyl-2-[(4-chloro-2-nitrophenyl)amino]but-2-enedioate 117a is obtained as an orange solid. Yield: 52%. ¹H NMR (200 MHz, CDCl₃): δ 9.69 (s, 1 HN), 7.43 (d, 1H, J=8 Hz, H⁵), 7.37 (d, 1H, J=2 Hz, H³), 6.99 (dd, 1H, J=2 and 8 Hz, H⁵), 5.66 (s, 1H vinylic), 3.80 (s, 3H, OCH₃), 3.77 (s, 3H, OCH₃).

Then, by replacing intermediate 1k in example 1.2 by the methyl-2-[(4-chloro-2-nitrophenyl)amino]but-2-enedioate 117a, the abovenamed product is obtained as a yellow solid. Yield: 73%. ¹H NMR (200 MHz, CDCl₃): δ 10.97 (s, 1 HO), 8.44 (d, 1H, J=2 Hz, H⁷), 8.40 (d, 1H, J=2 Hz, H⁵), 7.17 (s, 1H³), 4.03 (s, 3H, OCH₃).

### Methyl-6-bromo-8-nitro-4-hydroxy-quinoline-2-carboxylate 31a

By replacing 2-methoxybromoaniline in example 1.1 by 4-bromo-2-nitroaniline, methyl-2-[(4-bromo-2-nitrophenyl)amino]but-2-enedioate 31b is obtained as an orange solid. Yield: 49%. ¹H NMR (200 MHz, CDCl₃): δ 11.07 (s, 1 HN), 8.29 (d, 1H, J=2 Hz, H³), 7.55 (dd, 1H, J=2 and 9 Hz, H⁵), 6.63 (d, 1H, J=9 Hz, H⁶), 5.91 (s, 1H vinylic), 3.82 (s, 3H, OCH₃), 3.78 (s, 3H, OCH₃).

Then, by replacing intermediate 1k in example 1.2 by the methyl-2-[(4-bromo-2-nitrophenyl)amino]but-2-enedioate 31b, the abovenamed product is obtained as a yellow solid. Yield: 31%. ¹H NMR (300 MHz, CDCl₃): δ 11.67 (s, 1 HO), 8.84 (d, 1H, J=2 Hz, H⁷), 8.80 (d, 1H, J=2 Hz, H⁵), 7.07 (s, 1H³), 4.10 (s, 3H, OCH₃).

### Methyl-8-cyano-4-hydroxy-quinoline-2-carboxylate 35b

By replacing 2-methoxybromoaniline in example 1.1 by 2-cyanoaniline, methyl-2-[(2-cyanophenyl)amino]but-2-enedioate 35a is obtained as a yellow solid. Yield: 87%. ¹H NMR (200 MHz, CDCl₃): 9.92 (s, 1 HN), 7.58 (d, 1H, J=7 Hz, H³), 7.45 (t, 1H, J=7 Hz, H 4), 7.13 (t, 1H, J=7 Hz, H⁵), 6.82 (d, 1H, J=7 Hz, H⁶), 5.73 (s, 1H vinylic), 3.78 (s, 3H, OCH₃), 3.75 (s, 3H, OCH₃).

Then, by replacing intermediate 1k in example 1.2 by the methyl-2-[(2-cyanophenyl)amino]but-2-enedioate 35a, the abovenamed product is obtained as a white solid. Yield: 82%. ¹H NMR (300 MHz, CDCl₃): δ 11.67 (s, 1 HO), 8.84 (d, 1H, J=2 Hz, H⁷), 8.80 (d, 1H, J=2 Hz, H⁵), 7.07 (s, 1H³), 4.10 (s, 3H, OCH₃).

### Methyl-8-fluoro-4-hydroxy-quinoline-2-carboxylate 36b

By replacing 2-methoxybromoaniline in example 1.1 by 2-fluoroaniline, methyl-2-[(2-fluorophenyl)amino]but-2-enedioate 36a is obtained as a yellow solid. Yield: 58%. ¹H NMR (300 MHz, CDCl₃): δ 9.60 (s, 1 HN), 7.07 (m, 2H, H⁵ and H³), 6.94 (t, 1H, J=8 Hz, H⁴), 5.54 (s, 1H vinylic), 3.77 (s, 3H, OCH₃), 3.75 (s, 3H, OCH₃).

Then, by replacing intermediate 1k in example 1.2 by the methyl-2-[(2-fluorophenyl)amino]but-2-enedioate 36a, the abovenamed product is obtained as a white solid. Yield: 91%. ¹H NMR (200 MHz, CDCl₃): δ 9.09 (s, 1 HO), 8.10 (d, 1H, J=8 Hz, H⁷), 7.41 (d, 1H, J=10 Hz, H⁵), 7.32 (dd, 1H, J=8 and 10 Hz, H⁶), 6.98 (s, 1H³), 4.06 (s, 3H, OCH₃).

### Methyl-8-carboxamide-4-hydroxy-quinoline-2-carboxylate 37b

By replacing 2-methoxybromoaniline in example 1.1 by anthralinamide, methyl-2-[(2-carboxamidephenyl)amino]but-2-enedioate 37a is obtained as a white solid. Yield: 94. %. ¹H NMR (300 MHz, CDCl₃): δ 11.00 (s, 1 HN), 7.62 (d, 1H, J=8 Hz, H arom.), 6.36 (t, 1H, J=8 Hz, H arom.), 7.08 (t, 1H, J=8 Hz, H arom.), 6.75 (d, 1H, J=8 Hz, H arom.), 6.09 (m, 2H, CONH₂), 5.60 (s, 1H vinylic), 3.80 (s, 3H, OCH₃), 3.77 (s, 3H, OCH₃).

Then, by replacing intermediate 1k in example 1.2 by the methyl-2-[(2-carboxamidephenyl)amino]but-2-enedioate 37a the abovenamed product is obtained as a brown solid. Yield: 78%. ¹H NMR (200 MHz, CDCl₃): δ 9.09 (s, 1 HO), 8.10 (d, 1H, J=8 Hz, H⁷), 7.41 (d, 1H, J=10 Hz, H⁵), 7.32 (dd, 1H, J=8 and 10 Hz, H⁶), 6.98 (s, 1H³), 4.06 (s, 3H, OCH₃).

### Methyl-4-hydroxy-8-nitro-6-phenyl-quinoline-2-carboxylate 86b

By replacing 2-methoxy-4-bromoaniline in example 1.1 by 2-nitro-4-phenylaniline, the intermediate product methyl-2-[(3-nitro-1,1′-biphenyl-4-yl)amino]-but-2-enedioate 86a is obtained. Yield: 83%. ¹H-NMR (300 MHz, CDCl₃): δ 11.18 (broad s, 1H, NH), 8.39 (s, 1H arom.), 7.72 (d, 1H, J=8 Hz, 1H arom.), 7.60-7.39 (m, 5H arom.), 6.82 (d, 1H, J=8 Hz, 1H arom.), 5.87 (s, 1H vinylic), 3.83 (s, 3H, OCH₃), 3.79 (s, 3H, OCH₃).

Then, by replacing intermediate 1k in example 1.2 by the compound methyl-2-[(3-nitro-1,1′-biphenyl-4-yl)amino]-but-2-enedioate 86a (obtained previously), the abovenamed product is obtained. Yield: 65%. ¹H-NMR (200 MHz, CDCl₃): δ 11.76 (broad s, 1H, OH), 8.98 (s, 2H arom.), 7.75-7.46 (m, 5H arom.), 7.09 (s, 1H arom.), 4.10 -(s, 3H, OCH₃).

### Methyl-8-benzyloxy-4-hydroxy-5-trifluoromethyl-quinoline-2-carboxylate 98b

By replacing 2-methoxy-4-bromoaniline in example 1.1 by 2-benzyloxy-5-trifluoromethyl-aniline, the intermediate product methyl-2-[(2-benzyloxy-5-trifluoromethyl-phenyl)amino]-but-2-enedioate 98a is obtained. Yield: 86%. ¹H-NMR (300 MHz, CDCl₃): δ 9.83 (broad s, 1H, NH), 7.45-7.23 (m, 6H arom.), 6.99-76.95 (m, 2H arom.), 5.53 (s, 1H vinylic), 5.20 (s, 2H, OCH₂), 3.76 (s, 3H, OCH₃), 3.71 (s, 3H, OCH₃).

Then, by replacing intermediate 1k in example 1.2 by the compound methyl-2-[(2-benzyloxy-5-trifluoromethyl-phenyl)amino]-but-2-enedioate 98a (obtained previously), the abovenamed product is obtained. Yield: 58%. ¹H-NMR (200 MHz, CDCl₃): δ 9.46 (broad s, 1H, OH), 7.67 (d, 1H, J=8 Hz, 1H arom.), 7.47-7.42 (d, 5H arom.), 7.10 (d, 1H, J=8 Hz, 1H arom.), 6.97 (s, 1H arom.), 5.36 (s, 2H, OCH₂), 4.03 (s, 3H, OCH₃).

### Methyl-7-bromo-4-hydroxy-2-carboxylate 112b and Methyl-5-bromo-4-hydroxy-2-carboxylate 112b′

By replacing 2-methoxy-4-bromoaniline in example 1.1 by 2-benzyloxy-5-trifluoromethylaniline, the intermediate product methyl-2-[(3-bromo-phenyl)amino]-but-2-enedioate 112a is obtained Yield: 83%. ¹H-NMR (200 MHz, CDCl₃): δ 9.62 (broad s, 1H, NH), 7.28-7.05 (m, 3H arom.), 6.83-6.78 (m, 1H arom.), 5.48 (s, 1H vinylic), 3.75 (s, 3H, OCH₃), 3.74 (s, 3H, OCH₃).

Then, by replacing intermediate 1k in example 1.2 by the compound methyl-[(3-bromo-phenyl)amino]-but-2-enedioate 112a (obtained previously), a mixture of the two abovenamed products is obtained. Yield: 92%. The two isomers are separated after benzylation (see example 10, compounds 112c and 112d).

### EXAMPLE 2

### Methyl-4,8-dihydroxy-5-hydroxymethyl-quinoline-2-carboxylate 2h

To methyl-4-hydroxy-5-hydroxymethyl-8-benzyloxy-quinoline-2-carboxylate 2g (102 mg, 0.3 mmol) dissolved in degassed acetic acid, ethyl acetate or methanol (15 ml), add palladium/charcoal (10%) (30 mg) then place everything under hydrogen at atmospheric pressure overnight. Filter on celite, wash twice with CH₂Cl₂/MeOH: 8/2 then evaporate to dryness. Triturate in diethyl ether and filter to obtain the abovenamed product. Yield: 97%. ¹H-NMR (200 MHz, DMSO-d₆): δ 10.83 (broad s, 1H, OH), 9.43 (broad s, 1H, OH), 7.30-6.90 (m, 2H arom.), 6.51 (s, 1H arom.), 4.89 (s, 1H, OH), 3.97 (s, 3H, OCH₃), 2.69 (s, 2H, CH₂).

### Methyl-8-hydroxy-4-(toluene-4-sulfonylamino)-quinoline-2-carboxylate 15b

To methyl-8-benzyloxy-4-(toluene-4-sulfonylamino)-quinoline-2-carboxylate 15a (0.3 mmol) dissolved in degassed acetic acid, ethyl acetate or methanol (15 ml), add palladium/charcoal (10%) (30 mg) then place everything under hydrogen at atmospheric pressure overnight. Filter on celite, wash twice with CH₂Cl₂/MeOH: 8/2 then evaporate to dryness. Triturate in diethyl ether and filter to obtain the abovenamed product. Yield: 100%. ¹H-NMR (200 MHz, methanol-d₄): δ 8.14 (s, 1H arom.), 7.95 (d, 2H, J=9 Hz, 2H arom.), 7.80 (d, 1H, J=8 Hz, 1H arom.), 4.47 (m, 1H arom.), 7.42 (d, 2H, J=9 Hz, 2H arom.), 7.22 (d, 1H, J=8 Hz, 1H arom.), 4.17 (s, 3H, OCH₃), 2.46 (s, 3H, CH₃).

### Methyl-4,8-dihydroxy-5-phenyl-quinoline-2-carboxylate 18b

By replacing methyl-8-benzyloxy-4-(toluene-4-sulfonylamino)-quinoline-2-carboxylate 15a in example 2 by methyl-4-hydroxy-8-benzyloxy-5-phenyl-quinoline-2-carboxylate 18a and proceeding in the same manner, the abovenamed product is obtained. Yield: 84%. ¹H-NMR (200 MHz, CDCl₃): δ 9.30 (broad s, 1H, OH), 8.04 (m, 1H arom.), 7.63-7.22 (m, 6H arom.), 6.87 (s, 1H arom.), 4.02 (s, 3H, OCH₃).

### Methyl-4,8-dihydroxy-6-(4-methoxy-phenyl)-quinoline-2-carboxylate 18h

By replacing methyl-8-benzyloxy-4-(toluene-4-sulfonylamino)-quinoline-2-carboxylate 15a in example 2 by methyl-4-hydroxy-6-(4-methoxy-phenyl)-8-benzyloxy-quinoline-2-carboxylate 18g and proceeding in the same manner, the abovenamed product is obtained. Yield: 95%. ¹H-NMR (200 MHz, methanol-d₄): δ 7.93 (m, 1H arom.), 7.65 (m, 1H arom.), 7.60 (m, 1H arom.), 7.40 (m, 1H arom.), 7.02-6.98 (m, 3H arom.), 4.07 (s, 3H, OCH₃), 3.87 (s, 3H, OCH₃).

### Methyl-4,8-dihydroxy-6-(3-methyl-phenyl)-quinoline-2-carboxylate 18j

By replacing methyl-8-benzyloxy-4-(toluene-4-sulfonylamino)-quinoline-2-carboxylate 15a in example 2 by methyl-4-hydroxy-6-(3-methyl-phenyl)-8-benzyloxy-quinoline-2-carboxylate 18i and proceeding in the same manner, the abovenamed product is obtained. Yield: 51%. ¹H-NMR (300 MHz, methanol-d₄): δ 7.90 (m, 1H arom.), 7.54 (m, 2H arom.), 7.39 (m, 2H arom.), 7.23 (m, 1H arom.), 7.02 (m, 1H arom.), 4.10 (s, 3H, OCH₃), 2.45 (s, 3H, CH₃).

### Methyl-4,8-dihydroxy-6-(pyridin-3-yl)-quinoline-2-carboxylate 18p

By replacing methyl-8-benzyloxy-4-(toluene-4-sulfonylamino)-quinoline-2-carboxylate 15a in example 2 by methyl-8-benzyloxy-4-hydroxy-6-(pyridin-3-yl)-quinoline-2-carboxylate 18o and proceeding in the same manner, the abovenamed product is obtained. Yield: 60%. ¹H-NMR (300 MHz, CDCl₃): δ 9.35 (s, 1H arom.), 8.95 (m, 2H arom.), 8.17-7.96 (m, 2H arom.), 7.69 (m, 1H arom.), 7.18 (m, 1H arom.), 4.10 (s, 3H, OCH₃).

### Methyl-4,8-dihydroxy-6-benzyl-quinoline-2-carboxylate 23b

By replacing methyl-8-benzyloxy-4-(toluene-4-sulfonylamino)-quinoline-2-carboxylate 15a in example 2 by methyl-8-benzyloxy-6-benzyl-4-hydroxy-quinoline-2-carboxylate 23a and proceeding in the same manner, the abovenamed product is obtained. Yield: 95%. ¹H-NMR (300 MHz, methanol-d₄): δ 7.69-6.94 (m, 8H arom.), 4.09 (s, 3H, OCH₃), 4.07 (s, 2H, CH₂).

### Ethyl-8-hydroxy-3-oxo-3,4-dihydroquinoxaline-2-carboxylate 59b

By replacing methyl-8-benzyloxy-4-(toluene-4-sulfonylamino)-quinoline-2-carboxylate 15a in example 2 by ethyl-8-(benzyloxy)-3-oxo-3,4-dihydroquinoxaline-2-carboxylate 59a and proceeding in the same manner, the abovenamed product is obtained. Yield: 97%. ¹H-NMR (300 MHz, methanol-d₄): δ 6.63-6.58 (t, 1H, J=8 Hz, 1H arom.), 6.52-6.49 (d, 1H, J=8 Hz, 1H arom.), 6.39-6.37 (d, 1H, J=8 Hz, 1H arom.), 4.21-4.14 (q, 2H, J=7 Hz, OCH₂—CH₃), 1.24-1.20 (q, 3H, J=7 Hz, OCH₂—CH₃).

### Ethyl[8-(hydroxy)-3-oxo-3,4-dihydroquinoxaline-2(1H)-ylilene]acetate 60b

By replacing methyl-8-benzyloxy-4-(tolueene-4-sulfonylamino)-quinoline-2-carboxylate 15a in example 2 by ethyl[8-(benzyloxy)-3-oxo-3,4-dihydroquinoxaline-2(1H)-ylilene]acetate 60a and proceeding in the same manner, the abovenamed product is obtained. Yield: 62%. ¹H-NMR (200 MHz, DMSO-d₆): δ 10.25 (s, 1H, OH), 9.52 (broad s, 1H, NHCO), 6.55-6.28 (m, 3H arom.), 5.18 (s, 1H, C═CH), 4.13-4.03 (q, 2H, J=7 Hz, OCH₂—CH3), 1.23-1.16 (t, 3H, J=7 Hz, OCH₂CH₃).

### 3-ethyl-8-nitro-4-hydroxy-quinoline-2-carboxylic acid 57c

By replacing methyl-8-benzyloxy-4-(toluene-4-sulfonylamino)-quinoline-2-carboxylate 15a in example 2 by 3-ethynyl-8-nitro-4-hydroxy-quinoline-2-carboxylic acid 57b, the abovenamed product is obtained. Yield: 86%. ¹H NMR (300 MHz, d₆-DMSO): δ 12.02 (s, 1H, OH), 9.64 (s, 1H, OH), 8.01 (d, 1H, J=8 Hz, H arom.), 7.43 (t, 1H, J=8 Hz, 1H⁶), 7.34 (d, 1H, J=8 Hz, H arom.), 3.64 (s, 2H, NH₂), 2.24 (dd, 2H, J=8 and 10 Hz, CH₂), 1.34 (dd, 3H, J=8 and 10 Hz, CH₃).

### Methyl-3-phenylethyl-8-amino-4-hydroxy-quinoline-2-carboxylate 68b

By replacing methyl-8-benzyloxy-4-(toluene-4-sulfonylamino)-quinoline-2-carboxylate 15a in example 2 by methyl-3-phenylethynyl-8-nitro-4-hydroxy-quinoline-2-carboxylate 68a, the abovenamed product is obtained. Yield: 85%. ¹H NMR (300 MHz, CDCl₃): δ 9.16 (s, 1H, OH), 7.96 (d, 1H, J=8 Hz, H arom.), 7.18 (m, 7H, 7H arom.), 3.99 (s, 3H, OCH₃), 3.59 (s, 2H, NH₂), 3.34 (dd, 2H, J=8 and 10 Hz, CH₂), 2.84 (dd, 2H, J=8 and 10 Hz, CH₂).

### 3-(3-hydroxypropyl)-8-amino-4-hydroxy-quinoline-2-carboxylic acid 70c

By replacing methyl-8-benzyloxy-4-(toluene-4-sulfonylamino)-quinoline-2-carboxylate 15a in example 2 by benzyl-3-(3′-N-(terbutoxycarbonyl)aminoprop-1′-ynyl)-8-nitro-4-hydroxy-quinoline-2-carboxylate 70b, the abovenamed product is obtained. Yield: 70%. ¹H NMR (300 MHz, d₆-DMSO): δ 12.14 (s, 1H, OH), 9.42 (s, 1H, OH), 7.38 (t, 1H, J=8 Hz, H⁶), 7.24 (d, 1H, J=8 Hz, 1H arom), 6.80 (d, 1H, J=8 Hz, 1H arom.), 5.24 (s, 2 HN), 3.52 (s, 1H, OH), 3.22 (m, 2H, CH₂O), 3.35 (m, 3H, CH₂), 2.41 (m, 2H, CH₂), 1.80 (m, 2H, CH₂).

### 3-(3′-N-tert-butoxycarbonyl-propyl)-8-amino-4-hydroxy-quinoline-2-carboxylic acid 71c

By replacing methyl-8-benzyloxy-4-(toluene-4-sulfonylamino)-quinoline-2-carboxylate 15a in example 2 by benzyl-3-(3′-N-(terbutoxycarbonyl)aminoprop-1′-ynyl)-8-nitro-4-hydroxy-quinoline-2-carboxylate 71b, the abovenamed product is obtained. Yield: 70%. ¹H NMR (300 MHz, d₆-DMSO): δ 7.50 (t, 1H, J=8 Hz, H 6), 7.24 (m, 2H, 1 OH and 1H arom), 6.86 (d, 1H, J=9 Hz, 1H arom.), 6.64 (s, 2 HN), 4.41 (m, 2H, CH₂N), 3.35 (m, 3H, CH₂ and HN), 1.35 (m, 2H, CH₂), 1.66 (s, 9H, 3 CH₃).

### Methyl-5-phenyl-8-amino-4-hydroxy-quinoline-2-carboxylate 73b

By replacing methyl-8-benzyloxy-4-(toluene-4-sulfonylamino)-quinoline-2-carboxylate 15a in example 2 by methyl-5-phenyl-8-nitro-4-benzyloxy-quinoline-2-carboxylate 73a, the abovenamed product is obtained. Yield: 94%. ¹H NMR (300 MHz, CDCl₃): δ 10.45 (s, 1H, OH), 7.21 (m, 6H, 6H arom.), 7.12 (d, 1H, J=8 Hz, H arom.), 6.88 (d, 1H, J=8 Hz, H arom.), 4.96 (s, 2H, NH₂), 3.98 (s, 3H, OCH₃).

### Methyl-5-phenylethyl-8-amino-4-hydroxy-quinoline-2-carboxylate 74b

By replacing methyl-8-benzyloxy-4-(toluene-4-sulfonylamino)-quinoline-2-carboxylate 15a in example 2 by methyl-5-phenylethynyl-8-nitro-4-benzyloxy-quinoline-2-carboxylate 74a, the abovenamed product is obtained. Yield: 76%. ¹H NMR (300 MHz, CDCl₃): δ 9.15 (s, 1H, OH), 7.18 (m, 7H, 7H arom.), 6.88 (d, 1H, J=8 Hz, H arom.), 4.04 (s, 3H, OCH₃), 3.96 (s, 2H, NH₂), 3.54 (t, 2H, J=9 Hz, CH₂), 2.95 (t, 2H, J=9 Hz, CH₂).

### Methyl-5-hydroxypropyl-8-amino-4-hydroxy-quinoline-2-carboxylate 75b

By replacing methyl-8-benzyloxy-4-(toluene-4-sulfonylamino)-quinoline-2-carboxylate 15a in example 2 by methyl-5-(3′-benzyloxyprop-1′-ynyl)-8-nitro-4-benzyloxy-quinoline-2-carboxylate 75a, the abovenamed product is obtained. Yield: 64%. ¹H NMR (200 MHz, CDCl₃): δ 10.54 (s, 1H, OH), 7.32 (d, 1H, J=8 Hz, H arom.), 6.90 (d, 1H, J=8 Hz, H arom.), 6.88 (s, 1H, H³), 5.42 (s, 2H, NH₂), 4.00 (s, 3H, OCH₃), 3.86 (s, 1H, OH), 3.44 (m, 2H, CH₂O), 2.71 (m, 4H, 2 CH₂), 1.80 (m, 2H, CH₂).

### Methyl-5-(3′-N-(terbutoxycarbonyl)aminopropyl)-8-amino-4-hydroxy-quinoline-2-carboxylate 76b

By replacing methyl-8-benzyloxy-4-(toluene-4-sulfonylamino)-quinoline-2-carboxylate 15a in example 2 by methyl-5-(3′-N-(terbutoxycarbonyl)aminoprop-1′-ynyl)-8-nitro-4-benzyloxy-quinoline-2-carboxylate 76a, the abovenamed product is obtained. Yield: 79%. ¹H NMR (300 MHz, CDCl₃): δ 9.34 (s, 1H, OH), 7.40 (d, 1H, J=8 Hz, H arom.), 6.96 (d, 1H, J=8 Hz, H arom.), 6.90 (s, 1H, H³), 5.33 (s, 1H, NH), 4.06 (s, 3H, OCH₃), 3.47 (s, 2H, NH₂), 3.30 (m, 4H, 2 CH₂), 1.82 (m, 2H, CH₂).

### Methyl-5-piperidin-1-yl-8-amino-4-hydroxy-quinoline-2-carboxylate 77a

By replacing methyl-8-benzyloxy-4-(toluene-4-sulfonylamino)-quinoline-2-carboxylate 15a in example 2 by methyl-5-piperidin-1-yl-8-nitro-4-benzyloxy-quinoline-2-carboxylate 77a, the abovenamed product is obtained as a yellow solid. Yield: 82%. ¹H NMR (200 MHz, CDCl₃): δ 7.42 (s, 1H³), 7.22 (d, 1H, J=8 Hz, H arom.), 6.84 (d, 1H, J=8 Hz, H arom.), 5.09 (s, 2 HN), 4.00 (s, 3H, OCH₃), 3.17 (m, 2H, NCH₂), 2.88 (m, 2H, NCH₂), 1.85 (m, 6H, 3 CH₂).

### Methyl-5-piperazin-1-yl-8-amino-4-hydroxy-quinoline-2-carboxylate 78b

By replacing methyl-4-hydroxy-5-hydroxymethyl-8-benzyloxy-quinoline-2-carboxylate 2g in example 2 by methyl-5-(N-(N-benzyl)piperazinyl)-8-nitro-4-benzyloxy-quinoline-2-carboxylate 78a, the abovenamed product is obtained as an orange solid. Yield: 61%. ¹H NMR (200 MHz, d₆-DMSO): δ 10.82 (s, 1H, OH), 7.40 (s, 1H³), 7.12 (d, 1H, J=8 Hz, H arom.), 6.92 (d, 1H, J=8 Hz, H arom.), 5.50 (s, 2H, H₂N), 5.02 (s, 1H, NH), 3.99 (s, 3H, OCH₃), 3.10 (m, 2H, NCH₂), 2.98 (m, 2H, NCH₂), 2.70 (m, 6H, 3 CH₂), 2.51 (m, 2H, CH₂).

### Methyl-4,6-dihydroxy-8-amino-quinoline-2-carboxylate 79c

By replacing methyl-8-benzyloxy-4-(toluene-4-sulfonylamino)-quinoline-2-carboxylate 15a in example 2 by methyl-6-benzyloxy-8-nitro-4-hydroxy-quinoline-2-carboxylate 79b, the abovenamed product is obtained. Yield: 85%. ¹H NMR (200 MHz, d₆-DMSO): δ 11.10 (s, 1 OH), 9.66 (s, 1 OH), 7.33 (s, 1H³), 6.51 (d, 1H, J=2 Hz, H⁵), 6.44 (d, 1H, J=2 Hz, H⁷), 5.90 (s, 2 HN.), 3.86 (s, 3H, OCH₃).

### Methyl-6-phenylethyl-8-amino-4-hydroxy-quinoline-2-carboxylate 118b

By replacing methyl-8-benzyloxy-4-(toluene-4-sulfonylamino)-quinoline-2-carboxylate 15a in example 2 by methyl-6-phenylethynyl-8-nitro-4-benzyloxy-quinoline-2-carboxylate 118a, the abovenamed product is obtained. Yield: 92%. ¹H NMR (300 MHz, CDCl₃): δ 11.43 (s, 1H, OH), 7.43 (s, 1H, H arom.), 7.24 (m, 5H, 5H arom.), 7.15 (s, 1H, H arom.), 5.89 (s, 2H, NH₂), 3.93 (s, 3H, OCH₃), 2.95 (m, 4H, 2 CH₂).

### Methyl-6-(3′-hydroxypropyl)-8-amino-4-hydroxy-quinoline-2-carboxylate 119b

By replacing methyl-8-benzyloxy-4-(toluene-4-sulfonylamino)-quinoline-2-carboxylate 15a in example 2 by methyl-6-(3′-benzyloxypropyn-1′-yl)-8-nitro-4-benzyloxy-quinoline-2-carboxylate 119a, the abovenamed product is obtained. Yield: 78%. ¹H NMR (300 MHz, d₆-DMSO): δ 11.51 (s, 1 HO), 7.40 (s, 1H³), 7.09 (d, 1H, J=2 Hz, 1H arom.), 6.77 (d, 1H, J=2 Hz, 1H arom.), 5.86 (s, 2 HN), 4.48 (s, 1 HO), 3.94 (s, 3H, OCH₃), 3.42 (m, 2H, CH₂O), 2.73 (m, 2H, CH₂), 1.78 (m, 2H, CH₂).

### Methyl-6-(3′-N-(tert-butoxycarbonyl)aminopropyl)-8-amino-4-hydroxy-quinoline-2-carboxylate 121b

By replacing methyl-8-benzyloxy-4-(toluene-4-sulfonylamino)-quinoline-2-carboxylate 15a in example 2 by methyl-6-(3′-N-(terbutoxycarbonyl)aminoprop-1′-ynyl)-8-nitro-4-benzyloxy-quinoline-2-carboxylate 121a, the abovenamed product is obtained. Yield: 73%. ¹H NMR (300 MHz, DMSO-d₆): δ 9.22 (s, 1 HO), 7.70 (s, 1H³), 6.97 (m, 2H arom), 4.64 (s, 2 HN), 4.02 (s, 3H, OCH₃), 3.75 (s, 1 HN), 3.15 (m, 2H, CH₂N), 2.70 (m, 2H, CH₂), 1.84 (m, 2H, CH₂), 1.66 (s, 9H, 3 CH₃).

### Methyl-6-(3′-pyridinyl)ethyl-8-amino-4-hydroxy-quinoline-2-carboxylate 122b

By replacing methyl-8-benzyloxy-4-(toluene-4-sulfonylamino)-quinoline-2-carboxylate 15a in example 2 by methyl-6-(3′-pyridinylethynyl)-8-nitro-4-benzyloxy-quinoline-2-carboxylate 122a, the abovenamed product is obtained as a yellow solid. Yield: 82%. ¹H NMR (200 MHz, d₆-DMSO): δ 8.45 (s, 1H, H arom.), 8.40 (d, 1H, J=2 Hz, H arom.), 7.69 (d, 1H, J=5 Hz, 1H arom.), 7.34 (m, 2H, 2H arom.), 7.13 (s, 1H, 1H arom.), 6.83 (s, 1H, 1H arom.), 5.90 (s, 2 HN), 3.92 (s, 3H, OCH₃), 2.97 (m, 4H, 2 CH₂).

### Methyl-6-(5′-cyanopentyl)-8-amino-4-hydroxy-quinoline-2-carboxylate 123b

By replacing methyl-8-benzyloxy-4-(toluene-4-sulfonylamino)-quinoline-2-carboxylate 15a in example 2 by methyl-6-(5′-cyanopent-1′-ynyl)-8-nitro-4-benzyloxy-quinoline-2-carboxylate 123a, the abovenamed product is obtained as a yellow solid. Yield: 78%. ¹H NMR (200 MHz, d₆-DMSO): δ 11.42 (s, 1H, OH), 7.43 (s, 1H arom.), 7.11 (s, 1H arom.), 6.79 (s, 1H arom.), 5.48 (s, H₂N), 3.92 (s, 3H, OCH₃), 2.65 (t, 4H, J=7 Hz, 2 CH₂), 1.62 (m, 4H, 2 CH₂), 1.43 (m, 2H, CH₂).

### Methyl-6-cyano-8-amino-4-hydroxy-quinoline-2-carboxylate 120b

By replacing methyl-8-benzyloxy-4-(toluene-4-sulfonylamino)-quinoline-2-carboxylate 15a in example 2 by methyl-6-cyano-8-nitro-4-benzyloxy-quinoline-2-carboxylate 120a, the abovenamed product is obtained as an orange solid. Yield: 41%. ¹H NMR (200 MHz, d₆-DMSO): δ 12.13 (s, 1H, OH), 7.64 (s, 1H, H arom.), 7.65 (s, 1H³), 7.05 (s, 1H, H arom.), 3.95 (s, 3H, OCH₃).

### 6-N-(N-methylpiperazinyl)-8-amino-4-hydroxy-quinoline-2-carboxylic acid hydrochloride 124b

By replacing methyl-8-benzyloxy-4-(toluene-4-sulfonylamino)-quinoline-2-carboxylate 15a in example 2 by 6-N-(N-methylpiperazinyl)-8-nitro-4-benzyloxy-quinoline-2-carboxylic acid hydrochloride 124a, the abovenamed product is obtained as a yellow solid. Yield: 88%. ¹H NMR (200 MHz, d₆-DMSO): δ 12.12 (s, 1H, OH), 11.13 (s, 1H, OH), 7.28 (s, 1H³), 6.62 (s, 1H, H arom.), 6.59 (s, 1H, H arom.), 6.29 (s, 2H, H₂N), 3.42 (m, 5H, 1H and 2 NCH₂), 3.30 (s, 4H, 2 CH₂), 2.65 (m, 3H, CH₃).

### Methyl-6-(piperidin-1-yl)-8-amino-4-hydroxy-quinoline-2-carboxylate 125b

By replacing methyl-8-benzyloxy-4-(toluene-4-sulfonylamino)-quinoline-2-carboxylate 15a in example 2 by methyl-6-(piperidin-1-yl)-8-nitro-4-benzyloxy-quinoline-2-carboxylate 125a, the abovenamed product is obtained as a yellow solid. Yield: 82%. ¹H NMR (200 MHz, d₆-DMSO): δ 11.02 (s, 1H, OH), 7.35 (s, 1H³), 6.71 (d, 1H, J=2 Hz, H arom.), 6.57 (d, 1H, J=2 Hz, H arom.), 6.57 (s, 2 HN), 3.91 (s, 3H, OCH₃), 3.28 (m, 4H, 2 NCH₂), 1.66 (m, 6H, 3 CH₂).

### Methyl-6-(piperazin-1-yl)-8-amino-4-hydroxy-quinoline-2-carboxylate 126b

By replacing methyl-8-benzyloxy-4-(toluene-4-sulfonylamino)-quinoline-2-carboxylate 15a in example 2 by methyl-6-(4-benzyl-piperazin-1-yl)-8-nitro-4-benzyloxy-quinoline-2-carboxylate 126a, the abovenamed product is obtained as an orange solid. Yield: 58%. ¹H NMR (200 MHz, d₆-DMSO): δ 11.32 (s, 1H, OH), 7.30 (s, 1H³), 6.88 (d, 1H, J=2 Hz, H arom.), 6.62 (d, 1H, J=2 Hz, H arom.), 6.50 (s, 2H, H₂N), 5.32 (s, 1H, NH), 3.96 (s, 3H, OCH₃), 3.00 (m, 4H, 2 NCH₂), 2.60 (m, 6H, 3 CH₂).

### Methyl-6,8-diamino-4-hydroxy-quinoline-2-carboxylate 32c

By replacing methyl-4-hydroxy-5-hydroxymethyl-8-benzyloxy-quinoline-2-carboxylate 2g in example 2 by methyl-6-amino-8-nitro-4-benzyloxy-quinoline-2-carboxylate 32b, the abovenamed product is obtained as a yellow solid. Yield: 87%. ¹H NMR (200 MHz, d₆-DMSO): δ 11.82 (s, 1H, OH), 7.13 (s, 1H³), 7.03 (d, 1H, J=2 Hz, H arom.), 6.90 (d, 1H, J=2 Hz, H arom.), 5.20 (s, 2H, H₂N), 5.00 (s, 2H, NH₂), 3.99 (s, 3H, OCH₃).

### Methyl-6-(N-anilino)-8-amino-4-hydroxy-quinoline-2-carboxylate 33b

By replacing methyl-4-hydroxy-5-hydroxymethyl-8-benzyloxy-quinoline-2-carboxylate 2g in example 2 by methyl-6-(1-anilino)-8-nitro-4-benzyloxy-quinoline-2-carboxylate 33a, the abovenamed product is obtained as an orange solid. Yield: 81%. ¹H NMR (200 MHz, CDCl₃): δ 9.51 (s, 1H, OH), 7.48 (m, 2H, 2H arom.), 7.68 (s, 1H³), 7.40 (m, 3H, 3H arom.), 7.20 (m, 2H, 2H arom.), 5.74 (s, 1H, 1NH), 5.40 (s, 2H, NH₂), 4.01 (s, 3H, OCH₃).

### 7-phenyl-8-amino-4-hydroxy-quinoline-2-carboxylic acid 34d

By replacing methyl-8-benzyloxy-4-(toluene-4-sulfonylamino)-quinoline-2-carboxylate 15a in example 2 by methyl-6-(N-piperidinyl)-8-nitro-4-benzyloxy-quinoline-2-carboxylate 34c, the abovenamed product is obtained as a yellow solid. Yield: 32%. ¹H NMR (300 MHz, d₆-DMSO): δ 10.26 (s, 1H, OH), 7.42 (m, 6H, 6H arom.), 6.98 (d, 1H, J=2 Hz, H arom.), 6.48 (s, 1H³), 5.32 (s, 2 HN).

### 4-(N-methylamino)-8-amino-quinoline-2-carboxylic acid hydrochloride 38d

By replacing methyl-8-benzyloxy-4-(toluene-4-sulfonylamino)-quinoline-2-carboxylate 15a in example 2 by methyl-4-(N-methylamino)-8-amino-quinoline-2-carboxylate 38c, the abovenamed product is obtained as a yellow solid. Yield: 82%. ¹H NMR (300 MHz, d₆-DMSO): δ 9.46 (s, 1H, OH), 7.82 (d, 1H, J=8 Hz, 1H arom.), 7.49 (t, 1H, J=8 Hz, 1H⁶), 7.28 (d, 1H, J=8 Hz, H arom.), 7.15 (s, 1H³), 3.15 (s, 3 HN).

### 3-(N-morpholinomethyl)-4,8-dihydroxy-quinoline-2-carboxylic acid 39b

By replacing methyl-4-hydroxy-5-hydroxymethyl-8-benzyloxy-quinoline-2-carboxylate 2g in example 2 by 3-(N-morpholinomethyl)-8-benzyloxy-4-hydroxy-quinoline-2-carboxylic acid 39a, the abovenamed product is obtained as a beige solid. Yield: 30%. ¹H NMR (200 MHz, d₆-DMSO): δ 12.05 (s, 1H, OH), 11.03 (s, 1H, OH), 10.30 (s, 1H, OH), 7.55 (d, 1H, J=8 Hz, 1H arom.), 7.20 (m, 2H, 2H arom.), 4.51 (s, 2H, CH₂N), 3.75 (m, 8H, 4 CH₂).

### 3-(N-pyrrolidinomethyl)-8-benzyloxy-4-hydroxy-quinoline-2-carboxylic acid 40a

By replacing methyl-8-benzyloxy-4-(toluene-4-sulfonylamino)-quinoline-2-carboxylate 15a in example 2 by 3-(N-pyrrolidinomethyl)-8-benzyloxy-4-hydroxy-quinoline-2-carboxylic acid 40a, the abovenamed product is obtained as a beige solid. Yield: 30%. ¹H NMR (200 MHz, d₆-DMSO): δ 11.00 (s, 1H, OH), 10.69 (s, 1H, OH), 10.37 (s, 1H, OH), 7.56 (dd, 1H, J=2 and 8 Hz, 1H arom.), 7.16 (m, 2H, 2H arom.), 4.57 (s, 2H, CH₂N), 3.37 (m, 4H, 2 CH₂), 1.88 (m, 4H, 2 CH₂).

### Methyl-8-dimethylamino-4-hydroxy-quinoline-2-carboxylate 41b

By replacing methyl-8-benzyloxy-4-(toluene-4-sulfonylamino)-quinoline-2-carboxylate 15a in example 2 by methyl-8-dimethylamino-4-benzyloxy-quinoline-2-carboxylate 41a, the abovenamed product is obtained as a yellow solid. Yield: 74%. ¹H NMR (300 MHz, CDCl₃): δ 9.77 (s, 1H, OH), 8.05 (dd, 1H, J=2 and 8 Hz, 1H arom.), 7.45 (dd, 1H, J=2 and 8 Hz, H arom.), 7.31 (t, 1H, J=8 Hz, 1H⁶), 6.96 (s, 1H³), 4.03 (s, 3H, OCH₃), 2.78 (s, 6H, 2 NCH₃).

### Methyl-8-amino-4-hydroxy-6-phenyl-quinoline-2-carboxylate 86c

By replacing methyl-8-benzyloxy-4-(toluene-4-sulfonylamino)-quinoline-2-carboxylate 15a in example 2 by methyl-4-hydroxy-8-nitro-6-phenyl-quinoline-2-carboxylate 86b and proceeding in the same manner, the abovenamed product is obtained. Yield: 98%. ¹H-NMR (200 MHz, CDCl₃): δ 11.44 (broad s, 1H, OH), 7.73-7.40 (m, 7H arom.), 7.23 (s, 1H arom.), 6.07 (broad s, 2H, NH₂), 3.95 (s, 3H, OCH₃).

### Methyl-8-hydroxy-4-(piperazin-1-yl)-quinoline-2-carboxylate 87b

By replacing methyl-8-benzyloxy-4-(toluene-4-sulfonylamino)-quinoline-2-carboxylate 15a in example 2 by methyl-8-benzyloxy-4-(4-benzyl-piperazin-1-yl)-quinoline-2-carboxylate 87a and proceeding in the same manner, the abovenamed product is obtained. Yield: 93%. ¹H-NMR (200 MHz, CDCl₃): δ 7.51-7.48 (m, 3H arom.), 7.17-7.12 (m, 1H arom.), 3.95 (s, 3H, OCH₃), 3.29 (broad s, 2H, NH₂), 3.28-3.25 (m, 4H, CH₂—N—CH₂), 3.15-3.13 (m, 4H, CH₂—N—CH₂).

### Methyl-8-amino-4-phenyl-quinoline-2-carboxylate 92c

By replacing methyl-8-benzyloxy-4-(toluene-4-sulfonylamino)-quinoline-2-carboxylate 15a in example 2 by methyl-8-nitro-4-phenyl-quinoline-2-carboxylate 92b and proceeding in the same manner, the abovenamed product is obtained. Yield: 93%. ¹H-NMR (200 MHz, CDCl₃): δ 8.07 (s, 1H arom.), 7.53-7.21 (m, 7H arom.), 6.92 (d, 1H, J=8 Hz, 1H arom.), 4.05 (s, 3H, OCH₃).

### Methyl-8-amino-4-(hex-1-yl)-quinoline-2-carboxylate 93b

By replacing methyl-8-benzyloxy-4-(toluene-4-sulfonylamino)-quinoline-2-carboxylate 15a in example 2 by methyl-8-amino-4-(hex-1-ynyl)-quinoline-2-carboxylate 93a and proceeding in the same manner, the abovenamed product is obtained. Yield: 93%. ¹H-NMR (200 MHz, CDCl₃): δ 7.97(s, 1H arom.), 7.45-7.32 (m, 2H arom.), 6.92 (d, 1H, J=8 Hz, 1H arom.), 5.20 (broad s, 2H, NH₂), 4.03 (s, 3H, OCH₃), 3.05 (t, 2H, J=7 Hz, CH₂), 1.77-1.74 (m, 2H, CH₂), 1.36-1.31(m, 6H, 3×CH₂), 0.90 (t, 3H, J=7 Hz, CH₃).

### Methyl-8-amino-4-(2-phenyleth-1-yl)-quinoline-2-carboxylate 94b

By replacing methyl-8-benzyloxy-4-(toluene-4-sulfonylamino)-quinoline-2-carboxylate 15a in example 2 by methyl-8-amino-4-phenylethynyl-quinoline-2-carboxylate 94a and proceeding in the same manner, the abovenamed product is obtained. Yield: 90%. ¹H-NMR (300 MHz, CDCl₃): δ 7.97 (s, 1H arom.), 7.45-7.22 (m, 7H arom.), 6.94 (d, 1H, J=8 Hz, 1H arom.), 5.19 (broad s, 2H, NH₂), 4.03 (s, 3H, OCH₃), 3.41-3.36 (m, 2H, CH₂), 3.33-3.04 (m, 2H, CH₂).

### Methyl8-amino-4-(3-tert-butoxycarbonylamino-prop-1-yl)-quinoline-2-carboxylate 95b

By replacing methyl-8-benzyloxy-4-(toluene-4-sulfonylamino)-quinoline-2-carboxylate 15a in example 2 by methyl-8-nitro-4-(3-tert-butoxycarbonylamino-prop-1-ynyl)-quinoline-2-carboxylate 95a and proceeding in the same manner, the abovenamed product is obtained. Yield: 79%. ¹H-NMR (300 MHz, CDCl₃): δ 7.97 (s, 1H arom.), 7.43-7.27 (m, 2H arom.), 6.93 (d, 1H, J=8 Hz, 1H arom.), 5.30 (broad s, 1H, NH), 4.03 (s, 3H, OCH₃), 3.27-3.24 (m, 2H, CH₂), 3.09 (t, 2H, J=6 Hz, CH₂), 2.01-1.95 (m, 2H, CH₂), 1.46 (s, 9H, (CH₃)₃).

### Methyl-8-amino-4-(3-hydroxy-prop-1-yl)-quinoline-2-carboxylate 96b

By replacing methyl-8-benzyloxy-4-(toluene-4-sulfonylamino)-quinoline-2-carboxylate 15a in example 2 by methyl-4-(3-benzyloxy-prop-1-ynyl)-8-nitro-quinoline-2-carboxylate 96a and proceeding in the same manner, the abovenamed product is obtained. Yield: 48%. ¹H-NMR (200 MHz, CDCl₃): δ 8.01(s, 1H arom.), 7.43-7.27 (m, 2H arom.), 6.93 (d, 1H, J=8 Hz, 1H arom.), 5.18 (broad s, 2H, NH₂), 4.03 (s, 3H, OCH₃), 3.81-3.76 (m, 2H, CH₂), 3.19 (t, 2H, J=6 Hz, CH₂), 2.08-2.01 (m, 2H, CH₂).

### Methyl-4-(3-acetyl-aminoprop-1-ynyl)-8-amino-quinoline-2-carboxylate 97b

By replacing methyl-8-benzyloxy-4-(toluene-4-sulfonylamino)-quinoline-2-carboxylate 15a in example 2 by methyl-4-(3-acetyl-aminoprop-1-ynyl)-8-nitro-quinoline-2-carboxylate 97a and proceeding in the same manner, the abovenamed product is obtained. Yield: 54%. ¹H-NMR (200 MHz, CDCl₃): δ 7.97(s, 1H arom.), 7.45-7.27 (m, 2H arom.), 6.95 (d, 1H, J=8 Hz, 1H arom.), 6.67 (s, 1H, NH), 5.25 (broad s, 2H, NH₂), 4.38 (s, 2H, NCH₂), 4.03 (s, 3H, OCH₃), 2.41 (s, 3H, COCH₃).

### Methyl-8-hydroxy-4-(morpholin-1-yl)-quinoline-2-carboxylate 99b

By replacing methyl-8-benzyloxy-4-(toluene-4-sulfonylamino)-quinoline-2-carboxylate 15a in example 2 by methyl-8-benzyloxy-4-(morpholin-1-yl)-quinoline-2-carboxylate 99a and proceeding in the same manner, the abovenamed product is obtained. Yield: 92%. ¹H-NMR (300 MHz, CDCl₃): δ 7.64 (s, 1H arom.), 7.47 (m, 2H arom.), 7.18 (s, 1H arom.), 4.04 (s, 3H, OCH₃), 4.01-3.99 (m, 4H, CH₂—O—CH₂), 3.34-3.31 (m, 4H, CH₂—N—CH₂).

### Methyl-8-hydroxy-4-(piperidin-1-yl)-quinoline-2-carboxylate 100b

By replacing methyl-8-benzyloxy-4-(toluene-4-sulfonylamino)-quinoline-2-carboxylate 15a in example 2 by methyl-8-benzyloxy-4-(piperidin-1-yl)-quinoline-2-carboxylate 100a and proceeding in the same manner, the abovenamed product is obtained. Yield: 89%. ¹H-NMR (200 MHz, CDCl₃): δ 7.57 (s, 1H arom.), 7.45-7.42 (m, 2H arom.), 7.23 (m, 1H arom.), 4.04 (s, 3H, OCH₃), 3.36-3.31 (m, 4H, CH₂—N—CH₂), 1.86-1.71 (m, 6H, 3×CH₂).

### Methyl-8-amino-4-(piperidin-1-yl)-quinoline-2-carboxylate 101b

By replacing methyl-8-benzyloxy-4-(toluene-4-sulfonylamino)-quinoline-2-carboxylate 15a in example 2 by methyl-8-nitro-4-(pipeeridin-1-yl)-quinoline-2-carboxylate 101a and proceeding in the same manner, the abovenamed product is obtained. Yield: 77%. ¹H-NMR (200 MHz, CDCl₃): δ 7.59 (s, 1H arom.), 7.33-7.27 (s, 1H arom.), 6.88 (d, 1H, J=8 Hz, 1H arom.), 5.13 (broad s, 2H, NH₂), 4.02 (s, 3H, OCH₃), 3.25-3.21 (m, 4H, CH₂—N—CH₂), 1.90-1.68 (m, 6H, 3×CH₂).

### Methyl-4-hydroxy-8-(piperazin-1-yl)-quinoline-2-carboxylate 108a

By replacing methyl-8-benzyloxy-4-(toluene-4-sulfonylamino)-quinoline-2-carboxylate 15a in example 2 by methyl-4-benzyloxy-8-(4-benzyl-piperazin-1-yl)-quinoline-2-carboxylate 105a and proceeding in the same manner, the abovenamed product is obtained. Yield: 95%. ¹H-NMR (200 MHz, CDCl₃): δ 9.30 (broad s, 1H, OH), 7.84-7.81 (m, 1H arom.), 7.50-7.42 (m, 2H arom.), 6.99 (broad s, 1H arom.), 3.95 (s, 3H, OCH₃), 3.32-3.25 (m, 8H, 4×CH₂).

### Methyl-8-hydroxy-4-(4-methyl-piperazin-1-yl)-quinoline-2-carboxylate 110b

By replacing methyl-8-benzyloxy-4-(toluene-4-sulfonylamino)-quinoline-2-carboxylate 15a in example 2 by methyl-8-benzyloxy-4-(4-methyl-piperazin-1-yl)-quinoline-2-carboxylate 110a and proceeding in the same manner, the abovenamed product is obtained. Yield: 81%. ¹H-NMR (200 MHz, CDCl₃): δ 9.65 (broad s, 1H, OH), 7.55-7.44 (m, 3H arom.), 7.16-7.12 (m, 1H arom.), 4.03 (s, 3H, OCH₃), 3.29-3.25 (m, 4H, CH₂—N—CH₂), 2.67-2.63 (m, 4H, CH₂—N—CH₂), 2.31 (s, 3H, NCH₃).

### Methyl-7-acetylamino-4-hydroxy-quinoline-2-carboxylate 116b

By replacing methyl-8-benzyloxy-4-(toluene-4-sulfonylamino)-quinoline-2-carboxylate 15a in example 2 by methyl-7-acetylamino-4-benzyloxy-quinoline-2-carboxylate 116a and proceeding in the same manner, the abovenamed product is obtained. Yield: 82%. ¹H-NMR (200 MHz, CDCl₃): δ 12.06 (broad s, 1H, OH), 10.36 (broad s, 1H, NH), 8.46 (s, 1H arom.), 8.02 (d, 1H, J=8 Hz, 1H arom.), 7.34 (d, 1H, J=8 Hz, 1H arom.), 6.60 (s, 1H arom.), 3.96 (s, 3H, OCH₃), 2.14 (s, 3H, COCH₃).

### EXAMPLE 3

### Methyl-4,8-dihydroxy-5-bromo-quinoline-2-carboxylate 2c

Dissolve 172 mg (0.443 mmol) of methyl-4-hydroxy-5-bromo-8-benzyloxy-quinoline-2-carboxylate 2b in 3 ml of dichloromethane and add 0.18 ml (1.33 mmol) of dimethylaniline. Add 239 mg (1.79 mmol) of aluminium chloride powder and stir at room temperature for 1 to 2 hours. Evaporate to dryness, add 15 ml of 1 N hydrochloric acid and triturate. Filter the precipitate, triturate in 15 ml of diethyl ether and filter. One obtains 110 mg (0.37 mmol) of the abovenamed product. Yield: 84%. ¹H-NMR (300 MHz, DMSO-d₆): δ 11.44 (broad s, 1H, OH), 9.59 (broad s, 1H, OH), 7.40 (m, 1H arom.), 7.04 (m, 1H arom.), 6.56 (m, 1H arom.), 3.96 (s, 3H, OCH₃).

### Methyl-4,8-dihydroxy-6-bromo-quinoline-2-carboxylate 2m

By replacing methyl-4-hydroxy-5-bromo-8-benzyloxy-quinoline-2-carboxylate 2b in example 3 by methyl-4-hydroxy-6-bromo-8-benzyloxyquinoline-2-carboxylate 2l and proceeding in the same manner, the abovenamed product is obtained. Yield: 93%. ¹H-NMR (300 MHz, DMSO-d₆): δ 7.67 (s, 1H arom.), 7.27 (s, 1H arom.), 7.00 (broad s, 1H arom.).

### Methyl-4,8-dihydroxy-6-(4-chloro-phenyl)-quinoline-2-carboxylate 18l

By replacing methyl-4-hydroxy-5-bromo-8-benzyloxy-quinoline-2-carboxylate 2b in example 3 by methyl-4-hydroxy-6-(4-chloro-phenyl)-8-benzyloxy-quinoline-2-carboxylate 18k and proceeding in the same manner, the abovenamed product is obtained. Yield: 80%. ¹H-NMR (200 MHz, methanol-d₄): δ 8.04 (m, 1H arom.), 7.75-7.51 (m, 6H arom.), 4.15 (s, 3H, OCH₃).

### Methyl-4,8-dihydroxy-6-(3,4-dichloro-phenyl)-quinoline-2-carboxylate 18n

By replacing methyl-4-hydroxy-5-bromo-8-benzyloxy-quinoline-2-carboxylate 2b in example 3 by methyl-4-hydroxy-6-(3,4-dichloro-phenyl)-8-benzyloxy-quinoline-2-carboxylate 18m and proceeding in the same manner, the abovenamed product is obtained. Yield: 91%. ¹H-NMR (200 MHz, DMSO-d₆): δ 7.97 (s, 1H arom.), 7.82 (m, 1H arom.), 7.74 (m, 2H arom.), 7.47 (m, 1H arom.), 7.06 (m, 1H arom.), 4.14 (s, 3H, OCH₃).

### 8-hydroxy-5-bromo-quinoline-2-carboxylic acid 4b

By replacing methyl-4-hydroxy-5-bromo-8-benzyloxy-quinoline-2-carboxylate 2b in example 3 by benzyl-8-benzyloxy-5-bromo-quinoline-2-carboxylate 5b and proceeding in the same manner, the abovenamed product is obtained. Yield: 93%. ¹H-NMR (300 MHz, DMSO-d₆): δ 13.04 (broad s, 1H, COOH), 10.52 (broad s, 1H, OH), 8.62 (d, 1H, J=9 Hz, 1H arom.), 8.28 (d, 1H, J=9 Hz, 1H arom.), 7.97(d, 1H, J=8 Hz, 1H arom.), 7.19 (d, 1H, J=8 Hz, 1H arom.).

### Methyl-5,7-dichloro-4,8-dihydroxyquinoline-2-carboxylate 44c

By replacing methyl-4-hydroxy-5-bromo-8-benzyloxy-quinoline-2-carboxylate 2b in example 3 by methyl-8-benzyloxy-5,7-dichloro-4-hydroxyquinoline-2-carboxylate 44b and proceeding in the same manner, the abovenamed product is obtained. Yield: 88%. ¹H-NMR (200 MHz, DMSO-D₆): δ 7.57 (m, 1H arom.), 7.29 (m, 1H arom.), 3.96 (s, 3H, OCH₃).

### Methyl-3-bromo-4,8-dihydroxy-quinoline-2-carboxylate 52b

By replacing methyl-4-hydroxy-5-bromo-8-benzyloxy-quinoline-2-carboxylate 2b in example 3 by methyl-8-benzyloxy-3-bromo-4-hydroxyquinoline-2-carboxylate 52a and proceeding in the same manner, the abovenamed product is obtained. Yield: 80%. ¹H-NMR (300 MHz, DMSO-D₆): δ 11.07 (broad s, 2H, 2 OH), 7.60-7.57 (dd, J=1 Hz and J=8 Hz, 1H arom.), 7.29-7.24 (t, J=7.8 Hz, 1H arom.), 7.20-7.17 (dd, J=1 Hz and J=8 Hz, 1H arom.), 3.93 (s, 3H, OCH₃).

### Methyl-3,7-dibromo-4,8-dihydroxy-quinoline-2-carboxylate 56b

By replacing methyl-4-hydroxy-5-bromo-8-benzyloxy-quinoline-2-carboxylate 2b in example 3 by methyl-8-benzyloxy-3,7-dibromo-4-hydroxyquinoline-2-carboxylate 56a and proceeding in the same manner, the abovenamed product is obtained. Yield: 85%. ¹H-NMR (300 MHz, DMSO-D₆): δ 12.40 (broad s, 1H, OH), 10.67 (broad s, 1H, OH), 7.60-7.52 (m, 2H arom.), 3.94 (s, 3H, OCH₃).

### Methyl-4-(3-benzoyl-aminoprop-1-yl)-8-hydroxyquinoline-2-carboxylate 85d

By replacing methyl-4-hydroxy-5-bromo-8-benzyloxy-quinoline-2-carboxylate 2b in example 3 by methyl-8-benzyloxy-4-(3-benzoyl-aminoprop-1-yl)-quinoline-2-carboxylate 85c and proceeding in the same manner, the abovenamed product is obtained. Yield: 85%. ¹H-NMR (200 MHz, CDCl₃): 8.49 (s, 1H, OH.), 8.06 (s, 1H arom.), 7.76-7.20 (m, 8H arom.), 6.21 (broad s, 1H, NH), 4.05 (s, 3H, OCH₃), 3.67-3.57 (m, 2H, CH₂), 3.23 (t, 2H, J=7.8 Hz, CH₂), 2.19-2.12 (m, 2H, CH₂).

### EXAMPLE 4

### Methyl-4-hydroxy-8-amino-quinoline-2-carboxylate 2u

Dissolve methyl-4-hydroxy-8-nitro-quinoline-2-carboxylate 2r (295 mg, 1.21 mmol) in methanol (10 ml). Add Pd/C 10% (18 mg) and hydrogenate under atmospheric pressure for 2 hours at room temperature. Filter the solution on celite and evaporate the filtrate. Purify the crude reaction product by silica gel column chromatography, eluent: Hex/EtOAc, 1/1 then EtOAc. Yield: 93%. ¹H-NMR (200 MHz, DMSO-d₆): δ 7.37-7.28 (m, 3H arom.), 5.96 (s, 1H arom.), 3.93 (s, 3H, OCH₃).

### Methyl-4-hydroxy-5-methyl-8-amino-quinoline-2-carboxylate 2v

By replacing methyl-4-hydroxy-8-nitro-quinoline-2-carboxylate in example 4 by methyl-4-hydroxy-5-methyl-8-nitro-quinoline-2-carboxylate 2s, and proceeding in the same manner, the abovenamed product is obtained. Yield: 68%. ¹H-NMR (200 MHz, DMSO-d₆): δ 11.31 (broad s, 1H, OH), 7.04 (broad s, 1H arom.), 7.01 (d, 1H, J=8 Hz, 1H arom.), 6.81 (d, 1H, J=8 Hz, 1H arom.), 5.68 (s, 2H, NH₂), 3.90 (s, 3H, OCH₃), 2.60 (s, 3H, CH₃).

### Methyl-4-hydroxy-6-methyl-8-amino-quinoline-2-carboxylate 2w

By replacing methyl-4-hydroxy-8-nitro-quinoline-2-carboxylate in example 4 by methyl-4-hydroxy-6-methyl-8-nitro-quinoline-2-carboxylate 2t and proceeding in the same manner, the abovenamed product is obtained. Yield: 46%. ¹H-NMR (200 MHz, DMSO-d₆): δ 7.36 (s, 1H arom.), 7.09 (s, 1H arom.), 6.75 (s, 1H arom.), 5.88 (s, 2H, NH₂), 3.91 (s, 3H, OCH₃), 2.02 (s, 3H, CH₃).

### Methyl-4-hydroxy-6 amino-8-methoxy-quinoline-2-carboxylate 2q

By replacing methyl-4-hydroxy-8-nitro-quinoline-2-carboxylate in example 4 by methyl-4-hydroxy-8-meethoxy-6-nitro-quinoline-2-carboxylate 2p and proceeding in the same manner, the abovenamed product is obtained. Yield: 93%. ¹H-NMR (300 MHz, CDCl₃): δ 6.85 (d, 1H, J=2 Hz, 1H arom.), 6.79 (s, 1H arom.), 6.53 (d, 1H, J=2 Hz, 1H arom.), 3.95 (s, 3H, OCH₃), 3.88 (s, 3H, OCH₃), 3.84 (broad s, 2H, NH₂).

### Methyl-8-amino-quinoline-2-carboxylate 5f

By replacing methyl-4-hydroxy-8-nitro-quinoline-2-carboxylate in example 4 by methyl-4-chloro-8-nitro-quinoline-2-carboxylate 8c and proceeding in the same manner, the abovenamed product is obtained. Yield: 18%. ¹H-NMR (300 MHz, CDCl₃): δ 8.15 (m, 2H arom.), 7.43 (m, 1H arom.), 7.16 (d, 1H, J=8 Hz, 1H arom.), 6.95 (d, 1H, J=8 Hz, 1H arom.), 5.18 (broad s, 2H, NH₂), 4.04 (s, 3H, OCH₃).

### Methyl-8-amino-4-(toluene-4-sulfonylamino)-quinoline-2-carboxylate 15d

By replacing methyl-4-hydroxy-8-nitro-quinoline-2-carboxylate in example 4 by methyl-8-nitro-4-(toluene-4-sulfonylamino)-quinoline-2-carboxylate 15c and proceeding in the same manner, the abovenamed product is obtained. Yield: 73%. ¹H-NMR (300 MHz, CDCl₃): δ 8.19 (s, 1H arom.), 7.87 (d, 2H, J=7 Hz, 2H arom.), 7.63 (broad s, 1H, NH), 7.38 (m, 1H, 1H arom.), 7.26 (d, 2H, J=7 Hz, 2H arom.), 7.03 (broad d, 1H, J=8 Hz, 1H arom.), 6.90 (d, 1H, J=8 Hz, 1H arom.), 5.24 (broad s, 2H, NH₂), 4.02 (s, 3H, OCH₃), 2.36 (s, 3H, CH₃).

### Methyl-4-(N-methylamino)-8-amino-quinoline-2-carboxylate 132c

By replacing methyl-4-hydroxy-8-nitro-quinoline-2-carboxylate 2r in example 4 by methyl-4-(N-methylamino)-8-amino-quinoline-2-carboxylate 132b, the abovenamed product is obtained. Yield: 96%. ¹H NMR (200 MHz, CDCl₃): δ 7.32 (t, 1H, J=8 Hz, H⁶), 7.27 (s, 1H, H³), 6.93 (d, 1H, J=8 Hz, H arom.), 6.90 (d, 1H, J=8 Hz, H arom.), 5.10 (s, 3H, NH₂ and NH), 4.02 (s, 3H, OCH₃), 3.15 (d, 3H, J=3 Hz, CH₃).

### EXAMPLE 5

### Methyl-4-hydroxy-6-iodo-8-methoxy-quinoline-2-carboxylate 2j

Suspend methyl-4-hydroxy-6-amino-8-methoxy-quinoline-2-carboxylate 2q (126 mg, 0.51 mmol) in ice-water (2 ml). Add 0.3 ml of concentrated sulfuric acid. At 0° C., add sodium nitrite (39 mg, 0.57 mmol). Allow to stand at this temperature for 1 hour. Then add dropwise potassium iodide (115 mg, 0.69 mmol) dissolved in 2 ml of water. Heat the reaction medium at 70° C. for 1 hour. Extract the aqueous medium with methylene chloride. Dry on Na₂SO₄, filter, and evaporate the organic solvents. The crude reaction product is purified by silica gel column chromatography, eluent, CH₂Cl₂/MeOH: 9/1. Yield: 55%. ¹H-NMR (200 MHz, CDCl₃): δ 9.33 (broad s, 1H, OH), 8.26 (d, 1H, J=2 Hz, 1H arom.), 7.31 (d, 1H, J=2 Hz, 1H arom.), 6.98 (s, 1H, H arom.), 3.89 (s, 3H, OCH₃), 3.70 (s, 3H, OCH₃).

### EXAMPLE 6

### 4,8-dihydroxy-6-bromo-quinoline-2-carboxylic acid 3k

In a flask, mix potassium iodide (340 mg, 2.05 mmol), phosphoric acid (410 mg, 4.18 mmol) and methyl-4-hydroxy-6-bromo-8-methoxy-quinoline-2-carboxylate 2k (160 mg, 0.52 mmol). Heat this mixture at 60° C. so as to form a slurry then add 5 drops of 85% phosphoric acid in water. Heat at 160° C. for 36 hours. Allow to cool and add 10 ml of water. Stir under cold for 30 minutes then filter the precipitate. Wash the precipitate with isopropanol. Yield: 79%. ¹H-NMR (300 MHz, DMSO-d₆): δ 11.08 (broad s, 3H, COOH and 2×OH), 7.67 (s, 1H arom.), 7.27 (s, 1H arom.), 7.00 (broad s, 1H arom.).

### 4,8-dihydroxy-5-methyl-4-quinoline-2-carboxylic acid 3e

By replacing methyl-4-hydroxy-6-bromo-8-methoxy-quinoline-2-carboxylate 2k in example 6 by methyl-4-hydroxy-5-methyl-8-methoxy-quinoline-2-carboxylate 2e and proceeding in the same manner, the abovenamed product is obtained. Yield: 96%. ¹H-NMR (300 MHz, DMSO-d₆): δ 10.54 (broad s, 1H, COOH), 7.01-6.94 (m, 2H arom.), 6.73 (s, 1H arom.), 2.68 (s, 3H, CH₃).

### 4,8-dihydroxy-5,7-dichloro-quinoline-2-carboxylic acid 3i

By replacing methyl-4-hydroxy-6-bromo-8-methoxy-quinoline-2-carboxylate 2k in example 6 by methyl-4-hydroxy-5,7-dichloro-8-methoxy-quinoline-2-carboxylate 2i and proceeding in the same manner, the abovenamed product is obtained. Yield: 82%. ¹H-NMR (200 MHz, DMSO-d₆): δ 12.51 (broad s, 1H, COOH), 10.81 (broad s, 2H, 2×OH), 7.65 (s, 1H arom.), 7.43 (broad s, 1H arom.).

### 4,8-dihydroxy-6-iodo-quinoline-2-carboxylic acid 3j

By replacing methyl-4-hydroxy-6-bromo-8-methoxy-quinoline-2-carboxylate 2k in example 6 by methyl-4-hydroxy-6-iodo-8-methoxy-quinoline-2-carboxylate 2j and proceeding in the same manner, the abovenamed product is obtained. Yield: 54%. ¹H-NMR (300 MHz, DMSO): δ 11.06 (broad s, 1H, COOH), 7.87 (s, 1H arom.), 7.39 (s, 1H arom.), 6.90 (broad s, 1H arom.).

### 4,8-dihydroxy-6-methyl-quinoline-2-carboxylic acid 3n

By replacing methyl-4-hydroxy-6-bromo-8-methoxy-quinoline-2-carboxylate 2k in example 6 by methyl-4-hydroxy-6-methyl-8-methoxy-quinoline-2-carboxylate 2n and proceeding in the same manner, the abovenamed product is obtained. Yield: 95%. ¹H-NMR (300 MHz, DMSO-d₆): δ 7.37 (m, 1H arom.), 7.02 (m, 1H arom.), 6.97 (s, 1H arom.), 2.39 (s, 3H, CH₃).

### 8-hydroxy-4-phenyl-quinoline-2-carboxylic acid 10b

By replacing methyl-4-hydroxy-6-bromo-8-methoxy-quinoline-2-carboxylate 2k in example 6 by methyl-8-methoxy-4-phenyl-quinoline-2-carboxylate 9b and proceeding in the same manner, the abovenamed product is obtained. Yield: 62%. ¹H-NMR (200 MHz, DMSO-d₆): δ 12.90 (broad s, 1H, COOH), 10.31 (broad s, 1H, OH), 8.01 (s, 1H arom.), 7.64-7.59 (m, 6H arom.), 7.40-7.25 (m, 2H arom.).

### 8-hydroxy-4-(4-chloro-phenyl)-quinoline-2-carboxylic acid 10c

By replacing methyl-4-hydroxy-6-bromo-8-methoxy-quinoline-2-carboxylate 2k in example 6 by methyl-8-methoxy-4-(4-chlorophenyl)-quinoline-2-carboxylate 9c and proceeding in the same manner, the abovenamed product is obtained. Yield: 53%. ¹H-NMR (300 MHz, DMSO-d₆): δ 12.59 (broad s, 1H, COOH), 10.33 (broad s, 1H, OH), 8.02 (s, 1H arom.), 7.70-7.62 (m, 5H arom.), 7.37-7.26 (m, 2H arom.).

### 4,8-dihydroxy-6-pheenyl-quinoline-2-carboxylic acid 19f

By replacing methyl-4-hydroxy-6-bromo-8-methoxy-quinoline-2-carboxylate 2k in example 6 by methyl-4-hydroxy-6-phenyl-8-methoxy-quinoline-2-carboxylate 18f and proceeding in the same manner, the abovenamed product is obtained. Yield: 99%. ¹H-NMR (200 MHz, DMSO-d₆): δ 7.81-7.71 (m, 3H arom.), 7.52-7.45 (m, 4H arom.), 6.98 (s, 1H arom.).

### 4,8-dihydroxy-6-propyl-quinoline-2-carboxylic acid 19r

By replacing methyl-4-hydroxy-6-bromo-8-methoxy-quinoline-2-carboxylate 2k in example 6 by methyl-4-hydroxy-8-methoxy-6-propyl-quinoline-2-carboxylate 18r and proceeding in the same manner, the abovenamed product is obtained. Yield: 34%. ¹H-NMR (200 MHz, DMSO-d₆): δ 7.38 (s, 1H arom.), 7.03 (s, 2H arom.), 2.65 (t, 2H, CH₂), 1.63 (m, 2H, CH₂), 0.92 (t, 3H, CH₃).

### 8-hydroxy-5-phenyl-quinoline-2-carboxylic acid 19t

By replacing methyl-4-hydroxy-6-bromo-8-methoxy-quinoline-2-carboxylate 2k in example 6 by methyl-8-methoxy-5-phenyl-quinoline-2-carboxylate 18t and proceeding in the same manner, the abovenamed product is obtained. Yield: 65%. ¹H-NMR (200 MHz, DMSO-d₆): δ 11.15 (broad s, 1H, COOH), 8.43 (d, 1H, J=9 Hz, 1H arom.), 8.17 (d, 1H, J=9 Hz, 1H arom.), 7.63-7.47 (m, 6H arom.), 7.31 (d, 1H, J=8 Hz, 1H arom).

### 4,8-dihydroxy-6-phenethyl-quinoline-2-carboxylic acid 22a

By replacing methyl-4-hydroxy-6-bromo-8-methoxy-quinoline-2-carboxylate 2k in example 6 by methyl-4-hydroxy-8-meethoxy-6-phenethyl-quinoline-2-carboxylate 21a and proceeding in the same manner, the abovenamed product is obtained. Yield: 61%. ¹H-NMR (300 MHz, DMSO-d₆): δ 7.40 (d, 1H, J=2 Hz, 1H arom.), 7.29-7.14 (m, 5H arom.), 7.07 (d, 1H, J=2 Hz, 1H arom.), 6.96 (s, 1H arom.), 2.99-2.90 (m, 4H, 2CH₂).

### 4,8-dihydroxy-6-heptyl-quinoline-2-carboxylic acid 22b

By replacing methyl-4-hydroxy-6-bromo-8-methoxy-quinoline-2-carboxylate 2k in example 6 by methyl-4-hydroxy-8-methoxy-6-heptyl-quinoline-2-carboxylate 21b and proceeding in the same manner, the abovenamed product is obtained. Yield: 48%. ¹H-NMR (200 MHz, DMSO-d₆): δ 7.37 (s, 1H arom.), 7.02 (s, 2H arom.), 2.67 (t, 2H, CH₂), 1.60 (m, 2H, CH₂), 1.28 (m, 8H, 4×CH₂), 0.85 (m, 3H, CH₃).

### 4,8-dihydroxy-6-(benzylamino-methyl)-quinoline-2-carboxylic acid 26a

By replacing methyl-4-hydroxy-6-bromo-8-methoxy-quinoline-2-carboxylate 2k in example 6 by methyl-6-(benzylamino-methyl)-4-hydroxy-8-methoxy-quinoline-2-carboxylate 25a and proceeding in the same manner, the abovenamed product is obtained. Yield: 67%. ¹H-NMR (200 MHz, DMSO-d₆): δ 7.76 (s, 1H arom.), 7.45 (m, 5H arom.), 7.27 (m, 1H arom.), 6.99 (m, 1H arom.), 4.21 (m, 4H, 2×CH₂).

### 5-(4-chlorophenyl)-8-hydroxy-quinoline-2-carboxylic acid 81b

By replacing methyl-4-hydroxy-6-bromo-8-methoxy-quinoline-2-carboxylate 2k in example 6 by methyl-5-(4-chlorophenyl)-8-methoxydroxy-quinoline-2-carboxylate 81a and proceeding in the same manner, the abovenamed product is obtained. Yield: 60%. ¹H-NMR (200 MHz, DMSO-d₆): δ 13.05 (broad s, 1H, CO₂H), 10.40 (broad s, 1H, OH), 8.42 (d, 1H, J=9 Hz, 1H arom.), 8.18 (d, 1H, J=9 Hz, 1H arom.), 7.65-7.49 (m, 5H arom.), 7.32 (d, 1H, J=8 Hz, 1H arom).

### EXAMPLE 7

### 4-hydroxy-8-amino-quinoline-2-carboxylic acid 3u

Dissolve methyl-4-hydroxy-8-amino-quinoline-2-carboxylate 2u (150 mg, 0.69 mmol) in 2 N HCl solution in water (10 ml). Heat at 60° C. overnight. Allow to cool and filter the precipitate. Yield: 60%. ¹H-NMR (200 MHz, D₂O): δ 7.52 (d, 1H, J=8 Hz, 1H arom.), 7.17 (m, 1H arom.), 6.99 (d, 1H, J=8 Hz, 1H arom.), 6.93 (s, 1H arom.).

### 4,8-dihydroxy-6-(3-amino-propyl)-quinoline-2-carboxylic acid hydrochloride 22c

By replacing methyl-4-hydroxy-8-amino-quinoline-2-carboxylate 2u in example 7 by methyl-4,8-dihydroxy-6-(3-tert-butoxycarbonylamino-propyl)-quinoline-2-carboxylate 21c and proceeding in the same manner, the abovenamed product is obtained. Yield: 92%. ¹H-NMR (200 MHz, DMSO-d₆): δ 10.73 (broad s, 1H, COOH), 8.01 (broad s, 3H, NH₃⁺), 7.44 (s, 1H arom.), 7.11 (s, 2H arom.), 2.80 (m, 4H, 2CH₂), 1.92 (m, 2H, CH₂).

### 4,8-dihydroxy-6-(3-hydroxy-propyl)-quinoline-2-carboxylic acid 22d

By replacing methyl-4-hydroxy-8-amino-quinoline-2-carboxylate 2u in example 7 by methyl-4,8-dihydroxy-6-(3-hydroxy-propyl)-quinoline-2-carboxylate 21d and proceeding in the same manner, the abovenamed product is obtained. Yield: 50%. ¹H-NMR (200 MHz, DMSO-d₆): δ 7.59 (m, 1H arom.), 7.11 (m, 2H arom.), 3.60 (m, 2H, CH₂), 2.74 (m, 2H, CH₂), 1.94 (m, 2H, CH₂).

### 4,8-dihydroxy-6-(4-methoxy-phenyl)-quinoline-2-carboxylic acid 19g

By replacing methyl-4-hydroxy-8-amino-quinoline-2-carboxylate 2u in example 7 by methyl-4,8-dihydroxy-6-(4-methoxy-phenyl)-quinoline-2-carboxylate 18h and proceeding in the same manner, the abovenamed product is obtained. Yield: 90%. ¹H-NMR (300 MHz, DMSO-d₆): δ 7.83 (s, 1H arom.), 7.71-7.68 (m, 2H arom.), 7.55 (m, 1H arom.), 7.29 (s, 1H arom.), 7.09-7.06 (m, 2H arom.), 3.82 (s, 3H, OCH₃).

### 4,8-dihydroxy-6-(3-methyl-phenyl)-quinoline-2-carboxylic acid 19i

By replacing methyl-4-hydroxy-8-amino-quinoline-2-carboxylate 2u in example 7 by methyl-4,8-dihydroxy-6-(3-methyl-phenyl)-quinoline-2-carboxylate 18j and proceeding in the same manner, the abovenamed product is obtained. Yield: 90%. ¹H-NMR (200 MHz, DMSO-d₆): δ 7.81 (m, 1H arom.), 7.55-7.35 (m, 4H arom.), 7.25 (m, 1H arom.), 7.06 (s, 1H arom.), 2.43 (s, 3H, CH₃).

### 4,8-dihydroxy-6-(4-chloro-phenyl)-quinoline-2-carboxylic acid 19k

By replacing methyl-4-hydroxy-8-amino-quinoline-2-carboxylate 2u in example 7 by methyl-4-hydroxy-6-(4-chloro-phenyl)-8-benzyloxy-quinoline-2-carboxylate 18k and proceeding in the same manner, the abovenamed product is obtained. Yield: 70%. ¹H-NMR (200 MHz, DMSO-d₆): δ 7.79 (m, 1H arom.), 7.76-7.72 (m, 2H arom.), 7.58-7.54 (m, 2H arom.), 7.48 (m, 1H arom.), 6.92 (broad s, 1H arom.).

### 4,8-dihydroxy-6-(3,4-dichloro-phenyl)-quinoline-2-carboxylic acid 19m

By replacing methyl-4-hydroxy-8-amino-quinoline-2-carboxylate 2u in example 7 by methyl-8-benzyloxy-6-(3,4-dichloro-phenyl)-4-hydroxy-quinoline-2-carboxylate 18m and proceeding in the same manner, the abovenamed product is obtained. Yield: 80%. ¹H-NMR (200 MHz, DMSO-d₆): δ 7.97 (m, 1H arom.), 7.82 (m, 1H arom.), 7.74 (m, 2H arom.), 7.47 (m, 1H arom.), 7.06 (broad s, 1H arom.).

### 4,8-dihydroxy-6-(pyridin-3-yl)-quinoline-2-carboxylic acid 19o

By replacing methyl-4-hydroxy-8-amino-quinoline-2-carboxylate 2u in example 7 by methyl-8-benzyloxy-4-hydroxy-6-(pyridin-3-yl)-quinoline-2-carboxylate 18o and proceeding in the same manner, the abovenamed product is obtained. Yield: 70%. ¹H-NMR (200 MHz, DMSO-d₆): δ 8.90 (s, 1H arom.), 8.59 (m, 1H arom.), 8.15-8.09 (m, 1H arom.), 7.82 (m, 1H arom.), 7.55-7.45 (m, 2H arom.), 6.82 (m, 1H arom.).

### 4,8-dihydroxy-5-phenyl-quinoline-2-carboxylic acid 19a

By replacing methyl-4-hydroxy-8-amino-quinoline-2-carboxylate 2u in example 7 by methyl-4,8-dihydroxy-5-phenyl-quinoline-2-carboxylate 18b and proceeding in the same manner, the abovenamed product is obtained. Yield: 83%. ¹H-NMR (300 MHz, DMSO-d₆): δ 11.01(broad s, 1H, OH), 9.93 (broad s, 1H, OH), 7.24-7.10 (m, 6H arom.), 6.79 (m, 1H arom.), 6.40 (s, 1H arom.).

### 4,8-dihydroxy-5-bromo-quinoline-2-carboxylic acid 3c

By replacing methyl-4-hydroxy-8-amino-quinoline-2-carboxylate in example 7 by methyl-4,8-dihydroxy-5-bromo-quinoline-2-carboxylate 2c and proceeding in the same manner, the abovenamed product is obtained. Yield: 85%. ¹H-NMR (200 MHz, DMSO-d₆): δ 7.49 (d, 1H, J=8 Hz, 1H arom.), 7.04 (d, 1H, J=8 Hz, 1H arom.), 6.84 (s, 1H arom.).

### 8-methoxy-5-bromo-quinoline-2-carboxylic acid 4g

By replacing methyl-4-hydroxy-8-amino-quinoline-2-carboxylate in example 7 by methyl-8-methoxy-5-bromo-quinoline-2-carboxylate 5c and proceeding in the same manner, the abovenamed product is obtained. Yield: 88%. ¹H-NMR (200 MHz, DMSO-d₆): δ 13.2 (broad s, 1H, COOH), 8.59 (d, 1H, J=9 Hz, 1H arom.), 8.26 (d, 1H, J=9 Hz, 1H arom.) 7.99 (d, 1H, J=8 Hz, 1H arom.), 7.25 (d, 1H, J=8 Hz, 1H arom.), 4.04 (s, 3H, OCH₃).

### 4,8-dihydroxy-5-hydroxymethyl-4-quinoline-2-carboxylic acid 3g

By replacing methyl-4-hydroxy-8-amino-quinoline-2-carboxylate in example 7 by methyl-4,8-dihydroxy-5-hydroxymethyl-quinoline-2-carboxylate 2h and proceeding in the same manner, the abovenamed product is obtained. Yield: 82%. ¹H-NMR (300 MHz, DMSO-d₆): δ 7.13-7.07 (m, 2H arom.), 6.98 (s, 1H arom.), 2.72 (s, 2H, CH₂).

### 4-hydroxy-5-methyl-8-amino-quinoline-2-carboxylic acid 3v

By replacing methyl-4-hydroxy-8-amino-quinoline-2-carboxylate in example 7 by methyl-4-hydroxy-5-methyl-8-amino-quinoline-2-carboxylate 2v and proceeding in the same manner, the abovenamed product is obtained. Yield: 56%. ¹H-NMR (200 MHz, DMSO-d₆): δ 11.36 (broad s, 1H, COOH), 7.37 (broad s, 1H arom.), 7.00 (d poorly resolved, 1H arom.), 6.74 (d poorly resolved, 1H arom.), 2.67 (s, 3H, CH₃).

### 4-hydroxy-6-methyl-8-amino-quinoline-2-carboxylic acid 3w

By replacing methyl-4-hydroxy-8-amino-quinoline-2-carboxylate in example 7 by methyl-4-hydroxy-6-methyl-8-amino-quinoline-2-carboxylate 2w and proceeding in the same manner, the abovenamed product is obtained. Yield: 67%. ¹H-NMR (200 MHz, DMSO): δ 11.45 (broad s, 1H, COOH), 7.37 (broad s, 1H arom.), 7.02 (s, 1H arom.), 6.68 (s, 1H arom.), 2.34 (s, 3H, CH₃).

### 4-hydroxy-8-bromo-quinoline-2-carboxylic acid 3x

By replacing methyl-4-hydroxy-8-amino-quinoline-2-carboxylate in example 7 by methyl-4-hydroxy-8-bromo-quinoline-2-carboxylate 2x and proceeding in the same manner, the abovenamed product is obtained. Yield: 32%. ¹H-NMR (200 MHz, DMSO-d₆): δ 8.13 (m, 2H arom.), 7.42 (m, 1H arom.), 7.07 (m, 1H arom.).

### 8-hydroxy-4-phenethyl-quinoline-2-carboxylic acid 14a

By replacing methyl-4-hydroxy-8-amino-quinoline-2-carboxylate 2u in example 7 by methyl-8-hydroxy-4-phenethyl-quinoline-2-carboxylate 13a and proceeding in the same manner, the abovenamed product is obtained. Yield: 65%. ¹H-NMR (300 MHz, DMSO-d₆): δ 10.14 (broad s, 1H, COOH), 7.75(m, 1H arom.), 7.67-6.58 (m, 2H arom.), 7.22-7.17 (m, 3H arom.), 6.96 (m, 3H arom.), 3.45-3.34 (m, 4H, 2×CH₂).

### 8-hydroxy-4-(3-amino-propyl)-quinoline-2-carboxylic acid hydrochloride 14b

By replacing methyl-4-hydroxy-8-amino-quinoline-2-carboxylate 2u in example 7 by methyl-8-hydroxy-4-(3-tert-butoxycarbonylamino-propyl)-quinoline-2-carboxylate 13b and proceeding in the same manner, the abovenamed product is obtained. Yield: 83%. ¹H-NMR (300 MHz, DMSO-d₆): δ 10.26 (broad s, 1H, COOH), 8.07 (broad s, 3H, NH₃⁺), 8.03 (s, 1H arom.), 7.67 (m, 2H arom.), 7.22 (s, 1H arom.), 3.47 (m, 2H, CH₂), 2.93 (m, 2H, CH₂), 2.05 (m, 2H, CH₂).

### 8-hydroxy-4-(3-hydroxy-propyl)-quinoline-2-carboxylic acid 14c

By replacing methyl-4-hydroxy-8-amino-quinoline-2-carboxylate 2u in example 7 by methyl-8-hydroxy-4-(3-hydroxy-propyl)-quinoline-2-carboxylate 13c and proceeding in the same manner, the abovenamed product is obtained. Yield: 83%. ¹H-NMR (200 MHz, methanol-d₄): δ 8.12 (s, 1H arom.), 7.73-7.60 (m, 2H arom.), 7.20 (s, 1H arom.), 3.72 (m, 2H, CH₂), 3.34 (m, 2H, CH₂), 2.03 (m, 2H, CH₂).

### 4-amino-8-hydroxy-quinoline-2-carboxylic acid 17′a

By replacing methyl-4-hydroxy-8-amino-quinoline-2-carboxylate in example 7 by methyl-4-amino-8-hydroxy-quinoline-2-carboxylate 17a and proceeding in the same manner, the abovenamed product is obtained. Yield: 48%. ¹H-NMR (300 MHz, methanol-d₄): δ 7.94 (d, 1H, J=8 Hz, 1H arom.), 7.73 (m, 1H arom.), 7.58 (s, 1H arom.), 7.51 (d, 1H, J=8 Hz, 1H arom.).

### 4,8-diamino-quinoline-2-carboxylic acid 17′b

By replacing methyl-4-hydroxy-8-amino-quinoline-2-carboxylate in example 7 by methyl-4,8-diamino-quinoline-2-carboxylate 17b and proceeding in the same manner, the abovenamed product is obtained. Yield: 62%. ¹H-NMR (200 MHz, DMSO-d₆): δ 7.53-7.40 (m, 2H arom.), 7.20 (s, 1H arom.), 7.16 (d, 1H, J=9 Hz, 1H arom.).

### 4-hydroxy-8-(2H-tetrazol-5-yl)-quinoline-2-carboxylic acid hydrochloride 62b

By replacing methyl-4-hydroxy-8-amino-quinoline-2-carboxylate in example 7 by methyl-4-hydroxy-8-(2H-tetrazol-5-yl)-quinoline-2-carboxylate 62a and proceeding in the same manner, the abovenamed product is obtained. Yield: 64%. ¹H-NMR (200 MHz, DMSO-d₆+D₂O): δ 8.48-8.38 (m, 2H arom.), 7.56-7.48 (m, 1H arom.), 6.75 (s, 1H arom.).

### 3-phenylethyl-8-amino-4-hydroxy-quinoline-2-carboxylic acid 68c

By replacing methyl-4-hydroxy-8-amino-quinoline-2-carboxylate 3u in example 7 by methyl-3-phenylethyl-8-amino-4-hydroxy-quinoline-2-carboxylate 68b, the abovenamed product is obtained. Yield: 92%. ¹H NMR (300 MHz, CDCl₃): δ 12.14 (s, 1H, OH), 9.16 (s, 1H, OH), 7.36 (d, 1H, J=8 Hz, H arom.), 7.22 (m, 6H, 7H arom.), 6.89 (d, 1H, J=8 Hz, H arom.), 4.68 (s, 2H, NH₂), 3.21 (dd, 2H, J=8 and 10 Hz, CH₂), 2.78 (dd, 2H, J=8 and 10 Hz, CH₂).

### 3-(3′-aminopropyl)-8-amino-4-hydroxy-quinoline-2-carboxylic acid hydrochloride 71c

By replacing methyl 4-hydroxy-8-amino-quinoline-2-carboxylate 3u in example 7 by 3-(3′-N-(terbutoxycarbonyl)aminopropyl)-8-amino-4-hydroxy-quinoline-2-carboxylic 71b, the abovenamed product is obtained. Yield: 82%. ¹H NMR (300 MHz, d₆-DMSO): δ 7.50 (t, 1H, J=8 Hz, H⁶), 7.24 (m, 2H, 1 OH and 1H arom), 6.86 (d, 1H, J=9 Hz, 1H arom.), 6.64 (s, 2 HN), 4.41 (m, 2H, CH₂N), 3.35 (m, 3H, CH₂ and HN), 1.35 (m, 2H, CH₂), 1.66 (s, 9H, 3 CH₃).

### 5-phenylethyl-8-amino-4-hydroxy-quinoline-2-carboxylic acid 74c

By replacing methyl-4-hydroxy-8-amino-quinoline-2-carboxylate 3u in example 7 by methyl-5-phenylethyl-8-amino-4-hydroxy-quinoline-2-carboxylate 74b, the abovenamed product is obtained. Yield: 84%. ¹H NMR (300 MHz, CDCl₃): δ 9.15 (s, 1H, OH), 7.24 (m, 7H, 7H arom.), 7.04 (d, 1H, J=8 Hz, H arom.), 6.93 (d, 1H, J=8 Hz, H arom.), 4.96 (s, 2H, NH₂), 3.42 (t, 2H, J=9 Hz, CH₂), 2.83 (t, 2H, J=9 Hz, CH₂).

### 5-hydroxypropyl-8-amino-4-hydroxy quinoline-2-carboxylic acid 75c

By replacing methyl-4-hydroxy-8-amino-quinoline-2-carboxylate 3u in example 7 by methyl-5-(3′-hydroxypropyl)-8-amino-4-hydroxy-quinoline-2-carboxylate 75b, the abovenamed product is obtained. Yield: 46%. ¹H NMR (200 MHz, CDCl₃): δ 11.48 (s, 1H, 1 OH), 10.65 (s, 1H, OH), 7.18 (d, 1H, J=8 Hz, H arom.), 7.02 (s, 1H, H³), 6.80 (d, 1H, J=8 Hz, H arom.), 5.65 (s, 2H, NH₂), 3.24 (m, 2H, CH₂O), 2.62 (m, 4H, 2 CH₂), 1.71 (m, 2H, CH₂).

### Methyl-5-(3′-aminopropyl)-8-amino-4-hydroxy-quinoline-2-carboxylate acid hydrochloride 76c

By replacing methyl-4-hydroxy-8-amino-quinoline-2-carboxylate 3u in example 7 by methyl-5-(3′-N-(terbutoxycarbonyl)aminoprop-1′-ynyl)-8-nitro-4-benzyloxy-quinoline-2-carboxylate 76b, the abovenamed product is obtained. Yield: 71%. ¹H NMR (300 MHz, CDCl₃): δ 12.17 ‘s, 1H, OH), 9.22 (s, 1 HO), 8.75 (s, 3 HN), 7.14 (d, 1H, J=8 Hz, H arom.), 7.06 (s, 1H³), 6.89 (d, 1H, J=8 Hz, H arom.), 4.49 (s, 2 HN), 2.69 (m, 2H, CH₂N), 2.50 (m, 2H, CH₂), 1.71 (m, 2H, CH₂).

### 5-(piperidin-1-yl)-8-amino-4-hydroxy-quinoline-2-carboxylic acid hydrochloride 77c

By replacing methyl-4-hydroxy-8-amino-quinoline-2-carboxylate 3u in example 7 by methyl-5-(piperidin-1-yl)-8-amino-4-hydroxy-quinoline-2-carboxylate 77b, the abovenamed product is obtained as a yellow solid. Yield: 61%. ¹H NMR (200 MHz, CDCl₃): δ 8.76 (s, 1H, OH), 7.44 (s, 1H³), 7.12 (d, 1H, J=8 Hz, H arom.), 6.94 (d, 1H, J=8 Hz, H arom.), 5.39 (s, 2 HN), 2.97 (m, 2H, NCH₂), 2.77 (m, 2H, NCH₂), 1.69 (m, 6H, 3 CH₂).

### 5-pipeerazin-1-yl-8-amino-4-hydroxy-quinoline-2-carboxylic acid hydrochloride 78c

By replacing methyl-4-hydroxy-8-amino-quinoline-2-carboxylate 3u in example 7 by methyl-piperazin-1-yl-8-amino-4-hydroxy-quinoline-2-carboxylate 78b, the abovenamed product is obtained as an orange solid Yield: 72%. ¹H NMR (200 MHz, d₆-DMSO): δ 12.24 (s, 1H, OH), 10.82 (s, 1H, OH), 8.12 (s, 2H, NH₂⁺), 7.32 (s, 1H³), 7.15 (d, 1H, J=8 Hz, H arom.), 6.81 (d, 1H, J=8 Hz, H arom.), 5.12 (s, 2H, H₂N), 2.81 (m, 2H, NCH₂), 2.78 (m, 2H, NCH₂), 2.21 (m, 6H, 3 CH₂), 2.14 (m, 2H, CH₂).

### 6-hydroxy-8-amino-4-hydroxy-quinoline-2-carboxylic acid 79d

By replacing methyl-4-hydroxy-8-amino-quinoline-2-carboxylate 3u in example 7 by methyl-6-benzyloxy-8-nitro-4-hydroxy-quinoline-2-carboxylate 79c, the abovenamed product is obtained. Yield: 69%. ¹H NMR (200 MHz, d₆-DMSO): δ 11.01 (s, 1 OH), 9.57 (s, 1 OH), 7.15 (s, 1H³), 6.28 (d, 1H, J=2 Hz, H⁵), 6.16 (d, 1H, J=2 Hz, H⁷), 5.60 (s, 2 HN).

### 6-chloro-8-amino-4-hydroxy-quinoline-2-carboxylic acid 117d

By replacing methyl-4-hydroxy-8-amino-quinoline-2-carboxylate 3u in example 7 by methyl-6-chloro-8-amino-4-hydroxy-quinoline-2-carboxylate 117c, the abovenamed product is obtained. Yield: 78%. ¹H NMR (200 MHz, d₆-DMSO): δ 9.13 (m, 4H, 2 OH and NH₂), 7.48 (s, 1H³), 7.19 (d, 1H, J=2 Hz, H⁵), 6.86 (d, 1H, J=2 Hz, H⁷).

### 6-phenylethyl-8-amino-4-hydroxy-quinoline-2-carboxylic acid 118c

By replacing methyl-4-hydroxy-8-amino-quinoline-2-carboxylate 3u in example 7 by methyl-6-phenylethy-8-amino-4-hydroxy-quinoline-2-carboxylate 118b, the abovenamed product is obtained. Yield: 79%. ¹H NMR (200 MHz, d₆-DMSO): δ 13.24 (s, 1H, OH), 10.73 (s, 1H, OH), 7.24 (m, 6H, 6H arom.), 7.10 (s, 1H, H arom.), 6.83 (s, 1H, H arom.), 5.24 (s, 2H, NH₂), 2.90 (s, 4H, 2 CH₂).

### 6-(3′-hydroxypropyl)-8-amino-4-hydroxy-quinoline-2-carboxylic acid 119c

By replacing methyl-4-hydroxy-8-amino-quinoline-2-carboxylate 3u in example 7 by methyl-6-(3′-hydroxypropyl)-8-amino-4-hydroxy-quinoline-2-carboxylate 119b, the abovenamed product is obtained. Yield: 64%. ¹H NMR (200 MHz, d₆-DMSO): δ 12.14 (s, 1H, OH), 10.21 (s, 1 HO), 7.19 (d, 1H, J=2 Hz, 1H arom.), 7.08 (s, 1H³), 6.97 (d, 1H, J=2 Hz, 1H arom.), 5.16 (s, 2 HN), 4.18 (s, 1 HO), 3.21 (m, 2H, CH₂O), 2.51 (m, 2H, CH₂), 1.70 (m, 2H, CH₂).

### 6-(3′-aminopropyl)-8-amino-4-hydroxy-quinoline-2-carboxylic acid 121c

By replacing methyl-4-hydroxy-8-amino-quinoline-2-carboxylate 3u in example 7 by methyl-6-(3′-N-(terbutoxycarbonyl)aminopropyl)-8-amino-4-hydroxy-quinoline-2-carboxylate 121b, the abovenamed product is obtained. Yield: 52%. ¹H NMR (200 MHz, d₆-DMSO): δ 8.00 (m, 4H, OH and NH₃⁺), 7.37 (m, 1H, H arom), 7.35 (m, 1H, H arom), 7.03 (s, 1H³), 2.78 (m, 4H, CH₂ and H₂N), 2.50 (m, 2H, CH₂), 1.94 (m, 2H, CH₂).

### 6-(pyridin-3-yl)ethyl-8-amino-4-hydroxy-quinoline-2-carboxylic acid hydrochloride 122c

By replacing methyl-4-hydroxy-8-amino-quinoline-2-carboxylate 3u in example 7 by methyl-6-(pyridin-3-yl)ethyl-8-amino-4-hydroxy-quinoline-2-carboxylate 122b, the abovenamed product is obtained as a yellow solid. Yield: 78%. ¹H NMR (200 MHz, d₆-DMSO): δ 12.15 (s, 1H, OH), 10.45 (s, 1H, OH), 8.37 (s, 1H, H arom.), 8.40 (d, 1H, J=2 Hz, H arom.), 7.49 (d, 1H, J=4 Hz, 1H arom.), 7.15 (m, 3H, 3H arom.), 6.68 (s, 1H, 1H arom.), 5.41 (s, 2 UN), 2.80 (m, 4H, 2 CH₂).

### 6-piperidin-1-yl-8-amino-4-hydroxy-quinoline-2-carboxylic acid hydrochloride 126c

By replacing methyl-4-hydroxy-8-amino-quinoline-2-carboxylate 3u in example 7 by methyl-6-(N-piperidinyl)-8-amino-4-hydroxy-quinoline-2-carboxylate 126b, the abovenamed product is obtained as a yellow solid. Yield: 62%. ¹H NMR (200 MHz, d₆-DMSO): δ 13.42 (s, 1H, OH), 10.32 (s, 1H, OH), 7.01 (d, 1H, J=2 Hz, H arom.), 6.88 (s, 1H³), 6.61 (d, 1H, J=2 Hz, H arom.), 5.97 (s, 2 HN), 2.88 (m, 4H, 2 NCH₂), 1.62 (m, 6H, 3 CH₂).

### 6-piperazin-1-yl-8-amino-4-hydroxy-quinoline-2-carboxylic acid hydrochloride 125c

By replacing methyl-4-hydroxy-8-amino-quinoline-2-carboxylate 3u in example 7 by methyl-6-(N-piperazinyl)-8-amino-4-hydroxy-quinoline-2-carboxylate 125b, the abovenamed product is obtained as a brown solid. Yield: 48%. ¹H NMR (200 MHz, d₆-DMSO): δ 10.96 (s, 1H, OH), 6.98 (d, 1H, J=2 Hz, H arom.), 6.86 (s, 1H³), 6.72 (d, 1H, J=2 Hz, H arom.), 5.90 (s, 2H, H₂N), 2.89 (m, 4H, 2 NCH₂), 2.20 (m, 6H, 3 CH₂).

### 6,8-diamino-4-hydroxy-quinoline-2-carboxylic acid hydrochloride 32d

By replacing methyl-4-hydroxy-8-amino-quinoline-2-carboxylate 3u in example 7 by methyl-6,8-diamino-4-hydroxy-quinoline-2-carboxylate 32c, the abovenamed product is obtained as a purple solid. Yield: 61%. ¹H NMR (200 MHz, d₆-DMSO): δ 12.42 (s, 1 H, OH), 11.82 (s, 1H, OH), 8.00 (s, 3H, NH₃⁺), 7.23 (d, 1H, H arom.), 7.01 (s, 1H³), 6.83 (d, 1H, H arom.), 5.20 (s, 2H, H₂N).

### Methyl-6-(N-anilino)-8-amino-4-hydroxy-quinoline-2-carboxylate 33c

By replacing methyl-4-hydroxy-8-amino-quinoline-2-carboxylate 3u in example 7 by methyl-6-(1-anilino)-8-amino-4-hydroxy-quinoline-2-carboxylate 33b, the abovenamed product is obtained as an orange solid. Yield: 81%. ¹H NMR (200 MHz, CDCl₃): δ 13.14 (s, 1H, OH), 9.51 (s, 1H, OH), 8.24 (s, 1H, 2NH+), 7.78 (m, 1H, 1H arom.), 7.45 (s, 1H³), 7.36 (m, 4H, 4H arom.), 7.20 (m, 2H, 2H arom.), 4.72 (s, 2H, NH₂).

### 8-amino-4-phenyl-quinoline-2-carboxylic acid 92d

By replacing methyl-4-hydroxy-8-amino-quinoline-2-carboxylate 2u in example 7 by methyl-8-amino-4-phenyl-quinoline-2-carboxylate 92c and proceeding in the same manner, the abovenamed product is obtained. Yield: 93%. ¹H-NMR (200 MHz, DMSO-d₆): δ 7.97 (s, 1H arom.), 7.61-7.51 (m, 6H arom.), 7.26 (d, 2H, J=8 Hz, 2H arom.), 3.70 (broad s, 2H, NH₂).

### 8-amino-4-(hex-1-yl)-quinoline-2-carboxylic acid hydrochloride 93c

By replacing methyl-4-hydroxy-8-amino-quinoline-2-carboxylate 2u in example 7 by methyl-8-amino-4-(hex-1-yl)-quinoline-2-carboxylate 93b and proceeding in the same manner, the abovenamed product is obtained. Yield: 90%. ¹H-NMR (200 MHz, DMSO-d₆): δ 9.50 (broad s, 1H, OH), 7.96 (s, 1H arom.), 7.58-7.55 (m, 2H arom.), 7.24-7.20 (m, 1H arom.), 3.10 (t, 2H, J=7 Hz, CH₂), 1.69-1.66 (m, 2H, CH₂), 1.31-1.23 (m, 6H, 3×CH₂), 0.96 (t, 3H, J=7 Hz, CH₃).

### 8-amino-4-(2-phenyleth-1-yl)-quinoline-2-carboxylic acid hydrochloride 94c

By replacing methyl-4-hydroxy-8-amino-quinoline-2-carboxylate 2u in example 7 by methyl-8-amino-4-(2-phenyleth-1-yl)-quinoline-2-carboxylate 94b and proceeding in the same manner, the abovenamed product is obtained. Yield: 72%. ¹H-NMR (200 MHz, DMSO-d₆): δ 7.98 (s, 1H arom.), 7.55-7.17 (m, 8H arom.), 3.45-3.37 (m, 2H, CH₂), 3.07-2.99(m, 2H, CH₂).

### 8-amino-4-(3-amino-prop-1-yl)-quinoline-2-carboxylic acid hydrochloride 95c

By replacing methyl-4-hydroxy-8-amino-quinoline-2-carboxylate 2u in example 7 by methyl-8-amino-4-(3-tert-butoxycarbonylamino-prop-1-yl)-quinoline-2-carboxylate 95b and proceeding in the same manner, the abovenamed product is obtained. Yield: 84%. ¹H-NMR (200 MHz, DMSO-d₆): δ 8.13 (broad s, 3H, N⁺H₃), 8.03 (s, 1H arom.), 7.62-7.59 (m, 2H arom.), 7.28-7.26 (m, 1H arom.), 3.19 (t, 2H, J=7 Hz, CH₂), 2.93 (t, 2H, J=7 Hz, CH₂), 2.04-1.99 (m, 2H, CH₂).

### 8-amino-4-(3-hydroxy-prop-1-yl)-quinoline-2-carboxylic acid hydrochloride 96c

By replacing methyl-4-hydroxy-8-amino-quinoline-2-carboxylate 2u in example 7 by methyl-8-amino-4-(3-hydroxy-prop-1-yl)-quinoline-2-carboxylate 96b and proceeding in the same manner, the abovenamed product is obtained. Yield: 95%. ¹H-NMR (200 MHz, DMSO-d₆): δ 8.03 (s, 1H arom.), 7.62-7.59 (m, 2H arom.), 7.28-7.26 (m, 1H arom.), 6.15 (broad s, 3H, N⁺H₃), 3.51 (t, 2H, J=7 Hz, CH₂), 3.15 (t, 2H, J=7 Hz, CH₂), 1.89-1.82 (m, 2H, CH₂).

### 4-(3-acetyl-aminoprop-1-ynyl)-8-amino-quinoline-2-carboxylic acid hydrochloride 97c

By replacing methyl-4-hydroxy-8-amino-quinoline-2-carboxylate 2u in example 7 by methyl-4-(3-acetyl-aminoprop-1-ynyl)-8-amino-quinoline-2-carboxylate 97b and proceeding in the same manner, the abovenamed product is obtained. Yield: 84%. ¹H-NMR (200 MHz, DMSO-d₆): δ 8.01(s, 1H arom.), 7.66-7.57 (m, 2H arom.), 7.30 (d, 1H, J=8 Hz, 1H arom.), 6.93 (s, 1H, NH), 6.69 (broad s, 2H, NH₂), 4.58 (s, 2H, NCH₂), 2.34 (s, 3H, COCH₃).

### 8-hydroxy-4-(morpholin-1-yl)-quinoline-2-carboxylic acid hydrochloride 99c

By replacing methyl-4-hydroxy-8-amino-quinoline-2-carboxylate 2u in example 7 by methyl-8-hydroxy-4-(morpholin-1-yl)-quinoline-2-carboxylate 99b and proceeding in the same manner, the abovenamed product is obtained. Yield: 90%. ¹H-NMR (300 MHz, DMSO-d₆): δ 11.10 (broad s, 2H, OH), 7.56-754 (m, 3H arom.), 7.29-7.27 (m, 1H arom.), 3.89-3.87 (m, 4H, CH₂—O—CH₂), 3.55-3.53 (m, 4H, CH₂—N—CH₂).

### 8-hydroxy-4-(piperidin-1-yl)-quinoline-2-carboxylic acid hydrochloride 100c

By replacing methyl-4-hydroxy-8-amino-quinoline-2-carboxylate 2u in example 7 by methyl-8-hydroxy-4-(morpholin-1-yl)-quinoline-2-carboxylate 100b and proceeding in the same manner, the abovenamed product is obtained. Yield: 85%. ¹H-NMR (200 MHz, DMSO-d₆): δ 11.09 (broad s, 1H, OH), 8.84 (broad s, 1H, OH), 7.56-7.48 (m, 3H arom.), 7.33-7.29 (m, 1H arom.), 3.62-3.60 (m, 4H, CH₂—N—CH₂), 1.82-1.78 (m, 6H, 3×CH₂).

### 8-amino-4-(piperidin-1-yl)-quinoline-2-carboxylic acid hydrochloride 101c

By replacing methyl-4-hydroxy-5-hydroxymethyl-8-benzyloxy-quinoline-2-carboxylate 2g in example 2 by methyl-8-amino-4-(piperidin-1-yl)-quinoline-2-carboxylate 101b and proceeding in the same manner, the abovenamed product is obtained. Yield: 95%. ¹H-NMR (200 MHz, DMSO-d₆): δ 9.75 (broad s, 4H, NH₂+2×OH), 7.69-7.53 (m, 4H arom.), 3.33-3.30 (m, 4H, CH₂—N—CH₂), 1.77-1.59 (m, 6H, 3×CH₂).

### 4-hydroxy-8-(piperazin-1-yl)-quinoline-2-carboxylic acid hydrochloride 108b

By replacing methyl-4-hydroxy-5-hydroxymethyl-8-benzyloxy-quinoline-2-carboxylate 2g in example 2 by methyl-4-hydroxy-8-(piperazin-1-yl)-quinoline-2-carboxylate 108a and proceeding in the same manner, the abovenamed product is obtained. Yield: 89%. ¹H-NMR (200 MHz, DMSO-d₆): δ 9.47 (broad s, 2H, NH+OH), 7.91 (d, 1H, J=8 Hz, 1H arom.), 7.59 (d, 1H, J=8 Hz, 1H arom.), 7.44 (t, 1H, J=8 Hz, 1H arom.), 6.84 (s, 1H arom.), 3.39-3.32 (m, 4H, CH₂—N—CH₂), 3.29-3.26 (m, 4H, CH₂—N—CH₂).

### 8-hydroxy-4-(4-methyl-piperazin-1-yl)-quinoline-2-carboxylic acid hydrochloride 110c

By replacing methyl-4-hydroxy-5-hydroxymethyl-8-benzyloxy-quinoline-2-carboxylate 2g in example 2 by methyl-8-hydroxy-4-(4-methyl-piperazin-1-yl)-quinoline-2-carboxylate 110b and proceeding in the same manner, the abovenamed product is obtained. Yield: 65%. ¹H-NMR (300 MHz, DMSO-d₆): δ 11.38 (broad s, 1H, COOH), 7.62-7.48 (m, 3H arom.), 7.21 (m, 1H arom), 3.86-3.82 (m, 2H, CH₂), 3.29-3.26 (m, 6H, 3×CH₂), 2.86 (s, 3H, NCH₃).

### 4-hydroxy-8-phenylethyl-quinoline-2-carboxylic acid 111c

By replacing methyl-4-hydroxy-5-hydroxymethyl-8-benzyloxy-quinoline-2-carboxylate 2g in example 2 by methyl-4-hydroxy-8-phenylethyl-quinoline-2-carboxylate 111a and proceeding in the same manner, the abovenamed product is obtained. Yield: 90%. ¹H-NMR (200 MHz, DMSO-d₆): δ 9.54 (broad s, 2H, OH), 8.01 (d, 1H, J=8 Hz, 1H arom.), 7.60 (d, 1H, J=8 Hz, 1H arom.), 7.45-7.14 (m, 7H arom.), 3.41 (t, 2H, J=6.5 Hz, CH₂), 2.98 (t, 2H, J=6.5 Hz, CH₂).

### EXAMPLE 8

### 4-hydroxy-8-benzyloxy-quinoline-2-carboxylic acid 3y

Dissolve methyl-4-hydroxy-8-benzyloxy-quinoline-2-carboxylate 2a (300 mg, 0.97 mmol) in a 1:1 mixture of THF/water (20 ml). To this solution add lithium hydroxide hydrate (216 mg, 5.1 mmol). Allow to stand at room temperature overnight. Evaporate the THF then cool the medium to 0° C. Acidify to pH 2 with concentrated hydrochloric acid solution. Filter the precipitate formed. Yield: 30%. ¹H-NMR (200 MHz, DMSO-d₆): δ 8.90 (very broad s, 2H, COOH, OH), 7.70-7.29 (m, 8H arom.), 6.67 (s, 1H arom.), 5.40 (s, 2H, OCH₂).

### 8-amino-quinoline-2-carboxylic acid 4f

By replacing methyl-4-hydroxy-8-benzyloxy-quinoline-2-carboxylate in example 8 by methyl 8-amino-quinoline-2-carboxylate 5f and proceeding in the same manner, the abovenamed product is obtained. Yield: 85%. ¹H-NMR (300 MHz, DMSO-d₆): δ 12.84 (broad s, 1H, COOH), 8.37 (d, 1H, J=8 Hz, 1H arom.), 8.06 (d, 1H, J=8 Hz, 1H arom.), 7.44 (m, 1H arom.), 7.12 (d, 1H, J=7 Hz, 1H arom.), 6.90 (d, 1H, J=7 Hz, 1H arom.), 6.57 (broad s, 2H, NH₂).

### 8-benzylamino-4-hydroxy-quinoline-2-carboxylic acid 30a

By replacing methyl-4-hydroxy-8-benzyloxy-quinoline-2-carboxylate in example 8 by- methyl 8-benzylamino-4-hydroxy-quinoline-2-carboxylate 29a and proceeding in the same manner, the abovenamed product is obtained. Yield: 69%. ¹H-NMR (300 MHz, DMSO-d₆): δ 11.60 (very broad s, 2H, COOH and OH), 7.96 (broad s, 1H, NH), 7.44-6.61 (m, 9H arom.), 4.55 (s, 2H, OCH₂).

### 8-hydroxy-4-(toluene-4-sulfonylamino)-quinoline-2-carboxylic acid 16a

By replacing methyl-4-hydroxy-8-benzyloxy-quinoline-2-carboxylate in example 8 by methyl-8-hydroxy-4-(toluene-4-sulfonylamino)-quinoline-2-carboxylate 15b and proceeding in the same manner, the abovenamed product is obtained. Yield: 62%. ¹H-NMR (200 MHz, MeOD): δ 8.25 (s, 1H, H arom.), 7.97 (m, 3H arom.), 7.55 (m, 3H arom.), 7.35 (d, 1H, J=7 Hz, H arom.), 2.54 (s, 3H, CH₃).

### 8-bromo-4-hydroxy-6-isopropyl-quinoline-2-carboxylic acid 42c

By replacing methyl-4-hydroxy-8-benzyloxy-quinoline-2-carboxylate in example 8 by methyl-8-bromo-4-hydroxy-6-isopropyl-quinoline-2-carboxylate 42b and proceeding in the same manner, the abovenamed product is obtained. Yield: 68%. ¹H-NMR (300 MHz, DMSO): δ 8.09 (d, 1H, J=1.86 Hz, H arom.), 7.98 (d, 1H, J=1.86 Hz, 1H, H arom.), 7.6 (broad s, 1H arom.), 3.15-3.02 (sept, 1H, CH(CH₃)₂), 1.29 and 1.26 (2s, 6H, CH(CH₃)₂).

### 5,7-dichloro-4,8-dihydroxy-quinoline-2-carboxylic acid 44d

By replacing methyl-4-hydroxy-8-benzyloxy-quinoline-2-carboxylate in example 8 by methyl-5,7-dichloro-4,8-dihydroxy-quinoline-2-carboxylate 44c and proceeding in the same manner, the abovenamed product is obtained. Yield: 79%. ¹H-NMR (300 MHz, DMSO): δ 10.90 (broad s, 1H, OH), 7.64 (s, 1H arom.), 7.42 (broad s, 1H arom.).

### 8-benzyloxy-6-bromo-4-hydroxy-quinoline-2-carboxylic acid 46a

By replacing methyl-4-hydroxy-8-benzyloxy-quinoline-2-carboxylate in example 8 by methyl-8-benzyloxy-6-bromo-4-hydroxy-quinoline-2-carboxylate 3k and proceeding in the same manner, the abovenamed product is obtained. Yield: 79%. ¹H-NMR (300 MHz, DMSO): δ 7.76 (d, 1H arom.), 7.61-7.57 (m, 3H arom.), 7.49-7.40 (m, 3H arom.), 6.72 (broad s, 1H arom.), 5.42 (s, 2H, OCH₂).

### 8-benzyloxy-7-bromo-4-hydroxyquinoline-2-carboxylic acid 50c

By replacing methyl-4-hydroxy-8-benzyloxy-quinoline-2-carboxylate in example 8 by methyl-8-benzyloxy-7-bromo-4-hydroxy-quinoline-2-carboxylate 50b and proceeding in the same manner, the abovenamed product is obtained. Yield: 70%. ¹H-NMR (200 MHz, DMSO): δ 7.83-7.79 (d, 1H arom.), 7.70-7.65 (d, 1H arom.), 7.63-7.52 (m, 2H arom.), 7.47-7.34 (m, 3H arom.), 6.85 (broad s, 1H arom.), 5.32 (s, 2H, OCH₂).

### 3-bromo-4,8-dihydroxy-quinoline-2-carboxylic acid 52c

By replacing methyl-4-hydroxy-8-benzyloxy-quinoline-2-carboxylate in example 8 by methyl-3-bromo-4,8-dihydroxy-quinoline-2-carboxylate 52b and proceeding in the same manner, the abovenamed product is obtained. Yield: 44%. ¹H-NMR (200 MHz, DMSO): δ 12.02 (broad s, 1H, CO₂H), 10.94 (s, 1H, OH), 7.60-7.59 (dd, 1H, J=1 Hz and J=8 Hz, 1H arom.), 7.29-7.21 (t, 1H, J=8 Hz, 1H arom.), 7.17-7.12 (dd, 1H, J=1 Hz and J=8 Hz, 1H arom.).

### 8-benzyloxy-3-bromo-4-hydroxy-quinoline-2-carboxylic acid 53a

By replacing methyl-4-hydroxy-8-benzyloxy-quinoline-2-carboxylate in example 8 by methyl-8-benzyloxy-3-bromo-4-hydroxy-quinoline-2-carboxylate 52b and proceeding in the same manner, the abovenamed product is obtained. Yield: 63%. ¹H-NMR (300 MHz, DMSO): δ 12.06 (broad s, 1H, CO₂H), 7.70-7.68 (m, 1H arom.), 7.58-7.55 (m, 2H arom.), 7.45-7.30 (m, 5H arom.), 5.40 (s, 2H, OCH₂).

### -3,7-dibromo-4,8-dihydroxy-quinoline-2-carboxylic acid 56c

By replacing methyl-4-hydroxy-8-benzyloxy-quinoline-2-carboxylate in example 8 by methyl-3,7-dibromo-4,8-dihydroxy-quinoline-2-carboxylate 56b and proceeding in the same manner, the abovenamed product is obtained. Yield: 72%. ¹H-NMR (300 MHz, DMSO): δ 12.26 (broad s, 1H, CO₂H or OH), 10.62 (broad s, 1H, OH), 7.64-7.48 (m, 2H arom.).

### 8-hydroxy-3-oxo-3,4-dihydroquinoxaline-2-carboxylic acid 59c

By replacing methyl-4-hydroxy-8-benzyloxy-quinoline-2-carboxylate in example 8 by ethyl-8-hydroxy-3-oxo-3,4-dihydroquinoxaline-2-carboxylate 59b and proceeding in the same manner, the abovenamed product is obtained. Yield: 62%. ¹H-NMR (200 MHz, DMSO): δ 13.82 (broad s, 1H, COOH), 12.84 (broad s, 1H, NH), 10.46 (s, 1H, OH), 7.46 (t, 1H, J=8 Hz, 1H arom.), 6.79-6.75 (d, 2H, J=8 Hz, 2H arom.).

### [8-(hydroxy)-3-oxo-3,4-dihydroquinoxaline-2(1H)-yl]acetic acid 60c

By replacing methyl-4-hydroxy-8-benzyloxy-quinoline-2-carboxylate in example 8 by ethyl[8-(hydroxy)-3-oxo-3,4-dihydroquinoxaline-2(1H)-ylilene]acetate 60b and proceeding in the same manner, the abovenamed product is obtained. Yield: 65%. ¹H-NMR (200 MHz, DMSO-d₆): δ 12.19 (s, 1H, OH), 9.84 (broad s, 1H, NHCO), 7.30-7.22 (t, 1H, J=8 Hz, 1H arom.), 6.71-6.67 (d, 1H, J=8 Hz, 1H arom.), 6.69-6.65 (d, 1H, J=8 Hz, 1H arom.), 2.41 (s, 2H, CH₂).

### 6-bromo-8-cyano-4-hydroxy-quinoline-2-carboxylic acid 63c

By replacing methyl-4-hydroxy-8-benzyloxy-quinoline-2-carboxylate in example 8 by methyl-6-bromo-8-cyano-4-hydroxy-quinoline-2-carboxylate 63b and proceeding in the same manner, the abovenamed product is obtained. Yield: 68%. ¹H-NMR (200 MHz, DMSO-d₆): δ 12.83 (broad s, 2H, COOH and OH), 8.64-8.58 (m, 2H arom.), 7.51 (s, 1H arom.).

### 8-cyano-4-hydroxy-6-phenyl-quinoline-2-carboxylic acid 64b

By replacing methyl-4-hydroxy-8-benzyloxy-quinoline-2-carboxylate in example 8 by methyl-8-cyano-4-hydroxy-6-phenyl-quinoline-2-carboxylate 64a and proceeding in the same manner, the abovenamed product is obtained. Yield: 65%. ¹H-NMR (200 MHz, DMSO-d₆): δ 12.80 (broad s, 2H, COOH and OH), 8.77-8.65 (m, 2H arom.), 7.93-7.89 (m, 2H arom.), 7.60-7.48 (m, 4H arom.).

### 8-cyano-4-hydroxy-6-phenethyl-quinoline-2-carboxylic acid 65d

By replacing methyl-4-hydroxy-8-benzyloxy-quinoline-2-carboxylate in example 8 by methyl-8-cyano-4-hydroxy-6-phenethyl-quinoline-2-carboxylate 65c and proceeding in the same manner, the abovenamed product is obtained. Yield: 84%. ¹H-NMR (200 MHz, DMSO-d₆+TFA): δ 8.24 (s, 2H arom.), 7.47 (s, 1H arom.), 7.24 (m, 5H arom.), 3.18-2.99 (m, 4H, PhCH₂—CH₂).

### 3-bromo-8-cyano-4-hydroxy-quinoline-2-carboxylic acid 66b

By replacing methyl-4-hydroxy-8-benzyloxy-quinoline-2-carboxylate in example 8 by methyl-3-bromo-8-cyano-4-hydroxyquinoline-2-carboxylate 66a and proceeding in the same manner, the abovenamed product is obtained. Yield: 57%. ¹H-NMR (200 MHz, DMSO-d₆): δ 8.52-8.48 (m, 1H arom.), 8.37-8.34 (m, 1H arom.), 7.69-7.61 (m, 1H arom.).

### 8-cyano-4-hydroxy-3-phenylethynyl-quinoline-2-carboxylic acid 67c

By replacing methyl-4-hydroxy-8-benzyloxy-quinoline-2-carboxylate in example 8 by benzyl-4-benzyloxy-8-cyano-3-phenylethynyl-quinoline-2-carboxylate 67b and proceeding in the same manner, the abovenamed product is obtained. Yield: 56%. ¹H-NMR (300 MHz, DMSO-d₆): δ 8.86-8.83 (m, 1H arom.), 8.47-8.44 (m, 1H arom.), 8.20-8.18 (m, 2H arom.), 7.98 (m, 1H arom.), 7.61-7.53 (m, 3H arom.).

### 8-cyano-6-ethyl-4-hydroxy-quinoline-2-carboxylic acid 69d

By replacing methyl-4-hydroxy-8-benzyloxy-quinoline-2-carboxylate in example 8 by methyl-8-cyano-6-ethyl-4-hydroxy-quinoline-2-carboxylate 69c and proceeding in the same manner, the abovenamed product is obtained. Yield: 60%. ¹H-NMR (200 MHz, MeOD): δ 8.31 (m, 1H arom.), 8.13 (m, 1H arom.), 7.17 (m, 1H arom.), 2.89-2.84 (m, 2H, CH₂—CH₃), 1.39-1.34 (m, 3H, CH₂—CH₃).

### 8-fluoro-4-hydroxy-quinoline-2-carboxylic acid 36c

By replacing methyl-4-hydroxy-8-benzyloxy-quinoline-2-carboxylate 2a in example 8 by methyl-8-fluoro-4-hydroxy-quinoline-2-carboxylate 36b, the abovenamed product is obtained. Yield: 91%. ¹H NMR (300 MHz, d₆-DMSO): δ 11.61 (s, 1 OH), 10.81 (s, 1 OH), 7.93 (d, 1H, J=8 Hz, 1H⁵), 7.62 (t, 1H, J=8 Hz, H⁶), 7.48 (d, 1H, J=2 Hz, H⁷), 7.07 (s, 1H³).

### 8-carboxamide-4-hydroxy-quinoline-2-carboxylate acid 37c

By replacing the compound in example 8 by methyl-8-carboxamide-4-hydroxy-quinoline-2-carboxylate 37b, the abovenamed product is obtained as a white solid. Yield: 90%. ¹H NMR (200 MHz, d₆-DMSO): δ 14.70 (s, 1 OH), 13.28 (s, 1 OH), 8.50 (s, 1H, 1 NH), 8.32 (d, 1H, J=8 Hz, H arom.), 8.29 (d, 1H, J=8 Hz, H arom.), 7.99 (s, 1H, 1 NH), 7.49 (t, 1H, J=8 Hz, H⁶), 6.69 (s, 1H³).

### 3-bromo-8-amino-4-hydroxy-quinoline-2-carboxylic acid 45c

By replacing methyl-4-hydroxy-8-benzyloxy-quinoline-2-carboxylate 2a in example 8 by methyl-3-bromo-4-hydroxy-quinoline-2-carboxylate 45b, the abovenamed product is obtained as a white solid. Yield: 51%. ¹H NMR (200 MHz, d₆-DMSO): δ 7.40 (d, 1H, J=8 Hz, H⁷), 7.21 (t, 1H, J=8 Hz, H⁶), 6.98 (d, 1H, J=8 Hz, H⁵), 3.9 (s, 2H, 2 HN).

### 8-cyano-4-hydroxy-quinoline-2-carboxylate acid 35c

By replacing methyl-4-hydroxy-8-benzyloxy-quinoline-2-carboxylate 2a in example 8 by methyl-8-cyano-4-hydroxy-quinoline-2-carboxylate 35b, the abovenamed product is obtained as a white solid. Yield: 93%. ¹H NMR (200 MHz, d₆-DMSO): δ 13.19 (s, 1 OH), 12.80 (s, 1 OH), 8.45 (d, 1H, J=8 Hz, H⁷), 8.37 (d, 1H, J=8 Hz, H⁵), 7.71 (t, 1H, J=8 Hz, H⁶), 7.50 (s, 1H³).

### 5-phenyl-8-amino-4-hydroxy-quinoline-2-carboxylic acid 73c

By replacing methyl-4-hydroxy-8-benzyloxy-quinoline-2-carboxylate 2a in example 8 by methyl-5-phenyl-8-amino-4-hydroxy-quinoline-2-carboxylate 73b, the abovenamed product is obtained as a yellow solid. Yield: 78%. ¹H NMR (300 MHz, CDCl₃): δ 11.91 (s, 1H, OH), 9.95 (s, 1H, OH), 7.21 (m, 5H, 5H arom.), 7.18 (d, 1H, J=8 Hz, H arom.), 7.10 (d, 1H, J=8 Hz, H arom.), 7.00 (s, 1H, H³), 5.23 (s, 2H, NH₂).

### 6-(5′-cyanopentyl)-8-amino-4-hydroxy-quinoline-2-carboxylic acid 123c

By replacing methyl-4-hydroxy-8-benzyloxy-quinoline-2-carboxylate 2a in example 8 by methyl-6-(5′-cyanopentyl)-8-amino-4-hydroxy-quinoline-2-carboxylate 123b, the abovenamed product is obtained as a yellow solid. Yield: 78%. ¹H NMR (200 MHz, d₆-DMSO): δ 12.58 (s, 1H, OH), 11.40 (s, 1H, OH), 7.28 (s, 1H arom.), 6.89 (s, 1H arom.), 6.78 (s, 1H arom.), 5.02 (s, H₂N), 2.60 (t, 4H, J=7 Hz, 2 CH₂), 1.61 (m, 4H, 2 CH₂), 1.40 (m, 2H, CH₂).

### 6-cyano-8-amino-4-hydroxy-quinoine-2-carboxylic acid 120c

By replacing methyl-4-hydroxy-8-benzyloxy-quinoline-2-carboxylate 2a in example 8 by methyl-6-cyano-8-nitro-4-hydroxy-quinoline-2-carboxylate 120b, the abovenamed product is obtained. Yield: 42%. ¹H NMR (300 MHz, d₆-DMSO): δ 12.01 (s, 1 OH), 6.28 (s, 1H, H⁵), 7.24 (s, 1H³), 6.86 (s, 1H, H⁷), 6.63 (s, 2 HN).

### 6-N-(4-methylpiperazin-1-yl)-8-nitro-4-benzyloxy-quinoline-2-carboxylic acid hydrochloride 124b

By replacing methyl-4-hydroxy-8-benzyloxy-quinoline-2-carboxylate 2a in example 8 by methyl-6-(4-methyl-piperazin-1-yl)-8-nitro-4-benzyloxy-quinoline-2-carboxylate 124a, the abovenamed product is obtained. Yield: 86%. ¹H-NMR (300 MHz, d₆-DMSO): δ 11.96 (s, 1 OH), 8.30 (d, 1H, J=2 Hz, 1H arom.), 7.70 (s, 1H³), 7.50 (m, 6H, 6H arom.), 6.63 (s, 2H, CH₂benz.), 3.68 (m, 4H, 2 CH₂N), 3.34 (m, 5H, 1H and 2 CH₂N).

### 8-dimethylamino-4-hydroxy-quinoline-2-carboxylic acid 41d

By replacing methyl-4-hydroxy-8-benzyloxy-quinoline-2-carboxylate 2a in example 8 by methyl-8-dimethylamino-4-hydroxy-quinoline-2-carboxylate 41c, the abovenamed product is obtained. Yield: 66%. ¹H NMR (200 MHz, d₆-DMSO): 8.23 (d, 2H, J=8 Hz, 2H arom.), 7.75 (t, 1H, J=8 Hz, 1H⁶), 7.51 (s, 1H, H³), 3.26 (s, 6H, 2 NCH₃).

### 4-(3-benzoyl-aminoprop-1-yl)-8-hydroxyquinoline-2-carboxylic acid 85e

By replacing mehtyl-4-hydroxy-8-benzyloxy-quinoline-2-carboxylate 2a in example 8 by methyl-4-(3-benzoyl-aminoprop-1-yl)-8-hydroxy-quinoline-2-carboxylate 85d and proceeding in the same manner, the abovenamed product is obtained. Yield: 93%. ¹H-NMR (300 MHz, DMSO-d₆): δ 12.81(s, 1H, COOH), 10.18 (broad s, 1H, OH), 8.55 (t, 1H, J=5.6 Hz, NHCO), 8.08 (s, 1H arom.), 7.85-7.18 (m, 8H arom.), 3.39-3.31 (m, 4H, CH₂), 1.99-1.95 (m, 2H, CH₂).

### 8-amino-4-hydroxy-6-phenyl-quinoline-2-carboxylic acid 86d

By replacing methyl-4-hydroxy-8-benzyloxy-quinoline-2-carboxylate 2a in example 8 by methyl-8-amino-4-hydroxy-6-phenyl-quinoline-2-carboxylate 86c and proceeding in the same manner, the abovenamed product is obtained. Yield: 82%. ¹H-NMR (300 MHz, DMSO-d₆): δ 12.58 (s, 1H, COOH), 11.65 (broad s, 1H, OH), 7.73 (s, 1H arom.), 7.73 (s, 1H arom.), 7.51-7.41 (m, 5H arom.), 7.18 (s, 1H arom.), 6.62 (broad s, 2H, NH₂).

### EXAMPLE 9

### 8-benzyloxy-7-bromo-quinoline-2-carboxylic acid 4c

Dissolve sodium hydroxide (630 mg, 15.8 mmol) in 60 ml of methanol. Add to this solution 2-amino-3-benzyloxy-4-bromobenzaldehyde (600 mg, 1.96 mmol) and pyruvic acid (0.27 ml, 3.88 mmol). Heat this mixture at 60° C. overnight under a nitrogen atmosphere. Allow to cool and evaporate the methanol. Take up the oily red residue in water and wash with ethyl acetate. Take up the organic phase, adjust to pH 2 with concentrated hydrochloric acid solution. Filter the precipitate formed. Yield: 85%. ¹H-NMR (300 MHz, DMSO): δ 13.57 (broad s, 1H, COOH), 8.61 (d, 1H, J=8 Hz, 1H arom.), 8.19 (d, 1H, J=8 Hz, 1H arom.), 7.90 (d, 1H, J=9 Hz, 1H arom.), 7.78 (d, 1H, J=9 Hz, 1H arom.), 7.68 (m, 2H arom.), 7.39 (m, 3H arom.), 5.61 (s, 2H, OCH₂).

### EXAMPLE 10

### Benzyl 8-benzyloxy-7-bromo-quinoline-2-carboxylate 5d

In a dry flask under argon, dissolve 7-bromo-8-benzyloxy-quinoline-2-carboxylic acid 4c (300 mg, 0.84 mmol) in 10 ml of dry DMF. At room temperature add NaH (37 mg, 0.93 mmol). Stir for 10 minutes, then add dropwise to this mixture benzyl bromide (0.11 ml, 0.92 mmol). Run the reaction overnight then evaporate the DMF. Take up the oily residue in ethyl acetate and wash with water. Dry the organic phases on Na₂SO₄, filter and evaporate the solvents. Purify the crude reaction product by silica gel column chromatography, eluent: Hex/EtOAc, 4/1 and 3/1. Yield: 85%. ¹H-NMR (200 MHz, CDCl₃): δ 8.25 (m, 2H arom.), 7.78 (d, 1H, J=9 Hz, 1H arom.), 7.61 (m, 4H arom.), 7.49 (d, 1H, J=9 Hz, 1H arom.), 7.36 (m, 6H arom.), 5.65 (s, 2H, OCH₂), 5.53 (s, 2H, OCH₂).

### Benzyl 8-benzyloxy-quinoline-2-carboxylate 5a

By replacing 7-bromo-8-benzyloxy-quinoline-2-carboxylic acid 4c in example 10 by 8-hydroxy-quinoline-2-carboxylic acid and proceeding in the same manner, the abovenamed product is obtained. Yield: 78%. ¹H-NMR (200 MHz, CDCl₃): δ 8.25 (m, 2H arom.), 8.23-7.52 (m, 5H arom.), 7.39-7.38 (m, 7H arom.), 7.14 (m, 1H arom.), 5.51 (s, 2H, OCH₂), 5.43 (s, 2H, OCH₂).

### Methyl-4-benzyloxy-8-bromo-quinoline-2-carboxylate 45a′

By replacing 7-bromo-8-benzyloxy-quinoline-2-carboxylic acid 4c in example 10 by methyl-4-hydroxy-8-bromo-quinoline carboxylate 42b and proceeding in the same manner, the abovenamed product is obtained. Yield: 88%. ¹H-NMR (200 MHz, CDCl₃): δ 8.31-8.26 (dd, 1H arom.), 8.13-8.09 (dd, 1H arom.), 7.75 (s, 1H arom.), 7.52-7.3540 (m, 6H arom.), 5.38 (s, 2H, OCH₂), 4.08 (s, 3H, OCH₃).

### Benzyl-4-benzyloxy-8-bromo-quinoline-2-carboxylate 47a

By replacing 7-bromo-8-benzyloxy-quinoline-2-carboxylic acid 4c in example 10 by 8-bromo-4-hydroxy-quinoline-2-carboxylic acid 3x, and by doubling the quantities of NaH and benzyl bromide and proceeding in the same manner, the abovenamed product is obtained. Yield: 88%. ¹H-NMR (200 MHz, CDCl₃): δ 8.29-8.25 (dd, 1H arom.), 8.13-8.09 (dd, 1H arom.), 7.71 (s, 1H arom.), 7.59-7.35 (m, 1H arom.), 5.53 (s, 2H, OCH₂), 5.37 (s, 2H, OCH₂).

### Benzyl-4,8-dibenzyloxy-6-bromo-quinoline-2-carboxylate 46b

By replacing 7-bromo-8-benzyloxy-quinoline-2-carboxylic acid 4c in example 10 by 8-benzyloxy-6-bromo-4-hydroxy-quinoline-2-carboxylic acid 46a, and by doubling the quantities of NaH and benzyl bromide and proceeding in the same manner, the abovenamed product is obtained. Yield: 61%. ¹H-NMR (200 MHz, CDCl₃): δ 8.00 (d, 1H arom.), 7.71 (s, 1H arom.), 7.64-7.34 (m, 15H arom.), 7.27-7.25 (d, 1H arom.), 5.48 (s, 2H, OCH₂), 5.36 (s, 2H, OCH₂), 5.34 (s, 2H, OCH₂).

### Benzyl-4,8-dibenzyloxy-7-bromo-quinoline-2-carboxylate 50d

By replacing 7-bromo-8-benzyloxy-quinoline-2-carboxylic acid 4c in example 10 by 8-benzyloxy-7-bromo-4-hydroxy-quinoline-2-carboxylic acid 50c, and by doubling the quantities of NaH and benzyl bromide and proceeding in the same manner, the abovenamed product is obtained. Yield: 99%. ¹H-NMR (300 MHz, CDCl₃): δ 7.90-7.87 (d, 1H arom.), 7.72-7.68 (d, 1H arom.), 7.71 (s, 1H arom.), 7.65-7.28 (m, 15H arom.), 5.58 (s, 2H, OCH₂), 5.51 (s, 2H, OCH₂), 5.36 (s, 2H, OCH₂).

### Benzyl-3-bromo-4,8-dibenzyloxy-quinoline-2-carboxylate 53b

By replacing 7-bromo-8-benzyloxy-quinoline-2-carboxylic acid 4c in example 10 by 8-benzyloxy-3-bromo-4-hydroxy-quinoline-2-carboxylic acid 53a, and by doubling the quantities of NaH and benzyl bromide and proceeding in the same manner, the abovenamed product is obtained. Yield: 99%. ¹H-NMR (300 MHz, CDCl₃): δ 7.62-7.31 (m, 17H arom.), 7.12-7.09 (d, 1H arom.), 5.51 (s, 2H, OCH₂), 5.43 (s, 2H, OCH₂), 5.23 (s, 2H, OCH₂).

### Methyl-6-bromo-8-cyano-4-benzyloxy-quinoline-2-carboxylate 65a

By replacing 7-bromo-8-benzyloxy-quinoline-2-carboxylic acid 4c in example 10 by methyl-6-bromo-8-cyano-4-hydroxy-quinoline-2-carboxylate 63b and proceeding in the same manner, the abovenamed product is obtained. Yield: 96%. ¹H-NMR (300 MHz, CDCl₃): δ 8.63-8.62 (d, 1H, J=2 Hz, 1H arom.), 8.23-8.22 (d, 1H, J=2 Hz, 1H arom.), 7.80 (s, 1H arom.), 7.50-7.44 (m, 5H arom.), 5.39 (s, 2H, OCH₂), 4.08 (s, 3H, OCH₃).

### Benzyl-3-bromo-4-benzyloxy-8-cyano-quinoline-2-carboxylate 67a

By replacing 7-bromo-8-benzyloxy-quinoline-2-carboxylic acid 4c in example 10 by 3-bromo-8-cyano-4-hydroxy-quinoline-2-carboxylic acid 66b and proceeding in the same manner, the abovenamed product is obtained. Yield: 87%. ¹H-NMR (200 MHz, CDCl₃): δ 8.62-8.58 (m, 1H arom.), 8.03-8.00 (m, 1H arom.), 7.52-7.40 (m, 6H arom.), 5.56 (s, 2H, OCH₂).

### Methyl-5-bromo-8-nitro-4-benzyloxy-quinoline-2-carboxylate 72c

By replacing 7-bromo-8-benzyloxy-quinoline-2-carboxylic acid in example 10 by methyl-5-bromo-8-nitro-4-hydroxy-quinoline-2-carboxylate 72b, the abovenamed product is obtained. Yield: 94%. ¹H NMR (200 MHz, CDCl₃): δ 7.98 (d, 1H, J=8 Hz, H⁷), 7.85 (s, 1H³), 7.82 (d, 1H, J=8 Hz, H⁵), 7.61 (m, 2H arom.), 7.50 (m, 3H arom.), 5.46 (s, 2H benz.), 4.07 (s, 3H, OCH₃).

### Methyl-6-bromo-8-nitro-4-benzyloxy-quinoline-2-carboxylate 31e

By replacing 7-bromo-8-benzyloxy-quinoline-2-carboxylic acid in example 10 by methyl-6-bromo-8-nitro-4-hydroxy-quinoline-2-carboxylate 31b, the abovenamed product is obtained. Yield: 95%. ¹H NMR (300 MHz, CDCl₃): δ 8.65 (d, 1H, J=2 Hz, H⁷), 8.21 (d, 1H, J=2 Hz, H⁵), 7.83 (s, 1H³), 7.53 (m, 5H arom.), 5.44 (s, 2H benz.), 4.08 (s, 3H, OCH₃).

### Methyl-4-(N-methyl-toluene-4-sulfonamino)-8-nitro-quinoline-2-carboxylate 38b

By replacing 7-bromo-8-benzyloxy-quinoline-2-carboxylic acid in example 10 by methyl-4-(toluene-4-sulfonamino)-8-nitro-quinoline-2-carboxylate 15c and benzyl bromide by iodomethane, the abovenamed product is obtained. Yield: 89%. ¹H NMR (200 MHz, CDCl₃): δ 8.73 (dd, 1H, J=2 and 9 Hz, H arom.), 8.16 (dd, 1H, J=2 and 8 Hz, H arom.), 7.81 (dd, 1H, J=8 and 9 Hz, H⁶), 7.56 (s, 1H, H³), 7.52 (AB, 2H, J=7 Hz, 2H tosyl), 7.35 (AB, 2H, J=7 Hz, 2H tosyl), 4.01 (s, 3H, OCH₃), 3.30 (s, 3H, CH₃ tosyl), 2.50 (s, 3H, CH₃).

### Methyl-8-dimethylamino-4-benzyloxy-quinoline-2-carboxyalte 41a

By replacing 7-bromo-8-benzyloxy-quinoline-2-carboxylic acid in example 10 by methyl-4-benzyloxy-8-amino-quinoline-2-carboxylate and benzyl bromide by methyl iodide, the abovenamed product is obtained. Yield: 25%. ¹H NMR (200 MHz, CDCl₃): δ 7.82 (dd, 1H, J=2 and 9 Hz, H arom.), 7.65 (s, 1H, H³), 7.5 (m, 6H, 6H arom.), 7.09 (dd, 1H, J=2 and 8 Hz, H⁶), 5.32 (s, 2H, CH₂benz.), 4.03 (s, 3H, OCH₃), 3.19 (s, 6H, 2 NCH₃).

### EXAMPLE 11

### Benzyl-8-benzyloxy-5-bromo-quinoline-2-carboxylate 5b

Dissolve 0.74 g (2 mmol) of benzyl-8-benzyloxy-quinoline-2-carboxylate 5a in 25 ml of dichloromethane. Cool to −10° C. and add by small spatula tips, without allowing the temperature to rise above −5° C., 0.90 g (2.2 mmol) of 2,4,4,6-tetrabromocyclohexa-2,5-dienone. Allow to return to room temperature and stir overnight. Evaporate to dryness and perform chromatography on a silica gel column, eluent: EtOAc/Hex: 1/3 to obtain the abovenamed product. Yield: 86%. ¹H-NMR (200 MHz, CDCl₃): δ 8.66 (d, 1H, J=9 Hz, 1H arom.), 8.33 (d, 1H, J=9 Hz, 1H arom.) 7.81 (d, 1H, J=8 Hz, 1H arom.), 7.56 (m, 4H arom.), 7.40-7.30 (m, 6H arom.), 7.06 (d, 1H, J=8 Hz, 1H arom.), 5.55 (s, 2H, OCH₂), 5.45 (s, 2H, OCH₂).

### Methyl-8-methoxy-5-bromo-quinoline-2-carboxylate 5c

By replacing benzyl-8-benzyloxy-quinoline-2-carboxylate 5a in example 11 by methyl-8-methoxy-quinoline-2-carboxylate and proceeding in the same manner, the abovenamed product is obtained. Yield: 89%. ¹H-NMR (200 MHz, CDCl₃): δ 8.63 (d, 1H, J=9 Hz, 1H arom.), 8.31 (d, 1H, J=9 Hz, 1H arom.) 7.82 (d, 1H, J=8 Hz, 1H arom.), 6.97 (d, 1H, J=8 Hz, 1H arom.), 4.09 (s, 3H, OCH₃), 4.04 (s, 3H, OCH₃).

### EXAMPLE 12

### Methyl-4-chloro-8-methoxy-quinoline-2-carboxylate 8a

Dissolve 0.932 g (4 mmol) of methyl-4-hydroxy-8-methoxy-quinoline-2-carboxylate in 2 ml of POCl₃ and heat under reflux for 2 hours. Allow to cool and pour on a water-ice mixture. Extract twice with EtOAc, wash with saturated NaCl solution, dry on Na₂SO₄ and evaporate to dryness. Chromatograph on a silica column, eluent: CH₂Cl₂ to obtain the abovenamed product. Yield: 67%. ¹H-NMR (200 MHz, CDCl₃): δ 8.33 (s, 1H arom.), 7.85 (d, 1H, J=8 Hz, 1H arom.), 7.85 (t, 1H, J=8 Hz, 1H arom.), 7.16 (d, 1H, J=8 Hz, 1H arom.), 4.11 (s, 3H, OCH₃), 4.06 (s, 3H, OCH₃).

### Methyl-4-chloro-8-benzyloxy-quinoline-2-carboxylate 8b

By replacing methyl-4-hydroxy-8-methoxy-quinoline-2-carboxylate in example 12 by methyl-4-hydroxy-8-benzyloxy-quinoline-2-carboxylate 2a and proceeding in the same manner, the abovenamed product is obtained. Yield: 73%. ¹H-NMR (200 MHz, CDCl₃): δ 8.32 (s, 1H arom.), 7.83 (d, 1H, J=9 Hz, 1H arom.), 7.60-7.36 (m, 6H arom.), 7.17 (d, 1H, J=9 Hz, 1H arom.), 5.46 (s, 2H, OCH₂), 4.06 (s, 3H, OCH₃).

### Methyl-4-chloro-8-nitro-quinoline-2-carboxylate 8c

By replacing methyl-4-hydroxy-8-methoxy-quinoline-2-carboxylate in example 12 by methyl-4-hydroxy-8-nitro-quinoline-2-carboxylate 2r and proceeding in the same manner, the abovenamed product is obtained. Yield: 100%. ¹H-NMR (300 MHz, CDCl₃): δ 8.53 (d, 1H, J=8 Hz, 1H arom.), 8.40 (s, 1H arom.), 8.17 (d, 1H, J=8 Hz, 1H arom.), 7.84 (m, 1H arom.), 4.07 (s, 3H, OCH₃).

### EXAMPLE 13

### Methyl-8-benzyloxy-4-trifluoromethanesulfonyloxy-quinoline-2-carboxylate 11

Dissolve 1.11 g (3.8 mmol) of methyl-4-hydroxy-8-benzyloxy-quinoline-2-carboxylate 2a in 30 ml of dichloromethane. Add 2.2 ml (26 mmol) of pyridine. Cool to 0° C. and add by small amounts 5 ml (30 mmol) of triflic anhydride. Allow to return to room temperature and stir for 2 hours. Add a saturated NH₄Cl solution and extract with dichloromethane. Wash with saturated NaCl solution, dry on Na₂SO₄ and evaporate to dryness. Chromatograph on a silica gel column, eluent: EtOAc/Hex: 1/4 to obtain the abovenamed product. Yield: 73%. ¹H-NMR (200 MHz, CDCl₃): δ 8.20 (s, 1H arom.), 7.68-7.24 (m, 8H arom.), 5.48 (s, 2H, OCH₂), 4.08 (s, 3H, OCH₃).

### Methyl-8-nitro-4-trifluoromethanesulfonyloxy-quinoline-2-carboxylate 92a

By replacing methyl-4-hydroxy-8-benzyloxy-quinoline-2-carboxylate 2a in example 13 by methyl-4-hydroxy-8-nitro-quinoline-2-carboxylate 2r and proceeding in the same manner, the abovenamed product is obtained. Yield: 55%. ¹H-NMR (200 MHz, CDCl₃): δ 8.38-8.23 (m, 3H arom.), 7.93 (t, 1H, J=8 Hz, 1H arom.), 4.09 (s, 3H, OCH₃).

### EXAMPLE 14

### Methyl-4-hydroxy-6-(4-methoxy-phenyl)-8-benzyloxy-quinoline-2-carboxylate 18g

Dissolve 300 mg (0.77 mmol) of methyl-4-hydroxy-6-bromo-8-benzyloxy-quinoline-2-carboxylate 2l in 7 ml of degassed dimethylformamide. Add 40 mg (0.035 mmol) of tetrakistriphenylphosphine palladium (0), 383 mg (1.7 mmol, 2.2 eq) of tripotassium phosphate, 235 mg (1.55 mmol, 2 eq) of 4-methoxybenzene boronic acid. Heat at 115° C. for 16 hours, add water plus ice, stir for 1 hour then filter. Chromatograph the solid on a silica gel column, eluent: CH₂Cl₂/EtOAc: 9/1 to obtain the abovenamed product. Yield: 30%. ¹H-NMR (200 MHz, CDCl₃): δ 9.46 (broad s, 1H, OH), 8.1 (m, 1H arom.), 7.60-7.39 (m, 8H arom.), 7.01-6.97 (m, 3H arom.), 5.36 (s, 2H, OCH₂), 4.04 (s, 3H, OCH₃), 3.87 (s, 3H, OCH₃).

### Methyl-4-hydroxy-8-benzyloxy-5-phenyl-quinoline-2-carboxylate 18a

Starting with methyl-4-hydroxy-5-bromo-8-benzyloxy-quinoline-2-carboxylate 2b and replacing in example 14 4-methoxybenzene boronic acid by benzene boronic acid and proceeding in the same manner, the abovenamed product is obtained. Yield: 49%. ¹H-NMR (200 MHz, DMSO-d₆): δ 9.55 (broad s, 1H, OH), 7.62-7.04 (m, 11H arom.), 7.02 (d, 1H, J=8 Hz, 1H arom.), 6.54 (s, 1H arom.), 5.44 (s, 2H, OCH₂), 4.08 (s, 3H, OCH₃).

### Methyl-4-hydroxy-6-(3-methyl-phenyl)-8-benzyloxy-quinoline-2-carboxylate 18i

Starting with methyl-8-benzyloxy-6-bromo-4-hydroxyquinoline-2-carboxylate 2k and replacing in example 14 4-methoxybenzene boronic acid by par 3-methylbenzene boronic acid and proceeding in the same manner, the abovenamed product is obtained. Yield: 30%. ¹H-NMR (200 MHz, CDCl₃): δ 9.47 (broad s, 1H, OH), 8.15 (m, 1H arom.), 7.73-7.30 (m, 9H arom.), 7.21-7.17 (m, 1H arom.), 7.01 (m, 1H arom.), 5.36 (s, 2H, OCH₂), 4.04 (s, 3H, OCH₃), 2.43 (s, 3H, CH₃).

### Methyl-4-hydroxy-6-(3,4-dichloro-phenyl)-8-benzyloxy-quinoline-2-carboxylate 18m

Starting with methyl-8-benzyloxy-6-bromo-4-hydroxyquinoline-2-carboxylate 2l and replacing in example 14 4-methoxybenzene boronic acid by 3,4-dichlorobenzene boronic acid and proceeding in the same manner, the abovenamed product is obtained. Yield: 50%. ¹H-NMR (300 MHz, CDCl₃): δ 9.47 (broad s, 1H, OH), 8.05 (s, 1H arom.), 7.92 (s, 1H arom.), 7.76-7.72 (m, 3H arom.), 7.60-7.58 (m, 2H arom.), 7.47-7.45 (m, 3H arom.), 6.60 (s, 1H arom.), 5.51 (s, 2H, OCH₂), 4.04 (s, 3H, OCH₃).

### Methyl-8-benzyloxy-4-hydroxy-6-(pyridin-3-yl)-quinoline-2-carboxylate 18o

Starting with methyl-8-benzyloxy-6-bromo-4-hydroxyquinoline-2-carboxylate 2l and replacing in example 14 4-methoxybenzene boronic acid by 3-pyridine boronic acid 1,3-propanediol cyclic ester and proceeding in the same manner, the abovenamed product is obtained. Yield: 56%. ¹H-NMR (200 MHz, CDCl₃): δ 8.97 (broad s, 1H, OH), 8.58 (m, 1H arom.), 8.17 (m, 1H arom.), 7.94 (m, 1H arom.), 7.74 (m, 1H arom.), 7.58-7.47 (m, 7H arom.), 6.56 (s, 1H arom.), 5.52 (s, 2H, OCH₂), 4.04 (s, 3H, OCH₃).

### Methyl-4-hydroxy-6-(4-chloro-phenyl)-8-benzyloxy-quinoline-2-carboxylate 18k

Starting with methyl-8-benzyloxy-6-bromo-4-hydroxyquinoline-2-carboxylate 2l and replacing in example 14 4-methoxybenzene boronic acid by 4-chlorobenzene boronic acid and proceeding in the same manner, the abovenamed product is obtained. Yield: 90%. ¹H-NMR (300 MHz, CDCl₃): δ 9.50 (broad s, 1H, OH), 8.10 (m, 2H arom.), 7.71-7.64 (m, 2H arom.), 7.56-7.35 (m, 7H arom.), 7.02 (m, 1H arom.), 5.36 (s, 2H, OCH₂), 4.04 (s, 3H, OCH₃).

### Methyl-4-hydroxy-6-phenyl-8-methoxy-quinoline-2-carboxylate 18f

Starting with methyl-4-hydroxy-6-bromo-8-methoxy-quinoline-2-carboxylate 2l and replacing in example 14 4-methoxybenzene boronic acid by benzene boronic acid and proceeding in the same manner, the abovenamed product is obtained. Yield: 50%. ¹H-NMR (300 MHz, CDCl₃): δ 9.46 (broad s, 1H, OH), 7.90 (m, 1H arom.), 7.80 (m, 2H arom.), 7.60 (m, 1H arom.), 7.61-7.53 (m, 3H arom.), 6.73 (m, 1H arom.), 4.14 (s, 3H, OCH₃), 4.06 (s, 3H, OCH₃).

### Methyl-4-hydroxy-8-methoxy-6-propenyl-quinoline-2-carboxylate 18q

By replacing in example 14 4-methoxybenzene boronic acid by methylethylene catecholborane and proceeding in the same manner the abovenamed product is obtained. Yield: 70%. ¹H-NMR (200 MHz, CDCl₃): δ 9.45 (broad s, 1H, OH), 7.79 (m, 1H arom.), 7.12 (m, 1H arom.), 6.96 (m, 1H arom.), 6.45-6.36 (m, 2H vinylic), 4.05 (s, 3H, OCH₃), 4.03 (s, 3H, OCH₃), 1.94-1.91 (d, 3H, CH₃).

### Methyl-8-methoxy-5-phenyl-quinoline-2-carboxylate 18t

Starting with benzyl-8-methoxy-5-bromo-quinoline-2-carboxylate 5c and replacing in example 14 4-methoxybenzene boronic acid by benzene boronic acid and proceeding in the same manner, the abovenamed product is obtained. Yield: 59%. ¹H-NMR (300 MHz, CDCl₃): δ 8.38 (d, 1H, J=9 Hz, 1H arom.), 8.19 (d, 1H, J=9 Hz, 1H arom.), 7.56-7.44 (m, 6H arom.), 7.16 (d, 1H, J=8 Hz, 1H arom.), 4.14 (s, 3H, OCH₃), 4.07 (s, 3H, OCH₃).

### Methyl-8-methoxy-4-phenyl-quinoline-2-carboxylate 9b

Starting with methyl-4-chloro-8-methoxy-quinoline-2-carboxylate 8a and replacing in example 14 4-methoxybenzene boronic acid by benzene boronic acid and proceeding in the same manner, the abovenamed product is obtained. Yield: 55%. ¹H-NMR (300 MHz, CDCl₃): δ 8.19 (m, 1H arom.), 7.55-7.52 (m, 7H arom.), 7.11 (m, 1H arom.), 4.13 (s, 3H, OCH₃), 4.07 (s, 3H, OCH₃).

### Methyl 8-methoxy-4-(4-chlorophenyl)-quinoline-2-carboxylate 9c

Starting with methyl-4-chloro-8-methoxy-quinoline-2-carboxylate 8a and replacing in example 14 4-methoxybenzene boronic acid by 4-chlorobenzene boronic acid and proceeding in the same manner, the abovenamed product is obtained. Yield: 87%. ¹H-NMR (200 MHz, CDCl₃): δ 8.16 (s, 1H arom.), 7.56-7.48 (m, 7H arom.), 7.11 (m, 1H arom.), 4.13 (s, 3H, OCH₃), 4.07 (s, 3H, OCH₃).

### Methyl-8-benzyloxy-6-(furo-2-yl)-4-hydroxyquinoline-2-carboxylate 43a

Starting with methyl-8-benzyloxy-6-bromo-4-hydroxyquinoline-2-carboxylate 2l and replacing in example 14 4-methoxybenzene boronic acid by furan-2-boronic acid and proceeding in the same manner, the abovenamed product is obtained. Yield: 59%. ¹H-NMR (300 MHz, CDCl₃): δ 9.45 (broad s, 1H, OH), 8.18 (s, 1H arom.), 7.54-7.40 (m, 7H arom.), 6.98 (d, J=1.89 Hz, 1H arom.), 6.78 (d, J=3.45 Hz, 1H arom.), 6.51 (dd, J=1.86 Hz and J=3.42 Hz, 1H arom.), 5.35 (s, 2H, OCH₂), 4.03 (s, 3H, OCH₃).

### Benzyl-4,8-dibenzyloxy-6-(2-chlorophenyl)-quinoline-2-carboxylate 46c

Starting with benzyl-4,8-dibenzyloxy-6-bromoquinoline-2-carboxylate 46b and replacing in example 14 4-methoxybenzene boronic acid by 2-chloropheenyl boronic acid and proceeding in the same manner, the abovenamed product is obtained. Yield: 80%. ¹H-NMR (200 MHz, CDCl₃): δ 7.87-7.86 (d, 1H arom.), 7.74 (s, 1H arom.), 7.62-7.28 (m, 20H arom.), 5.51 (s, 2H, OCH₂), 5.42 (s, 2H, OCH₂), 5.36 (s, 2H, OCH₂).

### Benzyl-4,8-dibenzyloxy-6-(3-chlorophenyl)-quinoline-2-carboxylate 48a

Starting with benzyl-4,8-dibenzyloxy-6-bromoquinoline-2-carboxylate 46b and replacing in example 14 4-methoxybenzene boronic acid by 3-chlorophenyl boronic acid and proceeding in the same manner, the abovenamed product is obtained. Yield: 63%. ¹H-NMR (200 MHz, CDCl₃): δ 8.00-7.99 (d, 1H arom.), 7.74 (s, 1H arom.), 7.68-7.28 (m, 20H arom.), 5.50 (s, 2H, OCH₂), 5.47 (s, 2H, OCH₂), 5.39 (s, 2H, OCH₂).

### Benzyl-4,8-dibenzyloxy-7-phenyl-quinoline-2-carboxylate 50e

Starting with benzyl-4,8-dibenzyloxy-7-bromoquinoline-2-carboxylate 50d and replacing in example 14 4-methoxybenzene boronic acid by benzene boronic acid and proceeding in the same manner, the abovenamed product is obtained. Yield: 60%. ¹H-NMR (300 MHz, CDCl₃): δ 8.10-8.07 (d, 1H arom.), 7.72 (s, 1H arom.), 7.66-7.37 (m, 16H arom.), 7.18-7.15 (m, 5H arom.), 5.52 (s, 2H, OCH₂), 5.39 (s, 2H, OCH₂), 5.21 (s, 2H, OCH₂).

### Benzyl-4,8-dibenzyloxy-6-(2-methoxyphenyl)-quinoline-2-carboxylate 51a

Starting with benzyl-4,8-dibenzyloxy-6-bromoquinoline-2-carboxylate 46b and replacing in example 14 4-methoxybenzene boronic acid by 2-methoxyphenyl boronic acid and proceeding in the same manner, the abovenamed product is obtained. Yield: 70%. ¹H-NMR (200 MHz, CDCl₃): δ 7.96 (d, 1H arom.), 7.70 (s, 1H arom.), 7.57-7.33 (m, 20H arom.), 5.51 (s, 2H, OCH₂), 5.43 (s, 2H, OCH₂), 5.36 (s, 2H, OCH₂), 3.72 (s, 3H, OCH₃).

### Benzyl-4,8-dibenzyloxy-3-phenyl-quinoline-2-carboxylate 53c

Starting with benzyl-4,8-dibenzyloxy-3-bromoquinoline-2-carboxylate 53b and replacing in example 14 4-methoxybenzene boronic acid by benzene boronic acid and proceeding in the same manner, the abovenamed product is obtained. Yield: 20%. ¹H-NMR (300 MHz, CDCl₃): δ 7.74-7.71 (d, 1H arom.), 7.56-7.28 (m, 17H arom.), 7.10-7.07 (m, 5H arom.), 5.47 (s, 2H, OCH₂), 5.13 (s, 2H, OCH₂), 4.60 (s, 2H, OCH₂).

### Methyl-8-cyano-4-hydroxy-6-phenyl-quinoline-2-carboxylate 64a

Starting with methyl-6-bromo-8-cyano-4-hydroxy-quinoline-2-carboxylate 63b and replacing in example 14 4-methoxybenzene boronic acid by benzene boronic acid and proceeding in the same manner, the abovenamed product is obtained. Yield: 84%. ¹H-NMR (300 MHz, DMSO₆): δ 12.60 (broad s, 1H, OH), 8.78 (s, 1H arom.), 8.66 (s, 1H arom.), 7.68-7.48 (m, 5H arom.), 6.77 (s, 1H arom.), 3.98 (s, 3H, OCH₃).

### Methyl-5-phenyl-8-nitro-4-benzyloxy-quinoline-2-carboxylate 73a

By replacing methyl-4-hydroxy-6-bromo-8-benzyloxy-quinoline-2-carboxylate 2k in example 14 by methyl-5-bromo-8-nitro-4-benzyloxy-quinoline-2-carboxylate 72c and by replacing 4-methoxybenzene boronic acid by phenyl boronic acid, the abovenamed product is obtained. Yield: 74%. ¹H NMR (200 MHz, CDCl₃): δ 8.00 (d, 1H, J=8 Hz, H⁷), 7.42 (d, 1H, J=8 Hz, H⁵), 7.24 (m, 8H, 8H arom.), 7.73 (m, 2H, 2H arom.), 5.01 (s, 2H benz.), 5.04 (s, 2H, 2H benz.).

### Benzyl-7-phenyl-8-amino-4-benzyloxy-quinoline-2-carboxylate 34d

By replacing methyl-4-hydroxy-6-bromo-8-benzyloxy-quinoline-2-carboxylate 2k in example 14 by benzyl-7-iodo-8-amino-4-benzyloxy-quinoline-2-carboxylate 34c and by replacing 4-methoxybenzene boronic acid by phenyl boronic acid, the abovenamed product is obtained. Yield: 61%. ¹H NMR (200 MHz, CDCl₃): δ 7.61 (d, 1H, J=8 Hz, H⁷), 7.57 (s, 1H arom.), 7.48 (m, 5H arom.), 7.41 (m, 11H arom.), 5.49 (s, 2H benz.), 5.37 (s, 4H, 2 NH and 2H benz.).

### Methyl-5-(4-chlorophenyl)-8-methoxy-quinoline-2-carboxylate 81a

Starting with methyl-8-methoxy-5-bromo-quinoline-2-carboxylate 5c and replacing in example 14 4-methoxybenzene boronic acid by 3-chlorobenzene boronic acid and proceeding in the same manner, the abovenamed product is obtained. Yield: 74%. ¹H-NMR (300 MHz, CDCl₃): δ 8.32 (d, 1H, J=9 Hz, 1H arom.), 8.19 (d, 1H, J=9 Hz, 1H arom.), 7.53-7.36 (m, 5H arom.), 7.15 (d, 1H, J=8 Hz, 1H arom.), 4.14 (s, 3H, OCH₃), 4.07 (s, 3H, OCH₃).

### Benzyl-4,8-dibenzyloxy-6-(3,5-dichlorophenyl)-quinoline-2-carboxylate 88a

Starting with benzyl-4,8-dibenzyloxy-6-bromoquinoline-2-carboxylate 46b and replacing in example 14 4-methoxybenzene boronic acid by 3,5-dichlorobenzene boronic acid and proceeding in the same manner, the abovenamed product is obtained. Yield: 45%. ¹H-NMR (200 MHz, CDCl₃): δ 7.97 (s, 1H arom.), 7.74 (s, 1H arom.), 7.63-7.36 (m, 19H arom.), 5.50 (s, 2H, OCH₂), 5.47 (s, 2H, OCH₂), 5.30 (s, 2H, OCH₂).

### Benzyl-4,8-dibenzyloxy-6-(4-fluorophenyl)-quinoline-2-carboxylate 89a

Starting with benzyl-4,8-dibenzyloxy-6-bromoquinoline-2-carboxylate 46b and replacing in example 14 4-methoxybenzene boronic acid by 4-fluorobenzene boronic acid and proceeding in the same manner, the abovenamed product is obtained. Yield: 67%. ¹H-NMR (300 MHz, CDCl₃): δ 7.97 (s, 1H arom.), 7.73 (s, 1H arom.), 7.59-7.13 (m, 20H arom.), 5.50 (s, 2H, OCH₂), 5.46 (s, 2H, OCH₂), 5.38 (s, 2H, OCH₂).

### Methyl-8-nitro-4-phenyl-quinoline-2-carboxylate 92b

Starting with methyl-8-nitro-4-trifluoromethanesulfonyloxy-quinoline-2-carboxylate 92a and replacing in example 14 4-methoxybenzene boronic acid by benzene boronic acid and proceeding in the same manner, the abovenamed product is obtained. Yield: 47%. ¹H-NMR (200 MHz, CDCl₃): δ 8.27 (s, 1H arom.), 8.23-8.10 (m, 2H arom.), 7.71-7.54 (m, 6H arom.), 4.07 (s, 3H, OCH₃).

### EXAMPLE 15

### Methyl-4-hydroxy-8-methoxy-6-phenylethynyl-quinoline-2-carboxylate 20a

Dissolve 300 mg (0.96 mmol) of methyl-4-hydroxy-6-bromo-8-methoxy-quinoline-2-carboxylate 2k in 8 ml of degassed acetonitrile. Add 33 mg (0.17 mmol) of copper iodide, 19 mg (0.107 mmol, 0.11 eq) of palladium(II) chloride, 65 mg (0.247 mmol, 0.25 mmol) of triphenylphosphine, 352 μl (9.6 mmol, 10 eq) of phenylacetylene and 1.84 ml of triethylamine. Heat at 50° C. for 16 hours, evaporate to dryness and chromatograph using as eluent: CH₂Cl₂/EtOAc: 9/1 to obtain the abovenamed product. Yield: 68%. ¹H-NMR (200 MHz, CDCl₃): δ 9.42 (broad s, 1H, OH), 8.11 (s, 1H arom.), 7.56 (m, 2H arom.), 7.39-7.37 (m, 3H arom.), 7.20 (s, 1H arom.), 6.98 (s, 1H arom.), 4.08 (s, 3H, OCH₃), 4.05 (s, 3H, OCH₃).

### Methyl-8-benzyloxy-4-phenylethynyl-quinoline-2-carboxylate 12a

By replacing methyl-4-hydroxy-6-bromo-8-methoxy-quinoline-2-carboxylate 2k in example 15 by methyl-8-benzyloxy-4-trifluoromethanesulfonyloxy-quinoline-2-carboxylate 11, the abovenamed product is obtained. Yield: 52%. ¹H-NMR (200 MHz, CDCl₃): δ 8.40 (s, 1H arom), 7.98 (d, 1H, J=9 Hz, 1H arom.), 7.70-7.58 (m, 5H arom.), 7.46-7.36 (m, 6H arom.), 7.16 (d, 1H, J=8 Hz, 1H arom.), 5.47 (s, 2H, OCH₂), 4.08 (s, 3H, OCH₃).

### Methyl-8-benzyloxy-4-(3-tert-butoxycarbonylaminoprop-1-ynyl)-quinoline-2-carboxylate 12b

By replacing methyl-4-hydroxy-6-bromo-8-methoxy-quinoline-2-carboxylate 2k in example 15 by methyl-8-benzyloxy-4-trifluoromethanesulfonyloxy-quinoline-2-carboxylate 11 and by replacing phenylacetylene by N-Boc propargylamine and proceeding in the same manner, the abovenamed product is obtained. Yield: 34%. ¹H-NMR (200 MHz, CDCl₃): δ 8.28 (m, 1H arom.), 7.85 (d, 1H, J=8 Hz, 1H arom.), 7.82-7.34 (m, 6H arom.), 7.13 (d, 1H, J=8 Hz, 1H arom.), 5.45 (s, 2H, OCH₂), 4.90 (broad s, 1H, NH), 4.31 (d, 2H, J=6 Hz, CH₂), 4.05 (s, 3H, OCH₃), 1.49 (s, 9H, (CH₃)₃).

### Methyl-8-benzyloxy-4-(3-benzyloxyprop-1-ynyl)-quinoline-2-carboxylate 12c

By replacing methyl-4-hydroxy-6-bromo-8-methoxy-quinoline-2-carboxylate 2k in example 15 by methyl-8-benzyloxy-4-trifluoromethanesulfonyloxy-quinoline-2-carboxylate 11 and by replacing phenylacetylene by O-benzyl propargylic alcohol and proceeding in the same manner, the abovenamed product is obtained. Yield: 68%. ¹H-NMR (300 MHz, CDCl₃): δ 8.32 (s, 1H arom.), 7.86 (d, 1H, J=8 Hz, 1H arom.), 7.54-7.34 (m, 11H arom.), 7.14 (d, 1H, J=8 Hz, 1H arom.), 5.45 (s, 2H, OCH₂), 4.76 (s, 2H, OCH₂), 4.57 (s, 2H, OCH₂), 4.06 (s, 3H, OCH₃).

### Methyl-4-hydroxy-8-methoxy-6-(hept-1-ynyl)-quinoline-2-carboxylate 20b

By replacing phenylacetylene in example 15 by 1-heptyne and proceeding in the same manner, the abovenamed product is obtained. Yield: 47%. ¹H-NMR (200 MHz, CDCl₃): δ 9.38 (broad s, 1H, OH), 7.96 (m, 1H arom.), 7.07 (m, 1H arom.), 6.96 (m, 1H arom.), 4.05 (s, 3H, OCH₃), 4.03 (s, 3H, OCH₃), 2.42 (t, 2H, CH₂), 1.56 (m, 2H, CH₂), 1.42 (m, 4H, 2×CH₂), 0.94 (t, 3H, CH₃).

### Methyl-8-benzyloxy-6-(3-tert-butoxycarbonylamino-prop-1-ynyl)-4-hydroxy-quinoline-2-carboxylate 20c

By replacing methyl-4-hydroxy-6-bromo-8-methoxy-quinoline-2-carboxylate 2k in example 15 by methyl-4-hydroxy-6-bromo-8-benzyloxy-quinoline-2-carboxylate 2l and by replacing phenylacetylene by N-Boc propargylamine and proceeding in the same manner, the abovenamed product is obtained. Yield: 70%. ¹H-NMR (300 MHz, CDCl₃): δ 9.42 (broad s, 1H, OH), 7.99 (m, 1H arom.), 7.45 (m, 5H arom.), 7.16 (m, 1H arom.), 6.96 (m, 1H arom.), 5.27 (s, 2H, OCH₂), 4.18-4.15 (s, 2H, CH₂), 4.02 (s, 3H, OCH₃), 1.45 (s, 9H, (CH₃)₃).

### Methyl-8-benzyloxy-6-(3-benzyloxy-prop-1-ynyl)-4-hydroxy-quinoline-2-carboxylate 20d

By replacing methyl-4-hydroxy-6-bromo-8-methoxy-quinoline-2-carboxylate 2k in example 15 by methyl-4-hydroxy-6-bromo-8-benzyloxy-quinoline-2-carboxylate 2l and by replacing phenylacetylene by O-benzyl propargylic alcohol and proceeding in the same manner, the abovenamed product is obtained. Yield: 73%. ¹H-NMR (300 MHz, CDCl₃): δ 9.41 (broad s, 1H, OH), 8.04 (m, 1H arom.), 7.45-7.38 (m, 10H arom.), 7.19 (m, 1H arom.), 6.96 (m, 1H arom.), 5.27 (s, 2H, OCH₂), 4.69 (s, 2H, OCH₂), 4.42 (s, 2H, OCH₂), 4.03 (s, 3H, OCH₃).

### Benzyl-4-benzyloxy-8-(hex-1-ynyl)-quinoline-2-carboxylate 47b

By replacing methyl-4-hydroxy-6-bromo-8-methoxy-quinoline-2-carboxylate 2k in example 15 by benzyl-4-benzyloxy-8-bromo-quinoline-2-carboxylate 47a and by replacing phenylacetylene by 1-hexyne and proceeding in the same manner, the abovenamed product is obtained. Yield: 55%. ¹H-NMR (300 MHz, CDCl₃): δ 8.21-8.19 (d, 1H arom.), 7.90-7.87 (d, 1H arom.), 7.70 (s, 1H arom.), 7.58-7.38 (m, 11H arom.), 5.50 (s, 2H, OCH₂), 5.35 (s, 2H, OCH₂), 2.62-2.57 (t, 2H, CC—CH₂), 1.69-1.51 (m, 4H, CH₂—CH₂—CH₃), 0.99-0.94 (t, 3H, CH₂—CH₃).

### Benzyl-4,8-dibenzyloxy-6-(3-benzyloxy-prop-1-ynyl)-quinoline-2-carboxylate 49a

By replacing methyl-4-hydroxy-6-bromo-8-methoxy-quinoline-2-carboxylate 2k in example 15 by benzyl-4,8-dibenzyloxy-6-bromoquinoline-2-carboxylate 46b and by replacing phenylacetylene by O-benzyl propargylic alcohol and proceeding in the same manner, the abovenamed product is obtained. Yield: 95%. ¹H-NMR (300 MHz, CDCl₃): δ 7.98-7.97 (d, 1H arom.), 7.72 (s, 1H arom.), 7.56-7.33 (m, 20H arom.), 7.18-7.17 (d, 1H arom.), 5.49 (s, 2H, OCH₂), 5.36 (s, 2H, OCH₂), 5.34 (s, 2H, OCH₂), 4.68 (s, 2H, CH₂—OCH₂), 4.43 (s, 2H, CH₂—OCH₂).

### Benzyl-4,8-dibenzyloxy-7-(3-benzyloxy-prop-1-ynyl)-quinoline-2-carboxylate 54a

By replacing methyl-4-hydroxy-6-bromo-8-methoxy-quinoline-2-carboxylate 2k in example 15 by benzyl-4,8-dibenzyloxy-7-bromoquinoline-2-carboxylate 50c and by replacing phenylacetylene by O-benzyl propargylic alcohol and proceeding in the same manner, the abovenamed product is obtained. Yield: 38%. ¹H-NMR (200 MHz, CDCl₃): δ 7.97-7.92 (d, 1H arom.), 7.69 (s, 1H arom.), 7.57-7.23 (m, 21H arom.), 5.68 (s, 2H, OCH₂), 5.51 (s, 2H, OCH₂), 5.35 (s, 2H, OCH₂), 4.69 (s, 2H, CH₂—OCH₂), 4.48 (s, 2H, CH₂—OCH₂).

### Benzyl-4,8-dibenzyloxy-7-phenylethynyl-quinoline-2-carboxylate 55a

By replacing methyl-4-hydroxy-6-bromo-8-methoxy-quinoline-2-carboxylate 2k in example 15 by benzyl-4,8-dibenzyloxy-7-bromo-quinoline-2-carboxylate 50c, the abovenamed product is obtained. Yield: 58%. ¹H-NMR (200 MHz, CDCl₃): δ 7.99-7.94 (d, 1H arom.), 7.69-7.27 (m, 22H arom.), 5.73 (s, 2H, OCH₂), 5.52 (s, 2H, OCH₂), 5.36 (s, 2H, OCH₂).

### Methyl-4-benzyloxy-8-cyano-6-phenylethynyl-quinoline-2-carboxylate 65b

By replacing methyl-4-hydroxy-6-bromo-8-methoxy-quinoline-2-carboxylate 2k in example 15 by methyl-6-bromo-8-cyano-4-benzyloxy-quinoline-2-carboxylate 65a, the abovenamed product is obtained. Yield: 66%. ¹H-NMR (300 MHz, CDCl₃): δ 8.60 (d, 1H, J=2 Hz, 1H arom.), 8.26-8.25 (d, 1H, J=2 Hz, 1H arom.), 7.80 (s, 1H arom.), 7.60-7.39 (m, 10H arom.), 5.40 (s, 2H, OCH₂), 4.09 (s, 3H, OCH₃).

### Benzyl-4-benzyloxy-8-cyano-3-phenylethynyl-quinoline-2-carboxylate 67b

By replacing methyl-4-hydroxy-6-bromo-8-methoxy-quinoline-2-carboxylate 2k in example 15 by benzyl-3-bromo-4-benzyloxy-8-cyano-quinoline-2-carboxylate 67a, the abovenamed product is obtained. Yield: 9%. ¹H-NMR (200 MHz, CDCl₃): δ 8.63-8.58 (m, 1H arom.), 8.17-8.13 (m, 1H arom.), 7.97-7.41 (m, 11H arom.), 5.61 (s, 2H, OCH₂).

### Methyl-8-cyano-4-hydroxy-6-[(trimethylsilyl)ethynyl]-quinoline-2-carboxylate 69a

By replacing methyl-4-hydroxy-6-bromo-8-methoxy-quinoline-2-carboxylate 2k in example 15 by methyl-6-bromo-8-cyano-4-benzyloxy-quinoline-2-carboxylate 65a, the abovenamed product is obtained. Yield: 27%. ¹H-NMR (300 MHz, CDCl₃): δ 9.24 (broad s, 1H, OH), 8.60 (m, 1H arom.), 8.02 (m, 1H arom.), 7.01 (m, 1H arom.), 4.08 (s, 3H, OCH₃), 0.27 (s, 9H, Si(CH₃)₃).

### Methyl-3-trimethylsilylethynyl-8-nitro-4-hydroxy-quinoline-2-carboxylate 57a

By replacing methyl-4-hydroxy-6-bromo-8-methoxy-quinoline-2-carboxylate 2k in example 15 by methyl-3-bromo-8-nitro-4-hydroxy-quinoline-2-carboxylate 45a and by replacing phenylacetylene by trimethylsilylacetylene, the abovenamed product is obtained. Yield: 46%. ¹H NMR (200 MHz, CDCl₃): δ 8.65 (dd, 1H, J=1 and 8 Hz, H arom.), 8.12 (dd, 1H, J=1 and 8 Hz, H arom.), 7.83 (t, 1H, J=8 Hz, H arom.), 7.80 (m, 1H, OH), 4.14 (s, 3H, CH₃).

### Methyl-3-phenylethynyl-8-nitro-4-hydroxy-quinoline-2-carboxylate 68a

By replacing methyl-4-hydroxy-6-bromo-8-methoxy-quinoline-2-carboxylate 2k in example 15 by methyl-3-bromo-8-nitro-4-hydroxy-quinoline-2-carboxylate 45a, the abovenamed product is obtained. Yield: 63%. ¹H NMR (300 MHz, CDCl₃): δ 8.65 (dd, 1H, J=2 and 8 Hz, H arom.), 8.11 (dd, 1H, J=2 and 8 Hz, H arom.), 7.85 (m, 2H, H arom. and OH), 7.55 (m, 3H arom.), 4.16 (s, 3H, OCH₃).

### Benzyl-3-(3′-benzyloxypropyn-1′-yl)-8-nitro-4-hydroxy-quinoline-2-carboxylate 70a

By replacing methyl-4-hydroxy-6-bromo-8-methoxy-quinoline-2-carboxylate 2k in example 15 by benzyl-3-bromo-8-nitro-4-hydroxy-quinoline-2-carboxylate and by replacing phenylacetylene by 3-benzyloxyprop-1-yne, the abovenamed product is obtained. Yield: 43%. ¹H NMR (300 MHz, CDCl₃): δ 8.54 (d, 1H, J=8 Hz, H arom.), 8.10 (d, 1H, J=8 Hz, H arom.), 7.81 (t, 1H, J=8 Hz, H⁶), 7.40 (m, 10H, 10H arom.), 5.57 (s, 2H, CH₂benz.), 4.80 (s, 2H, CH₂benz.), 4.70 (s, 2H, CH₂O).

### Benzyl-3-(3′-N-(terbutoxycarbonyl)aminoprop-1′-ynyl)-8-nitro-4-hydroxy-quinoline-2-carboxylate 71a

By replacing methyl-4-hydroxy-6-bromo-8-methoxy-quinoline-2-carboxylate 2k in example 15 by benzyl-3-bromo-8-nitro-4-hydroxy-quinoline-2-carboxylate and by replacing phenylacetylene by 3-N-(terbutoxycarbonyl)aminoprop-1-yne, the abovenamed product is obtained. Yield: 43%. ¹H NMR (300 MHz, CDCl₃): δ 8.48 (d, 1H, J=8 Hz, H arom.), 8.08 (d, 1H, J=8 Hz, H arom.), 7.76 (t, 1H, J=8 Hz, H arom.), 7.46 (m, 5H arom.), 5.54 (s, 2H, CH₂benz.), 5.05 (s, 1H, 1 NH), 4.63 (d, 2H, J=6 Hz, CH₂N), 1.49 (s, 9H, 3 CH₃).

### Methyl-5-phenylethynyl-8-nitro-4-benzyloxy-quinoline-2-carboxylate 74a

By replacing methyl-4-hydroxy-6-bromo-8-mehoxy-quinoline-2-carboxylate 2k in example 15 by methyl-5-bromo-8-nitro-4-benzyloxy-quinoline-2-carboxylate 72c, the abovenamed product is obtained. Yield: 97%. ¹H NMR (200 MHz, CDCl₃): δ 8.03 (d, 1H, J=8 Hz, H⁷), 7.90 (d, 1H, J=8 Hz, H⁶), 7.86 (s, 1H³), 7.56 (m, 2H arom.), 7.32 (m, 6H arom.), 7.05 (m, 2H arom.), 5.46 (s, 2H benz.), 4.08 (s, 3H, OCH₃).

### Methyl-5-(3′-benzyloxypropyn-1′-yl)-8-nitro-4-benzyloxy-quinoline-2-carboxylate 74a

By replacing methyl-4-hydroxy-6-bromo-8-methoxy-quinoline-2-carboxylate 2k in example 15 by methyl-5-bromo-8-nitro-4-benzyloxy-quinoline-2-carboxylate 72c and by replacing phenylacetylene by 3-benzyloxyprop-1-yne, the abovenamed product is obtained. Yield: 59%. ¹H NMR (200 MHz, CDCl₃): δ 7.94 (d, 1H, J=8 Hz, 1H arom.), 7.79 (s, 1H³), 7.74 (d, 1H, J=8 Hz, 1H arom.), 7.51 (m, 2H arom.), 7.32 (m, 8H arom.), 5.33 (s, 2H benz.), 4.49 (s, 2H benz.), 4.04 (s, 3H, OCH₃), 3.83 (s, 2H, CH₂O).

### Methyl-5-(3′-N-(terbutoxycarbonyl)aminoprop-1′-ynyl)-8-nitro-4-benzyloxy-quinoline-2-carboxylate 75a

By replacing methyl-4-hydroxy-6-bromo-8-methoxy-quinoline-2-carboxylate 2k in example 15 by methyl-5-bromo-8-nitro-4-benzyloxy-quinoline-2-carboxylate 72c and by replacing phenylacetylene by 3-N-(terbutoxycarbonyl)aminoprop-1-yne, the abovenamed product is obtained. Yield: 89%. ¹H NMR (200 MHz, CDCl₃): δ 7.92 (d, 1H, J=8 Hz, 1H arom.), 7.79 (s, 1H³), 7.72 (d, 1H, J=8 Hz, 1H arom.), 7.57 (m, 2H arom.), 7.49 (m, 3H arom.), 5.33 (AB, 2H, H=6 Hz, 2H benz.), 4.05 (m, 1 HN), 4.04 (s, 3H, OCH₃), 3.65 (d, 2H, J=6 Hz, CH₂N), 1.44 (s, 9H, 3 CH₃).

### Methyl-6-phenylethynyl-8-nitro-4-benzyloxy-quinoline-2-carboxylate 118a

By replacing methyl-4-hydroxy-6-bromo-8-meethoxy-quinoline-2-carboxylate 2k in example 15 by methyl-6-bromo-8-nitro-4-benzyloxy-quinoline-2-carboxylate 31e, the abovenamed product is obtained. Yield: 85%. ¹H NMR (300 MHz, CDCl₃): δ 8.58 (d, 1H, J=2 Hz, 1H arom.), 8.20 (d, 1H, J=2 Hz, 1H arom.), 7.80 (s, 1H³), 7.49 (m, 5H arom.), 5.41 (s, 2H benz.), 4.07 (s, 3H, OCH₃).

### Methyl-6-(3′-benzyloxypropyn-1′-yl)-8-nitro-4-benzyloxy-quinoline-2-carboxylate 119a

By replacing methyl-4-hydroxy-6-bromo-8-methoxy-quinoline-2-carboxylate 2k in example 15 by methyl-6-bromo-8-nitro-4-benzyloxy-quinoline-2-carboxylate 31e and by replacing phenylacetylene by 3-benzyloxyprop-1-yne, the abovenamed product is obtained. Yield: 87%. ¹H NMR (300 MHz, CDCl₃): δ 8.51 (d, 1H, J=2 Hz, 1H arom.), 8.07 (d, 1H, J=2 Hz, 1H arom.), 7.79 (s, 1H³), 7.44 (m, 10H arom.), 5.39 (s, 2H benz.), 4.69 (s, 2H benz.), 4.69 (s, CH₂O), 4.02 (s, 3H, OCH₃).

### Methyl-6-(3′-N-(terbutoxycarbonyl)aminoprop-1′-ynyl)-8-nitro-4-benzyloxy-quinoline-2-carboxylate 121a

By replacing methyl-4-hydroxy-6-bromo-8-methoxy-quinoline-2-carboxylate 2k in example 15 by methyl-6-bromo-8-nitro-4-benzyloxy-quinoline-2-carboxylate 31e and by replacing phenylacetylene by 3-N-(terbutoxycarbonyl)aminoprop-1-yne, the abovenamed product is obtained. Yield: 87%. ¹H NMR (200 MHz, CDCl₃): δ 8.48 (d, 1H, J=2 Hz, 1H arom.), 8.06 (d, 1H, J=2 Hz, 1H arom.), 7.78 (s, 1H³), 7.50 (m, 5H arom.), 5.38 (s, 2H benz.), 4.82 (m, 1 HN), 4.19 (d, 2H, J=5 Hz, CH₂N), 4.04 (s, 3H, OCH₃), 1.68 (s, 9H, 3 CH₃).

### Methyl-6-(3′-pyridyl)ethynyl-8-nitro-4-benzyloxy-quinoline-2-carboxylate 122a

By replacing methyl-4-hydroxy-6-bromo-8-methoxy-quinoline-2-carboxylate 2k in example 15 by methyl-6-bromo-8-nitro-4-benzyloxy-quinoline-2-carboxylate 31e and by replacing phenylacetylene by 3-pyridylacetylene, the abovenamed product is obtained. Yield: 95%. ¹H NMR (300 MHz, CDCl₃): δ 8.83 (s, 1H arom.), 8.63 (m, 2H arom.), 8.20 (d, 1H, J=2 Hz, 1H arom.), 7.87 (dt, 1H, J=2 and 8 Hz, 1H arom.), 7.82 (s, 1H arom.), 7.48 (m, 5H arom.), 7.34 (dd, 1H, J=5 and 8 Hz, 1H arom.), 5.42 (s, 2H benz.), 4.06 (s, 3H, OCH₃).

### Methyl-6-(5′-cyanopent-1′-ynyl)-8-nitro-4-benzyloxy-quinoline-2-carboxylate 123a

By replacing methyl-4-hydroxy-6-bromo-8-methoxy-quinoline-2-carboxylate 2k in example 15 by methyl-6-bromo-8-nitro-4-benzyloxy-quinoline-2-carboxylate 31e and by replacing phenylacetylene by 5-cyanopent-1-yne, the abovenamed product is obtained. Yield: 91%. ¹H NMR (200 MHz, CDCl₃): δ 8.46 (s, 1H arom.), 8.06 (s, 1H arom.), 7.78 (s, 1H arom.), 7.49 (m, 5H arom.), 5.39 (s, 2H benz.), 4.04 (s, 3H, OCH₃), 2.64 (m, 4H, 2 CH₂), 2.01 (t, 2H, J=7 Hz, CH₂).

### Benzyl-8-benzyloxy-5-phenylethynyl-quinoline-2-carboxylate 80a

By replacing methyl-4-hydroxy-6-bromo-8-methoxy-quinoline-2-carboxylate 2k in example 15 by benzyl-8-benzyloxy-5-bromoquinoline-2-carboxylate 5b, the abovenamed product is obtained. Yield: 68%. ¹H-NMR (200 MHz, CDCl₃): δ 8.85-8.80 (d, 1H arom.), 8.35-8.31 (d, 1H arom.), 8.30-7.38 (m, 16H arom.), 7.15-7.11 (d, 1H arom.), 5.53 (s, 2H, OCH₂), 5.46 (s, 2H, OCH₂).

### Methyl-8-benzyloxy-4-(hex-1-ynyl)-quinoline-2-carboxylate 83a

By replacing methyl-4-hydroxy-6-bromo-8-methoxy-quinoline-2-carboxylate 2k in example 15 by methyl-8-benzyloxy-4-trifluoromethanesulfonyloxy-quinoline-2-carboxylate 11 and by replacing phenylacetylene by 1-hexyne and proceeding in the same manner, the abovenamed product is obtained. Yield: 84%. ¹H-NMR (200 MHz, CDCl₃): δ 8.26 (s, 1H arom.), 7.87 (d, 1H, J=8 Hz, 1H arom.), 7.57-7.27 (m, 6H arom.), 7.12 (d, 1H, J=8 Hz, 1H arom.), 5.45 (s, 2H, OCH₂), 4.05 (s, 3H, OCH₃), 2.60 (t, 2H, J=8 Hz, CH₂), 1.75-1.54 (m, 4H, 2×CH₂), 1.01 (t, 3H, J=7 Hz, CH₃).

### Methyl-8-benzyloxy-4-(5-benzyloxy-pent-1-ynyl)-quinoline-2-carboxylate 84a

By replacing methyl-4-hydroxy-6-bromo-8-methoxy-quinoline-2-carboxylate 2k in example 15 by methyl-8-benzyloxy-4-trifluoromethanesulfonyloxy-quinoline-2-carboxylate 11 and by replacing phenylacetylene by 5-benzyloxy-pent-1-ynyl and proceeding in the same manner, the abovenamed product is obtained. Yield: 84%. ¹H-NMR (200 MHz, CDCl₃): δ 8.24 (s, 1H arom.), 7.3 (d, 1H, J=7 Hz, 1H arom.), 7.54-7.27 (m, 11H arom.), 7.12 (d, 1H, J=8 Hz, 1H arom.), 5.44 (s, 2H, OCH₂), 4.57 (s, 2H, OCH₂), 4.05 (s, 3H, OCH₃), 3.69 (t, 2H, J=6 Hz, CH₂), 2.74 (t, 2H, J=7 Hz, CH₂), 2.05-1.98 (m, 2H, CH₂).

### Methyl-8-benzyloxy-7-(3-tert-butoxycarbonylamino-prop-1-ynyl)-4-hydroxy-quinoline-2-carboxylate 90a

By replacing methyl-4-hydroxy-6-bromo-8-methoxy-quinoline-2-carboxylate 2k in example 15 by methyl-8-benzyloxy-4-hydroxy-7-bromo-quinoline-2-carboxylate 50c and by replacing phenylacetylene by N-Boc propargylamine and proceeding in the same manner, the abovenamed product is obtained. Yield: 47%. ¹H-NMR (200 MHz, CDCl₃): δ 9.13 (broad s, 1H, OH), 7.94 (d, 1H, J=8 Hz, 1H arom.), 7.40-7.28 (m, 6H arom.), 6.86 (s, 1H arom.), 5.42 (s, 2H, OCH₂), 4.23 (d, 2H, J=6 Hz, 2H, NHCH₂), 3.98 (s, 3H, OCH₃), 1.47 (s, 9H, (CH₃)₃).

### Benzyl-4,8-dibenzyloxy-7-(hex-1-ynyl)-quinoline-2-carboxylate 91a

By replacing methyl-4-hydroxy-6-bromo-8-methoxy-quinoline-2-carboxylate 2k in example 15 by benzyl-4,8-dibenzyloxy-7-bromoquinoline-2-carboxylate 50c and by replacing phenylacetylene by 1-hexyne and proceeding in the same manner, the abovenamed product is obtained. Yield: 63%. ¹H-NMR (200 MHz, CDCl₃): δ 7.90 (d, 1H, J=8 Hz, 1H arom.), 7.66-7.28 (m, 17H arom.), 5.61 (s, 2H, OCH₂), 5.51 (s, 2H, OCH₂), 5.34 (s, 2H, OCH₂), 2.52 (t, 2H, J=7 Hz, CH₂), 1.62-1.53 (m, 4H, 2×CH₂), 0.96 (t, 3H, J=7 Hz, CH₃).

### Methyl-8-amino-4-(hex-1-ynyl)-quinoline-2-carboxylate 93a

By replacing methyl-4-hydroxy-6-bromo-8-methoxy-quinoline-2-carboxylate 2k in example 15 by methyl-8-nitro-4-trifluoromethanesulfonyloxy-quinoline-2-carboxylate 92a and by replacing phenylacetylene by 1-hexyne and proceeding in the same manner, the abovenamed product is obtained. Yield: 45%. ¹H-NMR (200 MHz, CDCl₃): δ 8.15 (s, 1H arom.), 7.58-7.41 (m, 2H arom.), 6.96 (d, 1H, J=8 Hz, 1H arom.), 5.18 (broad s, 2H, NH₂), 4.03 (s, 3H, OCH₃), 2.59 (t, 2H, J=7 Hz, CH₂), 1.74-1.49 (m, 4H, 2×CH₂), 0.99 (t, 3H, J=7 Hz, CH₃).

### Methyl-8-amino-4-phenylethynyl-quinoline-2-carboxylate 94a

By replacing methyl-4-hydroxy-6-bromo-8-methoxy-quinoline-2-carboxylate 2k in example 15 by methyl-8-nitro-4-trifluoromethanesulfonyloxy-quinoline-2-carboxylate 92a and proceeding in the same manner, the abovenamed product is obtained. Yield: 48%. ¹H-NMR (200 MHz, CDCl₃): δ 8.33 (s, 1H arom.); 7.73-7.45 (m, 7H arom.), 7.01 (d, 1H, J=8 Hz, 1H arom.), 5.18 (broad s, 2H, NH₂), 4.03 (s, 3H, OCH₃).

### Methyl-8-nitro-4-(3-tert-butoxycarbonylamino-prop-1-ynyl)-quinoline-2-carboxylate 95a

By replacing methyl-4-hydroxy-6-bromo-8-methoxy-quinoline-2-carboxylate 2k in example 15 by methyl-8-nitro-4-trifluoromethanesulfonyloxy-quinoline-2-carboxylate 92a and by replacing phenylacetylene by N-Boc propargylamine and proceeding in the same manner, the abovenamed product is obtained. Yield: 67%. ¹H-NMR (200 MHz, CDCl₃): δ 8.52 (d, 1H, J=8 Hz, 1H arom.), 8.34 (s, 1H arom.), 8.13 (d, 1H, J=8 Hz, 1H arom.), 7.76 (t, 1H, J=8 Hz, 1H arom.), 4.98 (broad s, 2H, NH₂), 4.34 (d, 2H, J=6 Hz, 2H, NHCH₂), 3.91 (s, 3H, OCH₃), 1.51 (s, 9H, (CH₃)₃).

### Methyl-4-(3-benzyloxy-prop-1-ynyl)-8-nitro-quinoine-2-carboxylate 96a

By replacing methyl-4-hydroxy-6-bromo-8-methoxy-quinoline-2-carboxylate 2k in example 15 by methyl-8-nitro-4-trifluoromethanesulfonyloxy-quinoline-2-carboxylate 92a and by replacing phenylacetylene by O-benzyl propargylic alcohol and proceeding in the same manner, the abovenamed product is obtained. Yield: 40%. ¹H-NMR (200 MHz, CDCl₃): δ 8.52 (d, 1H, J=8 Hz, 1H arom.), 8.38 (s, 1H arom.), 8.13 (d, 1H, J=8 Hz, 1H arom.), 7.77 (t, 1H, J=8 Hz, 1H arom.), 7.42-7.36 (m, 5H arom.), 4.76 (s, 2H, OCH₂), 4.59 (s, 2H, OCH₂), 4.06 (s, 3H, OCH₃).

### Methyl-4-(3-acetyl-aminoprop-1-ynyl)-8-nitro-quinoline-2-carboxylate 97a

By replacing methyl-4-hydroxy-6-bromo-8-methoxy-quinoline-2-carboxylate 2k in example 15 by methyl-8-nitro-4-trifluoromethanesulfonyloxy-quinoline-2-carboxylate 92a and by replacing phenylacetylene by N-acetyl propargylamine and proceeding in the same manner, the abovenamed product is obtained. Yield: 55%. ¹H-NMR (200 MHz, CDCl₃): δ 8.30 (d, 1H, J=8 Hz, 1H arom.), 8.15 (s, 1H arom.), 8.07 (d, 1H, J=8 Hz, 1H arom.), 7.75 (t, 1H, J=8 Hz, 1H arom.), 6.70 (s, H, NH), 4.49 (s, 2H, CH₂), 4.02 (s, 3H, OCH₃), 2.38 (s, 3H, COCH₃).

### Methyl-4-benzyloxy-8-phenylethynyl-quinoline-2-carboxylate 111a

By replacing methyl-4-hydroxy-6-bromo-8-methoxy-quinoline-2-carboxylate 2k in example 15 by methyl-8-bromo-4-benzyloxy-quinoline-2-carboxylate 47a and proceeding in the same manner, the abovenamed product is obtained. Yield: 48%. ¹H-NMR (200 MHz, CDCl₃): δ 8.29 (d, 1H, J=8 Hz, 1H arom.), 8.04 (d, 1H, J=8 Hz, 1H arom.), 7.76-7.39 (m, 11H arom.), 7.38 (s, 1H arom.), 5.38 (broad s, 2H, NH₂), 4.09 (s, 3H, OCH₃).

### EXAMPLE 16

### Methyl-8-benzyloxy-6-benzyl-4-hydroxy-quinoline-2-carboxylate 23a

To methyl-4-hydroxy-6-bromo-8-benzyloxy-quinoline-2-carboxylate 2l (282 mg, 0.726 mmol) in degassed tetrahydrofuran (10 ml), add benzyl zincic bromide (0.5 M/THF, 7.26 ml) and tetrakistriphenylphosphine palladium (0). Heat at 50° C. for 4 hours, wash with 2 N hydrochloric acid, wash with water and with a saturated sodium chloride solution. Dry on sodium sulfate, filter and evaporate to dryness. Triturate in diethyl ether, filter to obtain the abovenamed product. Yield: 49%. ¹H-NMR (300 MHz, methanol-d₄): δ 7.84 (m, 1H arom.), 7.70-7.15 (m, 10H arom.), 7.03 (m, 1H arom.), 6.96 (m, 1H arom.), 5.35 (s, 2H, OCH₂), 4.08 (m, 5H, OCH₃ and CH₂).

### EXAMPLE 17

### Methyl-4,8-dihydroxy-6-(3-hydroxy-propyl)-quinoline-2-carboxylate 21d

To methyl-8-benzyloxy-6-(3-benzyloxy-prop-1-ynyl)-4-hydroxy-quinoline-2-carboxylate 20d (132 mg, 0.246 mmol) dissolved in degassed dichloromethane, methanol or acetic acid (15 ml), add palladium/charcoal (10%) (30 mg) and place under hydrogen at 30 psi overnight. Filter on celite, wash twice with CH₂Cl₂/MeOH 8/2 then evaporate to dryness. Triturate in diethyl ether and filter to obtain the abovenamed product. Yield: 95%. ¹H-NMR (200 MHz, methanol-d₄): δ 7.56 (s, 1H arom.), 7.02 (m, 2H arom.), 4.06 (s, 3H, OCH₃), 3.60 (t, 2H, CH₂), 2.77 (m, 2H, CH₂), 1.89 (m, 2H, CH₂).

### Methyl-4,8-dihydroxy-6-(3-tert-butoxycarbonylamino-propyl)-quinoline-2-carboxylate 21c

By replacing methyl-8-benzyloxy-6-(3-benzyloxy-prop-1-ynyl)-4-hydroxyquinoline-2-carboxylate 20d in example 17 by methyl-8-benzyloxy-6-(3-tert-butoxycarbonylamino-prop-1-ynyl)-4-hydroxyquinoline-2-carboxylate 20c and proceeding in the same manner, the abovenamed product is obtained. Yield: 70%. ¹H-NMR (300 MHz, CDCl₃): δ 9.68 (broad s, 1H, OH), 7.60 (m, 1H arom.), 7.08 (m, 1H arom.), 6.98 (m, 1H arom.), 4.83 (broad s, 1H, NH), 4.03 (s, 3H, OCH₃), 3.11 (m, 2H, CH₂), 2.64 (m, 2H, CH₂), 1.79 (m, 2H, CH₂), 1.44 (s, 9H, (CH₃)₃).

### Methyl-4-hydroxy-8-methoxy-6-phenethyl-quinoline-2-carboxylate 21a

By replacing methyl-8-benzyloxy-6-(3-benzyloxy-prop-1-ynyl)-4-hydroxyquinoline-2-carboxylate 20d in example 17 by methyl-4-hydroxy-8-methoxy-6-phenylethynyl-quinoline-2-carboxylate 20a and proceeding in the same manner, the abovenamed product is obtained. Yield: 72%. ¹H-NMR (300 MHz, CDCl₃): δ 9.38 (broad s, 1H, OH), 7.78 (m, 1H arom.), 7.31-7.17 (m, 5H arom.), 6.98 (m, 1H arom.), 6.76 (m, 1H arom.), 4.04 (s, 3H, OCH₃), 3.94 (s, 3H, OCH₃), 3.01 (m², 4H, 2CH₂).

### Methyl-4-hydroxy-8-methoxy-6-heptyl-quinoline-2-carboxylate 21b

By replacing methyl-8-benzyloxy-6-(3-benzyloxy-prop-1-ynyl)-4-hydroxyquinoline-2-carboxylate 20d in example 17 by methyl-4-hydroxy-8-methoxy-6-(hept-1-ynyl)-quinoline-2-carboxylate 20b and proceeding in the same manner, the abovenamed product is obtained. Yield: 74%. ¹H-NMR (200 MHz, CDCl₃): δ 9.38 (broad s, 1H, OH), 7.71 (m, 1H arom.), 6.97-6.93 (m, 2H arom.), 4.03 (s, 6H, 2×OCH₃), 2.75-2.67 (m, 2H, CH₂), 1.72 (m, 2H, CH₂), 1.31 (m, 8H, 4×CH₂), 0.88 (m, 3H, CH₃).

### Methyl-4-hydroxy-8-methoxy-6-propyl-quinoline-2-carboxylate 18r

By replacing methyl-8-benzyloxy-6-(3-benzyloxy-prop-1-ynyl)-4-hydroxyquinoline-2-carboxylate 20d in example 17 by methyl-4-hydroxy-8-methoxy-6-propenyl-quinoline-2-carboxylate 18q and proceeding in the same manner, the abovenamed product is obtained. Yield: 82%. ¹H-NMR (200 MHz, CDCl₃): δ 9.38 (broad s, 1H, OH), 7.70 (m, 1H arom.), 6.96-6.92 (m, 2H arom.), 4.03 (s, 6H, OCH₃), 2.73-2.65 (t, 2H, CH₂), 1.76-1.65 (m, 2H, CH₂), 0.99-0.92 (t, 3H, CH₃).

### Methyl-8-hydroxy-4-phenethyl-quinoline-2-carboxylate 13a

By replacing methyl-8-benzyloxy-6-(3-benzyloxyprop-1-ynyl)-4-hydroxyquinoline-2-carboxylate 20d in example 17 by methyl-8-benzyloxy-4-phenylethynyl-quinoline-2-carboxylate 12a and proceeding in the same manner, the abovenamed product is obtained. Yield: 88%. ¹H-NMR (300 MHz, CDCl₃): δ 8.60 (broad s, 1H, OH), 8.01 (m, 1H arom.), 7.56 (m, 1H arom.), 7.30-7.22 (m, 7H arom.), 4.05 (s, 3H, OCH₃), 3.42(t, 2H, J=8 Hz, CH₂), 3.10(t, 2H, J=8 Hz, CH₂).

### Methyl-8-hydroxy-4-(3-tert-butoxycarbonylamino-propyl)-quinoline-2-carboxylate 13b

By replacing methyl-8-benzyloxy-6-(3-benzyloxyprop-1-ynyl)-4-hydroxyquinoline-2-carboxylate 20d in example 17 by methyl-8-benzyloxy-4-(3-tert-butoxycarbonylaminoprop-1-ynyl)-quinoline-2-carboxylate 12b and proceeding in the same manner, the abovenamed product is obtained. Yield: 90%. ¹H-NMR (300 MHz, CDCl₃): δ 8.07 (s, 1H arom.), 7.54-7.28 (m, 3H arom.), 4.05 (s, 3H, OCH₃), 3.26 (m, 2H, CH₂), 3.14 (m, 2H, CH₂), 2.10 (m, 2H, CH₂), 1.46 (s, 9H, (CH₃)₃).

### Methyl-8-hydroxy-4-(3-hydroxy-propyl)-quinoline-2-carboxylate 13c

By replacing methyl-8-benzyloxy-6-(3-benzyloxyprop-1-ynyl)-4-hydroxyquinoline-2-carboxylate 20d in example 17 by methyl-8-benzyloxy-4-(3-benzyloxyprop-1-ynyl)-quinoline-2-carboxylate 12c and proceeding in the same manner, the abovenamed product is obtained. Yield: 95%. ¹H-NMR (200 MHz, CDCl₃): δ 8.6 (broad s, 1H, OH), 8.11 (m, 1H arom.), 7.69-7.07 (m, 3H arom.), 4.05 (s, 3H, OCH₃), 3.56 (t, 2H, J=8 Hz, CH₂), 3.24 (t, 2H, J=8 Hz, CH₂), 3.10 (t, 2H, J=8 Hz, CH₂).

### 8-hexyl-4-hydroxy-quinoline-2-carboxylic acid 47c

By replacing methyl-8-benzyloxy-6-(3-benzyloxyprop-1-ynyl)-4-hydroxyquinoline-2-carboxylate 20d in example 17 by Benzyl-4-benzyloxy-8-(hex-1-ynyl)-quinoline-2-carboxylate 47b and proceeding in the same manner, the abovenamed product is obtained. Yield: 70%. ¹H-NMR (300 MHz, DMSO-d₆): δ 8.03-8.01 (m, 1H arom.), 7.66-7.62 (m, 1H arom.), 7.46-7.44 (m, 1H arom.), 7.05 (s, 1H arom.), 3.11-3.09 (t, 2H, Ph-CH₂), 1.69 (m, 2H, CH₂), 1.34 (m, 6H, 3CH₂), 0.88 (m, 3H, CH₃).

### 4,8-dihydroxy-6-(3-hydroxy-propyl)-quinoline-2-carboxylic acid 49b

By replacing methyl-8-benzyloxy-6-(3-benzyloxyprop-1-ynyl)-4-hydroxyquinoline-2-carboxylate 20d in example 17 by benzyl-4,8-dibenzyloxy-6-(3-benzyloxy-prop-1-ynyl)-quinoline-2-carboxylate 49a and proceeding in the same manner, the abovenamed product is obtained. Yield: 71%. ¹H-NMR (200 MHz, DMSO-d₆): δ 7.38 (s, 1H arom.), 7.02 (s, 1H arom.), 6.86 (s, 1H arom.), 3.45-3.39 (t, 2H, CH₂), 2.69 (t, 2H, CH₂), 1.76 (t, 2H, CH₂).

### 4,8-dihydroxy-7-(3-hydroxy-propyl)-quinoline-2-carboxylic acid 54b

By replacing methyl-8-benzyloxy-6-(3-benzyloxyprop-1-ynyl)-4-hydroxyquinoline-2-carboxylate 20d in example 17 by benzyl-4,8-dibenzyloxy-7-(3-benzyloxy-prop-1-ynyl)-quinoline-2-carboxylate 54a and proceeding in the same manner, the abovenamed product is obtained. Yield: 50%. ¹H-NMR (200 MHz, DMSO-d₆): δ 7.55 (d, 1H arom.), 7.12-7.09 (d, 1H arom.), 6.58 (s, 1H arom.), 3.50-3.42 (t, 2H, CH₂), 2.78-2.74 (t, 2H, CH₂), 1.77-1.72 (t, 2H, CH₂).

### 4,8-dihydroxy-7-phenethyl-quinoline-2-carboxylic acid 55b

By replacing methyl-8-benzyloxy-6-(3-benzyloxyprop-1-ynyl)-4-hydroxyquinoline-2-carboxylate 20d in example 17 by benzyl-4,8-dibenzyloxy-7-phenylethynyl-quinoline-2-carboxylate 55a and proceeding in the same manner, the abovenamed product is obtained. Yield: 87%. ¹H-NMR (300 MHz, DMSO-d₆): δ 9.95 (broad s, 1H, CO₂H), 7.55-7.52 (d, 1H arom.), 7.38-7.19 (m, 7H arom.), 3.11-2.94 (m, 4H, Ph-CH₂—CH₂).

### Methyl-8-cyano-4-hydroxy-6-phenethyl-quinoline-2-carboxylate 65c

By replacing methyl-8-benzyloxy-6-(3-benzyloxyprop-1-ynyl)-4-hydroxyquinoline-2-carboxylate 20d in example 17 by methyl-4-benzyloxy-8-cyano-6-phenylethynyl-quinoline-2-carboxylate 65b and proceeding in the same manner, the abovenamed product is obtained. Yield: 8%. ¹H-NMR (200 MHz, CDCl₃): δ 9.19 (broad s, 1H, OH), 8.40 (m, 1H arom.), 7.71 (m, 1H arom.), 7.33-7.12 (m, 5H arom.), 7.02 (s, 1H arom.), 4.08 (s, 3H, OCH₃), 3.07-2.98 (m, 4H, Ph-CH₂—CH₂).

### Methyl-8-cyano-6-ethyl-4-hydroxy-quinoline-2-carboxylate 69c

By replacing methyl-8-benzyloxy-6-(3-benzyloxyprop-1-ynyl)-4-hydroxyquinoline-2-carboxylate 20d in example 17 by methyl-8-cyano-6-ethynyl-4-hydroxy-quinoline-2-carboxylate 69b and proceeding in the same manner, the abovenamed product is obtained. Yield: 82%. ¹H-NMR (200 MHz, MeOD): δ 8.20 (m, 1H arom.), 7.99 (m, 1H arom.), 6.98 (m, 1H arom.), 3.98 (s, 3H, OCH₃), 2.78 (m, 2H, CH₂—CH₃), 1.26-1.23 (m, 3H, CH₂—CH₃).

### 8-hexyl-4-hydroxy-quinoline-2-carboxylic acid 47b

By replacing methyl-8-benzyloxy-6-(3-benzyloxyprop-1-ynyl)-4-hydroxyquinoline-2-carboxylate 20d in example 17 by benzyl-4-benzyloxy-8-(hex-1-ynyl)-quinoline-2-carboxylate 47a and proceeding in the same manner, the abovenamed product is obtained. Yield: 70%. ¹H-NMR (300 MHz, DMSO-d₆): δ 8.03-8.01 (m, 1H arom.), 7.66-7.62 (m, 1H arom.), 7.46-7.44 (m, 1H arom.), 7.05 (s, 1H arom.), 3.11-3.09 (t, 2H, Ph-CH₂), 1.69 (m, 2H, CH₂), 1.34 (m, 6H, 3CH₂), 0.88 (m, 3H, CH₃).

### 4,8-dihydroxy-6-(3-hydroxy-propyl)-quinoline-2-carboxylic acid 49b

By replacing methyl-8-benzyloxy-6-(3-benzyloxyprop-1-ynyl)-4-hydroxyquinoline-2-carboxylate 20d in example 17 by methyl-4,8-dibenzyloxy-6-(3-benzyloxy-prop-1-ynyl)-quinoline-2-carboxylate 49a and proceeding in the same manner, the abovenamed product is obtained. Yield: 71%. ¹H-NMR (200 MHz, DMSO-d₆): δ 7.38 (s, 1H arom.), 7.02 (s, 1H arom.), 6.86 (s, 1H arom.), 3.45-3.39 (t, 2H, CH₂), 2.69 (t, 2H, CH₂), 1.76 (t, 2H, CH₂).

### 4,8-dihydroxy-7-(3-hydroxy-propyl)-quinoline-2-carboxylic acid 54b

By replacing methyl-8-benzyloxy-6-(3-benzyloxyprop-1-ynyl)-4-hydroxyquinoline-2-carboxylate 20d in example 17 by benzyl-4,8-dibenzyloxy-7-(3-benzyloxy-prop-1-ynyl)-quinoline-2-carboxylate 54a and proceeding in the same manner, the abovenamed product is obtained. Yield: 50%. ¹H-NMR (200 MHz, DMSO-d₆): δ 7.55 (d, 1H arom.), 7.12-7.09 (d, 1H arom.), 6.58 (s, 1H arom.), 3.50-3.42 (t, 2H, CH₂), 2.78-2.74 (t, 2H, CH₂), 1.77-1.72 (t, 2H, CH₂).

### 4,8-dihydroxy-7-phenethyl-quinoline-2-carboxylic acid 55b

By replacing methyl-8-benzyloxy-6-(3-benzyloxyprop-1-ynyl)-4-hydroxyquinoline-2-carboxylate 20d in example 17 by benzyl-4,8-dibenzyloxy-7-phenylethynyl-quinoline-2-carboxylate 55a and proceeding in the same manner, the abovenamed product is obtained. Yield: 87%. ¹H-NMR (300 MHz, DMSO-d₆): δ 9.95 (broad s, CO₂H), 7.55-7.52 (d, 1H arom.), 7.38-7.19 (m, 7H arom.), 3.11-2.94 (m, 4H, Ph-CH₂—CH₂).

### 8-hydroxy-5-phenylethyl-quinoline-2-carboxylic acid 80b

By replacing methyl-8-benzyloxy-6-(3-benzyloxy-prop-1-ynyl)-4-hydroxy-quinoline-2-carboxylate 20d in example 17 by benzyl-8-benzyloxy-5-phenylethynyl-quinoline-2-carboxylate 80a and proceeding in the same manner, the abovenamed product is obtained. Yield: 55%. ¹H-NMR (300 MHz, DMSO-d₆): δ 10.15 (s, 1H, OH), 7.25-7.18 (m, 9H arom.), 2.82-2.61 (m, 4H arom.).

### Methyl-8-benzyloxy-4-(hex-1-yl)-quinoline-2-carboxylate 83b

By replacing methyl-8-benzyloxy-6-(3-benzyloxy-prop-1-ynyl)-4-hydroxy-quinoline-2-carboxylate 20d in example 17 by methyl-8-benzyloxy-4-(hex-1-ynyl)-quinoline-2-carboxylate 83a and proceeding in the same manner, the abovenamed product is obtained. Yield: 67%. ¹H-NMR (200 MHz, DMSO-d₆): δ 8.17 (s, 1H arom.), 7.58-7.32 (m, 7H arom.), 7.10 (d, 1H, J=8 Hz, 1H arom.), 5.45 (s, 2H, OCH₂), 4.05 (s, 3H, OCH₃), 3.10 (t, 2H, J=8 Hz, CH₂), 1.79-1.70 (m, 2H, CH₂), 1.39-1.27 (m, 6H, CH₂), 0.90 (t, 3H, J=7 Hz, CH₃).

### Methyl-8-benzyloxy-4-(5-hydroxy-pent-1-yl)-quinoline-2-carboxylate 84b

By replacing methyl-8-benzyloxy-6-(3-benzyloxy-prop-1-ynyl)-4-hydroxy-quinoline-2-carboxylate 20d in example 17 by methyl-8-benzyloxy-4-(5-benzyloxy-pent-1-ynyl)-quinoline-2-carboxylate 84a and proceeding in the same manner, the abovenamed product is obtained. Yield: 97%. ¹H-NMR (200 MHz, DMSO-d₆): δ 8.02 (s, 1H arom.), 7.53-7.333 (m, 7H arom.), 6.76 (d, 1H, J=8 Hz, 1H arom.), 4.51 (s, 2H, OCH₂), 4.05 (s, 3H, OCH₃), 3.49 (t, 2H, J=8 Hz, CH₂), 3.11 (t, 2H, J=8 Hz, CH₂), 1.77-1.65 (m, 6H, 3×CH₂).

### Methyl-8-benzyloxy-4-(3-tert-butoxycarbonylaminoprop-1-yl)-quinoline-2-carboxylate 85a

By replacing methyl-8-benzyloxy-6-(3-benzyloxy-prop-1-ynyl)-4-hydroxyquinoline-2-carboxylate 20d in example 17 by methyl-8-benzyloxy-4-(3-tert-butoxycarbonylaminoprop-1-ynyl)-quinoline-2-carboxylate 12b and proceeding in the same manner, the abovenamed product is obtained. Yield: 93%. [H-NMR (300 MHz, CDCl₃): δ 8.07 (s, 1H arom.), 7.59-7.27 (m, 7H arom.), 7.09 (d, 1H, J=8 Hz, 1H arom.), 5.45 (s, 2H, OCH₂), 4.65 (broad s, 1H, NH), 4.05 (s, 3H, OCH₃), 3.29-3.09 (m, 4H, CH₂), 2.02 (d, 2H, J=7.6 Hz, CH₂), 1.47 (s, 9H, (CH₃)₃).

### Methyl-4,8-dihydroxy-7-(3-tert-butoxycarbonylaminoprop-1-yl)-quinoline-2-carboxylate 90b

By replacing methyl-8-benzyloxy-6-(3-benzyloxy-prop-1-ynyl)-4-hydroxyquinoline-2-carboxylate 20d in example 17 by methyl-8-benzyloxy-7-(3-tert-butoxycarbonylamino-prop-1-ynyl)-4-hydroxy-quinoline-2-carboxylate 90a and proceeding in the same manner, the abovenamed product is obtained. Yield: 95%. ¹H-NMR (300 MHz, CDCl₃): δ 9.75 (broad s, 1H, OH), 7.82 (d, 1H, J=8 Hz, 1H arom.), 7.12 (d, 1H, J=8 Hz, 1H arom.), 7.04 (s, 1H arom.), 5.09 (broad s, 1H, NH), 4.04 (s, 3H, OCH₃), 3.12-3.08 (m, 2H, CH₂), 2.87-2.82 (m, 2H, CH₂), 1.92-1.90 (m, 2H, CH₂), 1.53 (s, 9H, (CH₃)₃).

### 4,8-dihydroxy-7-(hex-1-yl)-quinoline-2-carboxylic acid 91b

By replacing methyl-8-benzyloxy-6-(3-benzyloxy-prop-1-ynyl)-4-hydroxyquinoline-2-carboxylate 20d in example 17 by benzyl-4,8-dibenzyloxy-7-(hex-1-ynyl)-quinoline-2-carboxylate 91a and proceeding in the same manner, the abovenamed product is obtained. Yield: 50%. ¹H-NMR (200 MHz, CDCl₃): δ 9.90 (broad s, 1H, OH), 7.58-7.22 (m, 2H arom.), 7.22 (s, 1H arom.), 2.75-2.71 (m, 2H, CH₂), 1.66-1.62 (m, 2H, CH₂), 1.28-1.22 (m, 6H, 3×CH₂), 0.84(t, 3H, J=7 Hz, CH₃).

### Methyl-4-hydroxy-8-phenylethyl-quinoline-2-carboxylate 111 b

By replacing methyl-8-benzyloxy-6-(3-benzyloxy-prop-1-ynyl)-4-hydroxyquinoline-2-carboxylate 20d in example 17 by methyl-4-benzyloxy-8-phenylethynyl-quinoline-2-carboxylate 111a and proceeding in the same manner, the abovenamed product is obtained. Yield: 72%. ¹H-NMR (200 MHz, CDCl₃): δ 8.75 (broad s, 1H, OH), 8.24 (d, 1H, J=8 Hz, 1H arom.), 7.51 (d, 1H, J=8 Hz, 1H arom.), 7.31-7.15 (m, 6H arom.), 6.92 (s, 1H arom.), 4.09 (s, 3H, OCH₃), 3.16-3.04 (m, 4H, 2×CH₂).

### EXAMPLE 18

### Methyl-8-benzyloxy-4-(toluene-4-sulfonylamino)-quinoline-2-carboxylate 15a

Prepare a suspension of methyl-4-hydroxy-8-benzyloxy-quinoline-2-carboxylate 2a (350 mg, 1.13 mmol) in 1,2-dichloroethane (15 ml). At room temperature, add tosyl isocyanate (0.2 ml, 1.35 mmol), then heat under reflux for 12 hours. Evaporate the solvent and triturate the residue in petroleum ether. Filter the solid obtained. Yield: 73%. ¹H-NMR (300 MHz, CDCl₃): δ 9.97 (s, 1H, NH), 8.19 (s, 1H arom.), 8.05 (m, 3H arom.), 7.99-7.16 (m, 9H arom.), 5.30 (s, 2H, OCH₂), 4.06 (s, 3H, OCH₃), 2.41 (s, 3H, CH₃).

### Methyl-8-nitro-4-(toluene-4-sulfonylamino)-quinoline-2-carboxylate 15c

By replacing methyl-4-hydroxy-8-benzyloxy-quinoline-2-carboxylate 2a in example 18 by methyl-4-hydroxy-8-nitro-quinoline-2-carboxylate 2r and proceeding in the same manner, the abovenamed product is obtained. Yield: 60%. ¹H-NMR (200 MHz, CDCl₃): δ 12.21 (s, 1H, NH), 8.92-8.73 (m, 2H arom.), 8.28 (s, 1H arom.), 7.89 (m, 3H arom.), 7.41 (d, 2H, J=8 Hz, 2H arom.), 3.98 (s, 3H, OCH₃), 2.36 (s, 3H, CH₃).

### EXAMPLE 19

### Methyl-4-amino-8-hydroxy-quinoline-2-carboxylate 17a

Cool concentrated sulfuric acid (3 ml) to 0° C. Add methyl-8-hydroxy-4-(toluene-4-sulfonylamino)-quinoline-2-carboxylate 15b (100 mg, 0.27 mmol). Allow to react at 0° C. for 2 hours, then dilute with water (20 ml). Basify this solution to pH 6-7 with saturated sodium bicarbonate solution. Extract the aqueous phase with ethyl acetate. Dry on sodium sulfate and evaporate the organic phase. Yield: 100%. ¹H-NMR (200 MHz, MeOD): δ 7.70 (d, 1H, J=9 Hz, 1H arom.), 7.60-7.52 (m, 2H arom.), 7.26 (d, 1H, J=9 Hz, 1H arom.), 4.20 (s, 3H, OCH₃).

### Methyl-4,8-diamino-quinoline-2-carboxylate 17b

By replacing methyl-8-hydroxy-4-(toluene-4-sulfonylamino)-quinoline-2-carboxylate 15b in example 19 by methyl-8-amino-4-(toluene-4-sulfonylamino)-quinoline-2-carboxylate 15d and proceeding in the same manner, the abovenamed product is obtained. Yield: 79%. ¹H-NMR (200 MHz, CDCl₃): δ 7.36 (s, 1H arom.), 7.30 (m, 1H arom.), 7.02 (d, 1H, J=9 Hz, 1H arom.), 6.89 (d, 1H, J=9 Hz, 1H arom.), 5.17 (broad s, 2H, NH₂), 4.85 (broad s, 2H, NH₂), 4.00 (s, 3H, CH₃).

### 4-methylamino-8-nitro-quinoline-2-carboxylate 38b

By replacing 8-hydroxy-4-(toluene-4-sulfonylamino)-quinoline-2-carboxylic 15b in example 19 by methyl-4-(N-methyl-toluene-4-sulfonylamino)-8-nitro-quinoline-2-carboxylate 38a, the abovenamed product is obtained. Yield: 86%. ¹H NMR (300 MHz, CDCl₃): δ 7.96 (d, 1H, J=9 Hz, H arom.), 7.94 (d, 1H, J=8 Hz, H arom.), 7.52 (dd, 1H, J=8 and 9 Hz, H 6), 7.32 (s, 1H, H³), 5.50 (s, 1H, NH), 4.00 (s, 3H, OCH₃), 3.15 (s, 3H, CH₃).

### EXAMPLE 20

### 8-acetylamino-4-hydroxy-quinoline-2-carboxylic acid 28a

Prepare a suspension of methyl-4-hydroxy-8-amino-quinoline-2-carboxylate 2u (200 mg, 0.92 mmol) in acetonitrile (10 ml). At room temperature and under argon add triethylamine (0.13 ml, 0.93 mmol), then acetyl chloride (0.065 ml, 0.91 mmol). Stir for 3 hours, then evaporate the solvent. Take up the residue in water and wash with ethyl acetate. Dry the organic phase on Na₂SO₄, filter and evaporate the solvents. Purify the crude reaction product by silica gel column chromatography, eluent: EtOAc. Dissolve the O,N-diacetylated product (100 mg, 0.33 mmol) so obtained in a 1:1 mixture of THF/water (10 ml). Add 1 N sodium hydroxide (1 ml). Stir at room temperature overnight. Cool the solution to 0° C., then acidify to pH 3 with 2 N HCl. Filter the resulting precipitate. Yield: 50%. ¹H-NMR (300 MHz, MeOD): δ 8.25 (broad s, 1H arom.), 8.06 (d, 1H, J=8 Hz, 1H arom.), 7.50 (m, 1H arom.), 7.30 (s, 1H arom.), 5.36 (broad s, 1H, NH), 2.31 (s, 3H, CH₃).

### 8-pivaloylamino-4-hydroxy-quinoline-2-carboxylic acid 28b

By replacing acetyl chloride in example 20 by pivaloyl chloride and proceeding in the same manner, the abovenamed product is obtained. Yield: 20%. ¹H-NMR (200 MHz, DMSO-d₆): δ 12.72 and 12.24 (2 broad s, 2H, COOH and OH), 10.24 (broad s, 1H, NH), 8.52 (s, 1H arom.), 7.85 (d, 1H, J=9 Hz, 1H arom.), 7.60-7.47 (m, 2H arom.), 1.35 (s, 9H, C(CH₃)₃),

### 8-benzoylamino-4-hydroxy-quinoline-2-carboxylic acid 28c

By replacing acetyl chloride in example 20 by benzoyl chloride and proceeding in the same manner, the abovenamed product is obtained. Yield: 65%. ¹H-NMR (200 MHz, DMSO-d₆): δ 13.01 and 12.10 (2 broad s, 2H, COOH and OH), 10.84 (broad s, 1H, NH), 8.72 (s, 1H arom.), 8.00 (m, 3H arom.), 7.53 (m, 5H arom.).

### Methyl-8-benzyloxy-4-(3-benzoyl-aminoprop-1-yl)-quinoline-2-carboxylate 85c

By replacing methyl-4-hydroxy-8-amino-quinoline-2-carboxylate 2u in example 20 by methyl-4-(3-aminoprop-1-yl)-8-benzyloxy-quinoline-2-carboxylate 85b and acetyl chloride by benzoyl chloride and proceeding in the same manner, the abovenamed product is obtained. Yield: 93%. ¹H-NMR (200 MHz, CDCl₃): δ 8.08 (s, 1H arom.), 7.78-7.28 (m, 12H arom.), 7.10 (d, 1H, J=8 Hz, 1H arom.), 6.27 (broad s, 1H, NH), 5.44 (s, 2H, OCH₂), 4.04 (s, 3H, OCH₃), 3.67-3.57 (m, 2H, CH₂), 3.22 (t, 2H, J=7.8 Hz, CH₂), 2.19-2.11 (m, 2H, CH₂).

### EXAMPLE 21

### Methyl-8-benzylamino-4-hydroxy-quinoline-2-carboxylate 29a

Dissolve methyl-4-hydroxy-8-amino-quinoline-2-carboxylate 2u (200 mg, 0.92 mmol) in methanol (5 ml). At room temperature and under argon, add benzaldehyde (0.1 ml, 0.98 mmol). Heat under reflux overnight then cool the reaction mixture to 0° C. Add NaBH₄ (87 mg, 2.30 mmol) and react in the cold for 1 hour. Evaporate the methanol, take up the residue in cold water and extract with ethyl acetate. Adjust the aqueous phase to pH 5 with a saturated NH₄Cl solution. Re-extract this aqueous phase with ethyl acetate. Dry the organic phase on Na₂SO₄, filter and evaporate. The crude reaction product is purified by silica gel column chromatography, eluent: Hex/EtOAc: 1/1. Yield: 85%. ¹H-NMR (200 MHz, CDCl₃): δ 9.50 (very broad s, 1H, OH), 7.79 (broad s, 1H, NH), 7.37 (m, 8H arom.), 7.00 (s, 1H arom.), 4.47 (s, 2H, CH₂), 4.01 (s, 3H, OCH₃).

### Methyl-6-(benzylamino-methyl)-4-hydroxy-8-methoxy-quinoline-2-carboxylate 25a

By replacing methyl-4-hydroxy-8-amino-quinoline-2-carboxylate 2u in example 21 by methyl-4-hydroxy-6-formyl-8-methoxy-quinoline-2-carboxylate 2o and benzaldehyde by benzylamine and proceeding in the same manner, the abovenamed product is obtained. Yield: 42%. ¹H-NMR (200 MHz, CDCl₃): δ 7.80 (m, 1H arom.), 7.36-7.30 (m, 5H arom.), 7.23 (m, 1H arom.), 6.97 (m, 1H arom.), 4.05 (s, 3H, OCH₃), 4.04 (s, 3H, OCH₃), 3.91 (s, 2H, NCH₂), 3.83 (s, 2H, NCH₂).

### EXAMPLE 22

### 4,8-dihydroxy-6-(furo-2-yl)-quinoline-2-carboxylic acid 44b

Dissolve methyl-8-benzyloxy-4-hydroxy-6-(furo-2-yl)-quinoline-2-carboxylate 44a (143 mg, 0.381 mmol) in a mixture of HCl 37%/acetic acid (4 ml/2 ml). Heat at 110° C. overnight. Evaporate to dryness and take up in 2 M HCl then filter the precipitate formed. Yield: 80%. ¹H-NMR (200 MHz, DMSO-d₆): δ 7.87-7.82 (m, 2H arom.), 7.51 (m, 1H arom.), 7.09 (m, 2H arom.), 6.64 (m, 1H arom.).

### 4,8-dihydroxy-6-(4-chloro-phenyl)-quinoline-2-carboxylic acid 19k

By replacing methyl-8-benzyloxy-4-hydroxy-6-(furo-2-yl)-quinoline-2-carboxylate 44a in example 22 by methyl-4-hydroxy-6-(4-chloro-phenyl)-8-benzyloxy-quinoline-2-carboxylate 18k and proceeding in the same manner, the abovenamed product is obtained. Yield: 70%. ¹H-NMR (200 MHz, DMSO-d₆): δ 7.79 (m, 1H arom.), 7.76-7.72 (m, 2H arom.), 7.58-7.54 (m, 2H arom.), 7.48 (m, 1H arom.), 6.92 (broad s, 1H arom.).

### 4,8-dihydroxy-6-(3,4-dichloro-pheenyl)-quinoline-2-carboxylic acid 19m

By replacing methyl-8-benzyloxy-4-hydroxy-6-(furo-2-yl)-quinoline-2-carboxylate 44a in example 22 by methyl-8-benzyloxy-6-(3,4-dichloro-phenyl)-4-hydroxy-quinoline-2-carboxylate 18m and proceeding in the same manner, the abovenamed product is obtained. Yield: 80%. ¹H-NMR (200 MHz, DMSO-d₆): δ 7.97 (m, 1H arom.), 7.82 (m, 1H arom.), 7.74 (m, 2H arom.), 7.47 (m, 1H arom.), 7.06 (broad s, 1H arom.).

### 4,8-dihydroxy-6-(pyridin-3-yl)-quinoline-2-carboxylic acid 19o

By replacing methyl-8-benzyloxy-4-hydroxy-6-(furo-2-yl)-quinoline-2-carboxylate 44a in example 22 by methyl-8-benzyloxy-4-hydroxy-6-(pyridin-3-yl)-quinoline-2-carboxylate 18o and proceeding in the same manner, the abovenamed product is obtained. Yield: 70%. ¹H-NMR (300 MHz, DMSO-d₆): δ 8.37 (s, 1H arom.), 8.16-8.14 (m, 1H arom.), 8.07-8.05 (m, 1H arom.), 7.43 (m, 2H arom.), 7.03 (s, 1H arom.), 6.99 (m, 1H arom.).

### 4-hydroxy-8-phenyl-quinoline-2-carboxylic acid 45c

By replacing methyl-8-benzyloxy-4-hydroxy-6-(furo-2-yl)-quinoline-2-carboxylate 44a in example 22 by benzyl-4-benzyloxy-8-phenylquinoline-2-carboxylate 45b and proceeding in the same manner, the abovenamed product is obtained. Yield: 82%. ¹H-NMR (200 MHz, DMSO-d₆): δ 10.00 (broad s, 1H, OH), 8.20-8.17 (d, 1H arom.), 7.74-7.71 (d, 1H arom.), 7.70-7.52 (m, 6H arom.), 6.78 (s, 1H arom.).

### 4,8-dihydroxy-6-(2-chlorophenyl)-quinoline-2-carboxylic acid 46d

By replacing methyl-8-benzyloxy-4-hydroxy-6-(furo-2-yl)-quinoline-2-carboxylate 44a in example 22 by benzyl-4,8-dibenzyloxy-6-(2-chlorophenyl)-quinoline-2-carboxylate 46c and proceeding in the same manner, the abovenamed product is obtained. Yield: 87%. ¹H-NMR (300 MHz, DMSO-d₆): δ 7.62-7.57 (m, 5H arom.), 7.23 (d, 1H arom.), 7.00 (broad s, 1H arom.).

### 4,8-dihydroxy-6-(3-chlorophenyl)-quinoline-2-carboxylic acid 48b

By replacing methyl-8-benzyloxy-4-hydroxy-6-(furo-2-yl)-quinoline-2-carboxylate 44a in example 22 by benzyl-4,8-dibenzyloxy-6-(3-chlorophenyl)-quinoline-2-carboxylate 48a and proceeding in the same manner, the abovenamed product is obtained. Yield: 76%. ¹H-NMR (300 MHz, DMSO-d₆): δ 10.76 (broad s, 1H, COOH), 7.82-7.69 (m, 3H arom.), 7.59-7.46 (m, 3H arom.), 7.04 (broad s, 1H arom.).

### 4,8-dihydroxy-7-phenyl-quinoline-2-carboxylic acid 50f

By replacing methyl-8-benzyloxy-4-hydroxy-6-(furo-2-yl)-quinoline-2-carboxylate 44a in example 22 by benzyl-4,8-dibenzyloxy-7-phenyl-quinoline-2-carboxylate 50e and proceeding in the same manner, the abovenamed product is obtained. Yield: 69%. ¹H-NMR (200 MHz, DMSO-d₆): δ 10.18 (broad s, 1H, COOH), 7.78-7.63 (m, 3H arom.), 7.59-7.38 (m, 4H arom.), 7.19 (broad s, 1H arom.).

### 4,8-dihydroxy-6-(2-methoxyphenyl)-quinoline-2-carboxylic acid 51b

By replacing methyl-8-benzyloxy-4-hydroxy-6-(furo-2-yl)-quinoline-2-carboxylate 44a in example 22 by benzyl-4,8-dibenzyloxy-6-(2-methoxyphenyl)-quinoline-2-carboxylate 51a and proceeding in the same manner, the abovenamed product is obtained. Yield: 31%. ¹H-NMR (200 MHz, DMSO-d₆): δ 7.63 (d, J=1.7 Hz, 1H arom.), 7.43-7.35 (m, 2H arom.), 7.32 (d, J=1.7 Hz, 1H arom.), 7.18-7.04 (m, 2H arom.), 6.97 (broad s, 1H arom.), 3.80 (s, 3H, OCH₃).

### 4,8-dihydroxy-3-phenyl-quinoline-2-carboxylic acid 53d

By replacing methyl-8-benzyloxy-4-hydroxy-6-(furo-2-yl)-quinoline-2-carboxylate 44a in example 22 by benzyl-4,8-dibenzyloxy-3-phenyl-quinoline-2-carboxylate 53c and proceeding in the same manner, the abovenamed product is obtained. Yield: 75%. ¹H-NMR (300 MHz, DMSO-d₆): δ 10.87 (broad s, 1H, COOH), 7.58-7.56 (m, 1H arom.), 7.37-7.14 (m, 7H arom.).

### 4,8-dihydroxy-6-piperidin-2-yl-quinoline-2-carboxylic acid hydrochloride 61b

By replacing methyl-8-benzyloxy-4-hydroxy-6-(furo-2-yl)-quinoline-2-carboxylate 44a in example 22 by benzyl-4,8-dibenzyloxy-6-pipeeridin-2yl-quinoline-2-carboxylate 61a and proceeding in the same manner, the abovenamed product is obtained. Yield: 75%. ¹H-NMR (300 MHz, DMSO-d₆): δ 11.17 (broad s, 1H, N⁺H), 7.60-7.47 (m, 2H arom.), 7.18 (m, 1H arom.), 3.50 (s, 4H, CH₂—N—CH₂), 1.87-1.67 (m, 6H, CH₂—CH₂—CH₂).

### Sodium 7-bromo-4,8-dihydroxy-quinoline-2-carboxylate 50h

By replacing methyl-8-benzyloxy-4-hydroxy-6-(furo-2-yl)-quinoline-2-carboxylate 44a in example 22 by methyl-7-bromo-8-benzyloxy-4-hydroxy-quinoline-2-carboxylate 50b, the abovenamed product is obtained as a green solid. Yield: 36%. ¹H NMR (200 MHz, D₂O): δ 7.51 (d, 1H, J=7 Hz, H arom.), 7.09 (d, 1H, J=7 Hz, H arom.), 6.48 (s, 1H, H³).

### 8-hexyl-4-hydroxy-quinoline-2-carboxylic acid 47b

By replacing methyl-8-benzyloxy-6-(3-benzyloxyprop-1-ynyl)-4-hydroxyquinoline-2-carboxylate 20d in example 17 by benzyl-4-benzyloxy-8-(hex-1-ynyl)-quinoline-2-carboxylate 47a and proceeding in the same manner, the abovenamed product is obtained. Yield: 70%. ¹H-NMR (300 MHz, DMSO-d₆): δ 8.03-8.01 (m, 1H arom.), 7.66-7.62 (m, 1H arom.), 7.46-7.44 (m, 1H arom.), 7.05 (s, 1H arom.), 3.11-3.09 (t, 2H, Ph-CH₂), 1.69 (m, 2H, CH₂), 1.34 (m, 6H, 3CH₂), 0.88 (m, 3H, CH₃).

### 4,8-dihydroxy-6-(3-hydroxy-propyl)-quinoline-2-carboxylic acid 49b

By replacing methyl-8-benzyloxy-6-(3-benzyloxyprop-1-ynyl)-4-hydroxyquinoline-2-carboxylate 20d in example 17 by benzyl-4,8-dibenzyloxy-6-(3-benzyloxy-prop-1-ynyl)-quinoline-2-carboxylate 49a and proceeding in the same manner, the abovenamed product is obtained. Yield: 71%. ¹H-NMR (200 MHz, DMSO-d₆): δ 7.38 (s, 1H arom.), 7.02 (s, 1H arom.), 6.86 (s, 1H arom.), 3.45-3.39 (t, 2H, CH₂), 2.69 (t, 2H, CH₂), 1.76 (t, 2H, CH₂).

### 4,8-dihydroxy-7-(3-hydroxy-propyl)-quinoline-2-carboxylic acid 54b

By replacing methyl-8-benzyloxy-6-(3-benzyloxyprop-1-ynyl)-4-hydroxyquinoline-2-carboxylate 20d in example 17 by benzyl-4,8-dibenzyloxy-7-(3-benzyloxy-prop-1-ynyl)-quinoline-2-carboxylate 54a and proceeding in the same manner, the abovenamed product is obtained. Yield: 50%. ¹H-NMR (200 MHz, DMSO-d₆): δ 7.55 (d, 1H arom.), 7.12-7.09 (d, 1H arom.), 6.58 (s, 1H arom.), 3.50-3.42 (t, 2H, CH₂), 2.78-2.74 (t, 2H, CH₂), 1.77-1.72 (t, 2H, CH₂).

### 4,8-dihydroxy-7-phenethyl-quinoline-2-carboxylic acid 55b

By replacing methyl-8-benzyloxy-6-(3-benzyloxyprop-1-ynyl)-4-hydroxyquinoline-2-carboxylate 20d in example 17 by benzyl-4,8-dibenzyloxy-7-phenylethynyl-quinoline-2-carboxylate 55a and proceeding in the same manner, the abovenamed product is obtained. Yield: 87%. ¹H-NMR (300 MHz, DMSO-d₆): δ 9.95 (broad s, CO₂H), 7.55-7.52 (d, 1H arom.), 7.38-7.19 (m, 7H arom.), 3.11-2.94 (m, 4H, Ph-CH₂—CH₂).

### 8-hydroxy-4-(aminoprop-1-ynyl)-quinoline-2-carboxylic acid hydrochloride 82a

By replacing methyl-8-benzyloxy-4-hydroxy-6-(furo-2-yl)-quinoline-2-carboxylate 44a in example 22 by methyl-8-benzyloxy-4-(3-tert-butoxycarbonylaminoprop-1-ynyl)-quinoline-2-carboxylate 12b

and proceeding in the same manner, the abovenamed product is obtained. Yield: 78%. ¹H-NMR (300 MHz, DMSO-d₆): δ 10.34 (broad s, CO₂H), 8.82 (broad s, 2H, —NH₂), 8.26 (s, 1H arom.), 7.76-7.70 (m, 2H arom.), 7.29-7.27 (m, 1H arom.), 4.10 (s, 2H, —CH₂—).

### 4-(hex-1-yl)-8-hydroxy-quinoline-2-carboxylic acid 83c

By replacing methyl-8-benzyloxy-4-hydroxy-6-(furo-2-yl)-quinoline-2-carboxylate 44a in example 22 by methyl-8-benzyloxy-4-(hex-1-yl)-quinoline-2-carboxylate 83c and proceeding in the same manner, the abovenamed product is obtained. Yield: 70%. ¹H-NMR (200 MHz, DMSO-d₆): δ 7.96 (s, 1H arom.), 7.47-7.36 (m, 2H arom.), 7.08-7.05 (m, 1H arom.), 3.03 (m, 2H, CH₂—), 1.77 (m, 2H, CH₂—), 1.34-1.26 (m, 6H, CH₂—CH₂—CH₂), 0.88 (m, 3H, CH₃).

### 8-hydroxy-4-(5-hydroxy-pent-1-yl)-quinoline-2-carboxylic acid 84c

By replacing methyl-8-benzyloxy-4-hydroxy-6-(furo-2-yl)-quinoline-2-carboxylate 44a in example 22 by methyl-8-benzyloxy-4-(5-hydroxy-pent-1-yl)-quinoline-2-carboxylate 84c and proceeding in the sane manner, the abovenamed product is obtained. Yield: 80%. ¹H-NMR (200 MHz, DMSO-d₆): δ 8.03 (s, 1H arom.), 7.68-7.66 (m, 2H arom.), 7.24-7.22 (m, 1H arom.), 3.37 (m, 2H, CH₂—), 1.16 (m, 2H, CH₂—), 1.72-1.44 (m, 6H, 3×CH₂).

### 8-hydroxy-4-(piperazin-1-yl)-quinoline-2-carboxylic acid hydrochloride 87c

By replacing methyl-8-benzyloxy-4-hydroxy-6-(furo-2-yl)-quinoline-2-carboxylate 44a in example 22 by methyl-8-hydroxy-4-(piperazin-1-yl)-quinoline-2-carboxylate 87b and proceeding in the same manner, the abovenamed product is obtained. Yield: 74%. ¹H-NMR (200 MHz, DMSO-d₆): δ 9.93 (broad s, 1H, OH), 9.71 (broad s, 1H, NH), 7.68-7.66 (m, 2H arom.), 7.24-7.22 (m, 1H arom.), 3.58-3.55 (m, 4H, CH₂—N—CH₂), 3.36-3.34 (m, 4H, CH₂—N—CH₂).

### 6-(3,5-dichlorophenyl)-4,8-dihydroxy-quinoline-2-carboxylic acid 88b

By replacing methyl-8-benzyloxy-4-hydroxy-6-(furo-2-yl)-quinoline-2-carboxylate 44a in example 22 by benzyl-4,8-dibenzyloxy-6-(3,5-dichlorophenyl)-quinoline-2-carboxylate 88a and proceeding in the same manner, the abovenamed product is obtained. Yield: 90%. ¹H-NMR (200 MHz, DMSO-d₆): δ 10.93 (broad s, 1H, OH), 7.82 (s, 1H arom.), 7.76 (s, 2H arom.), 7.65 (s, 1H arom.), 7.48 (s, 1H arom.), 7.08 (s, 1H arom.).

### 6-(4-fluorophenyl)-4,8-dihydroxy-quinoline-2-carboxylic acid 89b

By replacing methyl-8-benzyloxy-4-hydroxy-6-(furo-2-yl)-quinoline-2-carboxylate 44a in example 22 by benzyl-4,8-dibenzyloxy-6-(4-fluorophenyl)-quinoline-2-carboxylate 89a and proceeding in the same manner, the abovenamed product is obtained. Yield: 95%. ¹H-NMR (200 MHz, DMSO-d₆): δ 10.94 (broad s, 1H, OH), 7.78-7.71 (m, 3H arom.), 7.45 (s, 1H arom.), 7.35-7.26 (m, 2H arom.), 7.02 (s, 1H arom.).

### 4,8-dihydroxy-7-(3-aminoprop-1-yl)-quinoline-2-carboxylic acid hydrochloride 90c

By replacing methyl-8-benzyloxy-4-hydroxy-6-(furo-2-yl)-quinoline-2-carboxylate 44a in example 22 by methyl-4,8-dihydroxy-7-(3-tert-butoxycarbonylaminoprop-1-yl)-quinoline-2-carboxylate 90b and proceeding in the same manner, the abovenamed product is obtained. Yield: 95%. ¹H-NMR (300 MHz, DMSO-d₆): δ 10.14 (broad s, 1H, OH), 8.02 (broad s, 3H, N⁺H₃), 7.60 (d, 1H, J=8 Hz, 1H arom.), 7.43-7.38 (m, 2H arom), 2.88-2.83 (m, 4H, 2×CH₂), 1.97-1.92 (m, 2H, CH₂).

### 4,8-dihydroxy-5-trifluoromethyl-quinoline-2-carboxylic acid 98c

By replacing methyl-8-benzyloxy-4-hydroxy-6-(furo-2-yl)-quinoline-2-carboxylate 44a in example 22 by methyl-8-benzyloxy-4-hydroxy-5-trifluoromethyl-quinoline-2-carboxylate 98b and proceeding in the same manner, the abovenamed product is obtained. Yield: 88%. ¹H-NMR (300 MHz, DMSO-d₆): δ 11.73 (broad s, 1H, COOH), 10.01 (broad s, 1H, OH), 7.78 (d, 1H, J=8 Hz, 1H arom.), 7.22 (d, 1H, J=8 Hz, 1H arom.), 7.05 (s, 1H arom.).

### 4-hydroxy-8-(piperidin-1-yl)-quinoline-2-carboxylic acid hydrochloride 102b

By replacing methyl-8-benzyloxy-4-hydroxy-6-(furo-2-yl)-quinoline-2-carboxylate 44a in example 22 by benzyl-4-benzyloxy-8-(piperidin-1-yl)-quinoline-2-carboxylate 102a and proceeding in the same manner, the abovenamed product is obtained. Yield: 86%. ¹H-NMR (300 MHz, DMSO-d₆): δ 8.12 (d, 1H, J=8 Hz, 1H arom.), 8.01 (d, 1H, J=8 Hz, 1H arom.), 7.62 (t, 1H, J=8 Hz, 1H arom.), 7.25 (s, 1H arom.), 3.43-3.39 (m, 4H, CH₂—N—CH₂), 2.01-1.71 (m, 6H, 3×CH₂).

### 4-hydroxy-8-(methyl)amino-quinoline-2-carboxylic acid hydrochloride 103b

By replacing methyl-8-benzyloxy-4-hydroxy-6-(furo-2-yl)-quinoline-2-carboxylate 44a in example 22 by benzyl-4-benzyloxy-8-[benzyl(methyl)amino]-quinoline-2-carboxylate 103a and proceeding in the same manner, the abovenamed product is obtained. Yield: 91%. ¹H-NMR (300 MHz, DMSO-d₆): δ 7.47-7.32 (m, 3H arom), 6.77 (d, 1H, J=8 Hz, 1H arom.), 2.92 (s, 3H, NCH₃).

### 4-hydroxy-8-(morpholin-1-yl)-quinoline-2-carboxylic acid hydrochloride 104b

By replacing methyl-8-benzyloxy-4-hydroxy-6-(furo-2-yl)-quinoline-2-carboxylate 44a in example 22 by methyl-4-benzyloxy-8-(morpholin-1-yl)-quinoline-2-carboxylate 104a and proceeding in the same manner, the abovenamed product is obtained. Yield: 68%. ¹H-NMR (300 MHz, DMSO-d₆): δ 7.94 (d, 1H, J=8 Hz, 1H arom.), 7.72 (d, 1H, J=8 Hz, 1H arom.), 7.49 (t, 1H, J=8 Hz, 1H arom.), 6.89 (s, 1H arom.), 3.92-3.90 (m, 4H, CH₂—O—CH₂), 3.13-3.11 (m, 4H, CH₂—N—CH₂).

### 8-(4-benzyl-piperazin-1-yl)-4-hydroxy-quinoline-2-carboxylic acid hydrochloride 105b

By replacing methyl-8-benzyloxy-4-hydroxy-6-(furo-2-yl)-quinoline-2-carboxylate 44a in example 22 by methyl-4-benzyloxy-8-(4-benzyl-pipeerazin-1-yl)-quinoline-2-carboxylate 105a and proceeding in the same manner, the abovenamed product is obtained. Yield: 96%. ¹H-NMR (300 MHz, DMSO-d₆): δ 7.91 (d, 1H, J=7.6 Hz, 1H arom.), 7.68-7.43 (m, 7H arom.), 6.91 (s, 1H arom.), 4.48 (s, 2H, NCH₂), 3.48-3.39 (m, 8H, 4×CH₂).

### 8-[phenyl(methyl)amino]-4-hydroxy-quinoline-2-carboxylic acid hydrochloride 106b

By replacing methyl-8-benzyloxy-4-hydroxy-6-(furo-2-yl)-quinoline-2-carboxylate 44a in example 22 by methyl-4-benzyloxy-8-[phenyl(methyl)amino]-quinoline-2-carboxylate 106a and proceeding in the same manner, the abovenamed product is obtained. Yield: 95%. ¹H-NMR (200 MHz, DMSO-d₆): δ 8.06 (d, 1H, J=8 Hz, 1H arom.), 7.67 (d, 1H, J=8 Hz, 1H arom.), 7.50 (t, 1H, J=8 Hz, H arom.), 7.22 (t, 2H, J=8 Hz, H arom.), 6.84 (t, 1H, J=8 Hz, 1H arom.), 6.74-6.67 (m, 3H arom.), 3.34 (s, 3H, NCH₃).

### 8-(4-methyl-piperazin-1-yl)₄-hydroxy-quinoline-2-carboxylic acid hydrochloride 107b

By replacing methyl-8-benzyloxy-4-hydroxy-6-(furo-2-yl)-quinoline-2-carboxylate 44a in example 22 by methyl-4-benzyloxy-8-(4-methyl-piperazin-1-yl)-4-hydroxy-quinoline-2-carboxylate 107a and proceeding in the same manner, the abovenamed product is obtained. Yield: 91%. ¹H-NMR (200 MHz, DMSO-d₆): δ 11.13 (broad s, 1H, CO₂H), 7.90 (d, 1H, J=8 Hz, 1H arom.), 7.57-7.40 (m, 2H arom.), 6.89 (s, 1H arom.), 3.60-3.40 (m, 4H, CH₂—N—CH₂), 3.39-3.27 (m, 4H, CH₂—N—CH₂), 2.92 (s, 3H, NCH₃).

### 4-hydroxy-8-(pyridin-2-yl-amino)-quinoline-2-carboxylic acid hydrochloride 109b

By replacing methyl-8-benzyloxy-4-hydroxy-6-(furo-2-yl)-quinoline-2-carboxylate 44a in example 22 by methyl-4-benzyloxy-8-(pyridin-2-yl-amino)-quinoline-2-carboxylate 109a and proceeding in the same manner, the abovenamed product is obtained. Yield: 97%. ¹H-NMR (200 MHz, DMSO-d₆): δ 11.01 (broad s, 1H, COOH), 10.01 (broad s, 2H, NH+OH), 8.20-8.01 (m, 4H arom.), 7.69-7.60 (m, 2H arom.), 7.55 (s, 1H arom.), 7.13 (t, 1H, J=8 Hz, 1H arom.).

### 8-hydroxy-4-[2-(1-piperazinyl)pyrimidinyl]-quinoline-2-carboxylic acid hydrochloride 128b

By replacing methyl-8-benzyloxy-4-hydroxy-6-(furo-2-yl)-quinoline-2-carboxylate 44a in example 22 by methyl-8-benzyloxy-4-[2-(1-piperazinyl)pyrimidinyl]-quinoline-2-carboxylate 128a and proceeding in the same manner, the abovenamed product is obtained. Yield: 72%. ¹H-NMR (200 MHz, DMSO-d₆): δ 9.56 (broad s, 1H, COOH), 8.50-8.44 (m, 2H arom.), 7.69-7.59 (m, 2H arom.), 7.55 (s, 1H arom.), 7.38-7.34 (m, 1H arom.), 6.80-6.75 (m, 1H arom.), 4.07-3.97 (m, 4H, 2×CH₂), 3.82-3.78 (m, 2H, CH₂), 3.17-3.13 (m, 2H, CH₂).

### EXAMPLE 23

### Sodium 8-hexyl-4-hydroxy-quinoline-2-carboxylate 47d

Place 4-hydroxy-8-hexyl-quinoline-2-carboxylic acid 47c (31 mg, 0.113 mmol) in 2 ml of water, then add 0.111 ml of 1 M sodium hydroxide. Stir for 1 hour then extract once with ethyl acetate. Evaporate the aqueous phase to dryness to obtain the abovenamed product. Yield: 45%. ¹H-NMR (300 MHz, D₂O): δ 8.03-8.01 (m, 1H arom.), 7.66-7.62 (m, 1H arom.), 7.47-7.44 (m, 1H arom.), 7.05 (s, 1H arom.), 3.11-3.09 (t, 2H, Ph-CH₂), 1.69 (m, 2H, CH₂), 1.34 (m, 6H, 3CH₂), 0.88 (m, 3H, CH₃).

### Sodium 4,8-dihydroxy-7-phenyl-quinoline-2-carboxylate 50g

By replacing 4-hydroxy-8-hexyl-quinoline-2-carboxylic acid 47c in example 23 by 4,8-dihydroxy-7-phenyl-quinoline-2-carboxylic acid 50f and proceeding in the same manner, the abovenamed product is obtained. Yield: 87%. ¹H-NMR (300 MHz, D₂O): δ 7.59-7.56 (m, 2H arom.), 7.41-7.27 (m, 5H arom.), 6.85 (s, 1H arom.).

### Sodium 4,8-dihydroxy-3-phenyl-quinoline-2-carboxylate 53e

By replacing 4-hydroxy-8-hexyl-quinoline-2-carboxylic acid 47c in example 23 by 4,8-dihydroxy-3-phenyl-quinoline-2-carboxylic acid 53d and proceeding in the same manner, the abovenamed product is obtained. Yield: 94%. ¹H-NMR (300 MHz, D₂O): δ 7.67-7.64 (m, 1H arom.), 7.41-7.17 (m, 5H arom.).

### Sodium 4-(hex-1-yl)-8-hydroxy-quinoline-2-carboxylate 83d

By replacing 4-hydroxy-8-phenyl-quinoline-2-carboxylic acid 47c in example 23 by 4-(hex-1-yl)-8-hydroxy-quinoline-2-carboxylic acid 83c and proceeding in the same manner, the abovenamed product is obtained. Yield: 93%. ¹H-NMR (200 MHz, DMSO-d₆): δ 7.94 (s, 1H arom.), 7.47-7.43 (m, 2H arom.), 7.06-7.03 (m, 1H arom.), 3.02 (t, 2H, J=7.3 Hz, CH₂.), 1.66 (m, 2H, CH₂), 1.35-1.22 (m, 6H, CH₂—CH₂—CH₂), 0.87 (t, 3H, J=6.6 Hz, CH₃).

### Ssodium 8-amino-4-hydroxy-6-phenyl-quinoline-2-carboxylate 86e

By replacing 4-hydroxy-8-phenyl-quinoline-2-carboxylic acid 47c in example 23 by 8-amino-4-hydroxy-6-phenyl-quinoline-2-carboxylic acid 86d and proceeding in the same manner, the abovenamed product is obtained. Yield: 96%. ¹H-NMR (200 MHz, DMSO-d₆): δ 10.31 (broad s, 1H, OH), 7.66-7.25 (m, 7H arom.), 6.49 (s, 1H arom.), 5.96 (broad s, 2H, NH₂).

### EXAMPLE 24

### Methyl-8-benzyloxy-3-bromo-4-hydroxy-quinoline-2-carboxylate 52a

Dissolve methyl-8-benzyloxy-4-hydroxy-quinoline-2-carboxylate 2a (1.323 g, 4.227 mmol) in 50 ml of dichloromethane, cool to −5° C. then add diisopropylamine (0.603 ml, 4.279 mmol). Stir for 5 minutes and add N-bromosuccinimide (0.762 g, 4.281 mmol). Stir for 1 hour then wash the organic phase with 2 M HCl, dry on Na₂SO₄ filter and evaporate to dryness to obtain the abovenamed product. Yield: 93%. ¹H-NMR (300 MHz, CDCl₃): δ 11.08 (broad s, 1H, OH), 7.71-7.69 (d, 1H, J=7.9 Hz, 1H arom.), 7.57-7.54 (d, 1H, J=7.53 Hz, 1H arom.), 7.45-7.35 (m, 6H arom.), 5.43 (s, 2H, OCH₂), 3.95 (s, 3H, OCH₃).

### Methyl-8-benzyloxy-3,7-dibromo-4-hydroxyquinoline-2-carboxylate 56a

By replacing methyl-8-benzyloxy-4-hydroxy-quinoline-2-carboxylate 2a in example 24 by methyl-8-benzyloxy-7-bromo-4-hydroxy-quinoline-2-carboxylate 50b and proceeding in the same manner, the abovenamed product is obtained. Yield: 53%. ¹H-NMR (300 MHz, CDCl₃): δ 9.21 (broad s, 1H, OH), 8.00-7.97 (d, 1H, J=8 Hz, 1H arom.), 7.57-7.54 (d, 1H, J=8 Hz, 1H arom.), 7.40-7.37 (m, 5H arom.), 5.24 (s, 2H, OCH₂), 4.01 (s, 3H, OCH₃).

### Methyl-3-bromo-8-cyano-4-hydroxyquinoline-2-carboxylate 66a

By replacing methyl-8-benzyloxy-4-hydroxy-quinoline-2-carboxylate 2a in example 24 by methyl-8-cyano-4-hydroxy-quinoline-2-carboxylate 35b and proceeding in the same manner, the abovenamed product is obtained. Yield: 81%. ¹H-NMR (300 MHz, CDCl₃): δ 9.64 (broad s, 1H, OH), 8.62-8.59 (d, 1H, J=8 Hz, 1H arom.), 8.04-8.01 (d, 1H, J=8 Hz, 1H arom.), 7.52-7.47 (t, 1H, J=8 Hz, 1H arom.), 4.14 (s, 3H, OCH₃).

### EXAMPLE 25

### 8-hydroxy-2-(2H-tetrazol-1-yl)-quinoline 58a

To 8-hydroxyquinoline-2-carbonitrile (100 mg, 0.587 mmol) in solution in 2 ml of dimethylformamide, add sodium nitride (50 mg, 0.769 mmol), ammonium chloride (40 mg, 0.748 mmol), then stir at 120° C. for 24 hours. Dilute the reaction mixture in 1 M HCl (30 ml), then filter the precipitate, wash with water and dry to obtain the abovenamed product. Yield: 69%. ¹H-NMR (200 MHz, DMSO-d₆): δ 9.70 (broad s, 1H, OH), 8.65-8.61 (d, 1H, J=8.5 Hz, 1H arom.), 8.31-8.27 (d, 1H, J=8.5 Hz, 1H arom.), 7.66-7.54 (m, 2H arom.), 7.28-7.23 (dd, 1H, J=2 Hz and 7 Hz, 1H arom.).

### Methyl-4-hydroxy-8-(2H-tetrazol-5-yl)-quinoline-2-carboxylate 62a

By replacing methyl-8-hydroxyquinoline-2-carbonitrile in example 25 by 8-cyano-4-hydroxy-quinoline-2-carboxylate 35b, without adding ammonium chloride and substituting acetic acid for the dimethylformamide, and proceeding in the same manner, the abovenamed product is obtained. Yield: 69%. ¹H-NMR (200 MHz, DMSO-d₆): δ 11.75 and 7.49 (2 broad s, 2H, NH and OH), 8.36-8.27 (m, 2H arom.), 7.24-7.16 (m, 1H arom.), 6.67 (s, 1H arom.), 3.85 (s, 3H, OCH₃).

### EXAMPLE 26

### Ethyl-8-(benzyloxy)-3-oxo-3,4-dihydroquinoxaline-2-carboxylate 59a

To 3-(benzyloxy)benzene-1,2-diamine (300 mg, 1.4 mmol) in solution in 2 ml of ethanol, add ethyl ketomalonate (244 mg, 1.4 mmol) and acetic acid (20 μl), then stir under reflux for 1 hour. Evaporate to dryness and chromatograph to obtain the abovenamed product. Yield: 34%. ¹H-NMR (200 MHz, DMSO-d₆): δ 11.70 (broad s, 1H, NH), 7.51-7.31 (m, 6H arom.), 6.96-6.92 (d, 1H, J=8 Hz, 1H arom.), 6.84-6.79 (d, 1H, J=8 Hz, 1H arom.), 5.38 (s, 1H, OCH₂-Ph), 4.56-4.46 (q, 2H, J=7 Hz, OCH₂—CH3), 1.50-1.42 (t, 3H, J=7 Hz, OCH2CH₃).

### EXAMPLE 27

### Ethyl[8-(benzyloxy)-3-oxo-3,4-dihydroquinoxaline-2(1H)-ylidene]acetate 60a

To 3-(benzyloxy)benzene-1,2-diamine (300 mg, 1.4 mmol) in solution in 2 ml of acetic acid, add diethyloxalacetate sodium salt (295 mg, 1.4 mmol) and stir under reflux for 2 hours. Evaporate to dryness and chromatograph to obtain the abovenamed product. Yield: 20%. ¹H-NMR (200 MHz, DMSO-d₆): δ 11.39 (broad s, 1H, NH), 8.70 (broad s, 1H, NH), 7.53-7.37 (m, 5H arom.), 6.93-6.84 (t, 1H, J=8 Hz, 1H arom.), 6.71-6.67 (d, 1H, J=8 Hz, 1H arom.), 6.57-6.53 (d, 1H, J=8 Hz, 1H arom.), 5.84 (s, 1H, C═CH), 5.29 (s, 1H, OCH₂-Ph), 4.30-4.20 (q, 2H, J=7 Hz, OCH₂—CH3), 1.35-1.28 (t, 3H, J=7 Hz, OCH₂CH₃).

### EXAMPLE 28

### Methyl-6-(4-methyl-piperazin-1-yl)-8-nitro-4-benzyloxy-quinoline-2-carboxylate 124a

In a degassed solution of anhydrous toluene (3 ml) containing cesium carbonate (220 mg, 0.672 mmol, 1.4 eq.), add methyl-6-bromo-8-nitro-4-benzyloxy-quinoline-2-carboxylate 31e (200 mg, 0.48 mmol), N-methylpiperazine (58 mg, 64 μl, 0.58 mmol, 1.2 eq.), bispalladium (0) dibenzylidene acetone (8.8 mg, 0.0096 mmol, 0.02 eq.) and rac-BINAP (18 mg, 0.0289 mmol, 0.06 eq.). Heat at 100° C. for 18 hours. Cool the solution and evaporate to dryness. Take up in dichloromethane (10 ml), wash with water (5 ml) and with a saturated sodium chloride solution (5 ml), dry on Na₂SO₄, filter and evaporate. Purify the residue by silic gel column chromatography (eluent: CH₂Cl₂/MeOH: 95/5). The abovenamed product is isolated as an orange solid. Yield: 64%. ¹H NMR (300 MHz, CDCl₃): δ 7.86 (d, 1H, J=2 Hz, H arom.), 7.68 (s, 1H³), 7.57 (d, 1H, J=2 Hz, H arom.), 7.46 (m, 5H arom.), 5.38 (s, 2H benz.), 4.01 (s, 3H, OCH₃), 3.40 (m, 4H, 2 NCH₂), 2.61 (m, 4H, 2 CH₂), 2.38 (s, 3H, NCH₃).

### Methyl-6-(N-(N-benzyl-piperazinyl))-8-nitro-4-benzyloxy-quinoline-2-carboxylate 125a

By replacing N-methylpiperazine in example 28 by N-benzylpiperazine, the abovenamed product is obtained as an orange solid. Yield: 73%. ¹H NMR (300 MHz, CDCl₃): δ 7.89 (d, 1H, J=2 Hz, H arom.), 7.71 (s, 1H³), 7.59 (d, 1H, J=2 Hz, H arom.), 7.40 (m, 10H arom.), 5.40 (s, 2H benz.), 4.05 (s, 3H, OCH₃), 3.63 (s, 2H benz.), 3.42 (m, 4H, 2 NCH₂), 2.68 (m, 4H, 2 CH₂).

### Methyl-6-(N-piperidinyl)-8-nitro-4-benzyloxy-quinoline-2-carboxylate 126a

By replacing N-methylpiperazine in example 28 by piperidine, the abovenamed product is obtained as an orange solid. Yield: 59%. ¹H NMR (200 MHz, CDCl₃): δ 7.86 (d, 1H, J=2 Hz, H arom.), 7.66 (s, 1H³), 7.53 (d, 1H, J=2 Hz, H arom.), 7.47 (m, 10H arom.), 5.38 (s, 2H benz.), 4.01 (s, 3H, OCH₃), 3.38 (m, 4H, 2 NCH₂), 1.70 (m, 6H, 3 CH₂).

### Methyl-6-diphenylmethylenamine-8-nitro-4-benzyloxy-quinoline-2-carboxylate 32a

By replacing N-methylpiperazine in example 28 by diphenylimine, the abovenamed product is obtained as an orange solid. Yield: 82%. ¹H NMR (200 MHz, CDCl₃): δ 7.77 (m, 2H, 2H arom.), 7.67 (s, 1H 3), 7.53-7.12 (m, 16H, 16H arom.), 5.31 (s, 2H benz.), 4.01 (s, 3H, OCH₃).

### Methyl-6-(N-anilino)-8-nitro-4-benzyloxy-quinoline-2-carboxylate 33a

By replacing N-methylpiperazine in example 28 by aniline, the abovenamed product is obtained as an orange solid. Yield: 84%. ¹H NMR (200 MHz, CDCl₃): δ 7.87 (m, 2H, 2H arom.), 7.68 (s, 1H³), 7.40 (m, 9H, 9H arom.), 6.24 (s, 1H, 1NH), 7.20 (m, 2H, 2H arom.), 5.36 (s, 2H benz.), 4.02 (s, 3H, OCH₃).

### Methyl-5-(piperidin-1-yl)-8-nitro-4-benzyloxy-quinoline-2-carboxylate 77a

By replacing in example 28 N-methylpiperazine by piperidine and methyl-6-bromo-8-nitro-4-benzyloxy-quinoline-2-carboxylate by methyl-5-bromo-8-nitro-4-benzyloxy-quinoline-2-carboxylate 72c, the abovenamed product is obtained as an orange solid. Yield: 61%. ¹H NMR (200 MHz, CDCl₃): δ 8.10 (d, 1H, J=8 Hz, H arom.), 7.70 (s, 1H³), 7.49 (m, 2H, 2H arom.), 7.47 (m, 3H arom.), 6.94 (d, 1H, J=8 Hz, H arom.), 5.37 (s, 2H benz.), 4.03 (s, 3H, OCH₃), 3.29 (m, 2H, NCH₂), 2.83 (m, 2H, NCH₂), 1.48 (m, 6H, 3 CH₂).

### Methyl-5-(4-benzyl-piperazin-1-yl)-8-nitro-4-benzyloxy-quinoline-2-carboxylate 78a

By replacing in example 28 N-methylpiperazine by N-benzylpiperazine and methyl-6-bromo-8-nitro-4-benzyloxy-quinoline-2-carboxylate by methyl-5-bromo-8-nitro-4-benzyloxy-quinoline-2-carboxylate 72c, the abovenamed product is obtained as an orange solid. Yield: 58%. ¹H NMR (300 MHz, CDCl₃): δ 8.07 (d, 1H, J=8 Hz, H arom.), 7.75 (s, 1H³), 7.52 (m, 5H, 5H arom.), 7.21 (m, 5H arom.), 7.96 (d, 1H, J=8 Hz, H arom.), 5.33 (s, 2H benz.), 4.04 (s, 3H, OCH₃), 3.20 (m, 2H, 2 NCH), 3.18 (s, 2H benz.), 2.93 (m, 2H, 2 NCH), 2.54 (m, 2H, 2 NCH), 1.96 (m, 2H, 2 NCH).

### Benzyl-4,8-dibenzyloxy-6-piperidin-2yl-quinoline-2-carboxylate 61a

By replacing in example 28 N-methylpiperazine by piperidine and methyl-6-bromo-8-nitro-4-benzyloxy-quinoline-2-carboxylate 72c by benzyl-4,8-dibenzyloxy-6-bromoquinoline-2-carboxylate 46b and proceeding in the same manner, the abovenamed product is obtained. Yield: 45%. ¹H-NMR (200 MHz, CDCl₃): δ 7.61-7.32 (m, 16H arom.), 7.06 (m, 1H arom.), 6.92 (m, 1H arom.), 5.46 (s, 2H, OCH₂), 5.37 (s, 2H, OCH₂), 5.34 (s, 2H, OCH₂), 3.28 (m, 4H, CH₂—N—CH₂), 1.72 (m, 6H, CH₂—CH₂—CH₂).

### Methyl-8-benzyloxy-4-(4-benzyl-piperazin-1-yl)-quinoline-2-carboxylate 87a

Starting with methyl-8-benzyloxy-4-trifluoromethanesulfonyloxy-quinoline-2-carboxylate 11 and by replacing in example 28 N-methylpiperazine by N-benzylylpiperazine and proceeding in the same manner, the abovenamed product is obtained. Yield: 62%. ¹H-NMR (200 MHz, CDCl₃): δ 7.70 (s, 1H arom.), 7.61-7.28 (m, 12H arom.), 7.04 (d, 1H, J=8 Hz, 1H arom.), 5.43 (s, 2H, OCH₂), 4.03 (s, 3H, OCH₃), 3.65 (s, 2H, NCH₂), 3.35-3.31 (m, 4H, CH₂—N—CH₂), 2.78-2.75 (m, 4H, CH₂—N—CH₂).

### Methyl-8-benzyloxy-4-(morpholin-1-yl)-quinoline-2-carboxylate 99a

Starting with methyl-8-benzyloxy-4-trifluoromethanesulfonyloxy-quinoline-2-carboxylate 11 and by replacing in example 28 N-methylpiperazine by morpholine and proceeding in the same manner, the abovenamed product is obtained. Yield: 96%. ¹H-NMR (300 MHz, CDCl₃): δ 7.73 (s, 1H arom.), 7.57-7.28 (m, 7H arom.), 7.06 (d, 1H, J=8 Hz, 1H arom.), 5.44 (s, 2H, OCH₂), 4.04 (s, 3H, OCH₃), 4.01-3.98 (m, 4H, CH₂—O—CH₂), 3.31-3.28 (m, 4H, CH₂—N—CH₂).

### Methyl-8-benzyloxy-4-(piperidin-1-yl)-quinoline-2-carboxylate 100a

Starting with methyl-8-benzyloxy-4-trifluoromethanesulfonyloxy-quinoline-2-carboxylate 11 and by replacing in example 28 N-methylpiperazine by piperidine and proceeding in the same manner, the abovenamed product is obtained. Yield: 62%. ¹H-NMR (300 MHz, CDCl₃): δ 7.69 (s, 1H arom.), 7.57-7.28 (m, 7H arom.), 7.04 (d, 1H, J=8 Hz, 1H arom.), 5.43 (s, 2H, OCH₂), 4.03 (s, 3H, OCH₃), 3.27-3.23 (m, 4H, CH₂—N—CH₂), 1.86-1.71 (m, 6H, 3×CH₂).

### Methyl-8-nitro-4-(piperidin-1-yl)-quinoline-2-carboxylate 101a

Starting with methyl-8-nitro-4-trifluoromethanesulfonyloxy-quinoline-2-carboxylate 92a and by replacing in example 28 N-methylpiperazine by piperidine and proceeding in the same manner, the abovenamed product is obtained. Yield: 76%. ¹H-NMR (300 MHz, CDCl₃): δ 8.22 (d, 1H, J=8 Hz, 1H arom.), 8.20 (d, 1H, J=8 Hz, 1H arom.), 7.72 (s, 1H arom.), 7.58 (t, 1H, J=8 Hz, 1H arom.), 4.03 (s, 3H, OCH₃), 3.31-3.28 (m, 4H, CH₂—N—CH₂), 1.92-1.57 (m, 6H, 3×CH₂).

### Benzyl-4-benzyloxy-8-(piperidin-1-yl)-quinoline-2-carboxylate 102a

Starting with benzyl-4-benzyloxy-8-bromo-quinoline-2-carboxylate 45a and by replacing in example 28 N-methylpiperazine by piperidine and proceeding in the same manner, the abovenamed product is obtained. Yield: 67%. ¹H-NMR (300 MHz, CDCl₃): δ 7.72-7.69 (dd, 1H arom.), 7.63 (s, 1H arom.), 7.58-7.35 (m, 11H arom.), 7.16-7.14 (dd, 1H arom.), 5.44 (s, 2H, OCH₂), 5.40 (s, 2H, OCH₂) 3.31-3.28 (m, 4H, CH₂—N—CH₂), 1.67-1.55 (m, 6H, 3×CH₂).

### Methyl-4-benzyloxy-8-benzyl(methyl)amino-quinoline-2-carboxylate 103a

Starting with methyl-4-benzyloxy-8-bromo-quinoline-2-carboxylate 45a′ and by replacing in example 28 N-methylpiperazine by N-benzylmethylamine and proceeding in the same manner, the abovenamed product is obtained. Yield: 75%. ¹H-NMR (300 MHz, CDCl₃): δ 7.87-7.83 (dd, 1H arom.), 7.68 (s, 1H arom.), 7.58-7.27 (m, 11H arom.), 7.09-7.06 (dd, 1H arom.), 5.37 (s, 2H, OCH₂), 4.90 (s, 2H, NCH₂), 3.97 (s, 3H, OCH₃), 2.91 (s, 3H, NCH₃).

### Methyl-4-benzyloxy-8-(morpholin-1-yl)-quinoline-2-carboxylate 104a

Starting with methyl-4-benzyloxy-8-bromo-quinoline-2-carboxylate 45a′ and by replacing in example 28 N-methylpiperazine by mopholine and proceeding in the same manner, the abovenamed product is obtained. Yield: 96%. ¹H-NMR (300 MHz, CDCl₃): δ 7.89 (d, 1H, J=8 Hz, 1H arom.), 7.67 (s, 1H arom.), 7.53-7.26 (m, 6H arom.), 7.15 (d, 1H, J=8 Hz, 1H arom.), 5.5 (s, 2H, OCH₂), 4.09-4.02 (m, 4H, CH₂—O—CH₂), 4.02 (s, 3H, OCH₃), 3.49-3.46 (m, 4H, CH₂—N—CH₂).

### Methyl-4-benzyloxy-8-(4-benzyl-piperazin-1-yl)-quinoline-2-carboxylate 105a

Starting with methyl-4-benzyloxy-8-bromo-quinoline-2-carboxylate 45a′ and by replacing in example 28 N-methylpiperazine by N-benzylpiperazine and proceeding in the same manner, the abovenamed product is obtained. Yield: 68%. ¹H-NMR (200 MHz, CDCl₃): δ 7.87 (d, 1H, J=7.5 Hz, 1H arom.), 7.51 (s, 1H arom.), 7.44-7.28 (m, 11H arom.), 7.14 (d, 1H, J=7.6 Hz, 1H arom.), 5.34 (s, 2H, OCH₂), 4.02 (s, 3H, OCH₃), 3.67 (s, 2H, NCH₂), 3.49-3.47 (m, 4H, CH₂—N—CH₂), 2.86-2.83 (m, 4H, CH₂—N—CH₂).

### Methyl-4-benzyloxy-8-phenyl(methyl)amino-quinoline-2-carboxylate 106a

Starting with methyl-4-benzyloxy-8-bromo-quinoline-2-carboxylate 45a′ and by replacing in example 28 N-methylpiperazine by N-phenyl,N-methylamine and proceeding in the same manner, the abovenamed product is obtained. Yield: 65%. ¹H-NMR (300 MHz, CDCl₃): δ 8.08 (d, 1H, J=8 Hz, 1H arom.), 7.67-7.44 (m, 8H arom.), 7.20-7.18 (m, 2H arom.), 6.93-6.081 (m, 3H arom.), 5.36 (s, 2H, OCH₂), 3.92 (s, 3H, OCH₃), 3.56 (s, 3H, NCH₃).

### Methyl-4-benzyloxy-8-(4-methyl-piperazin-1-yl)-quinoline-2-carboxylate 107a

By replacing methyl-4-benzyloxy-6-bromo-8-nitro-quinoline-2-carboxylate 31e in example 28 by benzyl-4-benzyloxy-8-bromo-quinoline-2-carboxylate 45a and proceeding in the same manner, the abovenamed product is obtained. Yield: 68%. ¹H-NMR (200 MHz, CDCl₃): δ 7.87 (d, 1H, J=7.5 Hz, 1H arom.), 7.54 (s, 1H arom.), 7.51-7.15 (m, 6H arom.), 7.16 (d, 1H, J=8 Hz, 1H arom.), 5.35 (s, 2H, OCH₂), 4.02 (s, 3H, OCH₃), 3.52-3.49 (m, 4H, CH₂—N—CH₂), 2.83-2.81 (m, 4H, CH₂—N—CH₂), 2.45 (s, 3H, NCH₃).

### Methyl-4-benzyloxy-8-(pyridin-2-yl-amino)-quinoline-2-carboxylate 109a

Starting with methyl-4-benzyloxy-8-bromo-quinoline-2-carboxylate 45a′ and by replacing in example 28 N-methylpiperazine by 2-aminopyridine and proceeding in the same manner, the abovenamed product is obtained. Yield: 80%. ¹H-NMR (300 MHz, CDCl₃): δ 9.22 (s, 1H, NH), 8.88 (d, 1H, J=8 Hz, 1H arom.), 8.38-8.35 (m, 1H arom.), 7.79-7.42 (m, 9H arom.), 7.06 (d, 1H, J=8 Hz, 1H arom.), 6.87-6.80 (m, 1H arom.), 5.38 (s, 2H, OCH₂), 4.08 (s, 3H, OCH₃).

### Methyl-8-benzyloxy-4-(4-methyl-piperazin-1-yl)-quinoline-2-carboxylate 110a

By replacing methyl-4-benzyloxy-6-bromo-8-nitro-quinoline-2 carboxylate 31e in example 28 by methyl-8-benzyloxy-4-trifluoromethanesulfonyloxy-quinoline-2-carboxylate 11 and proceeding in the same manner, the abovenamed product is obtained. Yield: 68%. ¹H-NMR (200 MHz, CDCl₃): δ 7.72 (s, 1H arom.), 7.60-7.54 (m, 3H arom.), 7.43-7.30 (m, 4H arom.), 7.04 (d, 1H, J=8 Hz, 1H arom.), 5.43 (s, 2H, OCH₂), 4.03 (s, 3H, OCH₃), 3.37-3.32 (m, 4H, CH₂—N—CH₂), 2.74-2.71 (m, 4H, CH₂—N—CH₂), 2.42 (s, 3H, NCH₃).

### Methyl-8-benzyloxy-4-[2-(1-piperazinyl)pyrimidinyl]-quinoline-2-carboxylate 128a

Starting with methyl-8-benzyloxy-4-trifluoromethanesulfonyloxy-quinoline-2-carboxylate 11 and by replacing in example 28 N-methylpiperazine by 2-(1-piperazinyl)pyrimidine and proceeding in the same manner, the abovenamed product is obtained. Yield: 46%. ¹H-NMR (200 MHz, CDCl₃): δ 8.36 (d, 2H, J=5 Hz, 1H arom.), 7.75 (s, 1H arom.), 7.70-7.35 (m, 7H arom.), 7.07 (d, 1H, J=8 Hz, 1H arom.), 6.58 (d, 1H, J=5 Hz, 1H arom.), 5.44 (s, 2H, OCH₂), 3.35-3.31 (m, 4H, 2×CH₂), 4.05 (s, 3H, OCH₃), 3.38-3.34 (m, 4H, 2×CH₂).

### EXAMPLE 29

### Methyl-3-bromo-8-amino-4-hydroxy-quinoline-2-carboxylate 45b

To a solution of methyl-3-bromo-8-nitro-4-hydroxy-quinoline-2-carboxylate 45a (100 mg, 0.3 mmol) in THF (2.5 ml), add a Na₂S₂O₄ solution (264 mg, 1.52 mmol, 5 eq.) in water (2 ml). Stir at room temperature for 3 hours, then add ethyl acetate (20 ml) and water (10 ml) and extract the organic phase, which is washed successively with water (10 ml) and saturated NaCl (10 ml). Dry the organic phase on Na₂SO₄, filter, evaporate to dryness and purify by silica gel chromatography (eluent: CH₂Cl₂/EtOAc: 55/45) to give the abovenamed product in the form of a yellow solid. Yield: 83%. ¹H NMR (200 MHz, d₆-DMSO): δ 11.31 (s, 1 HO), 7.41 (dd, 1H, J=1 and 8 Hz, H arom.), 7.23 (t, 1H, J=8 Hz, H arom.), 6.97 (dd, 1H, J=2 and 8 Hz, H arom.), 6.45 (s, 2 HN), 4.03 (s, 3H, OCH₃).

### Methyl-6-bromo-8-amino-4-hydroxy-quinoline-2-carboxylate 31c

By replacing methyl-3-bromo-8-nitro-4-hydroxy-quinoline-2-carboxylate 45a in example 21 by methyl-6-bromo-8-nitro-4-hydroxy-quinoline-2-carboxylate 31b, the abovenamed product is obtained as a white solid. Yield: 51%. ¹H NMR (200 MHz, d₆-DMSO): δ 11.80 (s, 1 HO), 7.47 (s, 1H³), 7.35 (d, 1H, J=2 Hz, H⁵), 6.97 (d, 1H, J=2 Hz, H⁷), 6.28 (s, 2 HN), 3.93 (s, 3H, OCH₃).

### Methyl-6-chloro-8-amino-4-hydroxy-quinoline-2-carboxylate 117c

By replacing methyl-3-bromo-8-nitro-4-hydroxy-quinoline-2-carboxylate 45a in example 21 by methyl-6-chloro-8-nitro-4-hydroxy-quinoline-2-carboxylate 117b, the abovenamed product is obtained as an orange solid. Yield: 59%. ¹H NMR (200 MHz, d₆-DMSO): δ 11.60 (s, 1 HO), 7.45 (s, 1H³), 7.17 (d, 1H, J=2 Hz, H⁵), 6.85 (d, 1H, J=2 Hz, H⁷), 6.27 (s, 2 HN), 3.90 (s, 3H, OCH₃).

### Benzyl-8-amino-4-benzyloxy-quinoline-2-carboxylate 34b

By replacing methyl-3-bromo-8-nitro-4-hydroxy-quinoline-2-carboxylate 45a in example 21 by benzyl-8-nitro-4-benzyloxy-quinoline-2-carboxylate 34a, the abovenamed product is obtained as a yellow solid. Yield: 80%. ¹H NMR (200 MHz, CDCl₃): δ 7.41 (m, 13H, 13H arom.), 6.94 (d, 1H, J=8 Hz, 1H⁶), 5.48 (s, 2H, CH₂benz.), 5.33 (s, 2H, CH₂benz.), 5.12 (s, 2H, NH₂).

### EXAMPLE 30

### Benzyl-7-iodo-8-amino-4-benzyloxy-quinoline-2-carboxylate 34c

To a solution of benzyl-8-amino-4-benzyloxy-quinoline-2-carboxylate 34b (100 mg, 0.32 mmol) in absolute ethanol (10 ml), add iodine (82 mg, 0.32 mmol, 1 eq.) and silver sulfate (101 mg, 0/32 mmol, 1 eq.) and stir at room temperature for 6 hours. Add sodium thiosulfate solution (10 ml) and evaporate the ethanol. Take up in dichloromethane (50 ml), wash the organic phase with water (10 ml) and dry on sodium sulfate, filter and evaporate to dryness. Purify the residue by silica gel column chromatography (eluent: CH₂Cl₂/MeOH: 98/2) to isolate the abovenamed product as a yellow product. Yield: 36%. ¹H NMR (200 MHz, CDCl₃): δ 7.73 (d, 1H, J=8 Hz, H⁷), 7.65 (s, 1H³), 7.43 (m, 10H arom.), 7.32 (d, 1H, J=8 Hz, H⁶), 5.62 (s, 2H, 2 NH), 5.35 (s, 2H benz.), 5.27 (s, 2H benz.).

### EXAMPLE 31

### Methyl-6-cyano-8-nitro-4-hydroxy-quinoline-2-carboxylate 120a

A solution of methyl-6-bromo-8-nitro-4-hydroxy-quinoline-2-carboxylate 31e (400 mg, 0.96 mmol), zinc cyanide (67.5 mg, 0.27 mmol, 0.6 eq.), diphenylphosphineferrocene (DPPF, 21.2 mg, 0.038 mg, 0.04 eq.), bispalladium(0)dibenzylideneacetone (Pd₂(dba)₃, (17.6 mg, 0.019 mmol, 0.02 eq.) and zinc powder (7.5 mg, 0.115 mmol, 0.12 eq.) in degassed N,N-dimethylacetamide (6 ml) is heated at 120° C. for 16 hours. Transfer the solution into 50 ml of ethyl acetate. Wash the organic phase with 2 M NH₄OH (50 ml). Extract the aqueous phase three times with ethyl acetate (3×10 ml). Combine the organic phases and wash with saturated NaCl solution, dry on Na₂SO₄, filter and evaporate to dryness. Purify the residue by silica gel chromatography (eluent: CH₂Cl₂/EtOAc: 99/1) to isolate the abovenamed product as a white solid. Yield: 66%. ¹H NMR (200 MHz, CDCl₃): δ 8.82 (d, 1H, J=2 Hz, H⁷), 8.21 (d, 1H, J=2 Hz, H⁵), 7.88 (s, 1H³), 7.49 (m, 5H arom.), 5.44 (s, 2H benz.), 4.06 (s, 3H, OCH₃).

### EXAMPLE 32

### Methyl-3-bromo-4-hydroxy-8-nitro-quinoline-2-carboxylate 45a

To a solution of methyl-4-hydroxy-8-nitro-2-quinoline-2-carboxylate 2s (0.5 g, 2 mmol) in CCl₄ (35 ml), add N-bromosuccinimide (0.39 g, 2.2 mmol, 1.1 eq.) and react under reflux for 2 hours. Evaporate the solvent and purify the residue by silica gel chromatography (eluent: EtOAc/CH₂Cl₂/Hexane: 1/1.6/2.4) to obtain the abovenamed product as a yellow solid. Yield=80%. ¹H NMR (300 MHz, CDCl₃): δ 12.15 (s, 1 HO), 8.80 (dd, 1H, J=2 and 8 Hz, H arom.), 8.76 (dd, 1H, J=2 and 8 Hz, H arom.), 7.56 (dt, 1H, J=2 and 8 Hz, H arom.), 4.19 (s, 3H, OCH₃).

### EXAMPLE 33

### Methyl-6-amino-8-nitro-4-benzyloxy-quinoline-2-carboxylate 32b

To a solution of methyl-6-diphenylmethylenamino-8-nitro-4-benzyloxy-quinoline-2-carboxylate 32a (210 mg, 0.386 mmol) in THF (10 ml), add at room temperature 2 M hydrochloric acid (4 ml) and stir for 2 hours. Filter the purple precipitate which forms and wash with water, then with ethyl acetate (2×2 ml). Dissolve the solid in dichloromethane (10 ml) and wash with a saturated sodium carbonate solution (2 ml), water (2 ml), then dry on sodium sulfate, filter and evaporate. The abovenamed product is isolated as an orange solid. Yield: 82%. ¹H NMR (300 MHz, CDCl₃): δ 7.67 (s, 1H³), 7.50 (m, 6H arom.), 7.59 (s, 1H, 1H arom.), 5.34 (s, 2H benz.), 4.30 (s, 2H, NH₂), 4.05 (s, 3H, OCH₃).

### EXAMPLE 34

### 3-ethynyl-8-nitro-4-hydroxy-quinoline-2-carboxylic acid 57b

To methyl-3-trimethylsilylethynyl-8-nitro-4-hydroxy-quinoline-2-carboxylate ester 57a (101 mg, 0.29 mmol) in a mixture of THF/MeOH (1:1.4 ml), add KOH solution (0.04 g, 0.71 mmol, 2.5 eq.) in water (1 ml). Stir the solution at room temperature for 4 hours and neutralize by adding 1 M HCl. Evaporate the THF and take up the residual solution in CH₂Cl₂. Extract the organic phase, wash with water, then with saturated NaCl. Dry on sodium sulfate, filter and evaporate. The abovenamed product is obtained as a beige solid. Yield: 63%. ¹H NMR (200 MHz, CDCl₃): δ 8.64 (d, 1H, J=8 Hz, H arom.), 8.25 (d, 1H, J=8 Hz, H arom.), 8.02 (s, 1H, OH), 7.90 (t, 1H, J=8 Hz, H arom.), 5.30 (s, 1H, CH), 4.14 (s, 3H, CH₃).

### Methyl-8-cyano-6-ethynyl-4-hydroxy-quinoline-2-carboxylate 69b

By replacing methyl-3-trimethylsilylethynyl-8-nitro-4-hydroxy-quinoline-2-carboxylate 57a in example 34 by methyl-8-cyano-4-hydroxy-6-[(trimethylsilyl)ethynyl]-quinoline-2-carboxylate 69a and proceeding in the same manner, the abovenamed product is obtained. Yield: 91%. ¹H-NMR (300 MHz, CDCl₃+MeOD): δ 8.49 (m, 1H arom.), 7.99 (m, 1H arom.), 7.04 (m, 1H arom.), 3.99 (s, 3H, OCH₃), 6.67 (s, 1H, CCH).

### EXAMPLE 35

### Benzyl-8-nitro-4-benzyloxy-quinoline-2-carboxylate 34a

To a solution of methyl-8-nitro-4-hydroxy-quinoline-2-carboxylate 2u (500 mg, 2 mmol) in MeOH (10 ml) add an aqueous 10% sodium hydroxide solution (2 ml) and stir at room temperature for 3 hours. Acidify to pH 3 by adding 1 M HCl and filter the yellow precipitate formed. Dry the solid and isolate 8-nitro-4-hydroxy-quinoline-2-carboxylic acid as a yellow solid. Yield: 78%. ¹H NMR (200 MHz, d₆-DMSO): δ 11.66 (s, 1H, OH), 8.74 (d, 1H, J=8 Hz, 1H arom.), 8.74 (d, 1H, J=8 Hz, 1H arom.), 7.61 (t, 1H, J=8 Hz, H⁶), 6.84 (s, 1H, H³). Dissolve the previous solid in DMF (10 ml) and add potassium carbonate (1.0 g, 7.22 mmol, 2.8 eq.) then benzyl bromide (0.96 g, 7.22 mmol, 2.8 eq.) and heat at 60° C. for 4 hours. Pour the reaction mixture into iced water (80 ml) and filter the solid formed, redissolve in CH₂Cl₂, wash with H₂O then with saturated NaCl and dry on Na₂SO₄, filter and evaporate to dryness. Crystallize by addition of Et₂O/Hexane 1/3. The abovenamed product is isolated as a white solid. Yield: 86%. ¹H NMR (200 MHz, CDCl₃): δ 8.52 (d, 1H, J=8 Hz, 1H arom.), 8.14 (d, 1H, J=8 Hz, 1H arom.), 7.79 (s, 1H, 1H³), 7.68 (t, 1H, J=8 Hz, 1H⁶), 7.55 (m, 10H, 10H arom.), 5.53 (s, 2H, CH₂benz.), 5.53 (s, 2H, CH₂benz.).

### EXAMPLE 36

### 3-(N-morpholinomethyl)-8-benzyloxy-4-hydroxy-quinoline-2-carboxylic acid 39a

To a solution of methyl-8-benzyloxy-4-hydroxy-quinoline-2-carboxylate 2a (300 mg, 0.97 mmol) in ethanol (20 ml), add at room temperature morpholine (0.25 mL, 2.87 mmol, 2.9 eq.) then formol (0.15 of a 37% solution in water, 1.62 mmol, 1.6 eq.) and heat under reflux for 24 hours. Evaporate the solvents and add shaved ice. Filter the precipitate formed. The abovenamed product is isolated as a pink solid. Yield: 55%. ¹H NMR (200 MHz, d₆-DMSO): δ 11.85 (s, 1H, OH), 10.37 (s, 1H, OH), 7.69 (d, 1H, J=8 Hz, 1H arom.), 7.46 (m, 7H, 7H arom.), 5.39 (s, 2H, CH₂benz.), 4.53 (s, 2H, CH₂N), 3.80 (m, 4H, 2 CH₂), 3.42 (m, 2H, 2 CH₂).

### 3-(N-pyrolidinomethyl)-8-benzyloxy-4-hydroxy-quinoline-2-carboxylic acid 39a

By replacing morpholine in example 36 by pyrolidine, the abovenamed product is obtained as a beige solid. Yield: 32%. ¹H NMR (200 MHz, d₆-DMSO): δ 10.60 (s, 1H, OH), 10.48 (s, 1H, OH), 7.69 (d, 1H, J=8 Hz, 1H arom.), 7.45 (m, 7H, 7H arom.), 5.40 (s, 2H, CH₂benz.), 4.57 (s, 2H, CH₂N), 3.28 (m, 4H, 2 CH₂), 1.98 (m, 4H, 2 CH₂).

### EXAMPLE 37

### Methyl-8-benzyloxy-4-(3-aminoprop-1-yl)-quinoline-2-carboxylate 85b

Dissolve 200 mg (0.444 mmol) of methyl-8-benzyloxy-4-(3-tert-butoxycarbonylaminoprop-1-yl)-quinoline-2-carboxylate 85a in 2 ml of dichloromethane and add 1 ml of trifluoroacetic acid. Stir at room temperature for 1 hour, evaporate to dryness and dry to obtain the abovenamed product. Yield: 93%. ¹H-NMR (300 MHz, DMSO-d₆): δ 8.05 (s, 1H arom.), 7.81-7.36 (m, 8H arom.), 5.43 (s, 2H, OCH₂), 3.96 (s, 3H, CH₃), 3.22 (t, 2H, J=7.5 Hz, CH₂), 2.95 (m, 2H, CH₂), 1.99 (m, 2H, CH₂).

### EXAMPLE 38

### Biological Activity of the Compounds

Screening Protocol

Screening comprises testing the synthetic compounds at a fairly low concentration (10 μM) for their ability to displace the binding of 200 nM [³H]-XA (using non-radiolabelled xanthurenic acid as control). If this concentration produces the same or greater displacement than the same concentration of non-radiolabelled XA, then an IC₅₀ is determined.

Binding is carried out in 50 mM Pipes buffer pH 7.4 at 0° C. (in ice) in the presence of synaptic membranes (from 0.1 to 0.3 mg of protein per tube), tritium-labelled xanthurenic acid ([³H]-XA) at 200 nM final concentration and
either buffer (to determine total binding)
or non-tritiated xanthurenic acid at 2 mM concentration (to determine nonspecific binding)
or non-tritiated xanthurenic acid at 10 μM concentration (positive control)
or the test compound at 10 μM final concentration.

The incubation time is 25 min. The filtration by which unbound [³H]-XA is separated from [³H]-XA bound to its binding site(s) is carried out by rapid aspiration of the incubation medium through Whatmann (GF/B) fiberglass filters which are then successively washed twice with cold 50 mM Pipes buffer pH 7.4 (3×3 ml altogether). The filters are placed in scintillation vials to which 5 ml of Rotiszint® (Roth) are added and the vials are counted in a Beckman LS6000sc liquid scintillation counter.

Specific binding is given as the difference between total binding and nonspecific binding. The percentage inhibition of binding that the synthetic compounds produce is calculated for each compound in relation to its positive control.

Competition Protocol—IC₅₀ Determination (50% Inhibitory Concentration)

Binding is carried out in 50 mM Pipes buffer pH 7.4 at 0° C. (in ice) in the presence of synaptic membranes (from 0.1 to 0.3 mg of protein per tube), tritium-labelled xanthurenic acid ([³H]-XA) at 200 nM final concentration and either buffer (to determine total binding), or non-radiolabelled xanthurenic acid at 2 mM concentration (to determine nonspecific binding) or different concentrations of a non-radiolabelled synthetic compound. If the compound reversibly binds to the binding site of [³H]-XA, the two ligands will compete with each other and a binding displacement curve for [³H]-XA can be plotted against the concentration of the competitor molecule. The incubation time is 25 minutes, followed by filtration.

Graphpad Prism software is used to compute the IC₅₀ which is the concentration of the compound that produces 50% inhibition of binding of xanthurenic acid. In a competition experiment between [³H]-XA and non-radiolabelled XA, a binding displacement curve is obtained which, after analysis by Graphpad Prism software, gives a two site binding model, one IC₅₀ at 300 nM and one IC₅₀ at 57 μM.

Results

The results obtained are shown in the following tables and in FIG. 1.

| | | % inhibition of XA | IC₅₀ |
|---|---|---|---|
| Compound | | binding at 50 μM* | (μM) |
| Xanthurenic acid | | 100 | 0.3 and 57 |
| 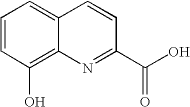 | | | 0.09 and 12.3 |
| | 3i | 80 | 4.6 |
| | 3u | 106 | 14.6 |
| | 16a | 77 | |
| | 3k | | 12 |
| | | % inhibition of XA | IC₅₀ |
| Compound | | binding at 10 μM* | (μM) |
| Xanthurenic acid | | 100 | 0.3 and 57 |
| Kynurenic acid | | Potentiation | |
| | | (+70% real increase | |
| | | in total binding) | |
| 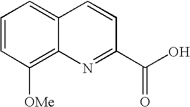 | | 13 | |
| | 3y | 120 | |
| | 28c | 48.5 | |
| | 28b | 0 | |
| | 3n | 21.2 | |
| | 19f | 163.1 | 5.2 |
| | 3e | 48 | |
| | 3c | 42 | |
| | 19a | 1.8 | |
| | 22a | 54 | |
| | 22b | 38 | |
| | 3v | 23.4 | |
| | 28a | 0 | |
| | 3w | 0 | |
| | 17'b | 7.7 | |
| | 4f | 0 | |
| | 3x | Potentiation | |
| | | (+200% real increase | |
| | | in total binding) | |
| | 26a | 0 | |
| | 3g | 84 | |
| | 4g | 0 | |
| | 19t | 50 | |
| | 10b | 94.5 | |
| | 19r | 58.1 | |
| | 22c | Potentiation | |
| | | (+100% real increase | |
| | | in total binding) | |
| | 19g | 54.5 | |
| | XA | 100 | |
| | 87c | 183 | |
| | 3i | 163 | |
| | 91c | 102 | |
| | 94c | 37 | |
| | 95c | 57 | |
| | 96c | 17 | |
| | 97c | 60 | |
| | 55b | 103 | |
| | 52c | 143 | |
| | 56c | 112 | |
| | 124b | 109 | |
| | 77c | 6 | |
| | 73c | 69 | |
| | 67c | 0 | |
| | 63c | 46 | |
| | 64b | 14 | |
| | 61b | 97 | |
| | 66b | 69 | |
| | 98c | 94 | |
| | 99c | 220 | |
| | 100c | 137 | |
| | 101c | 20 | |
| | 102b | 92 | |
| | 103b | 3 | |
| | 45c | 26 | |
| | 117d | 97 | |
| | 33c | 97 | |
| | 121c | 120 | |
| | 104b | 40 | |
| | 105b | 66 | |
| | 106b | 29 | |
| | 107b | 43 | |
| | 108b | 62 | |
| | 65d | 18 | |
| | 62b | 94 | |
| | 69d | 26 | |
| | 59c | 97 | |
| | 60c | 69 | |
| | 36c | 94 | |
| | 37c | 77 | |
| | 79d | 60 | |
| | 32d | 117 | |
| | 109b | 112 | |
| | 110c | 166 | |
| | 111c | 109 | |
| | 123c | 100 | |
| | 118c | 66 | |
| | 71c | 66 | |
| | 75c | 31 | |
| | 122c | 29 | |
| | 34d | 26 | |
| | 38d | 72 | |
| | 39b | 77 | |
| | 41d | 43 | |
| | 40a | 83 | |

These results demonstrate the properties of the inventive compounds to modulate the activity of xanthurenic acid, and thereby to modulate dopaminergic neurotransmission in particular.

The determinations at 50 μM or 10 μM were set arbitrarily to 100% inhibition of binding. Thus, compounds with values ≧100 are as potent as xanthurenic acid. For example, compound 19f is about 10 times more potent than xanthurenic acid (IC₅₀=5.2 and 57 μM, respectively).

Compounds which increase the binding of xanthurenic acid are potential allosteric modulators of the xanthurenic acid receptor. Examples include kynurenic acid and compounds 3x and 22c

Compound 3u is a quite potent inhibitor of xanthurenic acid binding (IC₅₀=14.6 μM). In in vitro (electrophysiology) experiments and in vivo (stereotypic behaviors) studies, this compound blocks the effects of xanthurenic acid without displaying an intrinsic effect. It is thus the first competitive antagonist acting on the xanthurenic acid receptor.

The dichlorinated derivative 3i was used as precursor to prepare the tritiated radioligand, which allowed development of the method to determine specific binding of xanthurenic acid, and represents an important tool in the search for better xanthurenic acid ligands.

The tritiated derivative was prepared by catalytic hydrogenation (palladium/charcoal) of the dichlorinated precursor 3i in the presence of tritium gas under 15 psi of pressure for 24 hours.

1-47. (canceled)

48. A method for treating a nervous system pathology, by administering to a subject in need of such treatment a compound represented by formula (I): 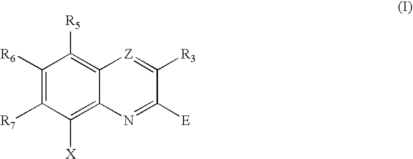
wherein
E is a COOH, COOR₁, CH₂OH, CHO, CH₂COOH, CH₂COOR₁ group or a group chosen from among the following: 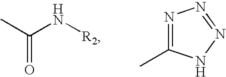
R₁ represents (i) a (C₁-C₁₂) alkyl group or (ii) a (C₆-C₁₈)aryl(C₁-C₁₂)alkyl group;
R₂ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a hydroxyl group;
R₃ is (i) a hydrogen atom, (ii) a halogen atom, (iii) a hydroxyl group, (iv) a (C₁-C₁₂) alkyl group, (v) a (C₆-C₁₈) aryl group, (vi) a (C₆-C₁₈)aryl(C₁-C₁₂)alkyl group or (vii) a (C₃-C₁₇) heteroaryl group;
Z is (i) a nitrogen atom or (ii) a CR₄ group;
R₄ represents (a) a hydrogen atom, (b) a (C₁-C₁₂) alkyl group, (c) a (C₁-C₁₂) alcyn-1-yl group, (d) a (C₆-C₁₈) aryl group, (e) a (C₆-C₁₈)aryl(C₁-C₁₂) alkyl group, (f) a OR₈ group, (g) a NR₉R_{9′} group, (h) a (C₁-C₁₇) heteroaryl group or (i) a (C₂-C₁₂) alcen-1-yl group;
R₅, R₆ and R₇ represent independently of each other (i) a hydrogen atom, (ii) a halogen atom, (iii) a (C₁-C₁₂) alkyl group, (iv) a (C₆-C₁₈) aryl group, (v) a (C₆-C₁₈)aryl(C₁-C₁₂)alkyl group, (vi) a NR₉R_{9′} group, (vii) a COR₁₀ group, (viii) a (C₂-C₁₂) alcen-1-yl group, (ix) a (C₂-C₁₂) alcyn-1-yl group, (x) a (C₁-C₁₇) heteroaryl group, (xi) a (C₃-C₁₇)heteroaryl(C₁-C₁₂)alkyl group, (xii) a cyano group or (xiii) a nitro group;
R₈ represents (i) a hydrogen atom, (ii) a (C₁-C₁₂) alkyl group, (iii) a (C₆-C₁₈)aryl(C₁-C₁₂)alkyl group;
R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) an acyl group, (vi) a tert-butyloxycarbonyl group, (vii) a (C₁-C₁₇) heteroaryl group or (viii) a (C₆-C₁₈) arylsulfonyl or (C₁-C₁₂) alkylsulfonyl group;
R_{9′} which may the same as or different from R₉ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) an acyl group, (vi) a tert-butyloxycarbonyl group, (vii) a (C₁-C₁₇) heteroaryl group or (viii) a (C₆-C₁₈) arylsulfonyl or (C₁-C₁₂) alkylsulfonyl group;
NR₉R_{9′} may also represent a cycloheteroalkyl group of the type: 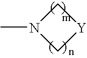
with n=2 or 3,
m=2 or 3 and
Y represents a CH₂, SO₂, or NR₁₁ group or else an oxygen or sulfur atom,
R₁₀ represents (i) a hydrogen atom, (ii) a (C₁-C₁₂) alkyl group or (iii) a (C₆-C₁₈) aryl group or (iv) a NHR₂ group;
R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;
X is (i) a halogen atom, (ii) a OR₈ group (iii) a NR₉R_{9′} group, (iv) a (C₆-C₁₈)aryl group, (v) a (C₆-C₁₈)aryl(C₁-C₁₂)alkyl group, (vi) (C₃-C₁₂) alkyl (vii) a (C₂-C₁₂)alcen-1-yl group, (viii) a (C₂-C₁₂)alcyn-1-yl group, (ix) a (C₁-C₁₇) heteroaryl group, (x) a COR₁₀ group, (xi) a cyano group or (xii) a nitro group,
it being understood that the alcen-1-yl and alcyn-1-yl groups are not substituted or are substituted by one or more substituents, which are the same or different, preferably chosen from among the groups OR₈, aryl or NR₉R_{9′}, the alkyl and alkoxy groups and portions are not substituted or are substituted by one or more substituents, which are the same or different, chosen from among hydroxyl, aryl, OR₈, NR₉R_{9′} groups, the aryl groups and portions are not substituted or are substituted by one or more substituents, which are the same or different, chosen from among the halogen atoms and (C₁-C₁₂) alkyl and (C₁-C₁₂) alkoxy groups, the heteroaryl groups are not substituted or are substituted by halogen atoms or (C₁-C₁₂) alkyl or (C₁-C₁₂) alkoxy groups,
and when they contain one or more asymmetrical carbon atoms, their racemates, enantiomers, diastereomers and when Z is CR₄ and R₄ is OR₈ wherein R₈ is hydrogen, their tautomeric form,
and their pharmaceutically acceptable salts.

49. A method according to claim 48, wherein X represents a halogen atom, preferably the bromine atom.

50. A method according to claim 48, wherein
E is a COOH, COOR₁, CHO, CH₂COOH, CH₂COOR₁ group or a group chosen from among the following: 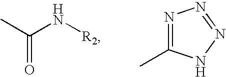
and/or
R₁ represents a (C₁-C₁₂) alkyl or (C₆-C₁₈)aryl(C₁-C₁₂)alkyl group; and/or
R₂ represents (i) a hydrogen, (ii) a (C₁-C₁₂) alkyl group, (iii) a (C₆-C₁₈) aryl group, (iv) a (C₆-C₁₈)aryl(C₁-C₁₂)alkyl group or (v) a hydroxyl group; and/or
R₃ is (i) a hydrogen atom, (ii) a halogen atom, (iii) a hydroxyl group, (iv) a (C₁-C₁₂) alkyl group, (vi) a (C₆-C₁₈) aryl(C₁-C₁₂) alkyl group; and/or
Z is (i) a nitrogen atom or (ii) a CR₄ group; and/or
R₄ represents (a) a hydrogen atom, (b) a (C₁-C₁₂) alkyl group, (c) a (C₂-C₁₂) alcyn-1-yl group, (d) a (C₆-C₁₈) aryl group, (e) a OR₈ group in which R₈ represents hydrogen, (f) a (C₁-C₁₇) heteroaryl group or (g) a NR₉R_{9′} group in which R₉ represents hydrogen, acyl or (C₆-C₁₈) arylsulfonyl and R_{9′} represents hydrogen, acyl or NR₉R_{9′} represents a cycloheteroalkyl group of the type: 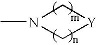
with n=2 or 3,
m=2 or 3 and
Y represents a CH₂, SO₂, or NR₁₁ group or else an oxygen or sulfur atom, and/or
R₅, R₆, R₇ represent independently of each other (i) a hydrogen atom, (ii) a halogen atom, (iii) a (C₁-C₁₂) alkyl group, (iv) a (C₆-C₁₈) aryl group, (v) a NR₉R_{9′} group in which R₉ represents hydrogen, and R_{9′} represents hydrogen, acyl or (C₆-C₁₈) arylsulfonyl or NR₉R_{9′} represents a cycloheteroalkyl group of the type: 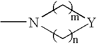
with n=2 or 3,
m=2 or 3 and
Y represents a CH₂, SO₂, or NR₁₁ group or else an oxygen or sulfur atom,
(vi) a COR₁₀ group in which R₁₀ represents hydrogen, (vii) a (C₂-C₁₂) alcen-1-yl group, (viii) a (C₂-C₁₂) alcyn-1-yl group, (ix) a (C₁-C₁₇) heteroaryl group, (x) a (C₆-C₁₈)aryl(C₁-C₁₂)alkyl group, (xi) a cyano group; and/or
X is (i) a halogen atom, (ii) a OR₈ group in which R₈ is a hydrogen atom, a (C₁-C₁₂) alkyl or (C₆-C₁₈)aryl(C₁-C₁₂)alkyl group, (iii) a (C₆-C₁₈)aryl(C₁-C₁₂)alkyl group, (iv) a (C₆-C₁₈) aryl group, (v) a (C₁-C₁₇) heteroaryl group, (vi) a NR₉R_{9′} group in which R₉ is hydrogen, (C₆-C₁₈)aryl(C₁-C₁₂)alkyl or acyl or (C₁-C₁₂) alkylsulfonyl and (C₆-C₁₈) arylsulfonyl and R_{9′} represents hydrogen, acyl or (C₆-C₁₈) arylsulfonyl or NR₉R_{9′} which represents a cycloheteroalkyl group of the type: 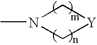
with n=2 or 3,
m=2 or 3 and
Y represents a CH₂, SO₂, or NR₁₁ group or else an oxygen or sulfur atom,
R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;
and their pharmaceutically acceptable salts.

51. A method according to claim 48, wherein
E is a COOH, COOR₁, CH₂OH, CHO, CH₂COOH, CH₂COOR₁ group or a group chosen from among the following: 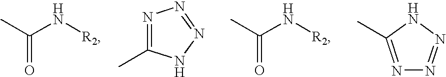
R₁ represents an unsubstituted (C₁-C₁₂) alkyl or benzyl group;
R₂ represents a hydroxyl group;
R₃ is (i) a hydrogen atom, (ii) a halogen atom, (iii) a hydroxyl group, (iv) a (C₁-C₁₂) alkyl group not substituted or substituted by amino or alkylamino, (v) a (C₆-C₁₈) aryl group;
Z is (i) a nitrogen atom or (ii) a CR₄ group in which R₄ represents (a) a hydrogen atom, (b) a (C₁-C₁₂) alkyl group not substituted or substituted by phenyl, hydroxyl or by NR₉R_{9′} in which R₉ is hydrogen or tert-butyloxycarbonyl and R_{9′} is hydrogen or else by NR₉R_{9′} which represents a cycloheteroalkyl group of the type: 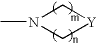
with n=2 or 3,
m=2 or 3 and
Y represents a CH₂, SO₂, or NR₁₁ group or else an oxygen or sulfur atom,
(c) a (C₂-C₁₂) alcyn-1-yl group not substituted or substituted by phenyl, hydroxyl, benzyloxy or by NR₉R_{9′} in which R₉ represents tert-butyloxycarbonyl and R_{9′} is hydrogen, or else by NR₉R_{9′} which represents a cycloheteroalkyl group of the type: 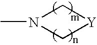
with n=2 or 3,
m=2 or 3 and
Y represents a CH₂, SO₂, or NR₁₁ group or else an oxygen or sulfur atom,
(d) a (C₆-C₁₈) aryl group not substituted or substituted by halogen, (e) a OR₈ group in which R₈ represents hydrogen, (f) a NR₉R_{9′} in which R₉ represents hydrogen or tosyl and R_{9′} is hydrogen; or a NR₉R_{9′} group which represents a cycloheteroalkyl group of the type: 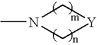
with n=2 or 3,
m=2 or 3 and
Y represents a CH₂, SO₂, or NR₁₁ group or else an oxygen or sulfur atom,
R₅, R₆, R₇ represent independently of each other (i) a hydrogen atom, (ii) a halogen atom, (iii) a (C₁-C₁₂) alkyl group not substituted or substituted by hydroxyl, phenyl or NR₉R_{9′} in which R₉ is hydrogen or tert-butyloxycarbonyl and R_{9′} represent hydrogen, (iv) a phenyl groupo not substituted or substituted by halogen, alkoxy, alkyl, (v) a (C₆-C₁₈)aryl(C₁-C₁₂)alkyl group, (vi) a NR₉R_{9′} group in which R₉ represents hydrogen, and R_{9′} represents hydrogen, (vii) a COR₁₀ group in which R₁₀ represents hydrogen, (viii) an unsubstituted (C₂-C₁₂) alcen-1-yl group, (ix) a (C₂-C₁₂) alcyn-1-yl group not substituted or substituted by phenyl, NR₉R_{9′} in which R₉ is hydrogen or tert-butyloxycarbonyl and R_{9′} represents hydrogen, OR₈ in which R₈ is hydrogen or tert-butoxycarbonyl, (x) a pyridyl group;
X is (i) a halogen atom, (ii) a OR₈ group in which R₈ is hydrogen, (C₁-C₆) alkyl or benzyl, (iii) a NR₉R_{9′} group in which R₉ is hydrogen, acetyl or benzoyl and R_{9′} represents hydrogen, or (iv) phenyl.
and their pharmaceutically acceptable salts.

52. A method according to claim 48, wherein Z is a CR₄ group.

53. A method according to claim 48, wherein Z represents CR₄ in which R₄ is a hydroxyl group, X represents a halogen atom, preferably bromine, and in particular at least one of the groups R₃, R₅, R₆ and R₇ is different from the hydrogen atom, advantageously with R₅ and R₇ not representing a halogen atom.

54. A method according to claim 48, wherein Z represents CR₄ in which R₄ is a hydroxyl group, X represents a hydroxyl group and advantageously at least one of the groups R₃, R₅, R₆ and R₇ is different from the hydrogen atom.

55. A method according to claim 48, wherein R₁ is an unsubstituted alkyl or benzyl group or R₂ is a hydroxyl group and, preferably, R₃ is (i) a hydrogen atom, (ii) a (C₁-C₁₂) alkyl group not substituted or substituted by amino or alkylamino, or (iii) a (C₆-C₁₈) aryl group and/or Z is (i) a nitrogen atom or (ii) a CR₄ group in which R₄ represents (a) a hydrogen atom, (b) a (C₁-C₁₂) alkyl group not substituted or substituted by phenyl or by NR₉R_{9′} in which R₉ is hydrogen or tert-butyloxycarbonyl and R_{9′} represents hydrogen, or else by NR₉R_{9′} which represents a cycloheteroalkyl group of the type: 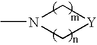
with n=2 or 3
m=2 or 3 and
Y represents a CH₂, SO₂, or NR₁₁ group or else an oxygen or sulfur atom,
(c) a (C₂-C₁₂) alcyn-1-yl group not substituted or substituted by phenyl, hydroxyl, benzyloxy or NR₉R_{9′} in which R₉ represent tert-butyloxycarbonyl and R_{9′} represents hydrogen, or NR₉R_{9′} represents a cycloheteroalkyl group of the type: 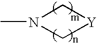
with n=2 or 3,
m=2 or 3 and
Y represents a CH₂, SO₂, or NR₁₁ group or else an oxygen or sulfur atom,
(d) a (C₆-C₁₈) aryl group not substituted or substituted by halogen, (e) a OR₈ group in which R₈ represents hydrogen, (f) a NR₉R_{9′} group in which R₉ represents hydrogen or tosyl and R_{9′} represents hydrogen or NR₉R_{9′} which represents a cycloheteroalkyl group of the type: 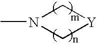
with n=2 or 3,
m=2 or 3 and
Y represents a CH₂, SO₂, or NR₁₁ group or else an oxygen or sulfur atom.
R₁₁ represents (i) a hydrogen atom, (ii) a (C₆-C₁₈) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group.

56. A method according to claim 48, where the compound represented by formula (I) is chosen from among the following compounds:
Methyl-4-hydroxy-8-benzyloxy-quinoline-2-carboxylate
Methyl-4-hydroxy-5-bromo-8-benzyloxy-quinoline-2-carboxylate
Methyl-4,8-dihydroxy-5-bromo-quinoline-2-carboxylate
Methyl-4-hydroxy-5-bromo-8-methoxy-quinoline-2 carboxylate
Methyl-4-hydroxy-5-methyl-8-methoxy-quinoline-2-carboxylate
Methyl-4-hydroxy-5-(1-hydroxy-ethyl)-8-methoxy-quinoline-2-carboxylate
Methyl-4-hydroxy-5-hydroxymethyl-8-benzyloxy-quinoline-2-carboxylate
Methyl-4,8-dihydroxy-5-hydroxymethyl-quinoline-2-carboxylate
Methyl-4-hydroxy-5,7-dichloro-8-methoxy-quinoline-2-carboxylate
Methyl-4-hydroxy-6-iodo-8-methoxy-quinoline-2-carboxylate
Methyl-4-hydroxy-6-bromo-8-methoxy-quinoline-2-carboxylate
Methyl-4-hydroxy-6-bromo-8-benzyloxy-quinoline-2-carboxylate
Methyl-4,8-dihydroxy-6-bromo-quinoline-2-carboxylate
Methyl-4-hydroxy-6-methyl-8-methoxy-quinoline-2-carboxylate
Methyl-4-hydroxy-6-formyl-8-methoxy-quinoline-2-carboxylate
Methyl-4-hydroxy-6-amino-8-methoxy-quinoline-2-carboxylate
Methyl-4-hydroxy-8-amino-quinoline-2-carboxylate
Methyl-4-hydroxy-5-methyl-8-amino-quinoline-2-carboxylate
Methyl-4-hydroxy-6-methyl-8-amino-quinoline-2-carboxylate
Methyl-4-hydroxy-8-bromo-quinoline-2-carboxylate
4,8-dihydroxy-quinoline-2-carboxylic acid
4,8-dihydroxy-5-bromo-quinoline-2-carboxylic acid
4,8-dihydroxy-5-methyl-quinoline-2-carboxylic acid
4,8-dihydroxy-5-hydroxymethyl-quinoline-2-carboxylic acid
4,8-dihydroxy-5,7-dichloro-quinoline-2-carboxylic acid
4,8-dihydroxy-6-iodo-quinoline-2-carboxylic acid
4,8-dihydroxy-6-bromo-quinoline-2-carboxylic acid
4,8-dihydroxy-methyl-quinoline-2-carboxylic acid
4,8-dihydroxy-6-formyl-quinoline-2-carboxylic acid
4-hydroxy-8-amino-quinoline-2-carboxylic acid
4-hydroxy-5-methyl-8-amino-quinoline-2-carboxylic acid
4-hydroxy-6-methyl-8-amino-quinoline-2-carboxylic acid
4-hydroxy-8-bromo-quinoline-2-carboxylic acid
4-hydroxy-8-benzyloxy-quinoline-2-carboxylic acid
8-hydroxy-5-bromo-quinoline-2-carboxylic acid
8-benzyloxy-7-bromo-quinoline-2-carboxylic acid
8-hydroxy-7-bromo-quinoline-2-carboxylic acid
3-methyl-8-methoxy-quinoline-2-carboxylic acid
8-amino-quinoline-2-carboxylic acid
8-methoxy-5-bromo-quinoline-2-carboxylic acid
Benzyl-8-benzyloxy-quinoline-2-carboxylate
Benzyl-8-benzyloxy-5-bromo-quinoline-2-carboxylate
Methyl-8-methoxy-5-bromo-quinoline-2-carboxylate
Benzyl-8-benzyloxy-7-bromo-quinoline-2-carboxylate
Methyl-8-methoxy-3-methyl-quinolin-2-carboxylate
Methyl-8-amino-quinoline-2-carboxylate
Methyl-4-chloro-8-methoxy-quinoline-2-carboxylate
Methyl-4-chloro-8-benzyloxy-quinoline-2-carboxylate
Methyl-8-methoxy-4-methyl-quinoline-2-carboxylate
Methyl-8-methoxy-4-phenyl-quinoline-2-carboxylate
Methyl-8-methoxy-4-(4-chloro-phenyl)-quinoline-2-carboxylate
8-hydroxy-4-methyl-quinoline-2-carboxylic acid
8-hydroxy-4-phenyl-quinoline-2-carboxylic acid
8-hydroxy-4-(4-chloro-phenyl)-quinoline-2-carboxylic acid
Methyl-8-benzyloxy-4-phenylethynyl-quinoline-2-carboxylate
Methyl-8-benzyloxy-4-(3-tert-butoxycarbonylaminoprop-1-ynyl)-quinoline-2-carboxylate
Methyl-8-benzyloxy-4-(3-benzyloxyprop-1-ynyl)-quinoline-2-carboxylate
Methyl-8-hydroxy-4-phenethyl-quinoline-2-carboxylate
Methyl-8-hydroxy-4-(3-tert-butoxycarbonylamino-propyl)-quinoline-2-carboxylate
Methyl-8-hydroxy-4-(3-hydroxy-propyl)-quinoline-2-carboxylate
8-hydroxy-4-phenethyl-quinoline-2-carboxylic acid
8-hydroxy-4-(3-amino-propyl)-quinoline-2-carboxylic acid
8-hydroxy-4-(3-hydroxy-propyl)-quinoline-2-carboxylic acid
Methyl-8-benzyloxy-4-(toluene-4-sulfonylamino)-quinoline-2-carboxylate
Methyl-8-hydroxy-4-(toluene-4-sulfonylamino)-quinoline-2 carboxylate
Methyl-8-amino-4-(toluene-4-sulfonylamino)-quinoline-2-carboxylate
8-hydroxy-4-(toluene-4-sulfonylamino)-quinoline-2-carboxylic acid
Methyl-4-amino-8-hydroxy-quinoline-2-carboxylate
Methyl-4,8-diamino-quinoline-2-carboxylate
4-amino-8-hydroxy-quinoline-2-carboxylic acid
4,8-diamino quinoline-2-caboxylic acid
Methyl-4-hydroxy-8-benzyloxyl-5-phenyl-quinoline-2-carboxylate
Methyl-4,8-dihydroxy-5-phenyl-quinoline-2-carboxylate
Methyl-4-hydroxy-8-methoxy-5-(4-chloro-phenyl)-quinoline-2-carboxylate
Methyl-4-hydroxy-6-phenyl-8-methoxy-quinoline-2-carboxylate
Methyl-4-hydroxy-6-(4-methoxy-phenyl)-8-methoxy-quinoline-2-carboxylate
Methyl-4,8 dihydroxy-6-(4-methoxy-phenyl)-quinoline-2-carboxylate
Methyl-4-hydroxy-6-(3-methyl-phenyl)-8-benzyloxy-quinoline-2-carboxylate
Methyl-4,8-dihydroxy-6-(3-methyl-phenyl)-quinoline-2-carboxylate
Methyl-4-hydroxy-6-(4 chloro-phenyl)-8-benzyloxy-quinoline-2-carboxylate
Methyl-4,8-dihydroxy-6-(4-chloro-phenyl)-quinoline-2-carboxylate
Methyl-8-benzyloxy-6-(3,4-dichloro-phenyl)-4-hydroxy-quinoline-2-carboxylate
Methyl-4,8-dihydroxy-6-(3,4-dichloro-phenyl)-quinoline-2-carboxylate
Methyl-8-benzyloxy-4-hydroxy-6-(pyridin-3-yl)-quinoline-2-carboxylate
Methyl-4,8-dihydroxy-6-(pyridin-3-yl)-quinoline-2-carboxylate
Methyl-4-hydroxy-8-methoxy-6-propenyl-quinoline-2-carboxylate
Methyl-4-hydroxy-8-methoxy-propyl-quinoline-2-carboxylate
Methyl-4-hydroxy-8-phenyl-quinoline-2-carboxylate
Methyl-8-methoxy-5-phenyl-quinoline-2-carboxylate
4,8-dihydroxy-5-phenyl-quinoline-2-carboxylic acid
4,8-dihydroxy-5-(4-chloro-phenyl)-quinoline-2-carboxylic acid
4,8-dihydroxy-6-phenyl-quinoline-2-carboxylic acid
4,8-dihydroxy-6-(4-methoxy-phenyl)-quinoline-2-carboxylic acid
4,8-dihydroxy-6-(3-methyl-phenyl)-quinoline-2-carboxylic acid
4,8-dihydroxy-6-(4-chlorophenyl)-quinoline-2-carboxylic acid
4,8-dihydroxy-6-(3,4-dichlorophenyl)-quinoline-2-carboxylic acid
4,8-dihydroxy-6-(pyridin-3-yl)-quinoline-2-carboxylic acid
4,8-dihydroxy-6-propyl-quinoline-2-carboxylic acid
4-hydroxy-8-phenyl-quinoline-2-carboxylic acid
8-hydroxy-5-phenyl-quinoline-2-carboxylic acid
Methyl-4-hydroxy-8-methoxy-6-phenylethynyl-quinoline-2-carboxylate
Methyl-4-hydroxy-8-methoxy-6-(hept-1-ynyl)-quinoline-2-carboxylate
Methyl-8-benzyloxy-6-(3-tert-butoxycarbonylamino-prop-1-ynyl)-4-hydroxy-quinoline-2-carboxylate
Methyl-8-benzyloxy-6-(3-benzyloxy-prop-1-ynyl)-6-hydroxy-quinoline-2-carboxylate
Methyl-4-hydroxy-8-methoxy-6-phenethyl-quinoline-2-carboxylate
Methyl-4-hydroxy-8-methoxy-6-heptyl-quinoline-2-carboxylate
Methyl-4,8-dihydroxy-6-(3-tert-butoxycarbonylamino-propyl)-quinoline-2-carboxylate
Methyl-4,8-dihydroxy-6-(3-hydroxy-propyl)-quinoline-2-carboxylate
4,8-dihydroxy-6-phenethyl-quinoline carboxylic acid
4,8-dihydroxy-6-heptyl-quinoline-2-carboxylic acid
4,8-dihydroxy-6-(3-amino-propyl)quinoline-2-carboxylic acid
4,8-dihydroxy-6-(3-hydroxy-propyl)-quinoline-2-carboxylic acid
Methyl-8-benzyloxy-6-benzyl-4-hydroxy-quinoline-2-carboxylate
Methyl-4,8-dihydroxy-6-benzyl-quinoline-2-carboxylate
4,8-dihydroxy-6-benzyl-quinoline-2-carboxylic acid
Methyl-6-(benzylamino-methyl)-4-hydroxy-8-methoxy-quinoline-2-carboxylate
4,8-dihydroxy-6-(benzylamino-methyl)-quinoline-2-carboxylic acid
8-acetylamino-4-hydroxy-quinoline-2-carboxylic acid
8-pivaloylamino-4-hydroxy-quinoline-2-carboxylic acid
8-benzylamino-4-hydroxy-quinoline-2-carboxylic acid
Methyl-8-benzylamino-4-hydroxy-quinoline-2-carboxylate
8-benzylamino-4-hydroxy-quinoline-2-carboxylic acid
8-benzyloxy-4-hydroxy-quinoline-2-carboxylic hydroxyamide acid
4,8 dihydroxy-quinoline-2-carboxylic hydroxyamide acid
8-methoxy-2-(2H-tetrazol-1-yl)-quinoline
8-hydroxy-2-(2H-tetrazol-1-yl)-quinoline
8-benzyloxy-quinoxazoline-2-carbaldehyde
8-hydroxy-quinoxazoline-2-carbaldehyde
(8-benzyloxy-quinoxazolin-2-yl)-methanol
(8-hydroxy-quinoxazolin-2-yl)-methanol
Methyl-8-methoxy-3-methylaminomethyl-quinoline-2-carboxylate
8-hydroxy-3-methylaminomethyl-quinoline-2-caboxylic acid
Methyl-3-benzyl-8-methoxy-quinoline-2-carboxylate
3-benzyl-8-methoxy-quinoline-2-carboxylic acid
Methyl-4-hydroxy-8-bromo-quinoline-2-carboxylate
Methyl-8-benzyloxy-5,7-dichloro-4-hydroxyquinoline-2-carboxylate
Methyl-8-benzyloxy-7-bromo-4-hydroxyquinoline-2-carboxylate
Methyl-6-bromo-8-cyano-4-hydroxy-quinoline-2-carboxylate
Ethyl-8-hydroxy-3-oxo-3,4-dihydroquinoxaline-2-carboxylate
Ethyl[8-(hydroxy)-3-oxo-3,4-dihydroquinoxaline-2(1H)-ylilene]acetate
Methyl-5,7-dichloro-4,8-dihydroxyquinoline-2-carboxylate
Methyl-3-bromo-4,8-dihydroxy-quinoline-2-carboxylate
Methyl-3,7-dibromo-4,8-dihydroxy-quinoline-2-carboxylate
8-bromo-4-hydroxy-6-isopropyl-quinoline-2-carboxylic acid
8-benzyloxy-6-bromo-4-hydroxy-quinoline-2-carboxylic acid
8-Benzyloxy-7-bromo-4-hydroxyquinoline-2-carboxylic acid
8-benzyloxy-3-bromo-4-hydroxy-quinoline-2-carboxylic acid
[8-(hydroxy)-3-oxo-3,4-dihydroquinoxaline-2(1H)-yl]acetic acid
6-bromo-8-cyano-4-hydroxy-quinoline-2-carboxylic acid
8-cyano-4-hydroxy-6-phenyl-quinoline-2-carboxylic acid
8-cyano-4-hydroxy-6-phenenyl-quinoline-2-carboxylic acid
3-bromo-8-cyano-4-hydroxy-quinoline-2-carboxylic acid
8-cyano-4-hydroxy-3-phenylethynyl-quinoline-2-carboxylic acid
8-cyano-6-ethyl-4-hydroxy-quinoline-2-carboxylic acid
Benzyl-4-benzyloxy-8-bromo-quinoline-2-carboxylate
Benzyl-4,8-dibenzyloxy-6-bromo-quinoline-2-carboxylate
Benzyl-4,8-dibenzyloxy-7-bromo-quinoline-2-carboxylate
Benzyl-3-bromo-4,8-dibenzyloxy-quinoline-2-carboxylate
Methyl-6-bromo-8-cyano-4-benzyloxy-quinoline-2-carboxylate
Benzyl-3-bromo-4-benzyloxy-8-cyano-quinoline-2-carboxylate
Methyl-8-benzyloxy-6-(furo-2-yl)-4-hydroxy-quinoline-2-carboxylate
Benzyl-4,8-dibenzyloxy-6-(2-chlorophenyl)-quinoline-2-carboxylate
Benzyl-4,8-dibenzyloxy-6-(3-chlorophenyl)-quinoline-2-carboxylate
Benzyl-4,8-dibenzyloxy-7-phenyl-quinoline-2-carboxylate
Benzyl-4,8-dibenzyloxy-6-(2-methoxyphenyl)-quinoline-2-carboxylate
Benzyl-4,8-dibenzyloxy-3-phenyl-quinoline-2-carboxylate
Methyl-8-cyano-4-hydroxy-6-phenyl-quinoline-2-carboxylate
Benzyl-4-benzyloxy-8-(hex-1-ynyl)-quinoline-2-carboxylate
Benzyl-4,8-dibenzyloxy-6-(3-benzyloxy-prop-1-ynyl)-quinoline-2-carboxylate
Benzyl-4,8-dibenzyloxy-7-(3-benzyloxy-prop-1-ynyl)-quinoline-2-carboxylate
Benzyl-4,8-dibenzyloxy-7-phenylethynyl-quinoline-2-carboxylate
Methyl-4-benzyloxy-8-cyano-6-phenylethynyl)-quinoline-2-carboxylate
Benzyl-4-benzyloxy-8-cyano-3-phenylethynyl-quinoline-2-carboxylate
Methyl-8-Cyano-4-hydroxy-6-[(trimethylsilyl)ethynyl]quinoline-2-carboxylate
8-hexyl-4-hydroxy-quinoline-2-carboxylic acid
4,8-dihydroxy-6-(3-hydroxy-propyl)-quinoline-2-carboxylic acid
4,8-dihydroxy-7-(3-hydroxy-propyl)-quinoline-2-carboxylic acid
Methyl-8-cyano-4-hydroxy-6-phenethyl-quinoline-2-carboxylate
Methyl-8-cyano-6-ethyl-4-hydroxy-quinoline-2-carboxylate
4,8-dihydroxy-6-(furo-2-yl)-quinoline-2-carboxylic acid
4-hydroxy-8-phenyl-quinoline-2-carboxylic acid
4,8-dihydroxy-6-(2-chlorophenyl)-quinoline-2-carboxylic acid
4,8-dihydroxy-6-(3-chlorophenyl)-quinoline-2-carboxylic acid
4,8-dihydroxy-7-phenyl-quinoline-2-carboxylic acid
4,8-dihydroxy-6-(2-methoxyphenyl)-quinoline-2-carboxylic acid
4,8-dihydroxy-3-phenyl-quinoline-2-carboxylic acid
4,8-dihydroxy-4-piperidin-2-yl-quinoline-2-carboxylic acid hydrochloride
Sodium 8-hexyl-4-hydroxy-quinoline-2-carboxylate
Sodium 4,8-dihydroxy-7-phenyl-quinoline-2-carboxylate
Sodium 4,8-dihydroxy-3-phenyl-quinoline-2-carboxylate
Methyl-8-benzyloxy-3-bromo-4-hydroxy-quinoline-2-carboxylate
Methyl-8-benzyloxy-3,7-dibromo-4-hydroxyquinoline-2-carboxylate
Methyl-3-bromo-8-cyano-4-hydroxyquinoline-2-carboxylate
Methyl-4-hydroxy-8-(2H-tetrazol-5-yl)-quinoline-2-carboxylate
Ethyl-8-(benzyloxy)-3-oxo-3,4-dihydroquinoxaline-2-carboxylate
Ethyl[8-(benzyloxy)-3-oxo-3,4-dihydroquinoxaline-2(1H)-ylilene]acetate
Benzyl-4,8-benzyloxy-6-piperidin-2yl-quinoline-2-carboxylate
Methyl-8-cyano-6-ethynyl-4-hydroxy-quinoline-2-carboxylate
Methyl-5-bromo-8-nitro-4-hydroxy-quinoline-2-carboxylate
Methyl-6-benzyloxy-8-nitro-4-hydroxy-quinoline-2-carboxylate
Methyl-6-chloro-8-nitro-4-hydroxy-quinoline-2-carboxylate
Methyl-6-bromo-8-nitro-4-hydroxy-quinoline-2-carboxylate
8-cyano-4-hydroxy-quinoline-2-carboxylate
Methyl-8-fluoro-4-hydroxy-quinoline-2-carboxylate
Methyl-8-carboxamide-4-hydroxy-quinoline-2-carboxylate
Methyl-3-phenylethyl-8-amino-4-hydroxy-quinoline-2-carboxylate
3-(3′-N-tert-butoxycarbonyl-propyl)-8-amino-4-hydroxy-quinoline-2-carboxylic acid
3-(3-hydroxypropyl)-8-amino-4-hydroxy-quinoline-2-carboxylic acid
3-ethyl-8-amino-4-hydroxy-quinoline-2-carboxylic acid
Methyl-5-phenyl-8-amino-4-hydroxy-quinoline-2-carboxylate
Methyl-5-phenylethyl-8-amino-4-hydroxy-quinoline-2-carboxylate
Methyl-5-(3′-N-(terbutoxycarbonyl)aminopropyl)-8-amino-4-hydroxy-quinoline-2-carboxylate
Methyl-5-hydroxypropyl-8-amino-4-hydroxy quinoline-2-carboxylate
Methyl-5-(N-piperidinyl)-8-amino-4-hydroxy-quinoline-2-carboxylate
Methyl-5-piperazinyl-8-amino-4-hydroxy-quinoline-2-carboxylate
Methyl-6-hydroxy-8-amino-4-hydroxy-quinoline-2-carboxylate
Methyl-6-phenylethyl-8-amino-4-hydroxy-quinoline-2-carboxylate
Methyl-6-(3′-hydroxypropyl)-8-amino-4-hydroxy-quinoline-2-carboxylate
Methyl-6-(3′-N-(tert-butoxycarbonyl)aminopropyl)-8-amino-4-hydroxy-quinoline-2-carboxylate
Methyl-6-(3′-pyridinyl)ethyl-8-amino-4-hydroxy-quinoline-2-carboxylate
Methyl-6-(5′-cyanopentyl)-8-amino-4-hydro-quinoline-2-carboxylate
Methyl-6-cyano-8-amino-4-hydroxy-quinoline-2-carboxylate
6-N-(N-methylpiperazinyl)-8-amino-4-hydroxy-quinoline-2-carboxylic acid hydrochloride
Methyl-6-N-piperidinyl-8-amino-4-hydroxy-quinoline-2-carboxylate
Methyl-6-piperazinyl-8-amino-4-hydroxy-quinoline-2-carboxylate
7-phenyl-8-amino-4-hydroxy-quinoline-2-carboxylic acid
4-(N-methylamino)-8-amino-quinoline-2-carboxylic acid hydrochloride
Methyl-8-dimethylamino-4-hydroxy-quinoline-2-carboxylate
3-(N-morpholinomethyl)-4,8-dihydroxy-quinoline-2-carboxylic acid
3-(N-pyrolidinomethyl)-4,8-dihydroxy-quinoline-2-carboxylic acid
Methyl-4-(N-methylamino)-8-amino-quinoline-2-carboxylate
6-hydroxy-8-amino-4-hydroxy-quinoline-2-carboxylic acid
6-chloro-8-amino-4-hydroxy-quinoline-2-carboxylic acid
6-phenylethyl-8-amino-4-hydroxy-quinoline-2-carboxylic acid
6-(3′-hydroxypropyl)-8-amino-4-hydroxy-quinoline-2-carboxylic acid
6-(3′-aminopropyl)-8-amino-4-hydroxy-quinoline-2-carboxylic acid
8-fluoro-4-hydroxy-quinoline-2-carboxylic acid
8-carboxamide-4-hydroxy-quinoline-2-caboxylic acid
3-bromo-8-amino-4-hydroxy-quinoline-2-carboxylic acid
8-cyano-4-hydroxy-quinoline-2-carboxylic acid
5-phenyl-8-amino-4-hydroxy-quinoline-2-carboxylic acid
6-(5′-cyanopentyl)-8-amino-4-hydroxy-quinoline-2-carboxylic acid
6-cyano-8-amino-4-hydroxy-quinoline-2-carboxylic acid
6-N-(N-methylpiperazinyl)-8-nitro-4-benzyloxy-quinoline-2-carboxylic acid hydrochloride
8-dimethylamino-4-hydroxy-quinoline-2-carboxylic acid
Methyl-5-bromo-8-nitro-4-benzyloxy-quinoline-2-carboxylate
Methyl-6-bromo-8-nitro-4-benzyloxy-quinoline-2-carboxylate
Methyl-7-bromo-4,8-dibenzyloxy-quinoline-2-carboxylate
Methyl-4-(N-methyl-toluene-4-sulfonamino)-8-nitro quinoline-2-carboxylate
Methyl-8-dimethylamino-4-benzyloxy-quinoline-2-carboxylate
Benzyl-7-phenyl-8-amino-4-benzyloxy-quinoline-2-carboxylate
Methyl-5-phenyl-8-nitro-4-benzyloxy-quinoline-2-carboxylate
Methyl-3-trimethylsilylethynyl-8-nitro-4-hydroxy-quinoline-2-carboxylate
Methyl-3-phenylethynyl-8-nitro-4-hydroxy-quinoline-2-carboxylate
Benzyl-3-(3′-benzyloxypropyn-1′-yl)-8-nitro-4-hydroxy-quinoline-2-carboxylate
Benzyl-3-(3′-N-terbutoxycarbonyl)aminoprop-1′-ynyl)-8-nitro-4-hydroxy-quinoline-2-carboxylate
Methyl-5-phenylethynyl-8-nitro-4-benzyloxy-quinoline-2-carboxylate
Methyl-5-(3′-benzyloxypropyn-1′-yl)-8-nitro-4-benzyloxy-quinoline-2-carboxylate
Methyl-5-(3′-N-(terbutoxycarbonyl)aminoprop-1′-ynyl)-8-nitro-4-benzyloxy-quinoline-2-carboxylate
Methyl-6-phenylethynyl-8-nitro-4-benzyloxy-quinoline-2-carboxylate
Methyl-6-(3′-pyridyl)ethynyl-8-nitro-4-benzyloxy-quinoline-2-carboxylate
Methyl-6-(5′-cyanopent-1′-ynyl)-8-nitro-4-benzyloxy-quinoline-2-carboxylate
Methyl-6-(3′-benzyloxypropyn-1′-yl)-8-nitro-4-benzyloxy-quinoline-2-carboxylate
Methyl-6-(3′-N-(terbutoxycarbonyl)aminoprop-1′-ynyl)-8-nitro-4-benzyloxy-quinoline-2-carboxylate
Methyl-4-methylamino-8-nitro-quinoline-2-carboxylate
Sodium 7-bromo-4,8-dihydroxy-quinoline-2-carboxylate
Methyl-6-(N-(N-methyl-piperazinyl))-8-nitro-4-benzyloxy-quinoline-2-carboxylate
Methyl-6-(N-(N-benzyl-piperazinyl))-8-nitro-4-benzyloxy-quinoline-2-carboxylate
Methyl-6-(N-piperidinyl)-8-nitro-4-benzyloxy-quinoline-2-carboxylate
Methyl-6-(N-diphenylimine)-8-nitro-4-benzyloxy-quinoline-2-carboxylate
Methyl-6-(N-anilino)-8-nitro-4-benzyloxy-quinoline-2-carboxylate
Methyl-5-(N-piperidinyl)-8-nitro-4-benzyloxy-quinoline-2-carboxylate
Methyl-5-(N-(N-benzyl-piperazinyl))-8-nitro-4-benzyloxy-quinoline-2-carboxylate
Methyl-3-bromo-8-amino-4-hydroxy-quinoline-2-carboxylate
Methyl-6-bromo-8-amino-4-hydroxy-quinoline-2-carboxylate
Methyl-6-chloro-8-amino-4-hydroxy-quinoline-2-carboxylate
Benzyl-8-amino-4-benzyloxy-quinoline-2-carboxylate
Benzyl-7-iodo-8-amino-4-benzyloxy-quinoline-2-carboxylate
Methyl-6-cyano-8-nitro-4-hydroxy-quinoline-2-carboxylate
Methyl-3-bromo-4-hydroxy-8-nitro-quinoline-2-carboxylate
Methyl-6-amino-8-nitro-4-benzyloxy-quinoline-2-carboxylate
3-ethynyl-8-nitro-4-hydroxy-quinoline-2-carboxylic acid
Benzyl-8-nitro-4-benzyloxy-quinoline-2-carboxylate
3-(N-morpholinomethyl)-8-benzyloxy-4-hydroxy-quinoline-2-carboxylic acid
3-(N-pyrolidinomethyl)-8-benzyloxy-4-hydroxy-quinoline-2-carboxylic acid
Methyl-5-piperazinyl-8-amino-4-hydroxy-quinoline-2-carboxylate
3-phenylethyl-8-amino-4-hydroxy-quinoline-2-carboxylic acid
3-(3′-aminopropyl)-8-amino-4-hydroxy-quinoline-2-carboxylic acid hydrochloride
5-phenylethyl-8-amino-4-hydroxy-quinoline-2-carboxylic acid
Methyl-5-(3′-aminopropyl)-8-amino-4-hydroxy-quinoline-2-carboxylate hydrochloride
5-hydroxypropyl-8-amino-4-hydroxy quinoline-2-carboxylic acid
5-(N-piperidinyl)-8-amino-4-hydroxy-quinoline-2-carboxylic acid hydrochloride
5-piperazinyl-8-amino-4-hydroxy-quinoline-2-carboxylic acid hydrochloride
6-(3′-pyridinyl)ethyl-8-amino-4-hydroxy-quinoline-2-carboxylic acid hydrochloride
6-N-piperidinyl-8-amino-4-hydroxy-quinoline-2-carboxylic acid hydrochloride
6-piperazinyl-8-amino-4-hydroxy-quinoline-2-carboxylic acid hydrochloride
6-anilino-8-amino-4-hydroxy-quinoline-2-carboxylic acid hydrochloride
6,8-diamino-4-hydroxy-quinoline-2-carboxylic acid hydrochloride
Methyl-6-anilino-8-amino-4-hydroxy-quinoline-2-carboxylate
Methyl-6,8-diamino-4-hydroxy-quinoline-2-carboxylate
Methyl-4-hydroxy-8-nitro-6-phenyl-quinoline-2-carboxylate
Methyl-8-amino-4-hydroxy-6-phenyl-quinoline-2-carboxylate
Methyl-8-hydroxy-4-(piperazin-1-yl)-quinoline-2-carboxylate
Methyl-8-amino-4-phenyl-quinoline-2-carboxylate
Methyl-8-amino-4-(hex-1-yl)-quinoline-2-carboxylate
Methyl-8-amino-4-(2-phenyleth-1-yl)-quinoline-2-carboxylate
Methyl-8-amino-4-(3-tert-butoxycarbonylamino-prop-1-yl)-quinoline-2-carboxylate
Methyl-8-amino-4-(3-hydroxy-prop-1-yl)-quinoline-2-carboxylate
Methyl-4-(3-acetyl-aminoprop-1-ynyl)-8-amino-quinoline-2-carboxylate
Methyl-8-hydroxy-4-(morpholin-1-yl)-quinoline-2-carboxylate
Methyl-8-benzyloxy-4-(piperidin-1-yl)-quinoline-2-carboxylate
Methyl-8-amino-4-(piperidin-1-yl)-quinoline-2 carboxylate
Methyl-4-hydroxy-8-(piperazin-1-yl)-quinoline-2-carboxylate
Methyl-8-hydroxy-4-(4-methyl-piperazin-1-yl)-quinoline-2-carboxylate
Methyl-7-(acetylamino)-4-hydroxy-quinoline-2-carboxylate
Methyl-4-(3-benzoyl-aminoprop-1-yl)-8-hydroxyquinoline-2-carboxylate
5-(4-chlorophenyl)-8-hydroxy-quinoline-2-carboxylic acid
8-amino-4-(hex-1-yl)-quinoline-2-carboxylic acid hydrochloride
8-amino-4-(2-phenyleth-1-yl)-quinoline-2-carboxylic acid hydrochloride
8-amino-4-(3-tert-butoxycarbonylaminoprop-1-yl)-quinoline-2-carboxylic acid hydrochloride
8-amino-4-(3-hydroxy-prop-1-yl)-quinoline-2-carboxylic acid hydrochloride
4-(3-acetylaminoprop-1-ynyl)₈-amino-quinoline-2-carboxylic acid hydrochloride
8-hydroxy-4-(morpholin-1-yl)-quinoline-2-carboxylic acid hydrochloride
8-hydroxy-4-(piperidin-1-yl)-quinoline-2-carboxylic acid hydrochloride
8-amino-(piperidin-1-yl)-quinoline-2-carboxylic acid hydrochloride
4-hydroxy-(piperazin-1-yl)-quinoline-2-carboxylic acid hydrochloride
8-hydroxy-4-(4-methyl-piperazin 1-yl)-quinoline-2-carboxylic acid hydrochloride
4-hydroxy-8-phenylethyl-quinoline-2-carboxylic acid
4-(3-(benzoylamino)prop-1-yl)-8-hydroxyquinoline-2-carboxylic acid
8-amino-4-hydroxy-6-phenyl-quinoline-2-carboxylic acid
Methyl-8-nitro-4-oxytrimethanelsulfonyl-quinoline-2-carboxylate
Methyl-5-(4-chlorophenyl)-8-methoxy-quinoline-2-carboxylate
Benzyl-4,8-dibenzyloxy-6-(3,5-chlorophenyl)-quinoline-2-carboxylate
Benzyl-4,8-dibenzyloxy-6-(4-fluorophenyl)-quinoline-2-carboxylate
Methyl-8-nitro-4-phenyl-quinoline-2-carboxylate
Benzyl-8-benzyloxy-5-phenylethynyl-quinoline-2-carboxylate
Methyl-8-benzyloxy-4-(hex-1-ynyl)-quinoline-2 carboxylate
Methyl-8-benzyloxy-4-(5-benzyloxy-pent-1-ynyl)-quinoline-2-carboxylate
Methyl-8-benzyloxy-7-(3-tert-butoxycarbonylaminoprop-1-ynyl)-hydroxy-quinoline-2-carboxylate
Benzyl-4,8-dibenzyloxy-7-(hex-1-ynyl)-quinoline-2-carboxylate
Methyl-8-amino-4-(hex-1-ynyl)-quinoline-2-carboxylate
Methyl-8-amino-4-phenylethynyl-quinoline-2-carboxylate
Methyl-8-nitro-4-(3-tert-butoxycarbonylamino-prop-1-ynyl)-quinoline-2-carboxylate
Methyl-4-(3-benzyloxy-prop-1-ynyl)-8-nitro-quinoline-2-carboxylate
Methyl-4-(3-acetyl-aminoprop-1-ynyl)-8-nitro-quinoline-2-carboxylate
Methyl-4-benzyloxy-8-phenylethynyl-quinoline-2-carboxylate
8-hydroxy-5-phenylethyl-quinoline-2-carboxylic acid
Methyl-8-benzyloxy-4-(hex-1-yl)-quinoline-2-carboxylate
Methyl-8-benzyloxy-4-(5-hydroxy-pent-1-yl)-quinoline-2-carboxylate
Methyl-8-benzyloxy-4-(3-tert-butoxycarbonylaminoprop-1-yl)-quinoline-2-carboxylate
Methyl-4,8-dihydroxy-7-(3-tert-butoxycarbonylaminoprop-1-yl)-quinoline-2-carboxylate
4,8-dihydroxy-7-(hex-1-yl)-quinoline-2-carboxylic acid
Methyl-4-hydroxy-8-phenylethyl-quinoline-2-carboxylate
8-hydroxy-5-phenylethyl-quinoline-2-carboxylic acid
Methyl-8-benzyloxy-4-(hex-1-yl)-quinoline-2-carboxylate
Methyl-8-benzyloxy-4-(5-hydroxy-pent-1-yl)-quinoline-2-carboxylate
Methyl-8-benzyloxy-4-(3-tert-butoxycarbonylaminoprop-1-yl)-quinoline-2-carboxylate
Methyl-4,8-dihydroxy-7-(3-tert-butoxycarbonylaminoprop-1-yl)-quinoline-2-carboxylate
4,8-dihydroxy-7-(hex-1-yl)-quinoline-2-carboxylic acid
Methyl-4-hydroxy-8-phenylethyl-quinoline-2-carboxylate
Sodium 4-(hex-1-yl)-8-hydroxy-quinoline-2-carboxylate de sodium
8-amino-4-hydroxy-6-phenyl-quinoline-2-carboxylic acid
Methyl-8-benzyloxy-4-(4-benzyl-piperazin-1-yl)-quinoline-2-carboxylate 87a
Methyl-8-benzyloxy-4-(morpholin-1-yl)-quinoline-2-carboxylate
Methyl-8-benzyloxy-4-(piperidin-1-yl)-quinolin-2-carboxylate
Methyl-8-nitro-4-(piperidin-1-yl)-quinoline-2-carboxylate
Benzyl-4-benzyloxy-8-(piperidin-1-yl)-quinoline-2-carboxylate
Benzyl-4-benzyloxy-8-[benzyl(methyl)amino]-quinoline-2-carboxylate
Methyl-4-benzyloxy-8-(morpholin-1-yl)-quinoline-2-carboxylate
Methyl-4-benzyloxy-8-(4-benzyl-piperazin-1-yl)-quinoline-2-carboxylate
Methyl-4-benzyloxy-8-[phenyl(methyl)amino]-quinoline-2-carboxylate
Methyl-4-benzyloxy-8-(4-methyl-piperazin-1-yl)-quinoline-2-carboxylate
Methyl-4-benzyloxy-8-(pyridin-2-ylamino)-quinoline-2-carboxylate
Methyl-8-benzyloxy-4-(4-methyl-piperazin-1-yl)-quinoline-2-carboxylate
Methyl-8-benzyloxy-4-(3-aminoprop-1-yl)-quinoline-2-carboxylate
Methyl-8-benzyloxy-4-(3-benzoyl-aminoprop-1-yl)-quinoline-2-carboxylate
4-(hex-1-yl)-8-hydroxy-quinoline-2-carboxylic acid
8-hydroxy-4-(5-hydroxy-pent-1-yl)-quinoline-2-carboxylic acid
6-(3,5-dichlorophenyl)-4,8-dihydroxy-quinoline-2-carboxylic acid
6-(4-fluorophenyl)-4,8-dihydroxy-quinoline-2-carboxylic acid
4-hydroxy-8-(piperidin-1-yl)-quinoline-2-carboxylic acid hydrochloride
8-(4-benzyl-piperazin-1-yl)-4-hydroxy-quinoline-2-carboxylic acid hydrochloride
8-[phenyl(methyl)amino]-4-hydroxy-quinoline-2-carboxylic acid hydrochloride
8-(4-methyl-piperazin-1-yl)-4-hydroxy-quinoline-2-carboxylic acid hydrochloride
and their pharmaceutically acceptable salts.

57. A method according to claim 48, for treating anxiety, depression, bipolar depression, ADH syndrome, fibromyalgia, impairment of memory or social interactions, as sedative or hypnotic, disorder of sleep or concentration for treating neurodegenerative diseases, such as Parkinson's disease, Alzheimer's disease or ALS, schizophrenia, epilepsy, some drug dependencies, particularly opioid, pain or obesity.

58. A method according to claim 48, for treating mental or neurological disorders, particularly anxiety, depression, impairment of memory or social interactions, as sedative or hypnotic, or for treating neurodegenerative diseases, such as Parkinson's disease, Alzheimer's disease or ALS, some drug dependencies, particularly opioid.

59. A pharmaceutical composition comprising, as active principle, at least one compound such as defined in claim 48.

60. A compound having general formula (I) such as defined in claim 48, where R₁ is an unsubstituted alkyl or benzyl group or R₂ is a hydroxyl group and preferably R₃ is (i) a hydrogen atom, (ii) a halogen atom, (iii) a hydroxyl group, (iv) a (C₁-C₁₂) alkyl group not substituted or substituted by amino, alkylamino, or (v) a (C₆-C₁₈) aryl group and/or Z is (i) a nitrogen atom or (ii) a CR₄ group in which R₄ represents (a) a hydrogen atom, (b) a (C₁-C₁₂) alkyl group not substituted or substituted by phenyl, hydroxyl or NR₉R_{9′} in which R₉ is hydrogen or tert-butyloxycarbonyl and R_{9′} is hydrogen, (c) a (C₁-C₁₂) alcyn-1-yl group not substituted or substituted by phenyl, hydroxyl, benzyloxy or NR₉R_{9′} in which R₉ represents tert-butyloxycarbonyl and R_{9′} is hydrogen, (d) a (C₆-C₁₈) aryl group not substituted or substituted by halogen, (e) a OR₈ group in which R₈ represents hydrogen, (f) a NR₉R_{9′} group in which R₉ represents hydrogen or tosyl and R_{9′} is hydrogen.

61. A compound chosen from among the following compounds:
Methyl-4-hydroxy-8-benzyloxy-quinoline-2-carboxylate
Methyl-4-hydroxy-5-bromo-8-benzyloxy-quinoline-2-carboxylate
Methyl-4,8-dihydroxy-5-bromo-quinoline-2-carboxylate
Methyl-4-hydroxy-5-bromo-8-methoxy-quinoline-2-carboxylate
Methyl-4-hydroxy-5-methyl-8-methoxy-quinoline-2-carboxylate
Methyl-4-hydroxy-5-(1-hydroxy-ethyl)-8-methoxy-quinoline-2-carboxylate
Methyl-4-hydroxy-5-hydroxymethyl-8-benzyloxy-quinoline-2-carboxylate
Methyl-4,8-dihydroxy-5-hydroxymethyl-quinoline-2-carboxylate
Methyl-4-hydroxy-5,7-dichloro-8-methoxy-quinoline-2-carboxylate
Methyl-hydroxy-6-iodo-8-methoxy-quinoline-2-carboxylate
Methyl-4-hydroxy-6-bromo-8-methoxy-quinoline-2-carboxylate
Methyl-4-hydroxy-6-bromo-8-benzyloxy-quinoline-2-carboxylate
Methyl-4,8-dihydroxy-6-bromo-quinoline-2-carboxylate
Methyl-4-hydroxy-6-methyl-8-methoxy-quinoline-2-carboxylate
Methyl-4-hydroxy-6-formyl-8-methoxy-quinoline-2-carboxylate
Methyl-4-hydroxy-6-amino-8-methoxy-quinoline-2-carboxylate
Methyl-4-hydroxy-8-amino-quinoline-2-carboxylate
Methyl-4-hydroxy-5-methyl-8-amino-quinoline-2-carboxylate
Methyl-4-hydroxy-6-methyl-8-amino-quinoline-2-carboxylate
4,8-dihydroxy-5-bromo-quinoline-2-carboxylic acid
4,8-dihydroxy-5-methyl-quinoline-2-carboxylic acid
4,8-dihydroxy-5-hydroxymethyl-quinoline-2-carboxylic acid
4,8-dihydroxy-6-iodo-quinoline-2-carboxylic acid
4,8-dihydroxy-6-bromo-quinoline-2-carboxylic acid
4,8-dihydroxy-6-methyl-quinoline-2-carboxylic acid
4,8-dihydroxy-6-formyl-quinoline-2-carboxylic acid
4-hydroxy-8-amino-quinoline-2-carboxylic acid
4-hydroxy-5-methyl-8-amino-quinoline-2-caboxylic acid
4-hydroxy-6-methyl-8-amino-quinoline-2-carboxylic acid
4-hydroxy-8-benzyloxy-quinoline-2-carboxylic acid
8-hydroxy-5-bromo-quinoline-2-carboxylic acid
8-hydroxy-7-bromo-quinoline-2-carboxylic acid
8-hydroxy-7-bromo-quinoline-2-carboxylic acid
3-methyl-8-methoxy-quinoline-2-carboxylic acid
8-amino-quinoline-2-carboxylic acid
8-methoxy-5-bromo-quinoline-2-carboxylic acid
Benzyl-8-benzyloxy-quinoline-2-carboxylate
Benzyl-8-benzyloxy-5-bromo-quinoline-2-carboxylate
Methyl-8-methoxy-5-bromo-quinoline-2-carboxylate
Benzyl-8-benzyloxy-7-bromo-quinoline-2-carboxylate
Methyl-8-methoxy-3-methyl-quinoline-2-carboxylate
Methyl-8-amino-quinoline-2-carboxylate
Methyl-4-chloro-8-methoxy-quinoline-2-carboxylate
Methyl-4-chloro-8-benzyloxy-quinoline-2-carboxylate
Methyl-8-methoxy-4-methyl-quinoline-2-carboxylate
Methyl-8-methoxy-4-phenyl-quinoline-2-carboxylate
Methyl-8-methoxy-4-(4-chloro-phenyl)-quinoline-2 carboxylate
8-hydroxy-4-methyl-quinoline-2-carboxylic acid
8-hydroxy-4-phenyl-quinoline-2-carboxylic acid
8-hydroxy-4-(4-chloro-phenyl)-quinoline-2-carboxylic acid
Methyl-8-benzyloxy-4-phenylethynyl-quinoline-2-carboxylate
Methyl-8-benzyloxy-4-(3-tert-butoxycarbonylaminoprop-1-ynyl)-quinoline-2-carboxylate
Methyl-8-benzyloxy-4-(3-benzyloxyprop-1-ynyl)-quinoline-2-carboxylate
Methyl-8-hydroxy-4-phenethyl-quinoline-2-carboxylate
Methyl-8-hydroxy-4-(3-tert-butoxycarbonylamino-propyl)-quinoline-2-carboxylate
Methyl-8-hydroxy-4-(3-hydroxy-propyl)-quinoline-2-carboxylate
8-hydroxy-4-phenethyl-quinoline-2-carboxylic acid
8-hydroxy-4-(3-amino-propyl)-quinoline-2-carboxylic acid
8-hydroxy-4-(3-hydroxy-propyl)-quinoline-2-carboxylic acid
Methyl-8-benzyloxy-4-(toluene-4-sulfonylamino)-quinoline-2-carboxylate
Methyl-8-hydroxy-4-(toluene-4-sulfonylamino)-quinoline-2-carboxylate
Methyl-8-amino-4-(toluene-4-sulfonylamino)-quinoline-2-carboxylate
8-hydroxy-4-(toluene-4-sulfonylamino)-quinoline-2-carboxylic acid
Methyl-4-amino-8-hydroxy-quinoline-2-carboxylate
Methyl-4,8-diamino-quinoline-2-carboxylate
4-amino-8-hydroxy-quinoline-2-carboxylic acid
4,8-diamino-quinoline-2-carboxylic acid
Methyl-4-hydroxy-8-benzyloxyl-5-phenyl-quinoline-2-carboxylate
Methyl-4,8-dihydroxy-5-phenyl-quinoline-2-carboxylate
Methyl-4-hydroxy-8-methoxy-5-(4-chloro-phenyl)-quinoline-2-carboxylate
Methyl-4-hydroxy-6-phenyl-8-methoxy-quinoline-2-carboxylate
Methyl-4-hydroxy-6-(4-methoxy-phenyl)-8-methoxy-quinoline-2-carboxylate
Methyl-4,8-dihydroxy-6-(4-methoxy-phenyl)-quinoline-2-carboxylate
Methyl-4-hydroxy-6-(3-methyl-phenyl)-8-benzyloxy-quinoline-2-carboxylate
Methyl-4,8-dihydroxy-6-(3-methyl-phenyl)-quinoline-2-carboxylate
Methyl-4-hydroxy-6-(4-chloro-phenyl)-8-benzyloxy-quinoline-2-carboxylate
Methyl-4,8-dihydroxy-6-(4-chloro-phenyl)-quinoline-2-carboxylate
Methyl-8-benzyloxy-6-(3,4-dichloro-phenyl)-4-hydroxy-quinoline-2-carboxylate
Methyl-4,8-dihydroxy-6-(3,4-dichloro-phenyl)-quinoline-2-carboxylate
Methyl-8-benzyloxy-4-hydroxy-6-(pyridin-3-yl)-quinoline-2-carboxylate
Methyl-4,8-dihydroxy-6-(pyridin-3-yl)-quinoline-2-carboxylate
Methyl-4-hydroxy-8-methoxy-6-propenyl-quinoline-2-carboxylate
Methyl-4-hydroxy-8-methoxy-6-propyl-quinoline-2-carboxylate
Methyl-4-hydroxy-8-phenyl-quinoline-2-carboxylate
Methyl-8-Methoxy-5-phenyl-quinoline-2-carboxylate
4,8-dihydroxy-5-phenyl-quinoline-2-carboxylic acid
4,8-dihydroxy-5-(4-chloro-phenyl)-quinoline-2-carboxylic acid
4,8-dihydroxy-6-phenyl-quinoline-2 carboxylic acid
4,8-dihydroxy-6-(4-methoxy-phenyl)-quinoline-2-carboxylic acid
4,8-dihydroxy-6-(3-methyl-phenyl)-quinoline-2-carboxylic acid,
4,8-dihydroxy-6-(4-chlorophenyl)-quinoline-2-carboxylic acid
4,8-dihydroxy-6-(3,4-dichlorophenyl)-quinoline-2-carboxylic acid
4,8-dihydroxy-6-(pyridin-3-yl)-quinoline-2-carboxylic acid
4,8-dihydroxy-6-propyl-quinoline-2-carboxylic acid
4-hydroxy-8-phenyl-quinoline-2-carboxylic acid
8-hydroxy-5-phenyl-quinoline-2-carboxylic acid
Methyl-4-hydroxy-8-methoxy-6-phenylethynyl-quinoline-2-carboxylate
Methyl-4-hydroxy-8-methoxy-6-(hept-1-ynyl)-quinoline-2-carboxylate
Methyl-8-benzyloxy-6-(3-tert-butoxycarbonylamino-prop-1-ynyl)-4-hydroxy-quinoline-2-carboxylate
Methyl-8-benzyloxy-6-(3-benzyloxy-prop-1-ynyl)-6-hydroxy-quinoline-2-carboxylate
Methyl-4-hydroxy-8-methoxy-6-phenethyl-quinoline-2-carboxylate
Methyl-4-hydroxy-8-methoxy-6-heptyl-quinoline-2-carboxylate
Methyl-4,8-dihydroxy-6-(3-tert-butoxycarbonylamino-propyl)-quinoline-2-carboxylate
Methyl-4,8-dihydroxy-6-(3-hydroxy-propyl)-quinoline-2-carboxylate
4,8-dihydroxy-6-phenethyl-quinoline carboxylic acid
4,8-dihydroxy-6-heptyl-quinoline-2-carboxylic acid
4,8-dihydroxy-6-(3-amino-propyl)-quinoline-2-carboxylic acid
4,8-dihydroxy-6-(3-hydroxy-propyl)-quinoline-2-carboxylic acid
Methyl-8-benzyloxy-6-benzyl-4-hydroxy-quinoline-2-carboxylate
Methyl-4,8 dDihydroxy-6-benzyl-quinoline-2-carboxylate
4,8-dihydroxy-6-benzyl-quinoline-2-carboxylic acid
Methyl-6-(benzylamino-methyl)-4-hydroxy-8-methoxy-quinoline-2 carboxylate
4,8-dihydroxy-6-(benzylamino-methyl)-quinoline-2-carboxylic acid
8-acetylamino-4-hydroxy-quinoline-2-carboxylic acid
8-pivaloylamino-4-hydroxy-quinoline-2-carboxylic acid
8-benzoylamino-4-hydroxy-quinoline-2-carboxylic acid
Methyl-8-benzylamino-4-hydroxy-quinoline-2-carboxylate
8-benzylamino-4-hydroxy-quinoline-2-carboxylic acid
8-benzyloxy-4-hydroxy-quinoline-2-carboxylic acid hydroxyamide
4,8-dihydroxy-quinoline-2-caboxylic acid hydroxyamide
8-methoxy-2-(2H-tetrazol-1-yl)-quinoline
8-hydroxy-2-(2H-tetrazol-1-yl)-quinoline
8-benzyloxy-quinoxazoline-2-carbaldehyde
(8-benzyloxy-quinoxazolin-2-yl)-methanol
(8-hydroxy-quinoxazolin-2-yl)-methanol
Methyl-8-methoxy-3-methylaminomethyl-quinoline-2-carboxylate
8-hydroxy-3-methylaminomethyl-quinoline-2-carboxylic acid
Methyl 3-benzyl-8-methoxy-quinoline-2-carboxylate
3-benzyl-8-methoxy-quinoline-2-carboxylic acid
Methyl-8-benzyloxy-5,7-dichloro-4-hydroxyquinoline-2-carboxylate
Methyl-8-benzyloxy-7-bromo-4-hydroxyquinoline-2 carboxylate
Methyl-6-bromo-8-cyano-4-hydroxy-quinoline-2-carboxylate
Ethyl-8-hydroxy-3-oxo-3,4-dihydroquinoxaline-2-carboxylate
Ethyl[8-(hydroxy)-3-oxo-3,4-dihydroquinoxaline-2(1H)-ylilene]acetate
Methyl-5,7-dichloro-4,8-dihydroxyquinoline-2-carboxylate
Methyl-3-bromo-4,8-dihydroxy-quinoline-2-carboxylate
Methyl-3,7-dDibromo-4,8-dihydroxy-quinoline-2-carboxylate
8-bromo-4-hydroxy-6-isopropyl-quinoline-2-carboxylic acid
8-benzyloxy-6-bromo-4-hydroxy-quinoline-2-caboxylic acid
8-benzyloxy-7-bromo-4-hydroxyquinoline-2-carboxylic acid
8-benzyloxy-3-bromo-4-hydroxy-quinoline-2-carboxylic acid
[8-(hydroxy-3-oxo-3,4-dihydroquinoline-2(1H)-yl]acetic acid
6-bromo-8-cyan-4-hydroxy-quinoline-2-carboxylic acid
8-cyano-4-hydroxy-6-phenyl-quinoline-2-carboxylic acid
8-cyano-4-hydroxy-6-phenenyl-quinoline-2-carboxylic acid
3-bromo-8-cyano-4-hydroxy-quinoline-2-carboxylic acid
8-cyano-4-hydroxy-3-phenylethynyl-quinoline-2-carboxylic acid
8-cyano-6-ethyl-4-hydroxy-quinoline-2-carboxylic acid
Benzyl-4-benzyloxy-8-bromo-quinoline-2-carboxylate
Benzyl-4,8-dibenzyloxy-6-bromo-quinoline-2-carboxylate
Benzyl-4,8-dibenzyloxy-7-bromo-quinoline-2-carboxylate
Benzyl-3-bromo-4,8-dibenzyloxy-quinoline-2-carboxylate
Methyl-6-bromo-8-cyano-4-benzyloxy-quinoline-2-carboxylate
Benzyl-3-bromo-4-benzyloxy-8-cyano-quinoline-2-carboxylate
Methyl-8-benzyloxy-6-(furo-2-yl)-4-hydroxyquinoline-2-carboxylate
Benzyl-4,8-dibenzyloxy-6-(2-chlorophenyl)-quinoline-2-carboxylate
Benzyl-4,8-dibenzyloxy-6-(3-chlorophenyl)-quinoline-2-carboxylate
Benzyl-4,8-dibenzyloxy-7-phenyl-quinoline-2-carboxylate
Benzyl-4,8-dibenzyloxy-6-(2-methoxyphenyl)-quinoline-2-carboxylate
Benzyl-4,8-dibenzyloxy-3-phenyl-quinoline-2-carboxylate
Methyl-8-cyano-4-hydroxy-6-phenyl-quinoline-2-carboxylate
Benzyl-4-benzyloxy-8-(hex-1-ynyl)-quinoline-2-carboxylate
Benzyl-4,8-dibenzyloxy-6-(3-benzyloxy-prop-1-ynyl)-quinoline-2-carboxylate
Benzyl-4,8-dibenzyloxy-7-(3-benzyloxy-prop-1-ynyl)-quinoline-2-carboxylate
Benzyl-4,8-Dibenzyloxy-7-phenylethynyl-quinoline-2-carboxylate
Methyl-4-benzyloxy-8-cyano-6-phenylethynyl-quinoline-2-carboxylate
Benzyl-4-benzyloxy-8-cyano-3-phenylethynyl-quinoline-2-carboxylate
Methyl-8-cyano-4-hydroxy-6-[(trimethylsilyl)ethynyl]-quinoline-2-carboxylate
8-hexyl-4-hydroxy-quinoline-2-carboxylic acid
4,8-dihydroxy-6-(3-hydroxy-propyl)-quinoline-2-carboxylic acid
4,8-dihydroxy-7-(3-hydroxy-propyl)-quinoline-2-carboxylic acid
Methyl-8-cyano-4-hydroxy-6-phenethyl-quinoline-2-carboxylate
Methyl-8-cyano-6-ethyl-4-hydroxy-quinoline-2-carboxylate
4,8-dihydroxy-6-(furo-2-yl)-quinoline-2-carboxylic acid
4-hydroxy-8-phenyl-quinoline-2-carboxylic acid
4,8-dihydroxy-6-(2-chlorophenyl)-quinoline-2-carboxylic acid
4,8-dihydroxy-6-(3-chlorophenyl)-quinoline-2-carboxylic acid
4,8-dihydroxy-7-phenyl-quinoline-2-carboxylic acid
4,8-dihydroxy-6-(2-methoxyphenyl)-quinoline-2-carboxylic acid
4,8-dihydroxy-3-phenyl-quinoline-2-carboxylic acid
4,8-dihydroxy-4-piperidin-2-yl-quinoline-2-carboxylic acid hydrochloride
Sodium 8-hexyl-4-hydroxy-quinoline-2-carboxylate
Sodium 4,8-dihydroxy-7-phenyl-quinoline-2-carboxylate
Sodium 4,8-dihydroxy-3-phenyl-quinoline-2-carboxylate
Methyl-8-benzyloxy-3-bromo-4-hydroxy-quinoline-2 carboxylate
Methyl-8-benzyloxy-3,7-dibromo-4-hydroxyquinoline-2-carboxylate
Methyl-3-bromo-8-cyano-4-hydroxyquinoline-2 carboxylate
Methyl-4-hydroxy-8-(2H-tetrazol-5-yl)-quinoline-2-carboxylate
Ethyl-8-(benzyloxy)-3-oxo-3,4-dihydroquinoxaline-2-carboxylate
Ethyl[8-(benzyloxy)-3-oxo-3,4-dihydroquinoxaline-2(1H)-ylilene]acetate
Benzyl-4,8-dibenzyloxy-6-piperidin-2yl-quinoline-2-carboxylate
Methyl-8-cyano-6-ethynyl-4-hydroxy-quinoline-2-carboxylate
Methyl-5-bromo-8-nitro-4-hydroxy-quinoline-2-carboxylate
Methyl-6-benzyloxy-8-nitro-4-hydroxy-quinoline-2-carboxylate
Methyl-6-chloro-8-nitro-4-hydroxy-quinoline-2-carboxylate
Methyl-6-bromo-8-nitro-4-hydroxy-quinoline-2-carboxylate
Methyl-8-cyano-4-hydroxy-quinoline-2-carboxylate
Methyl-8-carboxamide-4-hydroxy-quinoline-2-carboxylate
Methyl 3-phenylethyl-8-amino-4-hydroxy-quinoline-2-carboxylate
3-(3′-N-tert-butoxycarbonyl-propyl)-8-amino-4-hydroxy-quinoline-2-carboxylic acid
3-(3-hydroxypropyl)-8-amino-4-hydroxy-quinoline-2-carboxylic acid
3-ethyl-8-amino-4-hydroxy-quinoline-2-carboxylic acid
Methyl-5-phenyl-8-amino-4-hydroxy-quinoline-2-carboxylate
Methyl-5-phenylethyl-8-amino-4-hydroxy-quinoline-2 carboxylate
Methyl-5-(3′-N-(terbutoxycarbonyl)aminopropyl)-8-amino-4-hydroxy-quinoline-2-carboxylate
Methyl-5-hydroxypropyl-8-amino-4-hydroxy quinoline-2-carboxylate
Methyl-5-(N-piperidinyl)-8-amino-4-hydroxy-quinoline-2-carboxylate
Methyl-5-piperazinyl-8-amino-4-hydroxy-quinoline-2-carboxylate
Methyl-6-hydroxy-8-amino-4-hydroxy-quinoline-2-carboxylate
Methyl-6-phenylethyl-8-amino-4-hydroxy-quinoline-2-carboxylate
Methyl-6-(3′-hydroxypropyl)-8-amino-4-hydroxy-quinoline-2-carboxylate
Methyl-6-(3′-N-(tert-butoxycarbonyl)aminopropyl)-8-amino-4-hydroxy-quinoline-2-carboxylate
Methyl-6-(3′-pyridinyl)ethyl-8-amino-4-hydroxy-quinoline-2-carboxylate
Methyl-6-(5′-cyanopentyl)-8-amino-4-hydroxy-quinoline-2-carboxylate
Methyl-6-cyano-8-amino-4-hydroxy-quinoline-2-carboxylate
6-N-(N-methylpiperazinyl)-8-amino-4-hydroxy-quinoline-2-carboxylic acid hydrochloride
Methyl-6-N-piperidinyl-8-amino-4-hydroxy-quinoline-2-carboxylate
Methyl-6-piperazinyl-8-amino-4-hydroxy-quinoline-2-carboxylate
7-phenyl-8-amino-4-hydroxy-quinoline-2-carboxylic acid
4-(N-methylamino)-4-amino-quinoline-2-carboxylic acid hydrochloride
Methyl-8-dimethylamino-4-hydroxy-quinoline-2-carboxylate
3-(N-morpholinomethyl)-4,8-dihydroxy-quinoline-2-carboxylic acid
3-(N-pyrolidinomethyl)-4,8-dihydroxy-quinoline-2-carboxylic acid
Methyl-4-(N-methylamino)-8-amino-quinoline-2-carboxylate
6-hydroxy-8-amino-4-hydroxy-quinoline-2-carboxylic acid
6-chloro-8-amino-4-hydroxy-quinoline-2-carboxylic acid
6-phenylethyl-8-amino-4-hydroxy-quinoline-2-carboxylic acid
6-(3′-hydroxypropyl)-8-amino-4-hydroxy-quinoline-2-carboxylic acid
6-(3′-aminopropyl)-8-amino-4-hydroxy-quinoline-2-carboxylic acid
8-carboxamide-4-hydroxy-quinoline-2-carboxylic acid
3-bromo-8-amino-4-hydroxy-quinoline-2-carboxylic acid
8-cyano-4-hydroxy-quinoline-2-carboxylic acid
5-phenyl-8-amino-4-hydroxy-quinoline-2-carboxylic acid
6-(5′-cyanopentyl)-8-amino-4-hydroxy-quinoline-2-carboxylic acid
6-cyano-8-amino-4-hydroxy-quinoline-2-carboxylic acid
6-N-(N-methylpiperazinyl)-8-nitro-4-benzyloxy-quinoline-2-carboxylic acid hydrochloride
8-dimethylamino-4-hydroxy-quinoline-2-carboxylic acid
Methyl-5-bromo-8-nitro-4-benzyloxy-quinoline-2-carboxylate
Methyl-6-bromo-8-nitro-4-benzyloxy-quinoline-2-carboxylate
Methyl-7-bromo-4,8-dibenzyloxy-quinoline-2-carboxylate
Methyl-4-(N-methyl-toluene-4-sulfonamino)-8-nitro-quinoline-2-carboxylate
Methyl-8-dimethylamino-4-benzyloxy-quinoline-2-carboxyalte
Benzyl-7-phenyl-8-amino-4-benzyloxy-quinoline-2-carboxylate
Methyl-5-phenyl-8-nitro-4-benzyloxy-quinoline-2-carboxylate
Methyl-3-trimethylsilylethynyl-8-nitro-4-hydroxy-quinoline-2-carboxylate
Methyl-3-phenylethynyl-8-nitro-4-hydroxy-quinoline-2-carboxylate
Benzyl-3-(3′-benzyloxypropyn-1′-yl)-8-nitro-4-hydroxy-quinoline-2-carboxylate
Benzyl 3-(3′-N-(terbutoxycarbonyl)aminoprop-1′-ynyl)-8-nitro-4-hydroxy-quinoline-2 carboxylate
Methyl-5-phenylethynyl-8-nitro-4-benzyloxy-quinoline-2-carboxylate
Methyl-5-(3′-benzyloxypropyn-1′-yl)-8-nitro-4-benzyloxy-quinoline-2-carboxylate
Methyl-5-(3′-N-(terbutoxycarbonyl)aminoprop-1′-ynyl)-8-nitro-4-benzyloxy-quinoline-2-carboxylate
Methyl-6-phenylethynyl-8-nitro-4-benzyloxy-quinoline-2-carboxylate
Methyl-(3′-pyridyl)ethynyl-8-nitro-4-benzyloxy-quinoline-2-carboxylate
Methyl-6-(5′-cyanopent-1′-ynyl)-8-nitro-4-benzyloxy-quinoline-2-carboxylate
Methyl-6-(3′-benzyloxypropyn-1′-yl)-8-nitro-4-benzyloxy-quinoline-2-carboxylate
Methyl-6-(3′-N-(terbutoxycarbonyl)aminoprop-1′-ynyl)-8-nitro-4-benzyloxy-quinoline-2-carboxylate
Methyl-4-methylamino-8-nitro-quinoline-2-carboxylate
Sodium 7-bromo-4,8-dihydroxy-quinoline-2-carboxylate
Methyl-6-(N-(N-methyl-piperazinyl))-8-nitro-4-benzyloxy-quinoline-2-carboxylate
Methyl-6-(N-(N-benzyl-piperazinyl))-8-nitro-4-benzyloxy-quinoline-2-carboxylate
Methyl-6-(N-piperidinyl)-8-nitro-4-benzyloxy-quinoline-2-carboxylate
Methyl-6-(N-diphenylimine)-8-nitro-4-benzyloxy-quinoline-2-carboxylate
Methyl-6-(N-anilino)-8-nitro-4-benzyloxy-quinoline-2-carboxylate
Methyl-5-(N-piperidinyl)-8-nitro-4-benzyloxy-quinoline-2-carboxylate
Methyl-5-(N-(N-benzyl-piperazinyl))-8-nitro-4-benzyloxy-quinoline-2-carboxylate
Methyl-3-bromo-8-amino-4-hydroxy-quinoline-2-carboxylate
Methyl-6-bromo-8-amino-4-hydroxy-quinoline-2-carboxylate
Methyl-6-chloro-8-amino-4-hydroxy-quinoline-2-carboxylate
Benzyl-8-amino-4-benzyloxy-quinoline-2-carboxylate
Benzyl-7-iodo-8-amino-4-benzyloxy-quinoline-2-carboxylate
Methyl-6-cyano-8-nitro-4-hydroxy-quinoline-2-carboxylate
Methyl-3-bromo-4-hydroxy-8-nitro-quinoline-2-carboxylate
Methyl-6-amino-8-nitro-4-benzyloxy-quinoline-2-carboxylate
3-ethynyl-8-nitro-4-hydroxy-quinoline-2-carboxylic acid
Benzyl-8-nitro-4-benzyloxy-quinoline-2-carboxylate
3-(N-morpholinomethyl)-8-benzyloxy-4-hydroxy-quinoline-2-carboxylic acid
3-(N-pyrolidinomethyl)-8-benzyloxy-4-hydroxy-quinoline-2-carboxylic acid
Methyl-5-piperazinyl-8-amino-4-hydroxy-quinoline-2-carboxylate
3-phenylethyl-8-amino-4-hydroxy-quinoline-2-carboxylic acid
3-(3′-aminopropyl)-8-amino-4-hydroxy-quinoline-2-carboxylic acid hydrochloride
5-phenylethyl-8-amino-4-hydroxy-quinoline-2-carboxylic acid
Methyl-5-(3′-aminopropyl)-8-amino-4-hydroxy-quinoline-2-carboxylate hydrochloride
5-hydroxypropyl-8-amino-4-hydroxy quinoline-2-carboxylic acid
5-(N-piperidinyl)-8-amino-4-hydroxy-quinoline-2-carboxylic acid hydrochloride
5-piperazinyl-8-amino-4-hydroxy-quinoline-2-carboxylic acid hydrochloride
6-(3′-pyridinyl)ethyl-8-amino-4-hydroxy-quinoline-2-carboxylic acid hydrochloride
6-N-piperidinyl-8-amino-4-hydroxy-quinoline-2-carboxylic acid hydrochloride
6-piperazinyl-8-amino-4-hydroxy-quinoline-2-carboxylic acid hydrochloride
6-anilino-8-amino-4-hydroxy-quinoline-2-carboxylic acid hydrochloride
6,8-diamino-4-hydroxy-quinoline-2-carboxylic acid hydrochloride
Methyl-6-anilino-8-amino-4-hydroxy-quinoline-2-carboxylate
Methyl-6,8-diamino-4-hydroxy-quinoline-2-carboxylate
Methyl-4-hydroxy-8-nitro-6-phenyl-quinoline-2-carboxylate
Methyl-8-nitro-4-hydroxy-6-phenyl-quinoline-2-carboxylate
Methyl-8-hydroxy-4-(piperazin-1-yl)-quinoline-2-carboxylate
Methyl-8-amino-4-phenyl-quinoline-2-carboxylate
Methyl-8-amino-4-(hex-1-yl)-quinoline-2-carboxylate
Methyl-8-amino-4-(2-phenyleth-1-yl)-quinoline-2-carboxylate
Methyl-8-amino-4-(3-tert-butoxycarbonyl-prop-1-yl)-quinoline-2-carboxylate
Methyl-8-amino-4-(3-hydroxy-prop-1-yl)-quinoline-2-carboxylate
Methyl-4-(3-acetyl-aminoprop-1-ynyl)-8-amino-quinoline-2-carboxylate
Methyl-8-hydroxy-4-(morpholin-1-yl)-quinoline-2-carboxylate
Methyl-8-benzyloxy-4-(piperidin-1-yl)-quinoline-2 carboxylate
Methyl-8-amino-4-(piperidin-1-yl)-quinolin-2-carboxylate
Methyl-4-hydroxy-8-(piperazin-1-yl)-quinoline-2-carboxylate
Methyl-8-hydroxy-8-(4-methyl-piperazin-1-yl)-quinoline-2-carboxylate
Methyl-4-(3-benzoyl-aminoprop-1-yl)-8-hydroxyquinoline-2-carboxylate
5-(4-chlorophenyl)-8-hydroxy-quinoline-2-carboxylic acid
8-amino-4-(hex-1-yl)-quinoline-2-carboxylic acid hydrochloride
8-amino-4-(2-phenyleth-1-yl)-quinoline-2-carboxylic acid hydrochloride
8-amino-4-(3-tert-butoxycarbonylaminoprop-1-yl)-quinoline-2-carboxylic acid hydrochloride
8-amino-4-(3-hydroxy-prop-1-yl)-quinoline-2-carboxylic acid hydrochloride
4-(3-acetylaminoprop-1-ynyl)-8-amino-quinoline-2-carboxylic acid hydrochloride
8-hydroxy-4-(morpholin-1-yl)-quinoline-2-carboxylic acid hydrochloride
8-hydroxy-4-(piperidin-1-yl)-quinoline-2-carboxylic acid hydrochloride
8-amino-4-(piperidin-1-yl)-quinoline-2-carboxylic acid hydrochloride
4-hydroxy-8-(piperazin-1-yl)-quinoline-2-carboxylic acid hydrochloride
8-hydroxy-4-(methyl-piperazin-1-yl)-quinoline-2-carboxylic acid hydrochloride
4-hydroxy-8-phenylethyl-quinoline-2-carboxylic acid
4-(3-(benzoylamino)prop-1-yl)-8-hydroxyquinoline-2-carboxylic acid
8-amino-4-hydroxy-6-phenyl-quinoline-2-carboxylic acid
Methyl-8-nitro-4-oxytrimethanelsulfonyl-quinoline-2-carboxylate
Methyl-5-(4-chlorophenyl)-8-methoxy-quinoline-2-carboxylate
Benzyl-4,8-dibenzyloxy-6-(3,5-dichlorophenyl)-quinoline-2-carboxylate
Benzyl-4,8-dibenzyloxy-6-(4-fluorophenyl)-quinoline-2-carboxylate
Methyl-8-nitro-4-phenyl-quinoline-2-carboxylate
Benzyl-8-benzyloxy-5-phenylethynyl-quinoline-2-carboxylate
Methyl-8-benzyloxy-4-(hex-1-ynyl)-quinoline-2-carboxylate
Methyl-8-benzyloxy-4-(5-benzyloxy-pent-1-ynyl)-quinoline-2-carboxylate
Methyl-8-benzyloxy-7-(3-tert-butoxycarbonylaminoprop-1-ynyl)-4-hydroxy-quinoline-2-carboxylate
Benzyl-4,8-dibenzyloxy-7-(hex-1-ynyl)-quinoline-2-carboxylate
Methyl-8-amino-4-(hex-1-ynyl)-quinoline-2-carboxylate
Methyl-8-amino-4-phenylethynyl-quinoline-2-carboxylate
Methyl-8-nitro-4-(3-tert-butoxycarbonylamino-prop-1-ynyl)-quinoline-2-carboxylate
Methyl-4-(3-benzyloxy-prop-1-ynyl)-8-nitro-quinoline-2-carboxylate
Methyl-4-(3-acetyl-aminoprop-1-ynyl)-8-nitro-quinoline-2-carboxylate
Methyl-4-benzyloxy-8-phenylethynyl-quinoline-2-carboxylate
8-hydroxy-5-phenylethyl-quinoline-2-carboxylic acid
Methyl-8-benzyloxy-4-(hex-1-yl)-quinoline-2-carboxylate
Methyl-8-benzyloxy-4-(5-hydroxy-pent-1-yl)-quinoline-2-carboxylate
Methyl-8-benzyloxy-4-(3-tert-butoxycarbonylaminoprop-1-yl)-quinoline-2-carboxylate
Methyl-4,8-dihydroxy-7-(3-tert-butoxycarbonylaminoprop-1-yl)-quinoline-2-carboxylate
4,8-dihydroxy-7-(hex-1-yl)-quinoline-2 carboxylic acid
Methyl-4-hydroxy-8-phenylethyl-quinoline-2-carboxylate
8-hydroxy-5-phenylethyl-quinoline-2-carboxylic acid
Methyl-8-benzyloxy-4-(hex-1-yl)-quinoline-2-carboxylate
Methyl-8-benzyloxy-4-(5-hydroxy-pent-1-yl)-quinoline-2-carboxylate
Methyl-8-benzyloxy-4-(3-tert-butoxycarbonylaminoprop-1-yl]-quinoline-2-carboxylate
Methyl-4,8-dihydroxy-7-(3-tert-butoxycarbonylaminoprop-1-yl)-quinoline-2-carboxylate
4,8-dihydroxy-7-(hex-1-yl)-quinoline-2-carboxylic acid
Methyl-4-hydro-8-phenylethyl-quinoline-2-carboxylate
Sodium 4-(hex-1-yl)-8-hydroxy-quinoline-2-carboxylate
8-amino-4-hydroxy-6-phenyl-quinoline-2-carboxylic acid
Methyl-8-benzyloxy-4-(4-benzyl-piperazin-1-yl)-quinoline-2-carboxylate
Methyl-8-benzyloxy-4-(morpholin-1-yl)-quinoline-2-carboxylate
Methyl-8-benzyloxy-4-(piperidin-1-yl)-quinoline-2-carboxylate
Methyl-8-nitro-4-(piperidin-1-yl)-quinoline-2-carboxylate
Benzyl-4-benzyloxy-8-(piperidin-1-yl)-quinoline-2-carboxylate
Benzyl-4-benzyloxy-8-[benzyl(methyl)amino]-quinoline-2-carboxylate
Methyl-4-benzyloxy-8-(morpholin-1-yl)-quinoline-2-carboxylate
Methyl-4-benzyloxy-8-(4-benzyl-piperazin-1-yl)-quinoline-2-carboxylate
Methyl-4-benzyloxy-8-[phenyl(methyl)amino]-quinoline-2-carboxylate
Methyl-4-benzyloxy-8-(4-methyl-piperazin-1-yl)-quinoline-2-carboxylate
Methyl-4-benzyloxy-8-(pyridin-2-ylamino)-quinoline-2-carboxylate
Methyl-8-benzyloxy-4-(4-methyl-piperazin-1-yl)-quinoline-2-carboxylate
Methyl-8-benzyloxy-4-(3-aminoprop-1-yl)-quinoline-2-carboxylate
Methyl-8-benzyloxy-4-(3-benzoyl-aminoprop 1-yl)-quinoline-2-carboxylate
4-(hex-1-yl)-8-hydroxy-quinoline-2-carboxylic acid
8-hydroxy-4-(5-hydroxy-pent-1-yl)-quinoline-2-carboxylic acid
6-(3,5-dichlorophenyl)-4,8-dihydroxy-quinoline-2-carboxylic acid
6-(4-fluorophenyl)-4,8-dihydroxy-quinoline-2-carboxylic acid
4-hydroxy-8-(piperidin-1-yl)-quinoline-2-carboxylic acid hydrochloride
8-(4-benzyl-piperazin-1-yl)-4-hydroxy-quinoline-2-carboxylic acid hydrochloride
8-[phenyl(methyl)amino]-4-hydroxy-quinoline-2-carboxylic acid hydrochloride
8-(4-methyl-piperazin-1-yl)-4-hydroxy-quinoline-2-carboxylic acid hydrochloride
and their pharmaceutically acceptable salts.

62. A method for preparing a compound represented by formula (I) according to claim 48 for which Z is a nitrogen atom and E is a COOR₁ group wherein one reacts a derivative of formula: 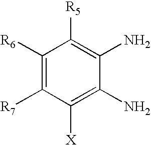
with a derivative R₁O₂C—CO—CO—R₃, in said formulas, R₁, R₃, R₅, R₆, R₇ and X are defined as in claim 54, isolates the product and optionally converts it to a pharmaceutically acceptable salt.

63. A method for preparing a compound represented by formula (I) according to claim 48 for which Z is a nitrogen atom, R₃ is hydroxyl and E is a CH₂COOR₁ group wherein one reacts a derivative of formula: 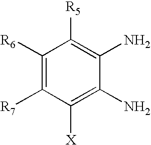
with a derivative R₁O₂C—CO—CH₂COOEt, in said formulas, R₁, R₅, R₆, R₇ and X are defined as in claim 54, isolates the product and optionally converts it to a pharmaceutically acceptable salt.

64. A method for preparing a compound represented by formula (I) according to claim 48 for which Z is a CR₄ group, R₄ is hydrogen and E is COOH wherein one reacts a derivative of formula: 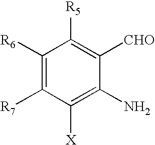
with a derivative R₃—CH₂—CO—COOH, in said formulas, R₃, R₅, R₆, R₇ and X are defined as in claim 54, isolates the product and optionally converts it to a pharmaceutically acceptable salt.

65. A method for preparing a compound represented by formula (I) according to claim 48 for which Z is a C—R₄ group, R₄ is OR₈, R₈ is hydrogen, R₃ is hydrogen E is COOR₁ and R₁ is methyl wherein one cyclizes a derivative of formula: 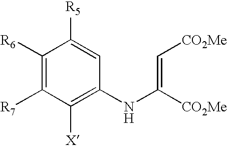
in which R₅, R₆ and R₇ are defined as in claim 48, X′ has the same definitions as X and Me represents a methyl group, isolates the product and optionally converts it to a pharmaceutically acceptable salt.

66. A method for preparing a compound represented by formula (I) according to claim 48 for which Z is a C—R₄ groups R₄ is OR₈, R₈ is hydrogen R₃ is an alkyl group, an aryl group, an arylalkyl group or a heteroaryl group, E is COOR₁ and R₁ is methyl wherein one reacts a derivative of formula: 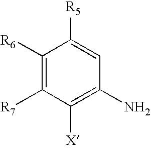
with a derivative MeO₂C—CO—C(R₃)CO₂Me, in said formulas R₅, R₆ and R₇ are defined as in claim 48, X′ has the same definitions as X and Me represents a methyl group, isolates the product and optionally converts it to a pharmaceutically acceptable salt.

67. A method for preparing a compound represented by formula (I) according to claim 48 for which Z is a C—R₄ group, R₄ is OR₈, R₈ is hydrogen, X is OR₈, R₈ is hydrogen, E is COOR₁ and R₁ is methyl wherein one hydrolyzes a corresponding compound represented by formula (I) for which X is OR₈ in which R₈ is methyl or benzyl, isolates the product and optionally converts it to a pharmaceutically acceptable salt.

68. A method for preparing a compound represented by formula (I) according to claim 48 for which Z is a C—R₄ group, R₄ is a halogen atom and E is a COOR₁ group wherein one halogenates a corresponding compound represented by formula (I) for which Z is a C—R₄ group and R₄ is a hydroxyl group, isolates the product and optionally converts it to a pharmaceutically acceptable salt.

69. A method for preparing a compound represented by formula (I) according to claim 48 for which Z is a CR₄ group, R₄ is a hydrogen atom, R₃ is a hydrogen atom and E is a COOR₁ group and the other substituents are not halogen wherein one hydrogenates a corresponding compound represented by formula (I) for which Z is a C—R₄ group, R₄ is a halogen atom, isolates the product and optionally converts it to a pharmaceutically acceptable salt.

70. A method for preparing a compound represented by formula (I) according to claim 48 for which Z is a C—R₄ group R₄ is an aryl, heteroaryl or arylalkyl group and E is COOR₁ and/or one of the substituents R₃, R₅, R₆, R₇ or X is an aryl, heteroaryl or arylalkyl group wherein one reacts a corresponding compound represented by formula (I) for which Z is a a C—R₄ group, R₄ is a halogen atom and/or one of the substituents R₃, R₅, R₆, R₇ or X is a halogen atom with a derivative of formula R_{4′}B(OH)₂ for which R_{4′} is an aryl, heteroaryl or arylalkyl group, isolates the product and optionally converts it to a pharmaceutically acceptable salt.

71. A method for preparing a compound represented by formula (I) according to claim 48 for which Z is a C—R₄ group, R₄ is a NR₉R_{9′} group and E is COOR₁ and/or one of the substituents R₃, R₅, R₆, R₇ or X is a NR₉R_{9′} group wherein one reacts a corresponding compound represented by formula (I) for which Z is a C—R₄ group, R₄ is a halogen atom and/or one of the substituents R₃, R₅, R₆, R₇ or X is a halogen atom with a derivative of formula NR₉R_{9′} for which R₉ is an alkyl, aryl, heteroaryl or arylalkyl group and R_{9′} is a hydrogen, alkyl, aryl heteroaryl or arylalkyl group or NR₉R_{9′} represents a cycloheteroalkyl group of the type: 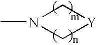
n=2, 3
m=2, 3
Y=CH₂, O, S, SO₂, NR₁₁
R₁₁ represents (i) a hydrogen atom, (ii) a (C₁-C₁₂) alkyl group, (iii) a (C₆-C₁₈) aryl group, (iv) a (C₆-C₁₈)aryl(C₁-C₁₂)alkyl group, (v) a (C₁-C₁₇) heteroaryl group, (vi) a (C₁-C₁₇)heteroaryl(C₁-C₁₂)alkyl group or (vii) a COR₁₀ group;
one isolates the product and optionally converts it to a pharmaceutically acceptable salt.

72. A method for preparing a compound represented by formula (I) according to claim 48 for which Z is a C—R₄ group R₄ is a cyano group and E is COOR₁ and/or one of the substituents R₃, R₅, R₆, R₇ or X is a cyano group wherein one reacts a corresponding compound represented by formula (I) for which Z is a C—R₄ group, R₄ is a halogen atom and/or one of the substituents R₃, R₅, R₆, R₇ or X is a halogen atom with a derivative of formula Zn(CN)₂, isolates the product and optionally converts it to a pharmaceutically acceptable salt.

73. A method for preparing a compound represented by formula (I) according to claim 48 for which Z is a CR₄ group R₄ est an alcyn-1-yl or (C₂-C₁₂)alkyl group and E is COOR₁ wherein one reacts a derivative of formula: 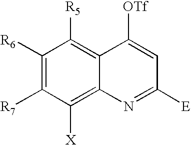
with a derivative ≡—R″ in said formulas R₃, R₅, R₆, R₇, X are defined as in claim 7, E is COOR₁, OTf represents a triflate group, R″ represents a hydrogen atom or an alkyl group, possibly followed by a reduction, isolates the product and optionally converts it to a pharmaceutically acceptable salt.

74. A method for preparing a compound represented by formula (I) according to claim 48 for which Z is a CR₄ group, R₄ is a NR₉R_{9′} group, R₉ is hydrogen or arylsulfonyl and R_{9′} represents hydrogen, and E is COOR₁ wherein one reacts a corresponding derivative of formula (I) for which Z is a CR₄ group, R₄ is a OR₈ group and R₈ is hydrogen with an arylsulfonylisocyanate possibly followed by deprotection, isolates the product and optionally converts it to a pharmaceutically acceptable salt.

75. A method for preparing a compound represented by formula (I) according to claim 48 for which E is a CHO group wherein one oxidizes a derivative of formula 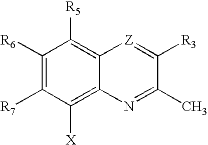
in which Z, R₃, R₅, R₆, R₇ and X are defined as in claim 7, isolates the product and optionally converts it to a pharmaceutically acceptable salt.

76. A method for preparing a compound represented by formula (I) according to claim 48 for which E is a —CH₂OH group wherein one reduces a corresponding compound represented by formula (I) for which E is CHO, isolates the product and optionally converts it to a pharmaceutically acceptable salt.

77. A method for preparing a compound represented by formula (I) according to claim 48 for which E is a COOH group wherein one oxidizes a corresponding compound represented by formula (I) for which E is a CHO group, isolates the product and optionally converts it to a pharmaceutically acceptable salt.

78. A method for preparing a compound represented by formula (I) according to claim 48 for which E is a COOH group wherein one hydrolyzes a corresponding compound represented by formula (I) for which E is a COOR₁ group, isolates the product and optionally converts it to a pharmaceutically acceptable salt.

79. A method for preparing a compound represented by formula (I) according to claim 48 for which E est a COOR₁ group, R₁ is alkyl wherein one esterifies a corresponding compound represented by formula (I) for which E is a COOH group, isolates the product and optionally converts it to a pharmaceutically acceptable salt.

80. A method for preparing a compound represented by formula (I) according to claim 48 for which E is a COOR₁ group R₁ is arylalkyl wherein one reacts a corresponding compound represented by formula (I) for which E is a COOH group with an arylalkyl halogenide, isolates the product and optionally converts it to a pharmaceutically acceptable salt.

81. A method for preparing a compound represented by formula (I) according to claim 48 for which E represents a CO—NHR₂ group wherein one reacts a corresponding compound represented by formula (I) for which E is a COOH or COOR₁ group, and an amine H₂NR₂ in which R₂ is defined as in claim 48, isolates the product and optionally converts it to a pharmaceutically acceptable salt.

82. A method for preparing a compound represented by formula (I) according to claim 48 for which E represents a tetrazolyl group wherein one reacts NaN₃ with a derivative of formula: 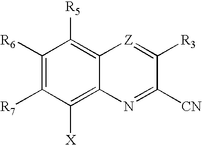
in which R₃, R₅, R₆, R₇ and X are defined as in claim 48 isolates the product and optionally converts it to a pharmaceutically acceptable salt.

83. A method for preparing a compound represented by formula (I) according to claim 48 for which Z is a CR₄ group, R₄ is a hydrogen atom and R₃ is an arylmethyl group wherein one rear a derivative of formula: 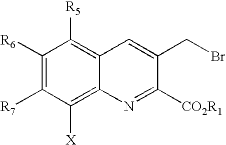
with a derivative R′″B(OH)₂, in said formulas R₁, R₅, R₆, R₇ and X are defined as in claim 48, R′″ is an aryl group, isolates the product and optionally converts it to a pharmaceutically acceptable salt.

84. A method for preparing a compound represented by formula (I) according to claim 48 for which Z is a CR₄ group, R₄ is a hydrogen atom and R₃ is a methyl group substituted by NR₉R_{9′} wherein one reacts a derivative of formula: 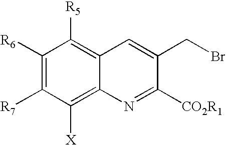
with an amine HNR₉R_{9′}, in said formulas R₁, R₅, R₆, R₇, R₉, R_{9′} and X are defined as in claim 48, isolates the product and optionally converts it to a pharmaceutically acceptable salt.

85. A method for preparing a compound represented by formula (I) according to claim 48 for which one of the substituents R₃, R₅, R₆, R₇ or X is an alcyn-1-yl or (C₂-C₁₂) alkyl group wherein one reacts a corresponding compound represented by formula (I) for which one of the substituents R₃, R₅, R₆, R₇ or X is a halogen atom, with a derivative of formula:
≡—R″
in which R″ represents a hydrogen atom or an alkyl group, possibly followed by a reduction, isolates the product and optionally converts it to a pharmaceutically acceptable salt.

86. A method for preparing a compound represented by formula (I) according to claim 48 for which one of the substituents R₃, R₅, R₆, R₇ or X is an alkyl or arylalkyl group wherein one reacts a corresponding compound represented by formula (I) for which one of the substituents R₃, R₅, R₆, R₇ or X is a halogen atom, with a derivative R′″ ZnX″ for which R′″ is an alkyl or arylalkyl group and X″ is a bromine or iodine atom, isolates the product and optionally converts it to a pharmaceutically acceptable salt.

87. A method for preparing a compound represented by formula (I) according to claim 48 for which Z is C—R₄, R₄, R₃, R₆, R₇ are hydrogen, X is OR₈, R₈ is benzyl, E is COOR₁, R₁ is benzyl and R₅ is a bromine atom wherein one brominates the corresponding compound represented by formula (I) for which Z is CR₄, R₄, R₃, R₅, R₆, R₇ are hydrogen, X is OR₈, R₈ is benzyl, E is COOR₁, R₁ is benzyl, isolates the product and optionally converts it to a pharmaceutically acceptable salt.

88. A method for preparing a compound represented by formula (I) according to claim 48 for which one of the substituents R₃, R₅, R₆, R₇ or X is a (C₁)alkyl group substituted by NR₉R_{9′}, R₉ is alkyl, aryl or arylalkyl and R_{9′} represents hydrogen, alkyl, aryl or arylalkyl, wherein one reacts a corresponding compound for which one of the substituents R₃, R₅, R₆, R₇ or X is a CHO group with an amine of formula HNR₉R_{9′}, in which R₉ is alkyl, aryl or arylalkyl and R_{9′} represents hydrogen, reduces it, isolates the product and optionally converts it to a pharmaceutically acceptable salt.

89. A method for preparing a compound represented by formula (I) according to claim 48 for which one of the substituents R₅, R₆, R₇ or X is a NR₉R_{9′} group for which R₉ is COR₁₀ wherein one reacts a corresponding compound represented by formula (I) for which one of the substituents R₅, R₆, R₇ or X is a NR₉R_{9′} group in which R₉ is hydrogen and R_{9′} represents hydrogen with a derivative R₁₀COCl for which R₁₀ is defined as in claim 54, isolates the product and optionally converts it to a pharmaceutically acceptable salt.

90. A method for preparing a compound represented by formula (I) according to claim 48 for which one of the substuents R₅, R₆, R₇ or X is a NR₉R_{9′} group and R₉ is an alkyl group possibly substituted by aryl or arylalkyl and R_{9′} represents hydrogen, wherein one reacts a corresponding compound represented by formula (I) for which one of the substituents R₅, R₆, R₇ or X is a NR₉R_{9′} group and R₉ is hydrogen with an aldehyde R_{9″}CHO, in which R_{9″} is a hydrogen atom or an alkyl group possibly substituted by aryl or arylalkyl, isolates the product and optionally converts it to a pharmaceutically acceptable salt.

91. A method for preparing a compound represented by formula (I) according to claim 48 containing a OR₈ group in which R₈ is alkyl or arylalkyl wherein one reacts a corresponding compound represented by formula (I) containing a OR₈ group in which R₈ is hydrogen with a derivative R₈Br for which R₈ is an alkyl or arylalkyl group, isolates the product and optionally converts it to a pharmaceutically acceptable salt.

92. A method for preparing a compound represented by formula (I) according to claim 48 containing a NR₉R_{9′} group in which R₉ is hydrogen and R_{9′} represents hydrogen, wherein one reduces a corresponding compound containing a nitro group, isolates the product and optionally converts it to a pharmaceutically acceptable salt.

93. A method for preparing a compound represented by formula (I) according to claim 48 for which one of the substituents R₅, R₆, R₇ or X is an iodine atom wherein on reacts a corresponding compound represented by formula (I) for which one of the substituents R₅, R₆, R₇ or X is a NR₉R_{9′} group or R₉ is hydrogen and R_{9′} represents hydrogen, with KI, isolates the product and optionally converts it to a pharmaceutically acceptable salt.

94. A method for preparing a compound represented by formula (I) according to claim 48 for which Z is C—R₄ in which R₄ is hydroxyl, R₃ is halogen, E is COOR₁, R₁ is alkyl or benzyl wherein one brominates the corresponding compound represented by formula (I) for which Z is CR₄ in which R₄ is hydroxyl, R₃ is hydrogen, E is COOR₁, R₁ is alkyl or benzyl, isolates the product and optionally converts it to a pharmaceutically acceptable salt.
